(19) United States
(12) Patent Application Publication
(10) Pub. No.: US 2024/0417432 A1
(43) Pub. Date: Dec. 19, 2024

(54) Antigenic OspA Polypeptides

(71) Applicant: SANOFI, Paris (FR)

(72) Inventors: Gary J. Nabel, Bridgewater, NJ (US); Chih-Jen Wei, Bridgewater, NJ (US); Heather Kamp, Bridgewater, NJ (US); Ronnie Wei, Bridgewater, NJ (US); Kurt Swanson, Bridgewater, NJ (US)

(73) Assignee: SANOFI, Paris (FR)

(21) Appl. No.: 18/640,574

(22) Filed: Apr. 19, 2024

Related U.S. Application Data
(62) Division of application No. 17/061,155, filed on Oct. 1, 2020, now Pat. No. 11,993,636.
Application No. 17/061,155 is a continuation of application No. PCT/US2019/025367, filed on Apr. 2, 2019.
(60) Provisional application No. 62/652,210, filed on Apr. 3, 2018.

(51) Int. Cl. C07K 14/20; C12N 15/10

(52) U.S. Cl. CPC C07K 14/20; C12N 15/102

(57) ABSTRACT

This disclosure relates to antigenic OspA polypeptides and their use in eliciting antibodies against OspA. Also disclosed are antigenic polypeptides comprising an OspA polypeptide and a ferritin protein.

This application is a divisional of U.S. patent application Ser. No. 17/061,155,filed Oct. 1, 2020, which is a continuation of International Application No. PCT/US2019/025367, filed Apr. 2, 2019, which claims the benefit of priority of U.S. Provisional Patent Application No. 62/652,210, filed Apr. 3, 2018, the entire contents of which are incorporated herein by reference.

The instant application contains a Sequence Listing which has been submitted electronically in XML format and is hereby incorporated by reference in its entirety. Said XML copy, created on Apr. 18, 2024, is named 01121-0033-01US-ST26.xml and is 174,961bytes in size.

Even with many successes in the field of vaccinology, new breakthroughs are needed to protect humans against many life-threatening infectious diseases. Many currently licensed vaccines rely on decade-old technologies to produce live-attenuated or inactivated killed pathogens, which carry inherent safety concerns and in many cases, stimulate only short-lived, weak immune responses that require the administration of multiple doses. While advances in genetic and biochemical engineering have made it possible to develop therapeutic agents to challenging disease targets, these applications to the field of vaccinology have not been fully realized. Recombinant protein technologies now allow the design of optimal antigens. Additionally, nanoparticles have increasingly demonstrated the potential for optimal antigen presentation and targeted drug delivery. Nanoparticles with multiple attached antigens have been shown to have increased binding avidity afforded by the multivalent display of their molecular cargos, and an ability to cross biological barriers more efficiently due to their nanoscopic size. Helicobacter pylori (H. pylori) ferritin nanoparticles fused to influenza virus haemagglutinin (HA) protein has allowed improved antigen stability and increased immunogenicity in mouse influenza models (see Kanekiyo et al., Nature 499:102-106 (2013)). This fusion protein self-assembled into an octahedrally-symmetric nanoparticle and presented 8 trimeric HA spikes to give a robust immune response in various pre-clinical models when used with an adjuvant.

Lyme borreliosis is a zoonotic disease caused by some bacterial species in the genus Borrelia and is transmitted to humans and canines by the bite of an infected Ixodes spp. tick. Lyme disease is a global public health problem, with cases reported from temperate climates across Europe, North America, and Asia. Outer surface protein A (OspA) of Borrelia is the major antigen that elicits an immune response. There are at least seven different serotypes (serotypes 1-7) of OspA that are found in Borrelia world-wide. Different genospecies of Borrelia exist worldwide, such that immunity to one genospecies may not confer immunity to other bacteria that can also cause Lyme borreliosis. Further, localized ranges of ticks that harbor Borrelia means that an OspA serotype that is associated with Lyme disease in patients in one geographic region might not be associated with Lyme disease in patients in another geographic region.

Here, a set of new polypeptides, nanoparticles, compositions, methods, and uses involving OspA polypeptides is presented. A modified OspA polypeptide was developed to provide protection from infection with Borrelia with reduced risk of stimulating an autoimmune reaction. Furthermore, self-adjuvanting antigenic polypeptides comprising an OspA polypeptide and ferritin were developed wherein immune-stimulatory moieties, such as adjuvants, were directly, chemically attached to the antigenic polypeptide. The direct conjugation of an immune-stimulatory moiety to the antigenic polypeptide allows for targeted co-delivery of the immune-stimulatory moiety and OspA polypeptide in a single macromolecular entity, which can greatly decrease the potential for systemic toxicity that is feared with traditional vaccines that comprise antigens and immune-stimulatory molecules such as adjuvants as separate molecules. The co-delivery of immune-stimulatory moieties together with OspA polypeptides in a macromolecular entity and their multivalent presentation may also reduce the overall dose needed to elicit protection, reducing manufacturing burdens and costs.

### SUMMARY

It is an object of this disclosure to provide compositions, kits, methods, and uses that can provide one or more of the advantages discussed above, or at least provide the public with a useful choice. Accordingly, the following embodiments are disclosed herein.

Embodiment 1 is an antigenic OspA polypeptide comprising an OspA serotype 1 polypeptide of Borrelia, wherein the polypeptide does not comprise the sequence of SEQ ID NO: 77.

Embodiment 2 is the antigenic OspA polypeptide of embodiment 1, wherein the polypeptide lacks a transmembrane domain or a portion of a transmembrane domain.

Embodiment 3 is the antigenic OspA polypeptide of any one of the preceding embodiments, wherein the polypeptide is non-lipidated.

Embodiment 4 is the antigenic OspA polypeptide of any one of the preceding embodiments, wherein there is at least one amino acid substitution relative to the sequence of SEQ ID NO: 77, wherein the substitution reduces identity to SEQ ID NO: 78, or is non-conservative and does not result in higher identity to SEQ ID NO: 78.

Embodiment 5 is the antigenic OspA polypeptide of embodiment 4, wherein the substitution reduces identity to SEQ ID NO: 78.

Embodiment 6 is the antigenic OspA polypeptide of any one of the preceding embodiments, wherein one or more of the amino acids of SEQ ID NO: 77 is replaced with the corresponding amino acid(s) of a non-serotype 1 OspA.

Embodiment 7 is the antigenic OspA polypeptide of embodiment 6, wherein the non-serotype 1 OspA is serotype 2, 3, 4, 5, 6, or 7 OspA.

Embodiment 8 is the antigenic OspA polypeptide of embodiment 6, wherein each of the amino acids of SEQ ID NO: 77 are replaced with the corresponding amino acids of a serotype 2, 3, 4, 5, 6, or 7 OspA.

Embodiment 9 is the antigenic OspA polypeptide of any one of the preceding embodiments, wherein the polypeptide further comprises a modification to reduce or eliminate glycosylation.

Embodiment 10 is the antigenic OspA polypeptide of embodiment 9, wherein the modification comprises a substitution of at least one asparagine.

Embodiment 11 is the antigenic OspA polypeptide of embodiment 10, wherein the at least one asparagine comprises any one, two, three, or more of N71, N190, N202, and N251 of OspA serotype 1.

Embodiment 12 is the antigenic OspA polypeptide of embodiment 11, wherein the at least one asparagine comprises N71, N190, N202, and N251 of OspA serotype 1.

Embodiment 13 is the antigenic OspA polypeptide of any one of embodiments 10-12, wherein the one or more asparagines are substituted with glutamine.

Embodiment 14 is the antigenic OspA polypeptide of any one of embodiments 10-13, wherein the polypeptide lacks an N-glycosylation site.

Embodiment 15 is the antigenic OspA polypeptide of any one of the preceding embodiments, wherein the OspA is from Borrelia burgdorferi, Borrelia mayonii, Borrelia afzelii, Borrelia garinii, or Borrelia bavariensis.

Embodiment 16 is the antigenic OspA polypeptide of any one of the preceding embodiments, comprising a sequence with at least 85%, 90%, 95%, 97%, 98%, 99%, 99.5%, or 100% identity to the sequence of any one of SEQ ID NOS: 1, 3, 4, or 53.

Embodiment 17 is the antigenic OspA polypeptide of any one of the preceding embodiments, comprising the sequence of any one of SEQ ID NO: 1-10 or 12-76.

Embodiment 18 is the antigenic OspA polypeptide of any one of the preceding embodiments, comprising an OspA ectodomain.

Embodiment 19 is the antigenic OspA polypeptide of any one of the preceding embodiments, comprising a sequence with at least 85%, 90%, 95%, 97%, 98%, 99%, 99.5%, or 100% identity to the sequence of any one of SEQ ID NOS: 94-102.

Embodiment 20 is the antigenic OspA polypeptide of any one of the preceding embodiments, further comprising a ferritin protein.

Embodiment 21 is the antigenic OspA polypeptide of embodiment 20, wherein the ferritin comprises a mutation replacing a surface-exposed amino acid with a cysteine.

Embodiment 22 is an antigenic OspA polypeptide comprising an OspA polypeptide and a ferritin, wherein the ferritin comprises a mutation replacing a surface-exposed amino acid with a cysteine.

Embodiment 23 is the antigenic OspA polypeptide of any one of embodiments 20-22, wherein the ferritin comprises one or more of E12C, S26C, S72C, A75C, K79C, S100C, and S111C mutations of H. pylori ferritin or one or more corresponding mutations in a non-H. pylori ferritin as determined by pairwise or structural alignment.

Embodiment 24 is the antigenic OspA polypeptide of any one of embodiments 20-22, wherein the ferritin comprises an E12C mutation of H. pylori ferritin or a corresponding mutation in a non-H. pylori ferritin as determined by pairwise or structural alignment.

Embodiment 25 is the antigenic OspA polypeptide of any one of embodiments 20-22, wherein the ferritin comprises an S26C mutation of H. pylori ferritin or a corresponding mutation in a non-H. pylori ferritin as determined by pairwise or structural alignment.

Embodiment 26 is the antigenic OspA polypeptide of any one of embodiments 20-22, wherein the ferritin comprises an S72C mutation of H. pylori ferritin or a corresponding mutation in a non-H. pylori ferritin as determined by pairwise or structural alignment.

Embodiment 27 is the antigenic OspA polypeptide of any one of embodiments 20-22, wherein the ferritin comprises an A75C mutation of H. pylori ferritin or a corresponding mutation in a non-H. pylori ferritin as determined by pairwise or structural alignment.

Embodiment 28 is the antigenic OspA polypeptide of any one of embodiments 20-22, wherein the ferritin comprises a K79C mutation of H. pylori ferritin or a corresponding mutation in a non-H. pylori ferritin as determined by pairwise or structural alignment.

Embodiment 29 is the antigenic OspA polypeptide of any one of embodiments 20-22, wherein the ferritin comprises an S100C mutation of H. pylori ferritin or a corresponding mutation in a non-H. pylori ferritin as determined by pairwise or structural alignment.

Embodiment 30 is the antigenic OspA polypeptide of any one of embodiments 20-22, wherein the ferritin comprises an S111C mutation of H. pylori ferritin or a corresponding mutation in a non-H. pylori ferritin as determined by pairwise or structural alignment.

Embodiment 31 is the antigenic OspA polypeptide of any one of embodiments 20-30, comprising one or more immune-stimulatory moieties linked to the ferritin via a surface-exposed amino acid, optionally wherein the surface-exposed amino acid is a cysteine resulting from a mutation.

Embodiment 31a is the antigenic OspA polypeptide of embodiment 31, wherein the immune-stimulatory moiety is an agonist of TLR2, optionally wherein the agonist is PAM2CSK4, FSL-1, or PAM3CSK4.

Embodiment 31b is the antigenic OspA polypeptide of embodiment 31, wherein the immune-stimulatory moiety is an agonist of TLR7/8, optionally wherein the agonist is a single-stranded RNA, an imidazoquinoline, a nucleoside analog, 3M-012, or SM7/8a.

Embodiment 31c is the antigenic OspA polypeptide of embodiment 31, wherein the immune-stimulatory moiety is an agonist of TLR9, optionally wherein the agonist is a CpH oligodeoxynucleotide (ODN), an ODN comprising one or more 6mer CpG motif comprising 5′ Purine (Pu)-Pyrimidine (Py)-C-G-Py-Pu 3′, an ODN comprising the sequence of SEQ ID NO: 210, or ISS-1018.

Embodiment 31d is the ferritin protein of embodiment 31c, wherein the agonist of TLR9 comprises a backbone comprising phosphorothioate linkages.

Embodiment 31e is the antigenic OspA polypeptide of embodiment 31, wherein the immune-stimulatory moiety is an agonist of STING, optionally wherein the agonist is a cyclic dinucleotide (CDN), cdA, cdG, cAMP-cGMP, and 2′-5′,3′-5′ cGAMP, or DMXAA.

Embodiment 32 is the antigenic OspA polypeptide of any one of embodiments 20-31e, wherein the ferritin comprises a mutation replacing a surface-exposed asparagine with a non-asparagine amino acid, optionally wherein the asparagine is at position 19 of H. pylori ferritin, or an analogous position in a non-H. pylori ferritin as determined by pairwise or structural alignment.

Embodiment 33 is the antigenic OspA polypeptide of any one of embodiments 20-32, wherein the ferritin comprises a mutation replacing an internal cysteine with a non-cysteine amino acid, optionally wherein the internal cysteine is at position 31 of H. pylori ferritin, or a position that corresponds to position 31 of H. pylori ferritin as determined by pair-wise or structural alignment.

Embodiment 34 is the antigenic OspA polypeptide of any one of embodiments 20-33, wherein the antigenic OspA polypeptide comprises a peptide linker between the OspA polypeptide and the ferritin.

Embodiment 35 is the antigenic OspA polypeptide of embodiment 34, wherein the peptide linker is N-terminal to the ferritin.

Embodiment 35a is the antigenic OspA polypeptide of embodiment 34, wherein the peptide linker is C-terminal to the ferritin.

Embodiment 36 is the antigenic OspA polypeptide of any one of the preceding embodiments, wherein the ferritin comprises an amino acid sequence with 80%, 85%, 90%, 95%, 98%, or 99% identity to any one of SEQ ID NOs: 201-207 or 211-215.

Embodiment 36a is the antigenic OspA polypeptide of embodiment 36, comprising an amino acid sequence with 80%, 85%, 90%, 95%, 98%, or 99% identity to any one of SEQ ID NOs: 201.

Embodiment 36b is the antigenic OspA polypeptide of embodiment 36, comprising an amino acid sequence with 80%, 85%, 90%, 95%, 98%, or 99% identity to any one of SEQ ID NOs: 202.

Embodiment 36c is the antigenic OspA polypeptide of embodiment 36, comprising an amino acid sequence with 80%, 85%, 90%, 95%, 98%, or 99% identity to any one of SEQ ID NOs: 203.

Embodiment 36d is the antigenic OspA polypeptide of embodiment 36, comprising an amino acid sequence with 80%, 85%, 90%, 95%, 98%, or 99% identity to SEQ ID NO: 215.

Embodiment 36e is the antigenic OspA polypeptide of embodiment 36, comprising an amino acid sequence with 80%, 85%, 90%, 95%, 98%, or 99% identity to SEQ ID NO: 204.

Embodiment 36f is the antigenic OspA polypeptide of embodiment 36, comprising an amino acid sequence with 80%, 85%, 90%, 95%, 98%, or 99% identity to SEQ ID NO: 205.

Embodiment 36g is the antigenic OspA polypeptide of embodiment 36, comprising an amino acid sequence with 80%, 85%, 90%, 95%, 98%, or 99% identity to SEQ ID NO: 206.

Embodiment 36h is the antigenic OspA polypeptide of embodiment 36, comprising an amino acid sequence with 80%, 85%, 90%, 95%, 98%, or 99% identity to SEQ ID NO: 207.

Embodiment 36i is the antigenic OspA polypeptide of embodiment 36, comprising an amino acid sequence with 80%, 85%, 90%, 95%, 98%, or 99% identity to SEQ ID NO: 211.

Embodiment 36j is the antigenic OspA polypeptide of embodiment 36, comprising an amino acid sequence with 80%, 85%, 90%, 95%, 98%, or 99% identity to SEQ ID NO: 212.

Embodiment 36k is the antigenic OspA polypeptide of embodiment 36, comprising an amino acid sequence with 80%, 85%, 90%, 95%, 98%, or 99% identity to SEQ ID NO: 213.

Embodiment 36l is the antigenic OspA polypeptide of embodiment 36, comprising an amino acid sequence with 80%, 85%, 90%, 95%, 98%, or 99% identity to SEQ ID NO: 214.

Embodiment 37 is a ferritin particle comprising the antigenic OspA polypeptide of any one of embodiments 20-361.

Embodiment 38 is the antigenic OspA polypeptide of any one of embodiments 1-19, further comprising a lumazine synthase protein.

Embodiment 39 is a lumazine synthase particle comprising the antigenic OspA polypeptide of embodiment 38.

Embodiment 40 is a composition comprising the antigenic OspA polypeptide, ferritin particle, or lumazine synthase particle of any one of the preceding embodiments, further comprising a pharmaceutically acceptable carrier.

Embodiment 41 is the composition of embodiment 40, further comprising an adjuvant, optionally wherein the adjuvant is AF03.

Embodiment 42 is the composition of embodiment 40 or 41, which comprises a first and second antigenic OspA polypeptide, wherein the first and second antigenic OspA polypeptides comprise OspA polypeptides of different serotypes.

Embodiment 43 is the composition of embodiment 42, comprising one, two, three, four, five, six, or seven antigenic OspA polypeptides selected from: an antigenic OspA polypeptide comprising an OspA serotype 1 polypeptide; an antigenic OspA polypeptide comprising an OspA serotype 2 polypeptide; an antigenic OspA polypeptide comprising an OspA serotype 3 polypeptide; an antigenic OspA polypeptide comprising an OspA serotype 4 polypeptide; an antigenic OspA polypeptide comprising an OspA serotype 5 polypeptide; an antigenic OspA polypeptide comprising an OspA serotype 6 polypeptide; and an antigenic OspA polypeptide comprising an OspA serotype 7 polypeptide.

Embodiment 44 is the composition of embodiment 43, comprising an antigenic OspA polypeptide comprising an OspA serotype 1 polypeptide; an antigenic OspA polypeptide comprising an OspA serotype 2 polypeptide; an antigenic OspA polypeptide comprising an OspA serotype 3 polypeptide; an antigenic OspA polypeptide comprising an OspA serotype 4 polypeptide; an antigenic OspA polypeptide comprising an OspA serotype 5 polypeptide; and an antigenic OspA polypeptide comprising an OspA serotype 7 polypeptide.

Embodiment 45 is the antigenic OspA polypeptide, ferritin particle, lumazine synthase particle, or composition of any one of embodiments 1-44 for use in a method of eliciting an immune response to Borrelia or in protecting a subject against Lyme Disease.

Embodiment 46 is a method of eliciting an immune response to Borrelia or protecting a subject against Lyme Disease comprising administering any one or more of the antigenic OspA polypeptide, ferritin particle, lumazine synthase particle, or composition of any one of embodiments 1-44 to a subject.

Embodiment 47 is the antigenic OspA polypeptide, ferritin particle, lumazine synthase particle, composition, or method of any one of embodiments 45-46, wherein the subject is human.

Embodiment 47a is the antigenic OspA polypeptide, ferritin particle, lumazine synthase particle, composition, or method of any one of embodiments 45-46, wherein the subject is a mammal, optionally wherein the mammal is a primate or domesticated mammal, further optionally wherein the primate is a non-human primate, monkey, macaque, rhesus or cynomolgus macaque, or ape, or the domesticated mammal is a dog, rabbit, cat, horse, sheep, cow, goat, camel, or donkey.

Embodiment 48 is a kit comprising any one or more of the antigenic OspA polypeptide, ferritin particle, lumazine synthase particle, and compositions of embodiments 1-44, optionally with instructions for use in immunizing subjects against Lyme Disease.

Embodiment 49 is a nucleic acid encoding the antigenic OspA polypeptide of any one of embodiments 1-36l, optionally wherein the nucleic acid is RNA.

Additional objects and advantages will be set forth in part in the description which follows, and in part will be obvious from the description, or may be learned by practice. The objects and advantages will be realized and attained by means of the elements and combinations particularly pointed out in the appended claims.

It is to be understood that both the foregoing general description and the following detailed description are exemplary and explanatory only and are not restrictive of the claims.

The accompanying drawings, which are incorporated in and constitute a part of this specification, illustrate several embodiments and together with the description, serve to explain the principles described herein.

### BRIEF DESCRIPTION OF THE DRAWINGS

FIGS. 1A-1D show exemplary designs of OspA-Ferritin nanoparticles. FIG. 1A. OspA genetically fused to ferritin to form a fusion protein. The OspA and ferritin sequences are separated by a glycine-serine linker (-GS-). FIG. 1B. A structure of the ectodomain of OspA is depicted. The C-terminus where OspA is attached to ferritin is indicated with an asterisk. FIG. 1C. An exemplary ferritin nanoparticle composed of 24 monomers of H. pylori ferritin. FIG. 1D. An exemplary OspA-ferritin fusion protein nanoparticle. Ferritin (light gray), the location of the glycine-serine linker (GS), and OspA (dark gray and black) are depicted (n: number of subunits).

FIGS. 2A-2D show expression and purification of an exemplary OspA-Ferritin. FIG. 2A. Size exclusion chromatography (SEC) profile of an exemplary OspA-Ferritin nanoparticle purified on a Superose 6 column. FIG. 2B. SDS-PAGE gel of a purified exemplary OspA-Ferritin from Expi293 cells. FIG. 2C. Dynamic Light Scattering (DLS) profile of exemplary OspA-Ferritin nanoparticles. Radius is 13 nm, % Pd (measure of normalized polydispersity) is 7.4, and mass is 100%. FIG. 2D. Composite image of an exemplary OspA-Ferritin constructed from class averaging of transmission electron micrographs of 318 particles at 67,000× magnification. Ferritin nanoparticles appear on transmission electron microscopy as a strong circular density with a hollow center. Each nanoparticle is surrounded by numerous, short shapes corresponding to OspA that appear circular or slightly oblong.

FIGS. 3A-3B show generation of alternative serotype OspA nanoparticles in E. coli. FIG. 3A. Biochemical analysis by SDS-PAGE of OspA-Ferritin Serotypes 1-5 and 7 purified by size exclusion chromatography. FIG. 3B. Transmission electron microscopy of OspA-Ferritin Serotypes 1-5 and 7 (98,000×).

FIG. 4 shows comparison of immunogenicity and duration of exemplary Serotype 1 OspA-Ferritin nanoparticles to RECOMBITEK® Lyme (liquid suspension of purified Outer surface protein A (OspA) of Borrelia burgdorferi). C3H mice (n=5) were immunized intramuscularly with 1 μg of the OspA-Ferritin+Ribi adjuvant (Sigma adjuvant system Cat #S6322-1vl) or RECOMBITEK® Lyme at week 0 and week 4. Antibody response was assessed by measuring endpoint titers via ELISA 2 weeks after the 2nd immunization (week 6) and 21 weeks after 2^{nd} immunization (week 25) with each composition.

FIGS. 5A-5C present information regarding exemplary OspA-Ferritin, wherein the OspA polypeptide is modified at an epitope of OspA serotype 1 that has homology with a fragment of the sequence of human leukocyte function-associated antigen-1 (hLFA-1). FIG. 5A. Structure showing the location of the LFA-1 homology site (amino acids 165-173 of SEQ ID NO: 83) within an OspA ectodomain. FIG. 5B. Dendrogram showing the relationship of OspA amino acids 165-173 of SEQ ID NO: 83 (B. burgdorferi Serotype 1 OspA) to corresponding sequences in hLFA-1 and other Borrelia species and serotypes. FIG. 5C. The nine-amino-acid segment (nonapeptide) at amino acids 165-173 of SEQ ID NO: 83(labeled “OspA”) is compared with the corresponding nonapeptides from Serotype 2 and Serotype 3 OspA (“S2” (SEQ ID NO: 79) and “S3” (SEQ ID NO: 80) respectively), a rationally designed substitute nonapeptide (“RD2”; SEQ ID NO: 81), and the corresponding nonapeptide from hLFA-1 (SEQ ID NO: 78). FIG. 5C discloses SEQ ID NOS 77, 79-81, and 78, respectively, in order of appearance. FIG. 5D. C3H mice (n=5) were immunized intramuscularly (IM) at week 0 and week 4 with 1 μg doses of OspA Serotype 1-ferritin nanoparticles with AddaVax™ adjuvant (squalene-based oil-in-water nano-emulsion; available from InvivoGen, Cat. No. vac-adx-10). The OspA sequence comprised the wild-type hLFA-1 homology site (i.e., amino acids 165-173 of SEQ ID NO: 83; “Serol”) or a substitute sequence as follows: SEQ ID NO: 81 (“RD”); SEQ ID NO: 80 (“Sero 3 Replacement”); SEQ ID NO: 79 (“Sero 2 replacement”). Antibody response was assessed via endpoint titer measured by ELISA 2 weeks after the 2^{nd} immunization of the indicated constructs.

FIGS. 6A-6B present information regarding exemplary OspA-Ferritin nanoparticles conjugated to an exemplary immune-stimulatory moiety: TLR 7/8 agonist (3M-012). FIG. 6A. A 2-step click chemistry strategy was used to attach the 3M-012 to ferritin. A DBCO-PEG4-maleimide linker was first attached to a surface exposed cysteine on ferritin. After excess linker was removed, azide-3M-012 was added. FIG. 6B. C3H mice (n=5) were immunized intramuscularly with 1 μg of the indicated composition at weeks 0and 4 and analyzed 2 weeks later. “Conjugate” indicates OspA-ferritin-3M-012 conjugated nanoparticle. “Admix” indicates a non-conjugated mix of the same OspA-ferritin administered with 29 ng or 20 μg 3M-012 or Alum. The 29 ng “admix” mixture of OspA-ferritin and 3M-012 represents the molar equivalent amount of 3M-012 on the conjugated nanoparticle.

FIGS. 7A-7C present information regarding exemplary OspA-ferritin nanoparticles conjugated to an exemplary immune-stimulatory moiety: ISS-1018 CpG (SEQ ID NO: 210). FIG. 7A. A 2-step click chemistry strategy was used to attach the CPG to ferritin. A DBCO-PEG4-maleimide linker was first attached to a surface exposed cysteine on ferritin. After excess linker was removed, azide-CpG was added. FIG. 7A discloses SEQ ID NO: 228. FIG. 7B. Biochemical analysis of CpG-conjugation by a SDS-PAGE gel reveals a shift in molecular weight after conjugation to CpG with 92% of OspA-Ferritin conjugated to CpG. FIG. 7C. C3H mice (n=5) were immunized intramuscularly with lug of the indicated composition and at weeks 0 and 4 and analyzed 2 weeks later. “Conjugate” indicates OspA-ferritin-CPG conjugated nanoparticle. “Admix” indicates a non-conjugated mix of the same OspA-ferritin administered with 339 ng or 50 μg CpG or Alum. The 339 ng “admix” mixture of OspA-ferritin and CPG represents the molar equivalent amount of CpG on the conjugated nanoparticle.

FIGS. 8A-8F compare antibody responses to serotype 1 (FIG. 8A), serotype 2 (FIG. 8B), serotype 3 (FIG. 8C), serotype 4 (FIG. 8D), serotype 5 (FIG. 8E), and serotype 7 (FIG. 8F) following administration of monovalent serotype-matched OspA-ferritin (1 μg per dose) (“Monovalent”) with Alum adjuvant or a hexavalent composition comprising each of serotype 1 OspA-ferritin, serotype 2 OspA-ferritin, serotype 3 OspA-ferritin, serotype 4OspA-ferritin, serotype 5 OspA-ferritin, and serotype 7 OspA-ferritin at 1 μg each per dose with Alum adjuvant (“Hexavalent”). C3H mice (n=5) were immunized intramuscularly at weeks 0 and 4, and antibody response was assessed via endpoint titer measured by ELISA 2 weeks later. ELISA plates were coated with the specified serotype of OspA.

FIGS. 9A-9G show antibody responses in mice to serotype 1 (FIG. 9A), serotype 2 (FIG. 9B), serotype 3 (FIG. 9C), serotype 4 (FIG. 9D), serotype 5 (FIG. 9E), serotype 6 (FIG. 9F), and serotype 7 (FIG. 9G) observed in mice following administration of conjugated and non-conjugated hexavalent OspA-ferritin nanoparticle compositions. Hexavalent compositions comprised each of serotype 1 OspA-ferritin, serotype 2 OspA-ferritin, serotype 3 OspA-ferritin, serotype 4 OspA-ferritin, serotype 5 OspA-ferritin, and serotype 7 OspA-ferritin as described for FIGS. 8A-F except that “Hexavalent-CPG” and “Hexavalent-3M-012” indicate that nanoparticles were chemically conjugated to CPG and 3M-012 (see FIGS. 7A and 6A and accompanying description). Antibody response was assessed via endpoint titer measured by ELISA 2 weeks later. ELISA plates were coated with the specified serotype of OspA.

FIGS. 10A-10G show antibody responses to serotypes 1-7, respectively, in Rhesus monkeys (n=3 per group) to hexavalent OspA-ferritin nanoparticle compositions, which were as described for FIGS. 9A-G except that doses were 60 μg total (10 μg each serotype) and contained non-conjugated AF03 adjuvant. Monkeys were immunized intramuscularly at week 0 and week 6. Antibody response was analyzed 2 weeks after immunization via endpoint titer measured by ELISA. RECOMBITEK® Lyme was used as a comparative reference at 10 μg dose. For all experiments, an ELISA plate was coated with the OspA serotype indicated in each panel.

FIGS. 10H-10N show antibody responses to serotypes 1-7, respectively, in Rhesus monkeys (n=3 per group) to hexavalent OspA-ferritin nanoparticle compositions, which were as described for FIGS. 10A-G except that no AF03 adjuvant was used and nanoparticles were instead conjugated to 3M-012 or CpG (see FIGS. 6A and 7A and accompanying description). Doses were 60 μg total (10 μg each serotype). Monkeys were immunized intramuscularly at week 0 and week 6. Antibody response was analyzed 2 weeks after immunization via endpoint titer measured by ELISA. For all experiments, an ELISA plate was coated with the OspA serotype indicated in each panel.

FIG. 11 shows results from tick challenge testing of 3M-012 conjugated OspA-ferritin compositions. Mice were immunized with a 1 μg dose of the indicated compositions at week 0 and week 4. The monovalent composition contained 1 μg of OspA-ferritin serotype 1 conjugated to 3M-012. The “Hexavalent-3M-012” composition was as described for FIGS. 9A-G. The control particle lacked an OspA polypeptide. Mice were challenged with 5-6 ticks infected with Borrelia burgdorferi N40 strain (serotype 1) for 5 days two weeks after the second immunization and sacrificed two weeks later. Tissue samples from the heart, ankle and ear were cultured in BSK media with antibiotics for B. burgdorferi for 6 weeks. Negative samples were tested by PCR for the presence of B. burgdorferi. A positive sample was positive either by culture or PCR.

FIG. 12 shows confirmation of conjugation of OspA-ferritin nanoparticles to TLR 7/8 agonist 3M-012 by mass spectrometry. Top panel shows unconjugated, and bottom panel shows conjugated constructs. The data shows a mass shift of 586.69 Daltons, consistent with the addition of 3M-012.

FIG. 13 shows antibody response in mice to non-glycosylated mutant OspA-ferritin (NG-RD) as compared to a glycosylated counterpart (RD) measured by ELISA across a dilution series as shown. RD=SEQ ID NO: 52. NG-RD=SEQ ID NO: 53. Mice were vaccinated with 1 μg doses at week zero and week 4.

FIG. 14 shows antibody response in mice to OspA-ferritin (SEQ ID NO: 52), OspA-ferritin glycosylation mutant N>Q (SEQ ID NO: 53), and glycosylation mutant S/T>A (SEQ ID NO: 63) compared to RECOMBITEK® Lyme and negative (Pre immune) controls, measured by ELISA across a dilution series as shown.

FIGS. 15A-15E show purification and characterization of OspA constructs comprising different linkers (GS, Gly-Ser linker; GS1, Gly-Gly-Gly-Ser linker (SEQ ID NO: 226); GS2, SEQ ID NO: 91 linker; GS5, SEQ ID NO: 92 linker; construct sequences were SEQ ID NOs: 53 and 60-62, respectively). FIG. 15A. Coomassie staining of purified OspA constructs comprising linkers as indicated. FIG. 15A discloses SEQ ID NOS 226 and 91-92, respectively, in order of appearance. FIG. 15B. Dynamic Light Scattering (DLS) of OspA-ferritin nanoparticle comprising GS1 (SEQ ID NO: 60). FIG. 15C. DLS of OspA-ferritin nanoparticle comprising GS2 (SEQ ID NO: 61). FIG. 15D. Electron micrograph (EM) of OspA-ferritin nanoparticle comprising GS5 (SEQ ID NO: 62). FIG. 15E. DLS of OspA-ferritin nanoparticle comprising GS5 (SEQ ID NO: 62).

FIG. 16 shows antibody response in mice to OspA-ferritin constructs comprising different linkers (Linker 1X GGGS (SEQ ID NO: 226) construct, SEQ ID NO: 60; Linker 2x GGGS (SEQ ID NO: 91) construct, SEQ ID NO: 61; Linker 5X GGGS (SEQ ID NO: 92) construct, SEQ ID NO: 62) compared to RECOMBITEK® Lyme and negative (Pre-immune) controls, measured by ELISA across a dilution series as shown.

FIGS. 17A-17C show characterization of a lumazine synthase OspA serotype 4 construct (SEQ ID NO: 18). FIG. 17A. DLS data. FIG. 17B. Coomassie gel of indicated fractions 22-64 of size exclusion chromatography (SEC) trace. FIG. 17C. EM data.

FIG. 18 shows antibody response in mice to a OspA serotype 4-ferritin construct (SEQ ID NO: 4) and an OspA serotype 4-lumazine synthase construct (SEQ ID NO: 18), with or without Alum.

FIGS. 19A-19C show characterization of a OspA serotype 1-lumazine synthase construct (SEQ ID NO: 12). FIG. 19A. EM data. FIG. 19B. Coomassie gel of indicated fractions 20-40 of the SEC trace. FIG. 19C. DLS data.

FIGS. 20A-20C show characterization of a OspA serotype 2-lumazine synthase construct (SEQ ID NO: 16). FIG. 20A. EM data. FIG. 20B. Coomassie gel of indicated fractions 27-56 of the SEC trace. FIG. 20C. DLS data.

FIGS. 21A-21B show characterization of a OspA serotype 3-lumazine synthase construct (SEQ ID NO: 17). FIG. 21A. Coomassie gel of indicated fractions 23-39 of the SEC trace. FIG. 21B. DLS data.

FIGS. 22A-22C show characterization of a OspA serotype 5-lumazine synthase construct (SEQ ID NO: 19). FIG. 22A. EM data. FIG. 22B. Coomassie gel of indicated fractions 22-38 of the SEC trace. FIG. 22C. DLS data.

FIGS. 23A-23C show characterization of a OspA serotype 7-lumazine synthase construct (SEQ ID NO: 21). FIG. 23A. EM data. FIG. 23B. Coomassie gel of indicated fractions 20-38 of the SEC trace. FIG. 23C. DLS data.

FIG. 24A-24G show antibody responses to serotypes 1-7, respectively, in C3H mice (n=5 per group) to heptavalent OspA-ferritin nanoparticle compositions of 1 μg each of OspA-ferritin nanoparticles corresponding to OspA serotypes 1-7 (total 7 μg) adjuvanted with either alum or AF03, or to RECOMBITEK® Lyme. For all experiments, an ELISA plate was coated with the OspA serotype indicated in each panel as “S X” where X is the serotype number.

FIG. 25 shows a time course of endpoint antibody titer in Rhesus monkeys. Monkeys were immunized intramuscularly at week 0 and week 6 with either hexavalent OspA-ferritin vaccine (containing OspA of serotypes 1, 2, 3, 4, 5, and 7 in separate nanoparticles) with AF03 adjuvant or RECOMBITEK®. ELISA plate was coated with OspA serotype 1.

### DETAILED DESCRIPTION

Provided herein are novel antigens for vaccination against Lyme disease. Lyme disease is caused by bacteria belonging to the Borrelia burgdorferi sensu lato (s.l.) complex (herein referred to as “Borrelia”). The antigens described herein include antigenic polypeptides, fusion proteins, nanoparticles, and compositions that can be used by themselves or with non-conjugated adjuvant to vaccinate subjects against Lyme disease. The fusion proteins may comprise antigenic polypeptides fused to ferritin. Ferritin may be wild-type or may comprise one or more mutations, e.g., a mutation replacing a surface-exposed amino acid with a cysteine so that immune-stimulatory moieties may be directly conjugated to the engineered surface-exposed cysteine. A cysteine resulting from such a mutation may eliminate or reduce the need for separately administered adjuvant, and also potentially reduce the amount of adjuvant/immune-stimulatory moiety needed to elicit an immune response to the antigen. Nucleic acids that encode the antigenic polypeptides described herein are also provided.

### A. Definitions

“Adjuvant,” as used herein, refers to a substance or vehicle that non-specifically enhances the immune response to an antigen. Adjuvants can include, without limitation, a suspension of minerals (e.g., alum, aluminum hydroxide, or phosphate) on which antigen is adsorbed; a water-in-oil or oil-in-water emulsion in which antigen solution is emulsified in mineral oil or in water (e.g., Freund's incomplete adjuvant). Sometimes killed mycobacteria is included (e.g., Freund's complete adjuvant) to further enhance antigenicity. Immuno-stimulatory oligonucleotides (e.g., a CpG motif) can also be used as adjuvants (for example, see U.S. Pat. Nos. 6,194,388; 6,207,646; 6,214,806; 6,218,371; 6,239,116;6,339,068; 6,406,705; and 6,429,199). Adjuvants can also include biological molecules, such as Toll-Like Receptor (TLR) agonists and costimulatory molecules. An adjuvant may be administered as a separate molecule in a composition or covalently bound (conjugated) to modified ferritin or an antigenic ferritin polypeptide.

As used herein, an “antigen” refers to an agent that elicits an immune response, and/or an agent that is bound by a T cell receptor (e.g., when presented by an MHC molecule) or to an antibody (e.g., produced by a B cell) when exposed or administered to an organism. In some embodiments, an antigen elicits a humoral response (e.g., including production of antigen-specific antibodies) in an organism. Alternatively, or additionally, in some embodiments, an antigen elicits a cellular response (e.g., involving T-cells whose receptors specifically interact with the antigen) in an organism. A particular antigen may elicit an immune response in one or several members of a target organism (e.g., mice, rabbits, primates, humans), but not in all members of the target organism species. In some embodiments, an antigen elicits an immune response in at least about 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% of the members of a target organism species. In some embodiments, an antigen binds to an antibody and/or T cell receptor, and may or may not induce a particular physiological response in an organism. In some embodiments, for example, an antigen may bind to an antibody and/or to a T cell receptor in vitro, whether or not such an interaction occurs in vivo. In some embodiments, an antigen reacts with the products of specific humoral or cellular immunity, including those induced by heterologous immunogens. Antigens include antigenic ferritin proteins comprising ferritin (e.g., comprising one or more mutations) and a non-ferritin polypeptide as described herein.

“Ferritin” or “ferritin protein,” as used herein, refers to a protein with detectable sequence identity to H. pylori ferritin (SEQ ID NO: 208 or 209) or another ferritin discussed herein, such as P. furiosus ferritin, Trichoplusia ni ferritin, or human ferritin, that serves to store iron, e.g., intracellularly or in tissues or to carry iron in the bloodstream. Such exemplary ferritins, including those that occur as two polypeptide chains, known as the heavy and light chains (e.g., T. ni and human ferritin), are discussed in detail below. In some embodiments, a ferritin comprises a sequence with at least 15%, 20%, 30%, 40%, 50%, 60%, 70%, 75%, 80%, 85%, 90%, 95%, 97%, 98%, 99%, or 99.5% identity to a ferritin sequence disclosed herein, e.g., in Table 1 (Sequence Table). A ferritin may be a fragment of a full-length naturally-occurring sequence. “Wild-type ferritin,” as used herein, refers to a ferritin whose sequence consists of a naturally-occurring sequence. Ferritins also include full-length ferritin or a fragment of ferritin with one or more differences in its amino acid sequence from a wild-type ferritin.

As used herein, a “ferritin monomer” refers to a single ferritin molecule (or, where applicable, a single ferritin heavy or light chain) that has not assembled with other ferritin molecules. A “ferritin multimer” comprises multiple associated ferritin monomers. A “ferritin protein” includes monomeric ferritin and multimeric ferritin.

As used herein, “ferritin particle,” refers to ferritin that has self-assembled into a globular form. Ferritin particles are sometimes referred to as “ferritin nanoparticles” or simply “nanoparticles”. In some embodiments, a ferritin particle comprises 24 ferritin monomers (or, where applicable, 24 total heavy and light chains).

“Hybrid ferritin,” as used herein, refers to ferritin comprising H. pylori ferritin with an amino terminal extension of bullfrog ferritin. An exemplary sequence used as an amino terminal extension of bullfrog ferritin appears as SEQ ID NO: 217. In hybrid ferritin, the amino terminal extension of bullfrog ferritin can be fused to H. pylori ferritin such that immune-stimulatory moiety attachment sites are distributed evenly on the ferritin particle surface. “Bullfrog linker” as used herein is a linker comprising the sequence of SEQ ID NO: 217. Hybrid ferritin is also sometimes referred to as “bfpFerr” or “bfp ferritin.” Any of the constructs comprising a bullfrog sequence can be provided without the bullfrog sequence, such as, for example, without a linker or with an alternative linker. Exemplary bullfrog linker sequences are provided in Table 1. Where Table 1 shows a bullfrog linker, the same construct may be made without a linker or with an alternative linker.

“Glycosylation,” as used herein, refers to the addition of a saccharide unit to a protein.

“Immune response,” as used herein, refers to a response of a cell of the immune system, such as a B cell, T cell, dendritic cell, macrophage or polymorphonucleocyte, to a stimulus such as an antigen or vaccine. An immune response can include any cell of the body involved in a host defense response, including for example, an epithelial cell that secretes an interferon or a cytokine. An immune response includes, but is not limited to, an innate and/or adaptive immune response. As used herein, a “protective immune response” refers to an immune response that protects a subject from infection (e.g., prevents infection or prevents the development of disease associated with infection). Methods of measuring immune responses are well known in the art and include, for example, by measuring proliferation and/or activity of lymphocytes (such as B or T cells), secretion of cytokines or chemokines, inflammation, antibody production and the like. An “antibody response” is an immune response in which antibodies are produced.

An “immune-stimulatory moiety,” as used herein, refers to a moiety that is covalently attached to a ferritin or antigenic ferritin polypeptide and that can activate a component of the immune system (either alone or when attached to ferritin or antigenic ferritin polypeptide). Exemplary immune-stimulatory moieties include agonists of toll-like receptors (TLRs), e.g., TLR 4, 7, 8, or 9. In some embodiments, an immune-stimulatory moiety is an adjuvant.

As used herein, the term “kit” refers to a packaged set of related components, such as one or more compounds or compositions and one or more related materials such as solvents, solutions, buffers, instructions, or desiccants.

“N-glycan,” as used herein, refers to a saccharide chain attached to a protein at the amide nitrogen of an N (asparagine) residue of the protein. As such, an N-glycan is formed by the process of N-glycosylation. This glycan may be a polysaccharide.

An “OspA ectodomain” as used herein refers to about amino acid residues 27-273 of B. burgdorferi OspA (UniProt Accession No. P0CL66) or the corresponding positions of a homolog thereof as identified by pairwise or structural alignment. Further examples of OspA ectodomains include positions 27-X of any of SEQ ID NOs: 83-89 where X is the C-terminal position of the relevant sequence, optionally wherein the C-terminal Lys is omitted. In some embodiments, an ectodomain further comprises at its N-terminus the 26th residue, or the 25th and 26th residues, of the corresponding full-length wild-type sequence; in SEQ ID NOs: 83-89, the 25th and 26th residues are Asp and Glu. Still further examples of OspA ectodomains include any of SEQ ID NOs: 94-102, optionally wherein the N-terminal 1, 2, or 3 residues (Met-Asp-Glu) are omitted, further optionally wherein the C-terminal Lys is omitted.

An “OspA transmembrane domain” as used herein refers to about amino acid residues 2-24 of B. burgdorferi OspA (UniProt Accession No. P0CL66) or the corresponding positions of a homolog thereof as identified by pairwise or structural alignment.

“Self-adjuvanting,” as used herein, refers to a composition or polypeptide comprising a ferritin and an immune-stimulatory moiety directly conjugated to the ferritin so that the ferritin and immune-stimulatory moiety are in the same molecular entity. An antigenic ferritin polypeptide comprising a non-ferritin polypeptide may be conjugated to an immune-stimulatory moiety to generate a self-adjuvanting polypeptide.

An “antigenic OspA polypeptide” is used herein to refer to a polypeptide comprising all or part of an OspA of sufficient length that the polypeptide is antigenic with respect to OspA. Full-length OspA comprises a transmembrane domain and an ectodomain, defined below. Antigenicity may be a feature of the OspA sequence as part of a construct further comprising a heterologous sequence, such as a ferritin or lumazine synthase protein. That is, if an OspA is part of a construct further comprising a heterologous sequence, then it is sufficient that the construct can serve as an antigen that generates anti-OspA antibodies, regardless of whether the OspA sequence without the heterologous sequence could do so.

Antigenic ferritin polypeptide” and “antigenic ferritin protein” are used interchangeably herein to refer to a polypeptide comprising a ferritin and a non-ferritin polypeptide, such as OspA, of sufficient length that the molecule is antigenic with respect to the non-ferritin polypeptide. The antigenic ferritin polypeptide may further comprise an immune-stimulatory moiety. Antigenicity may be a feature of the non-ferritin sequence as part of the larger construct. That is, it is sufficient that the construct can serve as an antigen against the non-ferritin polypeptide, regardless of whether the non-ferritin polypeptide without the ferritin (and immune-stimulatory moiety if applicable) could do so. In some embodiments, the non-ferritin polypeptide is an OspA polypeptide, in which case the antigenic ferritin polypeptide is also an “antigenic OspA polypeptide”. To be clear, however, an “antigenic OspA polypeptide” does not need to comprise ferritin. “Antigenic polypeptide” is used herein to refer to a polypeptide which is either or both of an antigenic ferritin polypeptide and an antigenic OspA polypeptide. The disclosure describes nucleic acid sequences and amino acid sequences having a certain degree of identity to a given nucleic acid sequence or amino acid sequence, respectively (a references sequence).

“Sequence identity” between two nucleic acid sequences indicates the percentage of nucleotides that are identical between the sequences. “Sequence identity” between two amino acid sequences indicates the percentage of amino acids that are identical between the sequences.

The terms “% identical”, “% identity” or similar terms are intended to refer, in particular, to the percentage of nucleotides or amino acids which are identical in an optimal alignment between the sequences to be compared. Said percentage is purely statistical, and the differences between the two sequences may be but are not necessarily randomly distributed over the entire length of the sequences to be compared. Comparisons of two sequences are usually carried out by comparing said sequences, after optimal alignment, with respect to a segment or “window of comparison”, in order to identify local regions of corresponding sequences. The optimal alignment for a comparison may be carried out manually or with the aid of the local homology algorithm by Smith and Waterman, 1981, Ads App. Math. 2, 482, with the aid of the local homology algorithm by Needleman and Wunsch, 1970, J. Mol. Biol. 48, 443, with the aid of the similarity search algorithm by Pearson and Lipman, 1988, Proc. Natl Acad. Sci. USA 88, 2444, or with the aid of computer programs using said algorithms (GAP, BESTFIT, FASTA, BLAST P, BLAST N and TFASTA in Wisconsin Genetics Software Package, Genetics Computer Group, 575 Science Drive, Madison, Wis.).

Percentage identity is obtained by determining the number of identical positions at which the sequences to be compared correspond, dividing this number by the number of positions compared (e.g., the number of positions in the reference sequence) and multiplying this result by 100.

In some embodiments, the degree of identity is given for a region which is at least about 50%, at least about 60%, at least about 70%, at least about 80%, at least about 90% or about 100% of the entire length of the reference sequence. For example, if the reference nucleic acid sequence consists of 200 nucleotides, the degree of identity is given for at least about 100, at least about 120, at least about 140, at least about 160, at least about 180,or about 200 nucleotides, in some embodiments in continuous nucleotides. In some embodiments, the degree of identity is given for the entire length of the reference sequence.

Nucleic acid sequences or amino acid sequences having a particular degree of identity to a given nucleic acid sequence or amino acid sequence, respectively, may have at least one functional property of said given sequence, e.g., and in some instances, are functionally equivalent to said given sequence. One important property includes the ability to act as a cytokine, in particular when administered to a subject. In some embodiments, a nucleic acid sequence or amino acid sequence having a particular degree of identity to a given nucleic acid sequence or amino acid sequence is functionally equivalent to said given sequence.

As used herein, a “subject” refers to any member of the animal kingdom. In some embodiments, “subject” refers to humans. In some embodiments, “subject” refers to non-human animals. In some embodiments, subjects include, but are not limited to, mammals, birds, reptiles, amphibians, fish, insects, and/or worms. In certain embodiments, the non-human subject is a mammal (e.g., a rodent, a mouse, a rat, a rabbit, a monkey, a dog, a cat, a sheep, cattle, a primate, and/or a pig). In some embodiments, a subject may be a transgenic animal, genetically-engineered animal, and/or a clone. In certain embodiments of the present invention the subject is an adult, an adolescent or an infant. In some embodiments, terms “individual” or “patient” are used and are intended to be interchangeable with “subject”.

A “surface-exposed” amino acid, as used herein, refers to an amino acid residue in a protein (e.g., a ferritin) with a side chain that can be contacted by solvent molecules when the protein is in its native three-dimensional conformation after multimerization, if applicable. Thus, for example, in the case of ferritin that forms a 24-mer, a surface-exposed amino acid residue is one whose side chain can be contacted by solvent when the ferritin is assembled as a 24-mer, e.g., as a ferritin multimer or ferritin particle.

As used herein, the term “vaccination” or “vaccinate” refers to the administration of a composition intended to generate an immune response, for example to a disease-causing agent. Vaccination can be administered before, during, and/or after exposure to a disease-causing agent, and/or to the development of one or more symptoms, and in some embodiments, before, during, and/or shortly after exposure to the agent. In some embodiments, vaccination includes multiple administrations, appropriately spaced in time, of a vaccinating composition.

### B. OspA polypeptides

OspA polypeptides are provided, which can be antigenic when administered alone, with adjuvant as a separate molecule, and/or as part of a nanoparticle (e.g., ferritin particle or lumazine synthase particle), which can be self-adjuvanting. In some embodiments, an OspA polypeptide comprises a modified outer surface protein A (OspA) of Borrelia. OspA exists in a number of serotypes, as defined by their reactivity with monoclonal antibodies against different epitopes of OspA (see Wilske et al., J Clin Microbio 31(2):340-350 (1993)). These serotypes are correlated with different genospecies of Borrelia bacteria. In some embodiments, the OspA is any one of serotypes 1-7. In some embodiments, the OspA is from Borrelia burgdorferi, Borrelia mayonii, Borrelia afzelii, Borrelia garinii, or Borrelia bavariensis. In some embodiments, the OspA is Borrelia burgdorferi OspA. In some embodiments, the Borrelia can be carried by a tick of the Ixodes genus. In some embodiments, the Borrelia is Borrelia burgdorferi, Borrelia mayonii, Borrelia afzelii, Borrelia garinii, or Borrelia bavariensis.

In some embodiments, the OspA polypeptide is an OspA serotype 1 polypeptide, such as an OspA serotype 1 ectodomain. The literature has reported that an epitope of OspA serotype 1 at amino acids 165-173 of SEQ ID NO: 83 has homology with a fragment of the sequence of human leukocyte function-associated antigen-1 (hLFA-1)—i.e., SEQ ID NO: 78 (see Gross, D. M., et al., Science 281(5377): p. 703-6 (1998)). Amino acids 165-173 of SEQ ID NO: 83 are shown as an isolated nonapeptide in SEQ ID NO: 77 and are referred to as the hLFA-1 homology site. SEQ ID NO: 83 is an exemplary wild-type serotype 1 OspA sequence, which is used herein as a reference sequence for discussion of amino acid positions in OspA. This homology site may play a role in the development of Lyme arthritis, including antibiotic-resistant Lyme arthritis. Described herein are antigenic OspA polypeptides comprising a modified OspA serotype 1 polypeptide of Borrelia, wherein the modified OspA does not comprise the sequence of SEQ ID NO: 77. Such polypeptides, when used to elicit antibodies, may have improved safety, e.g., reduced risk of triggering an autoimmune response. In some embodiments, the OspA serotype 1 polypeptide has one or more modifications that reduce identity with hLFA-1. Any modification to reduce homology to SEQ ID NO: 78, to reduce identity to SEQ ID NO: 78, or to introduce one or more non-conservative substitutions relative to SEQ ID NO: 78 is encompassed. In some embodiments, an antigenic polypeptide comprising OspA serotype 1 polypeptide of Borrelia, wherein the polypeptide does not comprise the sequence of SEQ ID NO: 77 is provided. In some embodiments, the antigenic polypeptide comprises the ectodomain of OspA serotype 1, wherein the ectodomain does not comprise the sequence of SEQ ID NO: 77. In some embodiments, the antigenic OspA serotype 1 polypeptide comprises a sequence with at least 85%, 90%, 95%, 97%, 98%, 99%, 99.5%, or 100% identity to the sequence of any one of SEQ ID NOS: 94-102.

“Reducing homology” encompasses reducing sequence identity and/or reducing sequence similarity, wherein each member of a set of amino acids listed as conservative substitutions in the Table below is considered similar to the listed original residue and to the other members of the set; for example, the first line of the table indicates that alanine, valine, leucine, and isoleucine are similar to each other, and the eighth line indicates that alanine and glycine are similar to each other. Similarity is not transitive, so for example, isoleucine and glycine are not considered similar. In some embodiments, a modified OspA comprises an OspA serotype 1 protein with reduced homology to hLFA-1 compared to wild-type OspA serotype 1. In some embodiments, a modified OspA comprises an OspA serotype 1 comprising a modification to any one or more of the amino acids of SEQ ID NO: 77. In some embodiments, the modification to SEQ ID NO: 77 is a non-conservative amino acid substitution. A non-conservative substitution is a substitution different from the conservative substitutions shown in the following Table.

TABLE 3
Conservative Amino Acid Substitutions

| Original | Conservative |
|---|---|
| Residue | Substitutions |
| Ala (A) | Val; Leu; Ile |
| Arg (R) | Lys; Gln; Asn |
| Asn (N) | Gln; His; Asp, Lys; Arg |
| Asp (D) | Glu; Asn |
| Cys (C) | Ser; Ala |
| Gln (Q) | Asn; Glu |
| Glu (E) | Asp; Gln |
| Gly (G) | Ala |
| His (H) | Asn; Gln; Lys; Arg |
| Ile (I) | Leu; Val; Met; Ala; Phe; Norleucine |
| Leu (L) | Norleucine; Ile; Val; Met; Ala; Phe |
| Lys (K) | Arg; Gln; Asn |
| Met (M) | Leu; Phe; Ile |
| Phe (F) | Trp; Leu; Val; Ile; Ala; Tyr |
| Pro (P) | Ala |
| Ser (S) | Thr |
| Thr (T) | Val; Ser |
| Trp (W) | Tyr; Phe |
| Tyr (Y) | Trp; Phe; Thr; Ser |
| Val (V) | Ile; Leu; Met; Phe; Ala; Norleucine |

In some embodiments, one or more of the amino acids of SEQ ID NO: 77 is replaced with the corresponding amino acid(s) of a non-serotype 1 OspA, such as serotype 2,3, 4, 5, 6, or 7 OspA. In some embodiments, each of the amino acids of SEQ ID NO: 77 are replaced with the corresponding amino acid(s) of a serotype 2, 3, 4, 5, 6, or 7 OspA. In some embodiments, the amino acids of SEQ ID NO: 77 are replaced with corresponding amino acids of serotype 2 (S2, SEQ ID NO: 79) or serotype 3 (S3, SEQ ID NO: 80).

In some embodiments, a modified OspA comprises SEQ ID NO: 81. In some embodiments, a modified OspA comprises SEQ ID NO: 82. SEQ ID NOS: 81 and 82 are intended to replace SEQ ID NO: 77 and thereby reduce homology to SEQ ID NO: 78.

In some embodiments, the polypeptide is a full-length OspA (e.g., including a transmembrane domain and an ectodomain, which may or may not comprise a modification to reduce homology to hLFA-1 as described herein).

In some embodiments, the polypeptide lacks a transmembrane domain. In some embodiments, the polypeptide lacks a portion of a transmembrane domain, e.g., the N-terminal 2, 3, 4, 5, 6, 7, 8, 9, 10, 11, 12, 13, 14, 15, 16, 17, 18, 19, 20, 21, 22, 23, or 24 amino acids of a wild-type OspA sequence. In some embodiments, the polypeptide lacks a segment including amino acid 17 of OspA serotype 1 or the corresponding position of a homolog thereof as identified by pairwise or structural alignment. In some embodiments, the polypeptide lacks at least amino acids 1-17 of OspA, such as OspA serotype 1, or the counterpart amino acids in a homolog thereof as identified by pairwise or structural alignment. In some embodiments, the polypeptide lacks at least the N-terminal 18, 19, 20, 21, 22, 23, or 24 amino acids of OspA, such as OspA serotype 1, or the counterpart amino acids in a homolog thereof as identified by pairwise or structural alignment. In some embodiments, the polypeptide lacks amino acids 1-25 of OspA, such as OspA serotype 1, or the counterpart amino acids in a homolog thereof as identified by pairwise or structural alignment. In some embodiments, the polypeptide lacks amino acids 1-26 of OspA serotype 1, or the counterpart amino acids in a homolog thereof as identified by pairwise or structural alignment. For the avoidance of doubt, lacking a transmembrane domain does not require that a polypeptide lack an N-terminal methionine; for example, a polypeptide in which the first residue is methionine and the second residue corresponds to residue 26 of a wild-type OspA, followed by residues corresponding to the 27th, 28th, etc., wild-type OspA residues, is considered to lack a transmembrane domain. In some embodiments, the polypeptide comprising an OspA lacks a lipidation site, such as the lipidation site contained within the transmembrane domain of wild-type OspA serotype 1. In some embodiments, the polypeptide lacks cysteine 17 of OspA serotype 1. In some embodiments, the polypeptide does not comprise a cysteine that corresponds to any of positions 1-25 of a wild-type OspA, e.g., any of SEQ ID NOs: 83-89. In some embodiments, the polypeptide lacks or has a substitution at cysteine 17 of OspA serotype 1. In some embodiments, the polypeptide lacks at least part of a wild-type OspA transmembrane domain, such that it lacks a lipidation site. In some embodiments, the polypeptide lacks amino acids that align to amino acids 1-17 of OspA serotype 1.

In some embodiments, the polypeptide does not comprise a palmitoyl group. In some embodiments, the polypeptide does not comprise a diacylglycerol group. In some embodiments, the polypeptide is non-lipidated. In some embodiments, the polypeptide lacks a lipidation site. In some embodiments, this lipidation site is contained within the transmembrane domain. In some embodiments, the lipidation site that is removed is cysteine 17 of OspA serotype 1. In some embodiments, the polypeptide lacks or has a substitution at cysteine 17 of OspA serotype 1.

In some embodiments, removal of an OspA lipidation site and/or transmembrane domain or portion thereof, and/or the lack of a palmitoyl and/or diacylglycerol group, allows easier protein purification, e.g., by improving the solubility of the protein and/or making the protein more amenable to purification by techniques such as ion exchange and other forms of chromatography.

In some embodiments, the polypeptide comprises a mammalian leader sequence (also known as a signal sequence). In some embodiments, the mammalian leader sequence results in secretion of the polypeptide when expressed in mammalian cells.

In some embodiments, the polypeptide lacks a glycosylation site. Modifications to remove glycosylation sites are described in detail herein. The OspA polypeptides according to this disclosure can comprise any such modification, which can be combined with any of the other modifications described herein, including modifications to the hLFA-1 homology site and/or deletion of part or all of a transmembrane domain. In some embodiments, the polypeptide does not comprise SEQ ID NO: 77 (e.g., has reduced homology to hLFA-1a) and has modifications to reduce glycosylation and/or lacks a transmembrane domain.

### 1. Modification of Glycosylation

N-linked glycosylation is the attachment of glycan to an amide nitrogen of an asparagine (Asn; N) residue of a protein. The process of attachment results in a glycosylated protein. Glycosylation can occur at any asparagine residue in a protein that is accessible to and recognized by glycosylating enzymes following translation of the protein, and is most common at accessible asparagines that are part of an NXS/TX site, wherein the second amino acid residue following the asparagine is a serine or threonine. A non-human glycosylation pattern can render a polypeptide undesirably reactogenic when used to elicit antibodies. Additionally, glycosylation of a polypeptide that is not normally glycosylated can alter its immunogenicity. For example, glycosylation can mask important immunogenic epitopes within a protein. Thus, to reduce or eliminate glycosylation, either asparagine residues or serine/threonine residues can be modified, for example, by substitution to another amino acid.

In some embodiments, a polypeptide comprising an OspA is modified to reduce or eliminate glycosylation. In some embodiments, one or more N-glycosylation sites in OspA are removed. In some embodiments, the removal of an N-glycosylation site decreases glycosylation of OspA. In some embodiments, the polypeptide has decreased glycosylation relative to wild-type OspA, such as wild-type serotype 1 OspA. In some embodiments, the removal of N-glycosylation sites eliminates glycosylation of OspA.

In some embodiments, one or more asparagines in OspA are replaced with a non-asparagine amino acid. In some embodiments, each asparagine in OspA is replaced with a non-asparagine amino acid. Any natural or non-natural amino acid found in proteins, e.g., glutamine, may be used to replace asparagine. In some embodiments, the modification to reduce or eliminate glycosylation modifies an NXS/TX glycosylation site (wherein the second residue following the N is an S or T). In some embodiments, the first X in the NXS/TX site is not proline and/or the second X in the NXS/TX site is not proline. In some embodiments, the modification to reduce or eliminate glycosylation is an N to Q substitution. In some embodiments, the modification to reduce or eliminate glycosylation is an S/T to A substitution.

A detailed discussion of positions that can be modified to reduce or eliminate glycosylation below. Position numbers refer to the positions in full-length OspA sequences provided as SEQ ID NOs: 83-89. It is understood that position numbers should be adjusted appropriately for partial and modified OspA sequences (e.g., if an N-terminal deletion results in a net shortening by 25 amino acid residues, then position numbers should be decremented by 25).

In some embodiments, the modification to reduce or eliminate glycosylation comprises a substitution of any one or more of N20, N71, N190, N202, and N251 of OspA serotype 1 (SEQ ID NO: 83). In some embodiments, the modification comprises modifications at each of N71, N190, N202, and N251 of OspA serotype 1. In some embodiments, the modification to reduce or eliminate glycosylation comprises one or more of N20Q, N71Q, N190Q, N202Q, or N251Q of OspA serotype 1. Corresponding amino acids can be found in OspA of different serotypes by pair-wise alignment. Thus, in some embodiments, the asparagine residues replaced in OspA of serotypes 2-7 are amino acid residues that align with N20, N71, N190, N202, and N251 of OspA serotype 1. In some embodiments, the modification to reduce or eliminate glycosylation comprises a substitution of any one or more of a Ser or Thr residue at position 22, 73, 192, 204, and 253 of OspA serotype 1. In some embodiments, the modification comprises a substitution of one or more of a Ser or Thr residue at position 22, 73, 192, 204, and 253 of OspA serotype 1 with an alanine.

In some embodiments, the modification to reduce or eliminate glycosylation comprises substitutions any one or more of N20, N71, N141, N164, N202, and N205 of OspA serotype 2 (SEQ ID NO: 84). In some embodiments, the modification comprises modifications at each of N20, N71, N141, N164, N202, and N205 of OspA serotype 2. In some embodiments, the modification to reduce or eliminate glycosylation comprises one or more of N20Q, N71Q, N141Q, N164Q, N202Q, or N205Q of OspA serotype 2. Analogous amino acids can be found in OspA of different serotypes by pair-wise alignment. Thus, in some embodiments, the asparagine residues replaced in OspA of serotypes 1 or 3-7 are amino acid residues that align with N20, N71, N141, N164, N202, and N205 of OspA serotype 2. In some embodiments, the modification to reduce or eliminate glycosylation comprises a substitution of any one or more of a Ser or Thr residue at position 22, 73, 143, 166, 204, and 207 of OspA serotype 2. In some embodiments, the modification comprises a substitution of one or more of a Ser or Thr residue at position 22, 73, 143, 166, 204, and 207 of OspA serotype 2 with an alanine.

In some embodiments, the modification to reduce or eliminate glycosylation comprises substitutions of any one or more of N20, N71, N95, N141, N191, and N203 of OspA serotype 3 (SEQ ID NO: 85). In some embodiments, the modification comprises modifications at each of N20, N20, N71, N95, N141, N191, and N203 of OspA serotype 3. In some embodiments, the modification to reduce or eliminate glycosylation comprises one or more of N20Q, N71Q, N95Q, N141Q, N191Q, or N203Q of OspA serotype 3. Analogous amino acids can be found in OspA of different serotypes by pair-wise alignment. Thus, in some embodiments, the asparagine residues replaced in OspA of serotypes 1-2 or 4-7 are amino acid residues that align with N20, N20, N71, N95, N141, N191, and N203 of OspA serotype 3. In some embodiments, the modification to reduce or eliminate glycosylation comprises a substitution of any one or more of a Ser or Thr residue at position 22, 73, 97, 143, 193, and 205 of OspA serotype 3. In some embodiments, the modification comprises a substitution of one or more of a Ser or Thr residue at position 22, 73, 97, 143, 193, and 205 of OspA serotype 3 with an alanine.

In some embodiments, the modification to reduce or eliminate glycosylation comprises substitutions of any one or more of N20, N71, N141, N202, N205, and N219 of OspA serotype 4 (SEQ ID NO: 86). In some embodiments, the modification comprises modifications at each of N20, N71, N141, N202, N205, and N219 of OspA serotype 4. In some embodiments, the modification to reduce or eliminate glycosylation comprises one or more of N20Q, N71Q, N141Q, N202Q, N205Q, or N219Q of OspA serotype 4. Analogous amino acids can be found in OspA of different serotypes by pair-wise alignment. Thus, in some embodiments, the asparagine residues replaced in OspA of serotypes 1-3 or 5-7 are amino acid residues that align with N20, N71, N141, N202, N205, and N219 of OspA serotype 4. In some embodiments, the modification to reduce or eliminate glycosylation comprises a substitution of any one or more of a Ser or Thr residue at position 22, 73, 143, 204, 207, and 221 of OspA serotype 4. In some embodiments, the modification comprises a substitution of one or more of a Ser or Thr residue at position 22, 73, 143, 204, 207, and 221 of OspA serotype 4 with an alanine.

In some embodiments, the modification to reduce or eliminate glycosylation comprises substitutions of any one or more of N20, N71, and N141 of OspA serotype 5.(Certain serotypes, including serotypes 5-7, contain fewer glycosylation sites than certain other OspA sequences such as serotype 1). In some embodiments, the modification comprises modifications at each of N20, N71, and N141 of OspA serotype 5 (SEQ ID NO: 87). In some embodiments, the modification to reduce or eliminate glycosylation comprises one or more of N20Q, N71Q, or N141Q of OspA serotype 5. Analogous amino acids can be found in OspA of different serotypes by pair-wise alignment. Thus, in some embodiments, the asparagine residues replaced in OspA of serotypes 1-4 or 6-7 are amino acid residues that align with N20, N71, and N141 of OspA serotype 5. In some embodiments, the modification to reduce or eliminate glycosylation comprises a substitution of any one or more of a Ser or Thr residue at position 22, 73, and 143 of OspA serotype 5. In some embodiments, the modification comprises a substitution of one or more of a Ser or Thr residue at position 22, 73, and 143 of OspA serotype 5 with an alanine.

In some embodiments, the modification to reduce or eliminate glycosylation comprises substitutions of any one or more of N20, N71, and N141 of OspA serotype 6 (SEQ ID NO: 88). In some embodiments, the modification comprises modifications at each of N20, N71, and N141 of OspA serotype 6. In some embodiments, the modification to reduce or eliminate glycosylation comprises one or more of N20Q, N71Q, or N141Q of OspA serotype 6. Analogous amino acids can be found in OspA of different serotypes by pair-wise alignment. Thus, in some embodiments, the asparagine residues replaced in OspA of serotypes 1-5 or 7 are amino acid residues that align with N20, N71, and N141 of OspA serotype 6. In some embodiments, the modification to reduce or eliminate glycosylation comprises a substitution of any one or more of a Ser or Thr residue at position 22, 73, and 143 of OspA serotype 6. In some embodiments, the modification comprises a substitution of one or more of a Ser or Thr residue at position 22, 73, and 143 of OspA serotype 6 with an alanine.

In some embodiments, the modification to reduce or eliminate glycosylation comprises substitutions of any one or more of N20, N71, N141, and N191 of OspA serotype 7 (SEQ ID NO: 89). In some embodiments, the modification comprises modifications at each of N20, N71, N141, and N191 of OspA serotype 7. In some embodiments, the modification to reduce or eliminate glycosylation comprises one or more of N20Q, N71Q, N141Q, or N191Q of OspA serotype 7. Analogous amino acids can be found in OspA of different serotypes by pair-wise alignment. Thus, in some embodiments, the asparagine residues replaced in OspA of serotypes 1-6 are amino acid residues that align with N20, N71, N141, and N191 of OspA serotype 7. In some embodiments, the modification to reduce or eliminate glycosylation comprises a substitution of any one or more of a Ser or Thr residue at position 22, 73, 143, and 193 of OspA serotype 7. In some embodiments, the modification comprises a substitution of one or more of a Ser or Thr residue at position 22, 73, 143, and 193 of OspA serotype 7 with an alanine.

### C. Antigenic OspA Polypeptides Comprising an OspA Polypeptide and Ferritin or Lumazine Synthase

In some embodiments, an antigenic polypeptide is provided, comprising an OspA polypeptide and ferritin or lumazine synthase. It is to be understood that such antigenic OspA polypeptides disclosed herein are antigenic with respect to the OspA component, e.g., they can be administered to a mammal, such as a human, to elicit the production of anti-OspA antibodies. The ferritin component of the antigenic polypeptide may be wild type ferritin of any species, or ferritin of any species which comprises one or more mutations, both as described herein. In some embodiments, the antigenic polypeptide comprises the amino acids of any one of SEQ ID NOS: 1-76. In some embodiments, the antigenic polypeptide comprises a sequence with at least 85%, 90%, 95%, 97%, 98%, 99%, 99.5%, or 100% identity to the sequence of any one of SEQ ID NOS: 1-76. The lumazine synthase component of the antigenic polypeptide may be lumazine synthase of any species.

In some embodiments, the antigenic polypeptide comprises a mammalian leader sequence (also known as a signal sequence). In some embodiments, the mammalian leader sequence results in secretion of the antigenic polypeptide when expressed in mammalian cells.

### 1. OspA Polypeptide Component

The antigenic OspA polypeptide can comprise any of the modified OspA polypeptides described herein.

For example, in some embodiments, the OspA component of the antigenic OspA polypeptide comprises the ectodomain of OspA. In some embodiments, the ectodomain of OspA is from any one of serotypes 1, 2, 3, 4, 5, 6, or 7. Exemplary wild-type OspA sequences are provided as SEQ ID NOS: 83-89. The N-terminal 25 amino acids thereof can be removed to, for example, remove the lipidation site. Accession numbers for exemplary OspA sequences are also provided in the examples. In some embodiments, the ectodomain is from Borrelia burgdorferi, Borrelia mayonii, Borrelia afzelii, Borrelia garinii, or Borrelia bavariensis. In some embodiments, the ectodomain is Borrelia burgdorferi.

In some embodiments, the ectodomain is a wild-type ectodomain.

In some embodiments, the ectodomain is a modified ectodomain, e.g., an ectodomain modified to reduce glycosylation. Introductory discussion of glycosylation is provided in the previous section and is not repeated here in the interest of brevity. The OspA component of the antigenic OspA polypeptide can comprise any of the modifications described herein to reduce or eliminate glycosylation, including without limitation removal of one or more N-glycosylation sites in the ectodomain are removed, e.g., by amino acid substitutions, as discussed in detail herein.

In some embodiments, the OspA ectodomain is present as part of a larger OspA sequence in the antigenic polypeptide, e.g., including part or all of a transmembrane domain. In some embodiments, the OspA ectodomain is present in a full-length OspA sequence within the antigenic polypeptide. In some embodiments, the full-length OspA sequence is a wild-type full-length OspA sequence. In some embodiments, the antigenic polypeptide does not comprise a transmembrane domain.

Any of the modifications discussed above can be combined in the antigenic polypeptide. In some embodiments, the antigenic polypeptide comprises at least one modification to reduce glycosylation and at least one further modification described herein.

In some embodiments, the OspA ectodomain of the antigenic polypeptide is any one of the modified OspA ectodomains described in section B above.

Any of the OspA ectodomains (or the sequence comprising the OspA ectodomain, such as full-length OspA) described herein can be combined in the antigenic polypeptide with any of the ferritins described below.

### 2. Ferritin Component

In some embodiments, a fusion protein comprising the antigenic OspA polypeptide described herein and ferritin is encompassed. The ferritin in the antigenic polypeptide can be wild-type or comprise one or more mutations (see the following section). In some embodiments, the ferritin is bacterial, insect, fungal, bird, or mammalian. In some embodiments, the ferritin is human. In some embodiments, the ferritin is bacterial. In some embodiments, the ferritin is Trichoplusia ni ferritin (PDB: 1Z60_X, SEQ ID NOS: 211 and 212).

In some embodiments, the ferritin is a light chain and/or heavy chain ferritin. In some embodiments, the ferritin is human heavy chain ferritin (FTH1, GENE ID No: 2495) or human light chain ferritin (FTL, GENE ID No: 2512). In some embodiments, the ferritin is a multimeric protein referred to herein as a “ferritin particle” or a “ferritin nanoparticle” comprising 24 total subunits of heavy chain ferritin and light chain ferritin.

In some embodiments, an antigenic OspA polypeptide comprises a light chain ferritin and an OspA polypeptide. In some embodiments, an antigenic polypeptide comprises a heavy chain ferritin and an OspA polypeptide. In some embodiments, an antigenic polypeptide comprising a light chain ferritin and an OspA polypeptide can assemble with a heavy chain ferritin that is not linked to a non-ferritin polypeptide. In some embodiments, an antigenic polypeptide comprising a heavy chain ferritin and an OspA polypeptide can assemble with a light chain ferritin that is not linked to a non-ferritin polypeptide. A ferritin not linked to a non-ferritin polypeptide may be referred as a “naked ferritin.”

In some embodiments, an antigenic polypeptide comprising a heavy chain ferritin and an OspA polypeptide can assemble with an antigenic polypeptide comprising a light chain ferritin and an OspA polypeptide to allow expression of 2 of the same or different antigens on a single ferritin particle. In some embodiments, the 2 different antigens are encoded by a single infectious agent. In some embodiments, the 2 different antigens are encoded by 2 different infectious agents, e.g., Borrelia of different serotypes or species.

In some embodiments, an antigenic polypeptide comprising a heavy chain ferritin and an OspA polypeptide can assemble with an antigenic polypeptide comprising a light chain ferritin and an OspA polypeptide to produce a bivalent composition. In some embodiments, the ferritin is H. pylori ferritin (see SEQ ID NO: 90 for an exemplary wild-type H. pylori ferritin sequence comprising an 8-amino acid extension from bullfrog ferritin at its N-terminus). In some embodiments, the lower sequence homology between H. pylori ferritin (or other bacterial ferritins) and human ferritin may decrease the potential for autoimmunity when used as a platform for constructing antigenic polypeptides (see Kanekiyo et al., Cell 162, 1090-1100 (2015)).

In some embodiments, the ferritin is Pyrococcus furiosus ferritin (NCBI seq WP_011011871.1), optionally with one or more mutations described herein.

In some embodiments, the ferritin comprises a sequence having greater than 70%, greater than 75%, greater than 80%, greater than 85%, greater than 90%, greater than 95%, greater than 97%, greater than 98%, or greater than 99% identity to a wild-type ferritin.

In some embodiments, the ferritin is an insect ferritin. In some embodiments, the ferritin is Trichoplusia Ni ferritin (PDB: 1Z60_X, SEQ ID NOS: 211 and 212), optionally with one or more mutations described herein.

### a) Ferritin Mutations

In some embodiments, the ferritin comprises one or more mutations are disclosed herein. In some embodiments, the one or more mutations comprise changes to the amino acid sequence of a wild-type ferritin and/or an insertion, e.g., at the N- or C-terminus. In some embodiments, one, two, three, four, five, or more different amino acids are mutated in the ferritin as compared to wild-type ferritin (in some embodiments, in addition to any N-terminal insertion). The one or more mutations can change functional properties of the ferritin, e.g., as discussed in detail below. In general, a mutation simply refers to a difference in the sequence (such as a substituted, added, or deleted amino acid residue or residues) relative to the corresponding wild-type ferritin.

### (1) Cysteine for Conjugation

In some embodiments, ferritin is mutated to provide a chemical handle for conjugation of an immune-stimulatory moiety and/or non-ferritin polypeptide. This can be achieved with a mutation replacing a surface-exposed non-cysteine amino acid with a cysteine. For the avoidance of doubt, language such as “replacing a surface-exposed amino acid with a cysteine” necessarily implies that the surface-exposed amino acid in the wild-type or pre-mutation sequence is not cysteine. Another approach for providing a chemical handle for conjugation of an immune-stimulatory moiety or non-ferritin polypeptide is to include a segment of amino acids, such as a linker, N- or C-terminal to the ferritin, wherein the segment of amino acids comprises a cysteine. In some embodiments, this cysteine (whether replacing a surface-exposed amino acid or in an N- or C-terminal linker) is unpaired, which means that it does not have an appropriate partner cysteine to form a disulfide bond. In some embodiments, this cysteine does not change the secondary structure of ferritin. In some embodiments, this cysteine does not change the tertiary structure of ferritin.

In some embodiments, this cysteine can be used to conjugate agents, such as immune-stimulatory moieties, to ferritin. In some embodiments, this cysteine provides a free thiol group that is reactive. In some embodiments, agents conjugated to this cysteine on ferritin are exposed on the surface of an assembled ferritin particle. In some embodiments, this cysteine can interact with molecules and cells of the subject after administration while the ferritin particle is assembled.

In some embodiments, the presence of this cysteine allows conjugation of one or more immune-stimulatory moieties, e.g., adjuvants. In some embodiments, conjugation of the immune-stimulatory moiety would not occur in the absence of this cysteine.

In some embodiments, the non-cysteine amino acid that is replaced with a cysteine is selected from E12, S72, A75, K79, S100, and S111 of H. pylori ferritin. Thus, in some embodiments, the surface-exposed amino acid that is replaced in favor of cysteine is an amino acid residue that corresponds to E12, S26, S72, A75, K79, S100, or S111 of H. pylori ferritin. Analogous amino acids can be found in non-H. pylori ferritin by pair-wise or structural alignment. In some embodiments, the non-cysteine amino acid that is replaced with a cysteine can be selected from an amino acid that corresponds to S3, S19, S33, I82, A86, A102, and A120 of human light chain ferritin. In some embodiments, the surface-exposed amino acid to be replaced with a cysteine is selected based on the understanding that if the native amino acid were replaced with cysteine, it would be reactive in an assembled ferritin multimer or particle and/or that this cysteine does not disrupt the stability of the ferritin multimer or particle and/or that this cysteine does not lead to reduction in expression levels of ferritin.

In some embodiments, the ferritin comprises an E12C mutation. In some embodiments, the E12C residue can be used to conjugate agents (e.g., immune-stimulatory moieties and/or non-ferritin polypeptides) to ferritin. In some embodiments, the E12C residue provides a free thiol group that is reactive. In some embodiments, agents conjugated to the E12C residue on ferritin monomers are expressed on the surface on an assembled ferritin multimer or particle. In some embodiments, twenty-four E12C residues (one from each monomer) are present on the surface of a ferritin multimer or particle.

In some embodiments, the ferritin comprises an S26C mutation. In some embodiments, the S26C residue can be used to conjugate agents (e.g., immune-stimulatory moieties and/or non-ferritin polypeptides) to ferritin. In some embodiments, the S26C residue provides a free thiol group that is reactive. In some embodiments, agents conjugated to the S26C residue on ferritin monomers are expressed on the surface on an assembled ferritin multimer or particle. In some embodiments, twenty-four S26C residues (one from each monomer) are present on the surface of a ferritin multimer or particle.

In some embodiments, the ferritin comprises an S72C mutation. In some embodiments, the S72C residue can be used to conjugate agents (e.g., immune-stimulatory moieties and/or non-ferritin polypeptides) to ferritin. In some embodiments, the S72C residue provides a free thiol group that is reactive. In some embodiments, agents conjugated to the S72C residue on ferritin monomers are expressed on the surface on an assembled ferritin multimer or particle. In some embodiments, twenty-four S72C residues (one from each monomer) are present on the surface of a ferritin multimer or particle.

In some embodiments, the ferritin comprises an A75C mutation. In some embodiments, the A75C residue can be used to conjugate agents (e.g., immune-stimulatory moieties and/or non-ferritin polypeptides) to ferritin. In some embodiments, the A75C residue provides a free thiol group that is reactive. In some embodiments, agents conjugated to the A75C residue on ferritin monomers are expressed on the surface on an assembled ferritin multimer or particle. In some embodiments, twenty-four A75C residues (one from each monomer) are present on the surface of a ferritin multimer or particle.

In some embodiments, the ferritin comprises an K79C mutation. In some embodiments, the K79C residue can be used to conjugate agents (e.g., immune-stimulatory moieties and/or non-ferritin polypeptides) to ferritin. In some embodiments, the K79C residue provides a free thiol group that is reactive. In some embodiments, agents conjugated to the K79C residue on ferritin monomers are expressed on the surface on an assembled ferritin multimer or particle. In some embodiments, twenty-four K79C residues (one from each monomer) are present on the surface of a ferritin multimer or particle.

In some embodiments, the ferritin comprises an S100C mutation. In some embodiments, the S100C residue can be used to conjugate agents (e.g., immune-stimulatory moieties and/or non-ferritin polypeptides) to ferritin. In some embodiments, the S100C residue provides a free thiol group that is reactive. In some embodiments, agents conjugated to the S100C residue on ferritin monomers are expressed on the surface on an assembled ferritin multimer or particle. In some embodiments, twenty-four S100C residues (one from each monomer) are present on the surface of a ferritin multimer or particle.

In some embodiments, the ferritin comprises an S111C mutation. In some embodiments, the S111C residue can be used to conjugate agents (e.g., immune-stimulatory moieties and/or non-ferritin polypeptides) to ferritin. In some embodiments, the S111C residue provides a free thiol group that is reactive. In some embodiments, agents conjugated to the S111C residue on ferritin monomers are expressed on the surface on an assembled ferritin multimer or particle. In some embodiments, twenty-four S111C residues (one from each monomer) are present on the surface of a ferritin multimer or particle.

### (2) Removal of Internal Cysteine

In some embodiments, the ferritin comprises a mutation replacing an internal cysteine with a non-cysteine amino acid. Removal of a native internal cysteine residue can ensure that there is only one unpaired cysteine per ferritin monomer and avoid undesired reactions such as disulfide formation and may result in a more stable and efficient result (e.g., adjuvant presentation). In some embodiments, C31 of H. pylori ferritin is replaced with a non-cysteine amino acid. In some embodiments, C31 of H. pylori ferritin is replaced with a serine (C31S), although any non-cysteine residue may be used, e.g., alanine, glycine, threonine, or asparagine. Analogous amino acids can be found in non-H. pylori ferritin by pair-wise or structural alignment. Thus, in some embodiments, the internal cysteine that is replaced in favor of non-cysteine is an amino acid residue that aligns with C31 of H. pylori ferritin. Exemplary ferritin sequences showing a C31S mutation are shown in SEQ ID NOS: 201-207. In some embodiments, when more than one internal cysteine is present in ferritin, two or more (e.g., each) internal cysteine is replaced with a non-cysteine amino acid, such as serine or an amino acid selected from serine, alanine, glycine, threonine, or asparagine.

### (3) Glycosylation

Human-compatible glycosylation can contribute to safety and efficacy in recombinant drug products. Regulatory approval may be contingent on demonstrating appropriate glycosylation as a critical quality attribute (see Zhang et al., Drug Discovery Today 21(5):740-765 (2016)). N-glycans can result from glycosylation of asparagine side chains and can differ in structure between humans and other organisms such as bacteria and yeast. Thus, it may be desirable to reduce or eliminate non-human glycosylation and/or N-glycan formation in ferritin according to the disclosure. In some embodiments, controlling glycosylation of ferritin improves the efficacy and/or safety of the composition, especially when used for human vaccination.

In some embodiments, ferritin is mutated to inhibit formation of an N-glycan. In some embodiments, a mutated ferritin has reduced glycosylation as compared to its corresponding wild type ferritin.

In some embodiments, the ferritin comprises a mutation replacing a surface-exposed asparagine with a non-asparagine amino acid. In some embodiments, the surface-exposed asparagine is N19 of H. pylori ferritin or a position that corresponds to position 31 of H. pylori ferritin as determined by pair-wise or structural alignment In some embodiments, mutating such an asparagine, e.g., N19 of H. pylori ferritin, decreases glycosylation of ferritin. In some embodiments, the mutation replaces the asparagine with a glutamine. In some embodiments, the ferritin is an H. pylori ferritin comprising an N19Q mutation. SEQ ID NOS: 201-207 are exemplary ferritin sequences comprising N19Q mutations.

A mammal exposed to a glycosylated protein produced in bacteria or yeast may generate an immune response to the glycosylated protein, because the pattern of glycosylation of a given protein in bacterial or yeast could be different from the pattern of glycosylation of the same protein in a mammal. Thus, some glycosylated therapeutic proteins may not be appropriate for production in bacteria or yeast.

In some embodiments, decreased glycosylation of ferritin by amino acid mutation facilitates protein production in bacteria or yeast. In some embodiments, decreased glycosylation of ferritin reduces the potential for adverse effects in mammals upon administration of mutated ferritin that is expressed in bacteria or yeast. In some embodiments, the reactogenicity in a human subject of a mutated ferritin produced in bacteria or yeast is lower because glycosylation is decreased. In some embodiments, the incidence of hypersensitivity responses in human subjects is lower following treatment with a mutated ferritin with reduced glycosylation compared to wildtype ferritin.

In some embodiments, degradation in a subject of a composition comprising a mutated ferritin with reduced glycosylation is slower compared with a composition comprising a wild-type ferritin, or a composition comprising a corresponding ferritin with wild-type glycosylation. In some embodiments, a composition comprising a mutated ferritin with reduced glycosylation has reduced clearance in a subject compared with a composition comprising a wild-type ferritin, or a composition comprising a corresponding ferritin with wild-type glycosylation. In some embodiments, a composition comprising a mutated ferritin with reduced glycosylation has a longer-serum half-life compared to wild-type ferritin, or a composition comprising a corresponding ferritin with wild-type glycosylation.

### (4) Combinations of Mutations

In some embodiments, a ferritin comprises more than one type of mutation described herein. In some embodiments, the ferritin comprises one or more mutations independently selected from: a mutation to decrease glycosylation, a mutation to remove an internal cysteine, and a mutation to generate a surface-exposed cysteine. In some embodiments, the ferritin comprises a mutation to decrease glycosylation, a mutation to remove an internal cysteine, and a mutation to generate a surface-exposed cysteine.

In some embodiments, the ferritin comprises an N19Q mutation, a C31S mutation, and a mutation to generate a surface-exposed cysteine. In some embodiments, the ferritin comprises an N19Q mutation, a C31S mutation, and an E12C mutation. In some embodiments, the ferritin comprises an N19Q mutation, a C31S mutation, and an S72C mutation. In some embodiments, the ferritin comprises an N19Q mutation, a C31S mutation, and an A75C mutation. In some embodiments, the ferritin comprises an N19Q mutation, a C31S mutation, and an K79C mutation. In some embodiments, the ferritin comprises an N19Q mutation, a C31S mutation, and an S100C mutation. In some embodiments, the ferritin comprises an N19Q mutation, a C31S mutation, and an S111C mutation. In some embodiments, the ferritin comprises mutations corresponding to any of the foregoing sets of mutations, wherein the corresponding mutations change an N to a Q, a C to an S, and a non-cysteine surface-exposed amino acid to a cysteine at positions determined by pair-wise alignment of the ferritin amino acid sequence to an H. pylori ferritin amino acid sequence (SEQ ID NO: 208 OR 209).

Exemplary ferritins comprising more than one type of mutation are provided in SEQ ID NOS: 201-207.

### 3. Structural Alignment

As discussed herein, positions of mutations corresponding to those described with respect to a given polypeptide (e.g, H. pylori ferritin) can be identified by pairwise or structural alignment. Structural alignment is relevant to large protein families such as ferritin where the proteins share similar structures despite considerable sequence variation and many members of the family have been structurally characterized, and can also be used to identify corresponding positions in different versions of other polypeptides described herein, such as Borrelia polypeptides (e.g., OspA). The protein databank (PDB) comprises 3D structures for many ferritins, including those listed below with their accession numbers.

2jd6, 2jd7-PfFR-Pyrococcus furiosus. 2jd8-PfFR+Zn. 3a68-soFR from gene SferH4-soybean. 3a9q-soFR from gene SferH4 (mutant). 3egm, 3bvf, 3bvi, 3bvk, 3bvl-HpFR-Heliobacter pylori. 5c6f-HpFR (mutant)+Fe. 1z4a, 1v1g-FR-Thermotoga maritime. 1s3q, 1sq3, 3kx9-FR-Archaeoglubus fulgidus. 1krq-FR-Campylobacter jejuni. 1eum-EcFR-Escherichia coli. 4reu-EcFR+Fe. 4xgs-EcFR (mutant)+Fe202. 4ztt-EcFR (mutant)+Fe2O+Fe2+Fe+O2. 1qgh-LiFR-Listeria innocua. 3qz3-VcFR-Vibrio cholerae. 3vnx-FR-Ulva pertusa. 4ism, 4isp, 4itt, 4itw, 4iwj, 4iwk, 4ixk, 3e6s-PnmFR-Pseudo-nitschia multiseries. 4zkh, 4zkw, 4zkx, 4zl5, 4zl6, 4zlw, 4zmc-PnmFR (mutant)+Fe. 1z6o-FR-Trichoplusia ni. 4cmy-FR+Fe-Chlorobaculum tepidum. Ferritin light chain (FTL). 1lb3, 1h96-mFTL-mouse. 1rcc, 1rcd, 1rci-bFTL+tartrate+Mg. 1rce, 1rcg-bFTL+tartrate+Mn. 3noz, 3np0, 3np2, 3o7r-hoFTL (mutant)-horse. 3o7s, 3u90-hoFTL. 4v1w-hoFTL-cryo EM. 3rav, 3rd0-hoFTL+barbiturate. Ferritin light+heavy chains: 5gn8-hFTH +Ca.

Structural alignment involves identifying corresponding residues across two (or more) polypeptide sequences by (i) modeling the structure of a first sequence using the known structure of the second sequence or (ii) comparing the structures of the first and second sequences where both are known, and identifying the residue in the first sequence most similarly positioned to a residue of interest in the second sequence. Corresponding residues are identified in some algorithms based on alpha-carbon distance minimization in the overlaid structures (e.g., what set of paired alpha carbons provides a minimized root-mean-square deviation for the alignment). When identifying positions in a non-H. pylori ferritin corresponding to positions described with respect to H. pylori ferritin, H. pylori ferritin can be the “second” sequence. Where a non-H. pylori ferritin of interest does not have an available known structure, but is more closely related to another non-H. pylori ferritin that does have a known structure than to H. pylori ferritin, it may be most effective to model the non-H. pylori ferritin of interest using the known structure of the closely related non-H. pylori ferritin, and then compare that model to the H. pylori ferritin structure to identify the desired corresponding residue in the ferritin of interest. There is an extensive literature on structural modeling and alignment; representative disclosures include U.S. Pat. Nos. 6,859,736; 8,738,343; and those cited in Aslam et al., Electronic Journal of Biotechnology 20 (2016) 9-13. For discussion of modeling a structure based on a known related structure or structures, see, e.g., Bordoli et al., Nature Protocols 4 (2009) 1-13, and references cited therein.

### 4. Lumazine Synthase

In some embodiments, the antigenic polypeptide comprises a lumazine synthase protein (see Ra et al., Clin Exp Vaccine Res 3:227-234 (2014)). In some embodiments, this protein is lumazine synthase serotype 1, 2, 3, 4, 5, 6, or 7. Exemplary lumazine synthase sequences are provided as SEQ ID NO: 216 and 219. In some embodiments, the lumazine synthase comprises a sequence with 80%, 85%, 90%, 95%, 98%, or 99% identity to SEQ ID NO: 216 or 219. Lumazine synthases can form higher-order structures, e.g., a 60-subunit lumazine synthase particle. Exemplary lumazine synthases are Aquifex aeolicus lumazine synthase and E. coli lumazine synthase. The lumazine synthase can be located C-terminal to the OspA (e.g., the sequence comprising the OspA ectodomain, such as full-length OspA) and can be separated from the OspA by a linker as discussed herein. Exemplary antigenic polypeptides comprising a lumazine synthase protein are SEQ ID NOS: 12-21. In some embodiments, the antigenic polypeptide comprises a sequence with 80%, 85%, 90%, 95%, 98%, or 99% identity to any one of SEQ ID NOs: 12-21.

### 5. Linker

In some embodiments, ferritin or lumazine synthase is joined (e.g., fused) to the OspA (e.g., the sequence comprising the OspA ectodomain, such as full-length OspA) via a linker.

In some embodiments, a linker separates the amino acid sequence of a non-ferritin polypeptide (e.g., OspA) from the amino acid sequence of ferritin. Any linker may be used. In some embodiments, the linker is a peptide linker, which can facilitate expression of the antigenic ferritin polypeptide as a fusion protein (e.g., from a single open reading frame). In some embodiments, the linker is a glycine-serine linker. In some embodiments, the glycine-serine linker is GS, GGGS (SEQ ID NO: 226), 2XGGGS (SEQ ID NO: 91) (i.e., GGGSGGGS (SEQ ID NO: 91)), or 5XGGGS (SEQ ID NO: 92). The linker may be N- or C-terminal to ferritin.

In some embodiments, the linker is 2, 3, 4, 5, 6, 7, 8, 9, or 10 amino acids in length. In some embodiments, the linker is about 2-4, 2-6, 2-8, 2-10, 2-12, or 2-14 amino acids in length. In some embodiments, the linker is at least 15 amino acids in length. In some embodiments, the linker is at least 25 amino acids in length. In some embodiments, the linker is at least 30 amino acids in length. In some embodiments, the linker is at least 35 amino acids in length. In some embodiments, the linker is at least 40 amino acids in length. In some embodiments, the linker is less than or equal to 60 amino acids in length. In some embodiments, the linker is less than or equal to 50 amino acids in length. In some embodiments, the linker is about 16, 28, 40, 46, or 47 amino acids in length. In some embodiments, the linker is flexible. In some embodiments, the linker comprises a cysteine, e.g., for use as a site for conjugation of an immune-stimulatory moiety (e.g., adjuvant); an exemplary linker comprising a cysteine is provided as SEQ ID NO: 225. In some embodiments, the linker comprises a sequence with at least 75%, 80%, 85%, 90%, or 95% identity to SEQ ID NO: 225, and further comprises a cysteine corresponding to the cysteine in SEQ ID NO: 225. In some embodiments, the linker comprises at least 25 amino acids (e.g., 25 to 60 amino acids), wherein a cysteine is located at a position ranging from the 8^{th} amino acid from the N-terminus to the 8^{th} amino acid from the C-terminus, or within 10 amino acids of the central residue or bond of the linker.

In some embodiments, the linker comprises glycine (G) and/or serine (S) amino acids. In some embodiments, the linker comprises or consists of glycine (G), serine (S), asparagine (N), and/or alanine (A) amino acids, and optionally a cysteine as discussed above. In some embodiments, the linker comprises an amino acid sequence with at least 80%, 85%, 90%, 95%, 97%, 98%, 99%, or 100% identity to SEQ ID NO: 222. In some embodiments, the linker is GGGGSGGGGSGGGGSG (SEQ ID NO: 220), GGSGSGSNSSASSGASSGGASGGSGGSG (SEQ ID NO: 221), GGSGSASSGASASGSSNGSGSGSGSNSSASSGASSGGASGGSGGSG (SEQ ID NO: 222), or GS. In some embodiments, the linker is FR1 (SEQ ID NO: 223) or FR2 (SEQ ID NO: 224).

In some embodiments, the ferritin comprises H. pylori ferritin with the amino terminal extension of bullfrog ferritin (which will be referred to as hybrid ferritin). In some embodiments, this hybrid ferritin forms multimers with non-ferritin polypeptide-attachment sites distributed evenly on the surface (see Kanekiyo 2015). In some embodiments, N-terminal fusion proteins with hybrid ferritin allow presentation of a non-ferritin polypeptide on the ferritin nanoparticle surface. In some embodiments, the non-ferritin polypeptide is a viral or bacterial polypeptide. In some embodiments, a ferritin comprises a glutamate at a position corresponding to position 13 of SEQ ID NO: 208 (hybrid ferritin, which comprises this glutamate) or position 6 in SEQ ID NO: 209 (wild-type H. pylori ferritin, in which position 6 is isoleucine). In combination with a bullfrog linker, this glutamate is thought to preserve the conserved salt bridge found in human and bullfrog ferritins (6R and 14E in both human light chain and bullfrog lower-subunit ferritins). See Kanekiyo et al., Cell 162, 1090-1100 (2015)).

In some embodiments, an antigenic OspA polypeptide is linked to ferritin via a cysteine-thrombin-histidine linker. In some embodiments, this linker is used to directly conjugate a moiety (e.g., immune-stimulatory moiety or non-ferritin polypeptide) to ferritin via click chemistry. An exemplary sequence comprising a cysteine-thrombin-histidine linker is SEQ ID NO: 218. Click chemistry suitable for conjugation reactions involving the cysteine-thrombin-histidine linker is discussed above.

In some embodiments, a linker comprising a cysteine as a conjugation site for an immune-stimulatory moiety such as an adjuvant is used in a construct comprising a ferritin molecule lacking an unpaired, surface-exposed cysteine, or in a construct comprising a ferritin molecule comprising an unpaired, surface-exposed cysteine.

In some embodiments, a construct does not comprise a linker. In some embodiments, a construct comprises one linker. In some embodiments, a construct comprises two or more than two linkers.

### 6. Immune-Stimulatory Moieties; Adjuvants; Conjugated Polypeptides

In some embodiments, a non-ferritin polypeptide (e.g., an OspA polypeptide) and/or an immune-stimulatory moiety, such as an adjuvant, is attached to a surface-exposed amino acid of ferritin or a linker. In some embodiments, the surface-exposed amino acid is a cysteine, e.g., resulting from a mutation discussed above. In some embodiments, the surface-exposed amino acid is a lysine, aspartate, or glutamate. Conjugation procedures using glutaraldehyde (for conjugation of a lysine with an amino-bearing linker or moiety) or a carbodiimide (e.g., 1-Cyclohexyl-3-(2-morpholin-4-yl-ethyl) carbodiimide or 1-Ethyl-3-(3-dimethyl-aminopropyl) carbodiimide (EDC; EDAC) for conjugating an aspartate or glutamate to an amino-bearing linker or moiety, or a lysine to a carboxyl-bearing linker or moiety) are described in, e.g., Chapter 4 of Holtzhauer, M., Basic Methods for the Biochemical Lab, Springer 2006, ISBN 978-3-540-32785-1, available from on the world wide web atspringer.com.

In some embodiments, an immune-stimulatory moiety, such as an adjuvant, is attached to a surface-exposed amino acid of ferritin. In some embodiments, more than one immune-stimulatory moiety, such as an adjuvant, is attached to a surface-exposed amino acid of ferritin. In some embodiments, twenty-four immune-stimulatory moieties are attached to a ferritin multimer or particle (e.g., one moiety for each monomer in the H. pylori ferritin particle). In some embodiments with multiple immune-stimulatory moieties attached to a ferritin nanoparticle, all of the immune-stimulatory moieties are identical. In some embodiments with multiple immune-stimulatory moieties attached to a ferritin nanoparticle, all of the immune-stimulatory moieties are not identical.

### a) Types of Immune-Stimulatory Moieties; Adjuvants

Any immune-stimulatory moiety that can be attached to a surface-exposed amino acid (e.g., cysteine) can be used in ferritins (or linkers) according to this disclosure. In some embodiments, the immune-stimulatory moiety is a B cell agonist.

In some embodiments, the immune-stimulatory moiety is not hydrophobic. In some embodiments, the immune-stimulatory moiety is hydrophilic. In some embodiments, the immune-stimulatory moiety is polar. In some embodiments, the immune-stimulatory moiety is capable of hydrogen bonding or ionic bonding, e.g., comprises a hydrogen bond donor, hydrogen bond acceptor, cationic moiety, or anionic moiety. A moiety is considered cationic or anionic if it would be ionized in aqueous solution at a physiologically relevant pH, such as pH 6, 7, 7.4, or 8.

In some embodiments, the immune-stimulatory moiety is an adjuvant. In some embodiments, the adjuvant comprises a pathogen associated molecular pattern (PAMP). In some embodiments, the adjuvant is a toll-like receptor (TLR) agonist or stimulator of interferon genes (STING) agonist. In some embodiments, the adjuvant activates TLR signaling in B and/or T cells. In some embodiments, the adjuvant regulates the adaptive immune response.

### (1) TLR2 Agonists

In some embodiments, the immune-stimulatory moiety is a TLR2 agonist. In some embodiments, the immune-stimulatory moiety stimulates TLR2 signaling. In some embodiments, the immune-stimulatory moiety is a synthetic small molecule ligand of TLR2. In some embodiments, the immune-stimulatory moiety is a synthetic small molecule agonist of TLR2 signaling.

In some embodiments, the TLR2 agonist is PAM2CSK4, FSL-1, or PAM3CSK4.

### (2) TLR7 8 Agonists

In some embodiments, the immune-stimulatory moiety is a TLR7 and/or TLR8 agonist (i.e., an agonist of at least one of TLR7 and TLR8). In some embodiments, the immune-stimulatory moiety stimulates TLR7 and/or TLR8 signaling. In some embodiments, the immune-stimulatory moiety is a synthetic small molecule ligand of TLR7 and/or TLR8. In some embodiments, the immune-stimulatory moiety is a synthetic small molecule agonist of TLR7 and/or TLR8 signaling.

In some embodiments, the TLR7 and/or TLR8 agonist is single-stranded (ssRNA). In some embodiments, the TLR7 and/or TLR8 agonist is an imidazoquinoline. In some embodiments, the TLR7 and/or TLR8 agonist is a nucleoside analog.

In some embodiments, the TLR7 and/or TLR8 agonist is an imidazoquinolinamine Toll-like receptor (TLR) agonist, such as 3M-012 (3M Pharmaceuticals). The structure of free 3M-012 is:

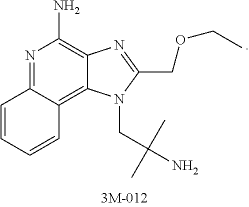 [SMILES: CCOCc1nc2c(N)nc3ccccc3c2n1CC(C)(C)N]

It is understood that an immune-stimulatory moiety such as 3M-012 or any moiety discussed herein can be conjugated to a ferritin by substituting an appropriate peripheral atom of the moiety (e.g., a hydrogen) with a bond to a ferritin described herein, e.g., at the sulfur of a surface-exposed cysteine or a linker attached to such a sulfur. Thus, when conjugated to a ferritin, the structure of the immune-stimulatory moiety will differ slightly from the structure of the free molecule.

In some embodiments the TLR7 and/or TLR8 agonist is SM7/8a. The structure of free SM7/8a is:

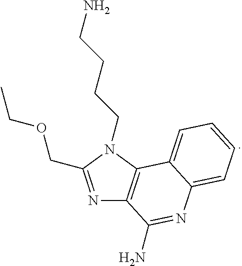 [SMILES: CCOCc1nc2c(N)nc3ccccc3c2n1CCCCN]

See, e.g., Lynn et al., Nat Biotechnol. 2015 November; 33(11):1201-10. doi: 10.1038/nbt.3371.

### (3) TLR9 Agonists

In some embodiments, the immune-stimulatory moiety is a TLR9 agonist. In some embodiments, the immune-stimulatory moiety stimulates TLR9 signaling. In some embodiments, the immune-stimulatory moiety is a synthetic small molecule ligand of TLR9. In some embodiments, the immune-stimulatory moiety is a synthetic small molecule agonist of TLR9 signaling.

In some embodiments, the TLR9 agonist is a CpG oligodeoxynucleotide (ODN). In some embodiments, the TLR9 agonist is an unmethylated CpG ODN. In some embodiments, the CpG ODN comprises a partial or complete phosphorothioate (PS) backbone instead of the natural phosphodiester (PO) backbone found in ordinary DNA.

In some embodiments, the CpG ODN is a Class B ODN, which comprises one or more 6mer CpG motif comprising 5′ Purine (Pu)-Pyrimidine (Py)-C-G-Py-Pu 3′; has a fully phosphorothioated (i.e., PS-modified) backbone; and has a length of 18-28 nucleotides. In some embodiments, the CpG ODN comprises the sequence of SEQ ID NO: 210, optionally comprising phosphorothioate linkages in the backbone.

In some embodiments, the TLR9 agonist comprises an immune-stimulatory sequence (ISS). In some embodiments the TLR9 agonist is ISS-1018 (Dynavax) (SEQ ID NO: 210).

### (4) STING Agonists

In some embodiments, the immune-stimulatory moiety is a STING (Stimulator of Interferon Genes Protein, also known as Endoplasmic Reticulum IFN Stimulator) agonist. In some embodiments, the immune-stimulatory moiety stimulates STING signaling. In some embodiments, the immune-stimulatory moiety is a synthetic small molecule ligand of STING. In some embodiments, the immune-stimulatory moiety is a synthetic small molecule agonist of STING signaling.

In some embodiments the STING agonist is a cyclic dinucleotide (CDN). See, e.g., Danilchanka et al., Cell 154:962-970 (2013). Exemplary CDNs include cdA, cdG, CAMP-cGMP, and 2′-5′,3′-5′ cGAMP (see Danilchanka et al. for structures). STING agonists also include synthetic agonists such as DMXAA

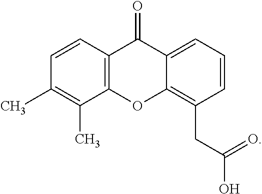 [SMILES: Cc1ccc2c(=O)c3cccc(CC(=O)O)c3oc2c1C]

### b) Conjugated Non-Ferritin Polypeptides

In some embodiments, an antigenic OspA polypeptide is conjugated to a surface-exposed amino acid of ferritin. In some embodiments, the antigenic OspA polypeptide is antigenic alone, whereas in some embodiments, the antigenic OspA polypeptide is antigenic because of its association with ferritin.

### 7. Conjugation

In some embodiments, a surface-exposed cysteine is used to conjugate an immune-stimulatory moiety, such as an adjuvant, or an antigenic OspA polypeptide to a ferritin. In some embodiments, a surface-exposed cysteine is used to conjugate a linker to the ferritin, which linker can be subsequently conjugated to an immune-stimulatory moiety, such as an adjuvant, or an antigenic OspA polypeptide. In some embodiments, a surface-exposed cysteine creates a chemical handle for conjugation reactions to attach an adjuvant, linker, or an antigenic OspA polypeptide.

In some embodiments, bioconjugates are produced, wherein an immune-stimulatory moiety, such as an adjuvant, or an antigenic OspA polypeptide is linked to a ferritin after reduction of an unpaired, surface-exposed cysteine of the ferritin. An unpaired surface-exposed cysteine is one that lacks a partner cysteine in an appropriate position to form a disulfide bond. In some embodiments, an unpaired cysteine comprises a free thiol side chain.

### a) Types of Conjugation Chemistries

Any type of chemistry can be used to conjugate the immune-stimulatory moiety, such as an adjuvant, or an antigenic OspA polypeptide to the ferritin, e.g., via reaction a surface-exposed amino acid such as cysteine or another amino acid such as Lys, Glu, or Asp.

In some embodiments, the conjugation is performed using click chemistry. As used herein, “click chemistry” refers to a reaction between a pair of functional groups that rapidly and selective react (i.e., “click”) with each other. In some embodiments, the click chemistry can be performed under mild, aqueous conditions. In some embodiments, a click chemistry reaction takes advantage of a cysteine on the surface of the ferritin, such as a cysteine resulting from mutation of a surface-exposed amino acid, to perform click chemistry using a functional group that can react with the cysteine.

A variety of reactions that fulfill the criteria for click chemistry are known in the field, and one skilled in the art could use any one of a number of published methodologies (see, e.g., Hein et al., Pharm Res 25(10):2216-2230 (2008)). A wide range of commercially available reagents for click chemistry could be used, such as those from Sigma Aldrich, Jena Bioscience, or Lumiprobe. In some embodiments, conjugation is performed using click chemistry as described in the Examples below.

In some embodiments, the click chemistry reaction occurs after reduction of the ferritin.

In some embodiments, the click chemistry may be a 1-step click reaction. In some embodiments, the click chemistry may be a 2-step click reaction.

In some embodiments, the reaction(s) comprises metal-free click chemistry. In some embodiments, the reaction(s) comprise thiol-maleimide and/or disulfide exchange.

### Metal-Free Click Chemistry

Metal-free click chemistry can be used for conjugation reactions to avoid potential oxidation of proteins. Metal-free click chemistry has been used to form antibody conjugates (see van Geel et al., Bioconjugate Chem. 2015, 26, 2233-2242).

In some embodiments, metal-free click chemistry is used in reactions to attach adjuvant to ferritin. In some embodiments, copper-free conjugation is used in reactions to attach adjuvant to ferritin. In some embodiments, the metal-free click chemistry uses bicyclo[6.1.0]nonyne (BCN). In some embodiments, the metal-free click chemistry uses dibenzoazacyclooctyne (DBCO). In some embodiments BCN or DBCO reacts with an azide group.

DBCO has high specificity for azide groups via a strain-promoted click reaction in the absence of a catalyst, resulting in high yield of a stable triazole. In some embodiments, DBCO reacts with azide in the absence of copper catalyst.

In some embodiments, metal-free click chemistry is used in a 1-step click reaction. In some embodiments, metal-free click chemistry is used in a 2-step click reaction.

### Thiol-Maleimide and Disulfide Exchange

Ferritins described herein can comprise a cysteine comprising a thiol, also known as a sulfhydryl, which is available for reaction with sulfhydryl-reactive chemical groups (or which can be made available through reduction). Thus, the cysteine allows chemoselective modification to add an immune-stimulatory moiety, such as an adjuvant, to the ferritin. Under basic conditions, the cysteine will be deprotonated to generate a thiolate nucleophile, which can react with soft electrophiles, such as maleimides and iodoacetamides. The reaction of the cysteine with a maleimide or iodoacetamide results in a carbon-sulfur bond.

In some embodiments, a sulfhydryl-reactive chemical group reacts with the surface-exposed cysteine or cysteine in the linker of the ferritin. In some embodiments, the sulfhydryl-reactive chemical group is a haloacetyl, maleimide, aziridine, acryloyl, arylating agent, vinylsulfone, pyridyl disulfide, or TNB-thiol.

In some embodiments, the sulfhydryl-reactive chemical group conjugates to the sulfhydryl of the cysteine by alkylation (i.e., formation of a thioether bond)). In some embodiments, the sulfhydryl-reactive chemical group conjugates to the sulfhydryl of the cysteine by disulfide exchange (i.e., formation of a disulfide bond).

In some embodiments, the reaction to conjugate an immune-stimulatory moiety, such as an adjuvant, to the ferritin is a thiol-maleimide reaction.

In some embodiments, the sulfhydryl-reactive chemical group is a maleimide. In some embodiments, reaction of a maleimide with the cysteine results in formation of a stable thioester linkage, e.g., that is not reversible. In some embodiments, the maleimide does not react with tyrosines, histidines, or methionines in the ferritin. In some embodiments, unreacted maleimides are quenched at the end of the reaction by adding a free thiol, e.g., in excess.

In some embodiments, the reaction to conjugate an immune-stimulatory moiety, such as an adjuvant, to the ferritin is a thiol-disulfide exchange, also known as a disulfide interchange. In some embodiments, the reaction involves formation of a mixed disulfide comprising a portion of the original disulfide. In some embodiments, the original disulfide is the cysteine introduced in the ferritin by mutation of a surface-exposed amino acid or addition of an N-terminal linker.

In some embodiments, the sulfhydryl-reactive chemical group is a pyridyl dithiol. In some embodiments, the sulfhydryl-reactive chemical group is a TNB-thiol group.

### b) Linkers

In some embodiments, an immune-stimulatory moiety, such as an adjuvant, or an antigenic OspA polypeptide is attached to the ferritin via a linker that is covalently bound to a surface-exposed amino acid such as a cysteine. In some embodiments, the linker comprises a polyethylene glycol, e.g., a PEG linker. In some embodiments, the polyethylene glycol (e.g., PEG) linker increases water solubility and ligation efficiency of the ferritin linked to the immune-stimulatory moiety, such as an adjuvant. The PEG linker is between 2 and 18 PEGs long, e.g., PEG4, PEG5, PEG6, PEG7, PEG8, PEG9, PEG10, PEG11, PEG12, PEG13, PEG14, PEG15, PEG16, PEG17, and PEG18.

In some embodiments, the linker comprises a maleimide. In some embodiments, the linker comprises the components of immune-stimulatory moiety (ISM)-linker-maleimide. In some embodiments, the ISM-linker-maleimide is conjugated to ferritin in a 1-step click chemistry reaction by reaction of the maleimide with a cysteine of the ferritin. In some embodiments, the ISM of the adjuvant-linker-maleimide is SM7/8a. In some embodiments, the linker of the ISM-linker-maleimide is PEG4. In some embodiments, the ISM-linker-maleimide is SM7/8a-PEG4-maleimide.

In some embodiments, a 2-step click chemistry protocol is used with a linker comprising a sulfhydryl-reactive chemical group at one end and an amine-reactive group at the other end. In such a 2-step click chemistry protocol, a sulfhydryl-reactive chemical group reacts with a cysteine of the ferritin, while the amine-reactive group reacts with a reagent attached to the ISM. In this way, the ISM is conjugated to the ferritin via a set of 2 click chemistry reagents.

In some embodiments of the 2-step click chemistry protocol, the sulfhydryl-reactive chemical group is maleimide. In some embodiments of the 2-step click chemistry protocol, the maleimide reacts with the cysteine introduced in the ferritin by mutation of a surface-exposed amino acid or addition of an N-terminal linker.

In some embodiments of the 2-step click chemistry protocol, the amine-reactive group is DBCO. In some embodiments of the 2-step click chemistry protocol, the DBCO reacts with an azide group attached to an ISM.

In some embodiments, a maleimide-linker-DBCO is used. In some embodiments, the maleimide-linker-DBCO is conjugated to ferritin after the ferritin is reduced. In some embodiments, the maleimide-linker-reagent is conjugated to ferritin by reaction of the maleimide with the cysteine of the ferritin in a first step. In some embodiments, the DBCO is used to link to an ISM attached to azide. In some embodiments, the ISM coupled to azide is ISS-1018. In some embodiments, the adjuvant coupled to azide is 3M-012 or CpG.

In some embodiments, a linker with a reactive group is added to the ISM. In some embodiments, the linker is a PEG4-azide linker or a PEG4-maleimide linker.

In some embodiments, a PEG4-azide linker is conjugated to 3M-012. An exemplary structure of 3M-012 conjugated to a PEG4-azide linker is:

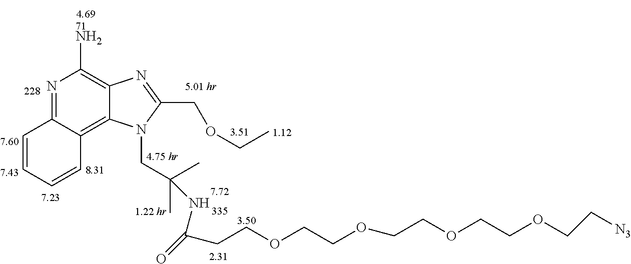 [SMILES: CCOCc1nc2c(N)nc3ccccc3c2n1CC(C)(C)NC(=O)CCOCCOCCOCCOCCN=[N+]=[N-]]

In some embodiments, a PEG4-azide linker is conjugated to SM7/8a. An exemplary structure of SM7/8a conjugated to a PEG4-azide linker is:

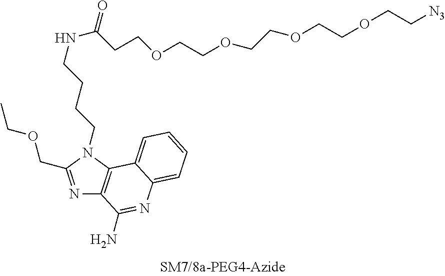 [SMILES: CCOCc1nc2c(N)nc3ccccc3c2n1CCCCNC(=O)CCOCCOCCOCCOCCN=[N+]=[N-]]

In some embodiments, a PEG4-maleimide linker is conjugated to SM7/8a. An exemplary structure of SM7/8a conjugated to a PEG4-maleimide linker is:

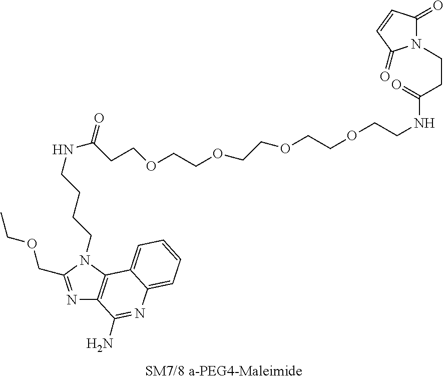 [SMILES: CCOCc1nc2c(N)nc3ccccc3c2n1CCCCNC(=O)CCOCCOCCOCCOCCNC(=O)CCN1C(=O)C=CC1=O]

In some embodiments, an azide group is conjugated to ISS-1018. An exemplary structure of ISS-1018 conjugated to an NHS ester-azide linker is:

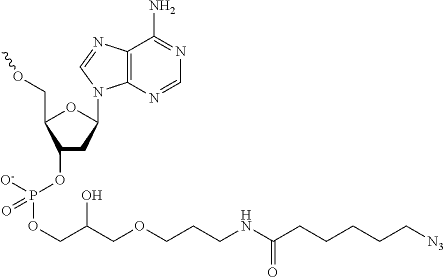

### D. Exemplary Compositions, Kits, Nucleic Acids, Uses, and Methods

In some embodiments, a composition comprising any one or more of the antigenic polypeptides described herein and a pharmaceutically acceptable vehicle, adjuvant, or excipient is provided.

In some embodiments, an antigenic polypeptide, or composition described herein is administered to a subject, such as a human, to immunize against infection caused by Borrelia, e.g., Lyme disease. In some embodiments, an antigenic polypeptide described herein is administered to a subject, such as a human, to produce a protective immune response to future infection with Borrelia. In some embodiments, a polypeptide comprising a modified OspA serotype 1 polypeptide as described herein is administered. In some embodiments, an antigenic polypeptide comprising an OspA and ferritin as described herein is administered.

In some embodiments, the protective immune response decreases the incidence of hospitalization. In some embodiments, the protective immune response decreases the incidence of acute or chronic Lyme disease, including joint inflammation, neurological symptoms, cognitive deficits, or heart rhythm irregularities.

In some embodiments, a composition comprises an OspA serotype 1 polypeptide. In some embodiments, a composition comprises an OspA serotype 2 polypeptide. In some embodiments, a composition comprises an OspA serotype 3 polypeptide. In some embodiments, a composition comprises an OspA serotype 4 polypeptide. In some embodiments, a composition comprises an OspA serotype 5 polypeptide. In some embodiments, a composition comprises an OspA serotype 6 polypeptide. In some embodiments, a composition comprises an OspA serotype 7 polypeptide. In some embodiments, a composition comprises only one OspA polypeptide. In some embodiments, a composition comprises on or more of an OspA serotype 1, 2, 3, 4, 5, 6, and 7.

In some embodiments, a composition comprises more than one OspA polypeptide, e.g., from more than one OspA serotype. In some embodiments, such a composition allows vaccination against multiple types of Borrelia, for example, one, two, three, four, five, six, or seven of serotypes 1-7. As the serotypes of OspA are related to genospecies of Borrelia (see Wilske 1993), vaccination with such a composition comprising OspA of multiple serotypes may produce immunity to a range of bacteria that can cause Lyme disease.

In some embodiments, a composition comprising OspA of multiple serotypes produces immunity against Borrelia that express different OspA serotypes. For example, it has been observed as discussed in the Examples that a composition comprising OspA polypeptides of serotypes 1-5 and 7 can elicit antibodies that recognize OspA serotype 6. In some embodiments, such a composition produces immunity against multiple genospecies of Borrelia.

In some embodiments, a composition comprises two different OspA serotypes. In some embodiments, a multivalent composition comprises three different OspA serotypes. In some embodiments, a multivalent composition comprises four different OspA serotypes. In some embodiments, a multivalent composition comprises five different OspA serotypes. In some embodiments, a multivalent composition comprises six different OspA serotypes. In some embodiments, a multivalent composition comprises seven different OspA serotypes.

In some embodiments, a composition comprises an OspA serotype 1 and any one or more of OspA serotypes 2-7. In some embodiments, a composition comprises an OspA serotype 2 and any one or more of OspA serotypes 1 and 3-7. In some embodiments, a composition comprises an OspA serotype 3 and any one or more of OspA serotypes 1-2 and 4-7. In some embodiments, a composition comprises an OspA serotype 4 and any one or more of OspA serotypes 1-3 and 5-7. In some embodiments, a composition comprises an OspA serotype 5 and any one or more of OspA serotypes 1-4 and 6-7. In some embodiments, a composition comprises an OspA serotype 6 and any one or more of OspA serotypes 1-5 and 7. In some embodiments, a composition comprises an OspA serotype 7 and any one or more of OspA serotypes 1-6. In some embodiments, a composition comprises at least 2, 3, 4, 5, or 6 of OspA polypeptides of serotypes 1-5 and 7.

In some embodiments, any one or more of the antigenic polypeptides, or compositions described herein are provided for use in immunizing against infection caused by Borrelia, e.g., Lyme disease. In some embodiments, any one or more of the antigenic polypeptides, or compositions described herein are provided for use in producing a protective immune response to future infection with Borrelia. In some embodiments, any one or more of the antigenic polypeptides, or compositions described herein are for use in a mammal, such as a primate (e.g., non-human primate, such as a monkey (e.g., a macaque, such as rhesus or cynomolgus) or ape), rodent (e.g., mouse or rat), or domesticated mammal (e.g., dog, rabbit, cat, horse, sheep, cow, goat, camel, or donkey).

### 1. Adjuvants

An adjuvant may be administered together with the antigenic polypeptides described herein to a subject, wherein such administration produces a higher titer of antibodies against the OspA in the subject as compared to administration of the OspA without the adjuvant. An adjuvant may promote earlier, more potent, or more persistent immune response to the OspA.

In some embodiments, a composition comprises one adjuvant. In some embodiments, a composition comprises more than one adjuvant. In some embodiments, a composition does not comprise an adjuvant.

In some embodiments, an adjuvant comprises aluminum. In some embodiments, an adjuvant is aluminum phosphate. In some embodiments, an adjuvant is Alum (Alyhydrogel '85 2%; Brenntag—Cat #21645-51-2).

In some embodiments, an adjuvant is an organic adjuvant. In some embodiments, an adjuvant is an oil-based adjuvant. In some embodiments, an adjuvant comprises an oil-in-water nanoemulsion.

In some embodiments, an adjuvant comprises squalene. In some embodiments, the adjuvant comprising squalene is Ribi (Sigma adjuvant system Cat #S6322-1vl), AddaVax™ (squalene-based oil-in-water nano-emulsion), MF59, AS03, or AF03 (see U.S. Pat. No. 9,703,095). In some embodiments, the adjuvant comprising squalene is a nanoemulsion.

In some embodiments, an adjuvant comprises a polyacrylic acid polymer (PAA). In some embodiments, the adjuvant comprising PAA is SPA09 (see WO 2017218819).

In some embodiments, an adjuvant comprises non-metabolizable oils. In some embodiments, the adjuvant is Incomplete Freund's Adjuvant (IFA).

In some embodiments, an adjuvant comprises non-metabolizable oils and killed Mycobacterium tuberculosis. In some embodiments, the adjuvant is Complete Freund's Adjuvant (CFA).

In some embodiments, an adjuvant is a lipopolysaccharide. In some embodiments, an adjuvant is monophosphoryl A (MPL or MPLA).

### 2. Subjects

In some embodiments, the subject is a mammal. In some embodiments, the subject is a human.

In some embodiments, the subject is an adult (greater than or equal to 18 years of age). In some embodiments, the subject is a child or adolescent (less than 18 years of age). In some embodiments, the subject is elderly (greater than 60 years of age). In some embodiments, the subject is a non-elderly adult (greater than or equal to 18 years of age and less than or equal to 60 years of age).

In some embodiments, more than one administration of the composition is administered to the subject. In some embodiments, a booster administration improves the immune response.

In some embodiments, any one or more of the antigenic polypeptides, or compositions described herein are for use in a mammal, such as a primate (e.g., non-human primate, such as a monkey (e.g., a macaque, such as rhesus or cynomolgus) or ape), rodent (e.g., mouse or rat), or domesticated mammal (e.g., dog, rabbit, cat, horse, sheep, cow, goat, camel, or donkey).

In some embodiments, the composition is suitably formulated for an intended route of administration. Examples of suitable routes of administration include intramuscular, transcutaneous, subcutaneous, intranasal, oral, or transdermal.

In some embodiments, more than one administration of the composition is administered to the subject. In some embodiments, a booster administration improves the immune response.

### 3. Pharmaceutical Compositions

In various embodiments, a pharmaceutical composition comprising an antigenic ferritin polypeptide described herein and/or related entities is provided. In some embodiments, the pharmaceutical composition is an immunogenic composition (e.g., a vaccine) capable of eliciting an immune response such as a protective immune response against Borrelia.

For example, in some embodiments, the pharmaceutical compositions may comprise one or more of the following: (1) an antigenic ferritin protein comprising (i) a mutation replacing a surface-exposed amino acid with a cysteine and (ii) an antigenic OspA polypeptide; (2) an antigenic ferritin protein comprising (i) a mutation replacing a surface exposed amino acid with a cysteine and an immune-stimulatory moiety linked to the cysteine; and (ii) an antigenic OspA polypeptide; (3) antigenic ferritin protein comprising (i) a surface-exposed cysteine, (ii) a peptide linker N-terminal to the ferritin protein, and (iii) an antigenic OspA polypeptide N-terminal to the peptide linker; (4) an antigenic ferritin protein comprising: (i) a mutation replacing a surface exposed amino acid with a cysteine and an immune-stimulatory moiety linked to the cysteine, (ii) a mutation replacing the internal cysteine at position 31 of H. pylori ferritin, or a mutation of an internal cysteine at a position that is analogous to position 31 of a non-H. pylori ferritin as determined by pair-wise or structural alignment, with a non-cysteine amino acid, (iii) a mutation replacing a surface-exposed asparagine with a non-asparagine amino acid, and (iv) an antigenic OspA polypeptide; or (5) a ferritin particle comprising any of the foregoing ferritin proteins.

In some embodiments, the present invention provides pharmaceutical compositions comprising antibodies or other agents related to the antigenic polypeptides described herein. In an embodiment, the pharmaceutical composition comprises antibodies that bind to and/or compete with an antigenic polypeptide described herein. Alternatively, the antibodies may recognize viral particles or bacteria comprising the non-ferritin polypeptide component of an antigenic polypeptide described herein.

In some embodiments, the pharmaceutical compositions as described herein are administered alone or in combination with one or more agents to enhance an immune response, e.g., an adjuvant described above. In some embodiments, a pharmaceutical composition further comprises an adjuvant described above.

In some embodiments, the pharmaceutical composition further comprises a pharmaceutically acceptable carrier or excipient. As used herein, the term “carrier” refers to a diluent, adjuvant, excipient, or vehicle with which a pharmaceutical composition is administered. In exemplary embodiments, carriers can include sterile liquids, such as, for example, water and oils, including oils of petroleum, animal, vegetable, or synthetic origin, such as, for example, peanut oil, soybean oil, mineral oil, sesame oil and the like. In some embodiments, carriers are or include one or more solid components. Pharmaceutically acceptable carriers can also include, but are not limited to, saline, buffered saline, dextrose, glycerol, ethanol, and combinations thereof. As used herein, an excipient is any non-therapeutic agent that may be included in a pharmaceutical composition, for example to provide or contribute to a desired consistency or stabilizing effect. Suitable pharmaceutical excipients include, but are not limited to, starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. In various embodiments, the pharmaceutical composition is sterile.

In some embodiments, the pharmaceutical composition contains minor amounts of wetting or emulsifying agents, or pH buffering agents. In some embodiments, the pharmaceutical compositions of may include any of a variety of additives, such as stabilizers, buffers, or preservatives. In addition, auxiliary, stabilizing, thickening, lubricating, and coloring agents can be included.

In various embodiments, the pharmaceutical composition may be formulated to suit any desired mode of administration. For example, the pharmaceutical composition can take the form of solutions, suspensions, emulsion, drops, tablets, pills, pellets, capsules, capsules containing liquids, gelatin capsules, powders, sustained-release formulations, suppositories, emulsions, aerosols, sprays, suspensions, lyophilized powder, frozen suspension, desiccated powder, or any other form suitable for use. General considerations in the formulation and manufacture of pharmaceutical agents may be found, for example, in Remington's Pharmaceutical Sciences, 19th ed., Mack Publishing Co., Easton, PA, 1995; incorporated herein by reference.

The pharmaceutical composition can be administered via any route of administration. Routes of administration include, for example, oral, intradermal, intramuscular, intraperitoneal, intravenous, subcutaneous, intranasal, mucosal, epidural, sublingual, intranasal, intracerebral, intravaginal, transdermal, rectally, by intratracheal instillation, bronchial instillation, inhalation, or topically. Administration can be local or systemic. In some embodiments, administration is carried out orally. In another embodiment, the administration is by parenteral injection. In some instances, administration results in the release of the antigenic ferritin polypeptide described herein into the bloodstream. The mode of administration can be left to the discretion of the practitioner.

In some embodiments, the pharmaceutical composition is suitable for parenteral administration (e.g. intravenous, intramuscular, intraperitoneal, and subcutaneous). Such compositions can be formulated as, for example, solutions, suspensions, dispersions, emulsions, and the like. They may also be manufactured in the form of sterile solid compositions (e.g. lyophilized composition), which can be dissolved or suspended in sterile injectable medium immediately before use. For example, parenteral administration can be achieved by injection. In such embodiments, injectables are prepared in conventional forms, i.e., either as liquid solutions or suspensions, solid forms suitable for solution or suspension in liquid prior to injection, or as emulsions. In some embodiments, injection solutions and suspensions are prepared from sterile powders, lyophilized powders, or granules.

In a further embodiment, the pharmaceutical composition is formulated for delivery by inhalation (e.g., for direct delivery to the lungs and the respiratory system). For example, the composition may take the form of a nasal spray or any other known aerosol formulation. In some embodiments, preparations for inhaled or aerosol delivery comprise a plurality of particles. In some embodiments, such preparations can have a mean particle size of about 1, about 2, about 3, about 4, about 5, about 6, about 7, about 8, about 9, about 10,about 11, about 12, or about 13 microns. In some embodiments, preparations for inhaled or aerosol delivery are formulated as a dry powder. In some embodiments, preparations for inhaled or aerosol delivery are formulated as a wet powder, for example through inclusion of a wetting agent. In some embodiments, the wetting agent is selected from the group consisting of water, saline, or other liquid of physiological pH.

In some embodiments, the pharmaceutical composition in accordance with the invention are administered as drops to the nasal or buccal cavity. In some embodiments, a dose may comprise a plurality of drops (e.g., 1-100, 1-50, 1-20, 1-10, 1-5, etc.).

The present pharmaceutical composition may be administered in any dose appropriate to achieve a desired outcome. In some embodiments, the desired outcome is the induction of a long-lasting adaptive immune response against a pathogen, such as the source of a non-ferritin polypeptide present in an antigenic ferritin polypeptide present in the composition. In some embodiments, the desired outcome is a reduction in the intensity, severity, frequency, and/or delay of onset of one or more symptoms of infection. In some embodiments, the desired outcome is the inhibition or prevention of infection. The dose required will vary from subject to subject depending on the species, age, weight, and general condition of the subject, the severity of the infection being prevented or treated, the particular composition being used, and its mode of administration.

In some embodiments, pharmaceutical compositions in accordance with the invention are administered in single or multiple doses. In some embodiments, the pharmaceutical compositions are administered in multiple doses administered on different days (e.g., prime-boost vaccination strategies). In some embodiments, the pharmaceutical composition is administered as part of a booster regimen.

In various embodiments, the pharmaceutical composition is co-administered with one or more additional therapeutic agents. Co-administration does not require the therapeutic agents to be administered simultaneously, if the timing of their administration is such that the pharmacological activities of the additional therapeutic agent and the active ingredient(s) in the pharmaceutical composition overlap in time, thereby exerting a combined therapeutic effect. In general, each agent will be administered at a dose and on a time schedule determined for that agent.

### 4. Nucleic Acid/RNA

Also provided is a nucleic acid encoding an antigenic OspA polypeptide described herein. In some embodiments, the nucleic acid is an mRNA. Any nucleic acid capable of undergoing translation resulting in a polypeptide is considered an mRNA for purposes of this disclosure.

### 5. Kits

Also provided herein are kits comprising one or more antigenic polypeptides, nucleic acids, antigenic ferritin particles, antigenic lumazine synthase particles, compositions, or pharmaceutical compositions described herein. In some embodiments, a kit further comprises one or more of a solvent, solution, buffer, instructions, or desiccant.

This description and exemplary embodiments should not be taken as limiting. For the purposes of this specification and appended claims, unless otherwise indicated, all numbers expressing quantities, percentages, or proportions, and other numerical values used in the specification and claims, are to be understood as being modified in all instances by the term “about,” to the extent they are not already so modified. “About” indicates a degree of variation that does not substantially affect the properties of the described subject matter, e.g., within 10%, 5%, 2%, or 1%. Accordingly, unless indicated to the contrary, the numerical parameters set forth in the following specification and attached claims are approximations that may vary depending upon the desired properties sought to be obtained. At the very least, and not as an attempt to limit the application of the doctrine of equivalents to the scope of the claims, each numerical parameter should at least be construed considering the number of reported significant digits and by applying ordinary rounding techniques.

It is noted that, as used in this specification and the appended claims, the singular forms “a,” “an,” and “the,” and any singular use of any word, include plural referents unless expressly and unequivocally limited to one referent. As used herein, the term “include” and its grammatical variants are intended to be non-limiting, such that recitation of items in a list is not to the exclusion of other like items that can be substituted or added to the listed items. The term “or” is used in the inclusive sense, i.e., equivalent to “and/or,” unless the context dictates otherwise.

TABLE 1
(Sequence Table): Description of sequences

| | Sequences | |
|---|---|---|
| | Key for sequences 1-102: | |
| | Leader sequences are sometimes underlined | |
| | Modified LFA-1 site is Italicized and underlined (if present) | |
| | Mutations other than modified LFA-1 site are in BOLD and curvy underlined | |
| | Linker is double underlined | |
| Description | Bullfrog sequence is Italicized and curvy underlined (if present) | |
| (“ferritin” refers to H. pylori | Human heavy chain ferritin sequence is in BOLD (if present) | |
| ferritin optionally with one or more | Transmembrane domain is italicized (if present) | |
| mutations unless otherwise indicated) | lipidation site is in Bold, italicized, and underlined (if present) | SEQ ID NO: |
| Serotype 1 RD2 OspA-ferritin | MDEKNSVSVDLPGEMKVLVSKEKNKDGKYDLIATVDKLELKGTSDKNNGSGVLEGVKADKSKVKLTISDDLGQTTLEVFKEDGKTLVSKKVTSK | 1 |
| | DKSSTEEKFNEKGEVSEKIITRADGTRLEYTGIKSDGSGKAKEVLKGYTLEGQLSDEKTTLVVKEGTVTLSKNISKSGEVSVELNDTDSSAATK | |
| | 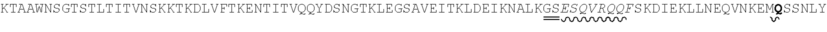 | |
| | 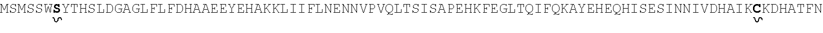 | |
| | FLQWYVAEQHEEEVLFKDILDKIELIGNENHGLYLADQYVKGIAKSRKS | |
| Serotype 1 RD1 OspA-ferritin | MDEKNSVSVDLPGEMKVLVSKEKNKDGKYDLIATVDKLELKGTSDKNNGSGVLEGVKADKSKVKLTISDDLGQTTLEVFKEDGKTLVSKKVTSK | 2 |
| | DKSSTEEKFNEKGEVSEKIITRADGTRLEYTGIKSDGSGKAKEVLKGYDLKGELSSEKTTLVVKEGTVTLSKNISKSGEVSVELNDTDSSAATK | |
| | 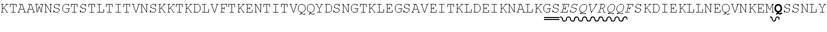 | |
| | 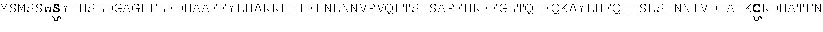 | |
| | FLQWYVAEQHEEEVLFKDILDKIELIGNENHGLYLADQYVKGIAKSRKS | |
| Serotype 1 OspA-ferritin; LFA-1 | MDEKNSVSVDLPGEMKVLVSKEKNKDGKYDLIATVDKLELKGTSDKNNGSGVLEGVKADKSKVKLTISDDLGQTTLEVFKEDGKTLVSKKVTSK | 3 |
| replacement Serotype 2 | DKSSTEEKFNEKGEVSEKIITRADGTRLEYTGIKSDGSGKAKEVLKGFTLEGKVANEKTTLVVKEGTVTLSKNISKSGEVSVELNDTDSSAATK | |
| | 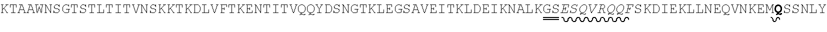 | |
| | 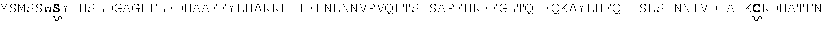 | |
| | FLQWYVAEQHEEEVLFKDILDKIELIGNENHGLYLADQYVKGIAKSRKS | |
| Serotype 1 OspA-ferritin; LFA-1 | MDEKNSVSVDLPGEMKVLVSKEKNKDGKYDLIATVDKLELKGTSDKNNGSGVLEGVKADKSKVKLTISDDLGQTTLEVFKEDGKTLVSKKVTSK | 4 |
| replacement Serotype 3 | DKSSTEEKFNEKGEVSEKIITRADGTRLEYTGIKSDGSGKAKEVLKGFALEGTLTDEKTTLVVKEGTVTLSKNISKSGEVSVELNDTDSSAATK | |
| | 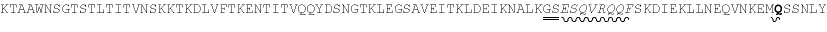 | |
| | 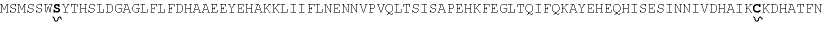 | |
| | FLQWYVAEQHEEEVLFKDILDKIELIGNENHGLYLADQYVKGIAKSRKS | |
| Serotype 2 OspA-ferritin | MDEKNSASVDLPGEMKVLVSKEKDKDGKYSLKATVDKIELKGTSDKDNGSGVLEGTKDDKSKAKLTIADDLSKTTFELFKEDGKTLVSRKVSSK | 5 |
| | DKTSTDEMFNEKGELSAKTMTRENGTKLEYTEMKSDGTGKAKEVLKNFTLEGKVANDKVTLEVKEGTVTLSKEIAKSGEVTVALNDTNTTQATK | |
| | 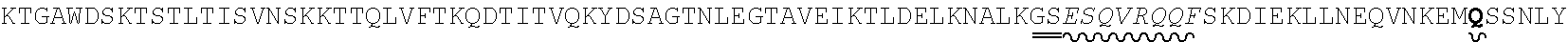 | |
| | 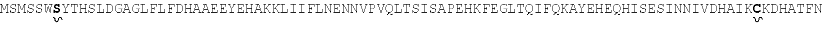 | |
| | FLQWYVAEQHEEEVLFKDILDKIELIGNENHGLYLADQYVKGIAKSRKS | |
| Serotype 3 OspA-ferritin | MDEKNSVSVDLPGGMKVLVSKEKDKDGKYSLMATVEKLELKGTSDKSNGSGVLEGEKADKSKAKLTISQDLNQTTFEIFKEDGKTLVSRKVNSK | 6 |
| | DKSSTEEKFNDKGKLSEKVVTRANGTRLEYTEIKNDGSGKAKEVLKGFALEGTLTDGGETKLTVTEGTVTLSKNISKSGEITVALNDTETTPAD | |
| | 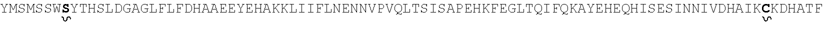 | |
| | 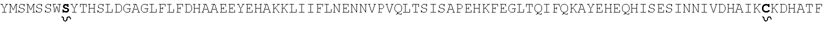 | |
| | NFLQWYVAEQHEEEVLFKDILDKIELIGNENHGLYLADQYVKGIAKSRKS | |
| Serotype 4 OspA-ferritin | MDEKNSVSVDLPGEMKVLVSKEKDKDGKYSLMATVDKLELKGTSDKSNGSGTLEGEKSDKSKAKLTISEDLSKTTFEIFKEDGKTLVSKKVNSK | 7 |
| | DKSSIEEKFNAKGELSEKTILRANGTRLEYTEIKSDGTGKAKEVLKDFALEGTLAADKTTLKVTEGTVVLSKHIPNSGEITVELNDSNSTQATK | |
| | 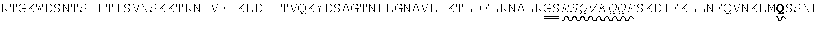 | |
| | 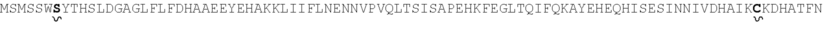 | |
| | FLQWYVAEQHEEEVLFKDILDKIELIGNENHGLYLADQYVKGIAKSRKS | |
| Serotype 5 OspA-ferritin | MDEKNSVSVDLPGGMKVLVSKEKDKDGKYSLMATVEKLELKGTSDKNNGSGTLEGEKTDKSKVKLTIAEDLSKTTFEIFKEDGKTLVSKKVTLK | 8 |
| | DKSSTEEKFNEKGEISEKTIVRANGTRLEYTDIKSDGSGKAKEVLKDFTLEGTLAADGKTTLKVTEGTVVLSKNILKSGEITVALDDSDTTQAT | |
| | 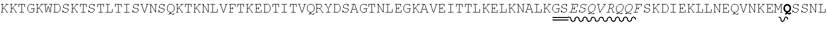 | |
| | 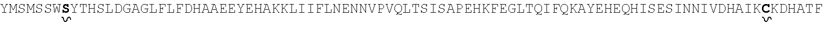 | |
| | NFLQWYVAEQHEEEVLFKDILDKIELIGNENHGLYLADQYVKGIAKSRKS | |
| Serotype 6 OspA-ferritin | MDEKNSVSVDLPGGMTVLVSKEKDKDGKYSLEATVDKLELKGTSDKNNGSGTLEGEKTDKSKVKSTIADDLSQTKFEIFKEDGKTLVSKKVTLK | 9 |
| | DKSSTEEKFNGKGETSEKTIVRANGTRLEYTDIKSDGSGKAKEVLKDFTLEGTLAADGKTTLKVTEGTVVLSKNILKSGEITAALDDSDTTRAT | |
| | 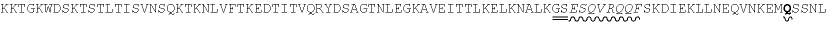 | |
| | 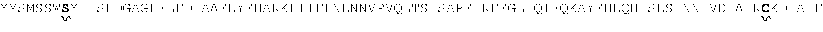 | |
| | NFLQWYVAEQHEEEVLFKDILDKIELIGNENHGLYLADQYVKGIAKSRKS | |
| Serotype 7 OspA-ferritin | MDEKNSVSVDLPGEMKVLVSKEKDKDGKYSLEATVDKLELKGTSDKNNGSGVLEGVKAAKSKAKLTIADDLSQTKFEIFKEDGKTLVSKKVTLK | 10 |
| | DKSSTEEKFNDKGKLSEKVVTRANGTRLEYTEIQNDGSGKAKEVLKSLTLEGTLTADGETKLTVEAGTVTLSKNISESGEITVELKDTETTPAD | |
| | 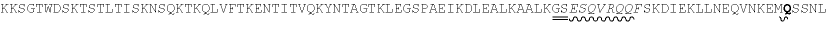 | |
| | 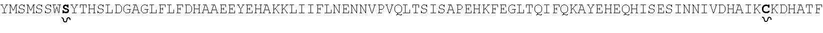 | |
| | NFLQWYVAEQHEEEVLFKDILDKIELIGNENHGLYLADQYVKGIAKSRKS | |
| Serotype 4 OspA-Cysteine-Thrombin-His | MDEKNSVSVDLPGEMKVLVSKEKDKDGKYSLMATVDKLELKGTSDKSNGSGTLEGEKSDKSKAKLTISEDLSKTTFEIFKEDGKTLVSKKVNSK | 11 |
| | DKSSIEEKFNAKGELSEKTILRANGTRLEYTEIKSDGTGKAKEVLKDFALEGTLAADKTTLKVTEGTVVLSKHIPNSGEITVELNDSNSTQATK | |
| | KTGKWDSNTSTLTISVNSKKTKNIVFTKEDTITVQKYDSAGTNLEGNAVEIKTLDELKNALKCLVPRGSLEHHHHHH | |
| Serotype 1 OspA-lumazine synthase; | MDEKNSVSVDLPGEMKVLVSKEKNKDGKYDLIATVDKLELKGTSDKNNGSGVLEGVKADKSKVKLTISDDLGQTTLEVFKEDGKTLVSKKVTSK | 12 |
| LFA-1 replacement RD2 | DKSSTEEKFNEKGEVSEKIITRADGTRLEYTGIKSDGSGKAKEVLKGYTLEGQLSDEKTTLVVKEGTVTLSKNISKSGEVSVELNDTDSSAATK | |
| | KTAAWNSGTSTLTITVNSKKTKDLVFTKENTITVQQYDSNGTKLEGSAVEITKLDEIKNALKGGGSMQIYEGKLTAEGLRFGIVASRFNHALVD | |
| | RLVEGAIDCIVRHGGREEDITLVRVPGSWEIPVAAGELARKEDIDAVIAIGVLIRGATPHFDYIASEVSKGLANLSLELRKPITFGVITADTLE | |
| | QAIERAGTKHGNKGWEAALSAIEMANLFKSLR | |
| Serotype 1 OspA-lumazine synthase; | MDEKNSVSVDLPGEMKVLVSKEKNKDGKYDLIATVDKLELKGTSDKNNGSGVLEGVKADKSKVKLTISDDLGQTTLEVFKEDGKTLVSKKVTSK | 13 |
| LFA-1 replacement RD1 | DKSSTEEKFNEKGEVSEKIITRADGTRLEYTGIKSDGSGKAKEVLKGYDLKGELSSEKTTLVVKEGTVTLSKNISKSGEVSVELNDTDSSAATK | |
| | KTAAWNSGTSTLTITVNSKKTKDLVFTKENTITVQQYDSNGTKLEGSAVEITKLDEIKNALKGGGSMQIYEGKLTAEGLRFGIVASRFNHALVD | |
| | RLVEGAIDCIVRHGGREEDITLVRVPGSWEIPVAAGELARKEDIDAVIAIGVLIRGATPHFDYIASEVSKGLANLSLELRKPITFGVITADTLE | |
| | QAIERAGTKHGNKGWEAALSAIEMANLFKSLR | |
| Serotype 1 OspA-lumazine synthase; | MDEKNSVSVDLPGEMKVLVSKEKNKDGKYDLIATVDKLELKGTSDKNNGSGVLEGVKADKSKVKLTISDDLGQTTLEVFKEDGKTLVSKKVTSK | 14 |
| LFA-1 replacement Sero2 | DKSSTEEKFNEKGEVSEKIITRADGTRLEYTGIKSDGSGKAKEVLKGFTLEGKVANEKTTLVVKEGTVTLSKNISKSGEVSVELNDTDSSAATK | |
| | KTAAWNSGTSTLTITVNSKKTKDLVFTKENTITVQQYDSNGTKLEGSAVEITKLDEIKNALKGGGSMQIYEGKLTAEGLRFGIVASRFNHALVD | |
| | RLVEGAIDCIVRHGGREEDITLVRVPGSWEIPVAAGELARKEDIDAVIAIGVLIRGATPHFDYIASEVSKGLANLSLELRKPITFGVITADTLE | |
| | QAIERAGTKHGNKGWEAALSAIEMANLFKSLR | |
| Serotype 1 OspA-lumazine synthase; | MDEKNSVSVDLPGEMKVLVSKEKNKDGKYDLIATVDKLELKGTSDKNNGSGVLEGVKADKSKVKLTISDDLGQTTLEVFKEDGKTLVSKKVTSK | 15 |
| LFA-1 replacement Sero3 | DKSSTEEKFNEKGEVSEKIITRADGTRLEYTGIKSDGSGKAKEVLKGFALEGTLTDEKTTLVVKEGTVTLSKNISKSGEVSVELNDTDSSAATK | |
| | KTAAWNSGTSTLTITVNSKKTKDLVFTKENTITVQQYDSNGTKLEGSAVEITKLDEIKNALKGGGSMQIYEGKLTAEGLRFGIVASRFNHALVD | |
| | RLVEGAIDCIVRHGGREEDITLVRVPGSWEIPVAAGELARKEDIDAVIAIGVLIRGATPHFDYIASEVSKGLANLSLELRKPITFGVITADTLE | |
| | QAIERAGTKHGNKGWEAALSAIEMANLFKSLR | |
| Serotype 2 OspA-lumazine synthase | MDEKNSASVDLPGEMKVLVSKEKDKDGKYSLKATVDKIELKGTSDKDNGSGVLEGTKDDKSKAKLTIADDLSKTTFELFKEDGKTLVSRKVSSK | 16 |
| | DKTSTDEMFNEKGELSAKTMTRENGTKLEYTEMKSDGTGKAKEVLKNFTLEGKVANDKVTLEVKEGTVTLSKEIAKSGEVTVALNDTNTTQATK | |
| | KTGAWDSKTSTLTISVNSKKTTQLVFTKQDTITVQKYDSAGTNLEGTAVEIKTLDELKNALKGGGSMQIYEGKLTAEGLRFGIVASRFNHALVD | |
| | RLVEGAIDCIVRHGGREEDITLVRVPGSWEIPVAAGELARKEDIDAVIAIGVLIRGATPHFDYIASEVSKGLANLSLELRKPITFGVITADTLE | |
| | QAIERAGTKHGNKGWEAALSAIEMANLFKSLR | |
| Serotype 3 OspA-lumazine synthase | MDEKNSVSVDLPGGMKVLVSKEKDKDGKYSLMATVEKLELKGTSDKSNGSGVLEGEKADKSKAKLTISQDLNQTTFEIFKEDGKTLVSRKVNSK | 17 |
| | DKSSTEEKFNDKGKLSEKVVTRANGTRLEYTEIKNDGSGKAKEVLKGFALEGTLTDGGETKLTVTEGTVTLSKNISKSGEITVALNDTETTPAD | |
| | KKTGEWKSDTSTLTISKNSQKPKQLVFTKENTITVQNYNRAGNALEGSPAEIKDLAELKAALKGGGSMQIYEGKLTAEGLRFGIVASRFNHALV | |
| | DRLVEGAIDCIVRHGGREEDITLVRVPGSWEIPVAAGELARKEDIDAVIAIGVLIRGATPHFDYIASEVSKGLANLSLELRKPITFGVITADTL | |
| | EQAIERAGTKHGNKGWEAALSAIEMANLFKSLR | |
| Serotype 4 OspA-lumazine synthase | MDEKNSVSVDLPGEMKVLVSKEKDKDGKYSLMATVDKLELKGTSDKSNGSGTLEGEKSDKSKAKLTISEDLSKTTFEIFKEDGKTLVSKKVNSK | 18 |
| | DKSSIEEKFNAKGELSEKTILRANGTRLEYTEIKSDGTGKAKEVLKDFALEGTLAADKTTLKVTEGTVVLSKHIPNSGEITVELNDSNSTQATK | |
| | KTGKWDSNTSTLTISVNSKKTKNIVFTKEDTITVQKYDSAGTNLEGNAVEIKTLDELKNALKGGGSMQIYEGKLTAEGLRFGIVASRFNHALVD | |
| | RLVEGAIDCIVRHGGREEDITLVRVPGSWEIPVAAGELARKEDIDAVIAIGVLIRGATPHFDYIASEVSKGLANLSLELRKPITFGVITADTLE | |
| | QAIERAGTKHGNKGWEAALSAIEMANLFKSLR | |
| Serotype 5 OspA-lumazine synthase | MDEKNSVSVDLPGGMKVLVSKEKDKDGKYSLMATVEKLELKGTSDKNNGSGTLEGEKTDKSKVKLTIAEDLSKTTFEIFKEDGKTLVSKKVTLK | 19 |
| | DKSSTEEKFNEKGEISEKTIVRANGTRLEYTDIKSDGSGKAKEVLKDFTLEGTLAADGKTTLKVTEGTVVLSKNILKSGEITVALDDSDTTQAT | |
| | KKTGKWDSKTSTLTISVNSQKTKNLVFTKEDTITVQKYDSAGTNLEGKAVEITTLEKLKDALKGGGSMQIYEGKLTAEGLRFGIVASRFNHALV | |
| | DRLVEGAIDCIVRHGGREEDITLVRVPGSWEIPVAAGELARKEDIDAVIAIGVLIRGATPHFDYIASEVSKGLANLSLELRKPITFGVITADTL | |
| | EQAIERAGTKHGNKGWEAALSAIEMANLFKSLR | |
| Serotype 6 OspA-lumazine synthase | MDEKNSVSVDLPGGMTVLVSKEKDKDGKYSLEATVDKLELKGTSDKNNGSGTLEGEKTDKSKVKSTIADDLSQTKFEIFKEDGKTLVSKKVTLK | 20 |
| | DKSSTEEKFNGKGETSEKTIVRANGTRLEYTDIKSDGSGKAKEVLKDFTLEGTLAADGKTTLKVTEGTVVLSKNILKSGEITAALDDSDTTRAT | |
| | KKTGKWDSKTSTLTISVNSQKTKNLVFTKEDTITVQRYDSAGTNLEGKAVEITTLKELKNALKGGGSMQIYEGKLTAEGLRFGIVASRFNHALV | |
| | DRLVEGAIDCIVRHGGREEDITLVRVPGSWEIPVAAGELARKEDIDAVIAIGVLIRGATPHFDYIASEVSKGLANLSLELRKPITFGVITADTL | |
| | EQAIERAGTKHGNKGWEAALSAIEMANLFKSLR | |
| Serotype 7 OspA-lumazine synthase | MDEKNSVSVDLPGEMKVLVSKEKDKDGKYSLEATVDKLELKGTSDKNNGSGVLEGVKAAKSKAKLTIADDLSQTKFEIFKEDGKTLVSKKVTLK | 21 |
| | DKSSTEEKFNDKGKLSEKVVTRANGTRLEYTEIQNDGSGKAKEVLKSLTLEGTLTADGETKLTVEAGTVTLSKNISESGEITVELKDTETTPAD | |
| | KKSGTWDSKTSTLTISKNSQKTKQLVFTKENTITVQKYNTAGTKLEGSPAEIKDLEALKAALKGGGSMQIYEGKLTAEGLRFGIVASRFNHALV | |
| | DRLVEGAIDCIVRHGGREEDITLVRVPGSWEIPVAAGELARKEDIDAVIAIGVLIRGATPHFDYIASEVSKGLANLSLELRKPITFGVITADTL | |
| | EQAIERAGTKHGNKGWEAALSAIEMANLFKSLR | |
| Serotype 1 OspA; LFA-1 replacement | MDEKNSVSVDLPGEMKVLVSKEKNKDGKYDLIATVDKLELKGTSDKNNGSGVLEGVKADKSKVKLTISDDLGQTTLEVFKEDGKTLVSKKVTSK | 22 |
| RD2-Human Heavy chain ferritin | DKSSTEEKFNEKGEVSEKIITRADGTRLEYTGIKSDGSGKAKEVLKGYTLEGQLSDEKTTLVVKEGTVTLSKNISKSGEVSVELNDTDSSAATK | |
| | KTAAWNSGTSTLTITVNSKKTKDLVFTKENTITVQQYDSNGTKLEGSAVEITKLDEIKNALKGSMTTASTSQVRQNYHQDSEAAINRQINLELY | |
| | ASYVYLSMSYYFDRDDVALKNFAKYFLHQSHEEREHAEKLMKLQNQRGGRIFLQDIKKPDCDDWESGLNAMECALHLEKNVNQSLLELHKLATD | |
| | KNDPHLCDFIETHYLNEQVKAIKELGDHVTNLRKMGAPESGLAEYLFDKHTLGDSDNES | |
| OspA Serotype1; LFA-1 replacement | MDEKNSVSVDLPGEMKVLVSKEKNKDGKYDLIATVDKLELKGTSDKNNGSGVLEGVKADKSKVKLTISDDLGQTTLEVFKEDGKTLVSKKVTSK | 23 |
| RD1-Human Heavy chain ferritin | DKSSTEEKFNEKGEVSEKIITRADGTRLEYTGIKSDGSGKAKEVLKGYDLKGELSSEKTTLVVKEGTVTLSKNISKSGEVSVELNDTDSSAATK | |
| | KTAAWNSGTSTLTITVNSKKTKDLVFTKENTITVQQYDSNGTKLEGSAVEITKLDEIKNALKGSMTTASTSQVRQNYHQDSEAAINRQINLELY | |
| | ASYVYLSMSYYFDRDDVALKNFAKYFLHQSHEEREHAEKLMKLQNQRGGRIFLQDIKKPDCDDWESGLNAMECALHLEKNVNQSLLELHKLATD | |
| | KNDPHLCDFIETHYLNEQVKAIKELGDHVTNLRKMGAPESGLAEYLFDKHTLGDSDNES | |
| OspA Serotype1; LFA-1 replacement | MDEKNSVSVDLPGEMKVLVSKEKNKDGKYDLIATVDKLELKGTSDKNNGSGVLEGVKADKSKVKLTISDDLGQTTLEVFKEDGKTLVSKKVTSK | 24 |
| Serotype 2-Human Heavy chain | DKSSTEEKFNEKGEVSEKIITRADGTRLEYTGIKSDGSGKAKEVLKGFTLEGKVANEKTTLVVKEGTVTLSKNISKSGEVSVELNDTDSSAATK | |
| ferritin | KTAAWNSGTSTLTITVNSKKTKDLVFTKENTITVQQYDSNGTKLEGSAVEITKLDEIKNALKGSMTTASTSQVRQNYHQDSEAAINRQINLELY | |
| | ASYVYLSMSYYFDRDDVALKNFAKYFLHQSHEEREHAEKLMKLQNQRGGRIFLQDIKKPDCDDWESGLNAMECALHLEKNVNQSLLELHKLATD | |
| | KNDPHLCDFIETHYLNEQVKAIKELGDHVTNLRKMGAPESGLAEYLFDKHTLGDSDNES | |
| OspA Serotype1; LFA-1 replacement | MDEKNSVSVDLPGEMKVLVSKEKNKDGKYDLIATVDKLELKGTSDKNNGSGVLEGVKADKSKVKLTISDDLGQTTLEVFKEDGKTLVSKKVTSK | 25 |
| Serotype 3-Human Heavy chain | DKSSTEEKFNEKGEVSEKIITRADGTRLEYTGIKSDGSGKAKEVLKGFALEGTLTDEKTTLVVKEGTVTLSKNISKSGEVSVELNDTDSSAATK | |
| ferritin | KTAAWNSGTSTLTITVNSKKTKDLVFTKENTITVQQYDSNGTKLEGSAVEITKLDEIKNALKGSMTTASTSQVRQNYHQDSEAAINRQINLELY | |
| | ASYVYLSMSYYFDRDDVALKNFAKYFLHQSHEEREHAEKLMKLQNQRGGRIFLQDIKKPDCDDWESGLNAMECALHLEKNVNQSLLELHKLATD | |
| | KNDPHLCDFIETHYLNEQVKAIKELGDHVTNLRKMGAPESGLAEYLFDKHTLGDSDNES | |
| OspA Serotype2-Human Heavy chain | MDEKNSASVDLPGEMKVLVSKEKDKDGKYSLKATVDKIELKGTSDKDNGSGVLEGTKDDKSKAKLTIADDLSKTTFELFKEDGKTLVSRKVSSK | 26 |
| ferritin | DKTSTDEMFNEKGELSAKTMTRENGTKLEYTEMKSDGTGKAKEVLKNFTLEGKVANDKVTLEVKEGTVTLSKEIAKSGEVTVALNDTNTTQATK | |
| | KTGAWDSKTSTLTISVNSKKTTQLVFTKQDTITVQKYDSAGTNLEGTAVEIKTLDELKNALKGSMTTASTSQVRQNYHQDSEAAINRQINLELY | |
| | ASYVYLSMSYYFDRDDVALKNFAKYFLHQSHEEREHAEKLMKLQNQRGGRIFLQDIKKPDCDDWESGLNAMECALHLEKNVNQSLLELHKLATD | |
| | KNDPHLCDFIETHYLNEQVKAIKELGDHVTNLRKMGAPESGLAEYLFDKHTLGDSDNES | |
| OspA Serotype 3-Human Heavy chain | MDEKNSVSVDLPGGMKVLVSKEKDKDGKYSLMATVEKLELKGTSDKSNGSGVLEGEKADKSKAKLTISQDLNQTTFEIFKEDGKTLVSRKVNSK | 27 |
| ferritin | DKSSTEEKFNDKGKLSEKVVTRANGTRLEYTEIKNDGSGKAKEVLKGFALEGTLTDGGETKLTVTEGTVTLSKNISKSGEITVALNDTETTPAD | |
| | KKTGEWKSDTSTLTISKNSQKPKQLVFTKENTITVQNYNRAGNALEGSPAEIKDLAELKAALKGSMTTASTSQVRQNYHQDSEAAINRQINLEL | |
| | YASYVYLSMSYYFDRDDVALKNFAKYFLHQSHEEREHAEKLMKLQNQRGGRIFLQDIKKPDCDDWESGLNAMECALHLEKNVNQSLLELHKLAT | |
| | DKNDPHLCDFIETHYLNEQVKAIKELGDHVTNLRKMGAPESGLAEYLFDKHTLGDSDNES | |
| OspA Serotype 4-Human Heavy chain | MDEKNSVSVDLPGEMKVLVSKEKDKDGKYSLMATVDKLELKGTSDKSNGSGTLEGEKSDKSKAKLTISEDLSKTTFEIFKEDGKTLVSKKVNSK | 28 |
| ferritin | DKSSIEEKFNAKGELSEKTILRANGTRLEYTEIKSDGTGKAKEVLKDFALEGTLAADKTTLKVTEGTVVLSKHIPNSGEITVELNDSNSTQATK | |
| | KTGKWDSNTSTLTISVNSKKTKNIVFTKEDTITVQKYDSAGTNLEGNAVEIKTLDELKNALKGSMTTASTSQVRQNYHQDSEAAINRQINLELY | |
| | ASYVYLSMSYYFDRDDVALKNFAKYFLHQSHEEREHAEKLMKLQNQRGGRIFLQDIKKPDCDDWESGLNAMECALHLEKNVNQSLLELHKLATD | |
| | KNDPHLCDFIETHYLNEQVKAIKELGDHVTNLRKMGAPESGLAEYLFDKHTLGDSDNES | |
| OspA Serotype 5-Human Heavy chain | MDEKNSVSVDLPGGMKVLVSKEKDKDGKYSLMATVEKLELKGTSDKNNGSGTLEGEKTDKSKVKLTIAEDLSKTTFEIFKEDGKTLVSKKVTLK | 29 |
| ferritin | DKSSTEEKFNEKGEISEKTIVRANGTRLEYTDIKSDGSGKAKEVLKDFTLEGTLAADGKTTLKVTEGTVVLSKNILKSGEITVALDDSDTTQAT | |
| | KKTGKWDSKTSTLTISVNSQKTKNLVFTKEDTITVQKYDSAGTNLEGKAVEITTLEKLKDALKGSMTTASTSQVRQNYHQDSEAAINRQINLEL | |
| | YASYVYLSMSYYFDRDDVALKNFAKYFLHQSHEEREHAEKLMKLQNQRGGRIFLQDIKKPDCDDWESGLNAMECALHLEKNVNQSLLELHKLAT | |
| | DKNDPHLCDFIETHYLNEQVKAIKELGDHVTNLRKMGAPESGLAEYLFDKHTLGDSDNES | |
| OspA Serotype 6-Human Heavy chain | MDEKNSVSVDLPGGMTVLVSKEKDKDGKYSLEATVDKLELKGTSDKNNGSGTLEGEKTDKSKVKSTIADDLSQTKFEIFKEDGKTLVSKKVTLK | 30 |
| ferritin | DKSSTEEKFNGKGETSEKTIVRANGTRLEYTDIKSDGSGKAKEVLKDFTLEGTLAADGKTTLKVTEGTVVLSKNILKSGEITAALDDSDTTRAT | |
| | KKTGKWDSKTSTLTISVNSQKTKNLVFTKEDTITVQRYDSAGTNLEGKAVEITTLKELKNALKGSMTTASTSQVRQNYHQDSEAAINRQINLEL | |
| | YASYVYLSMSYYFDRDDVALKNFAKYFLHQSHEEREHAEKLMKLQNQRGGRIFLQDIKKPDCDDWESGLNAMECALHLEKNVNQSLLELHKLAT | |
| | DKNDPHLCDFIETHYLNEQVKAIKELGDHVTNLRKMGAPESGLAEYLFDKHTLGDSDNES | |
| OspA Serotype 7-Human Heavy chain | MDEKNSVSVDLPGEMKVLVSKEKDKDGKYSLEATVDKLELKGTSDKNNGSGVLEGVKAAKSKAKLTIADDLSQTKFEIFKEDGKTLVSKKVTLK | 31 |
| ferritin | DKSSTEEKFNDKGKLSEKVVTRANGTRLEYTEIQNDGSGKAKEVLKSLTLEGTLTADGETKLTVEAGTVTLSKNISESGEITVELKDTETTPAD | |
| | KKSGTWDSKTSTLTISKNSQKTKQLVFTKENTITVQKYNTAGTKLEGSPAEIKDLEALKAALKGSMTTASTSQVRQNYHQDSEAAINRQINLEL | |
| | YASYVYLSMSYYFDRDDVALKNFAKYFLHQSHEEREHAEKLMKLQNQRGGRIFLQDIKKPDCDDWESGLNAMECALHLEKNVNQSLLELHKLAT | |
| | DKNDPHLCDFIETHYLNEQVKAIKELGDHVTNLRKMGAPESGLAEYLFDKHTLGDSDNES | |
| OspA Serotype 1; LFA-1 replacement | MDEKNSVSVDLPGEMKVLVSKEKNKDGKYDLIATVDKLELKGTSDKNNGSGVLEGVKADKSKVKLTISDDLGQTTLEVFKEDGKTLVSKKVTSK | 32 |
| RD2-Pyrococcus furiosus ferritin | DKSSTEEKFNEKGEVSEKIITRADGTRLEYTGIKSDGSGKAKEVLKGYTLEGQLSDEKTTLVVKEGTVTLSKNISKSGEVSVELNDTDSSAATK | |
| | KTAAWNSGTSTLTITVNSKKTKDLVFTKENTITVQQYDSNGTKLEGSAVEITKLDEIKNALKGSMLSERMLKALNDQLNRELYSAYLYFAMAAY | |
| | FEDLGLEGFANWMKAQAEEEIGHALRFYNYIYDRNGRVELDEIPKPPKEWESPLKAFEAAYEHEKFISKSIYELAALAEEEKDYSTRAFLEWFI | |
| | NEQVEEEASVKKILDKLKFAKDSPQILFMLDKELSARAPKLPGLLMQGGE | |
| OspA Serotype 1; LFA-1 replacement | MDEKNSVSVDLPGEMKVLVSKEKNKDGKYDLIATVDKLELKGTSDKNNGSGVLEGVKADKSKVKLTISDDLGQTTLEVFKEDGKTLVSKKVTSK | 33 |
| RD1-Pyrococcus furiosus ferritin | DKSSTEEKFNEKGEVSEKIITRADGTRLEYTGIKSDGSGKAKEVLKGYDLKGELSSEKTTLVVKEGTVTLSKNISKSGEVSVELNDTDSSAATK | |
| | KTAAWNSGTSTLTITVNSKKTKDLVFTKENTITVQQYDSNGTKLEGSAVEITKLDEIKNALKGSMLSERMLKALNDQLNRELYSAYLYFAMAAY | |
| | FEDLGLEGFANWMKAQAEEEIGHALRFYNYIYDRNGRVELDEIPKPPKEWESPLKAFEAAYEHEKFISKSIYELAALAEEEKDYSTRAFLEWFI | |
| | NEQVEEEASVKKILDKLKFAKDSPQILFMLDKELSARAPKLPGLLMQGGE | |
| OspA Serotype 1; LFA-1 replacement | MDEKNSVSVDLPGEMKVLVSKEKNKDGKYDLIATVDKLELKGTSDKNNGSGVLEGVKADKSKVKLTISDDLGQTTLEVFKEDGKTLVSKKVTSK | 34 |
| Serotype2-Pyrococcus furiosus | DKSSTEEKFNEKGEVSEKIITRADGTRLEYTGIKSDGSGKAKEVLKGFTLEGKVANEKTTLVVKEGTVTLSKNISKSGEVSVELNDTDSSAATK | |
| ferritin | KTAAWNSGTSTLTITVNSKKTKDLVFTKENTITVQQYDSNGTKLEGSAVEITKLDEIKNALKGSMLSERMLKALNDQLNRELYSAYLYFAMAAY | |
| | FEDLGLEGFANWMKAQAEEEIGHALRFYNYIYDRNGRVELDEIPKPPKEWESPLKAFEAAYEHEKFISKSIYELAALAEEEKDYSTRAFLEWFI | |
| | NEQVEEEASVKKILDKLKFAKDSPQILFMLDKELSARAPKLPGLLMQGGE | |
| OspA Serotype 1; LFA-1 replacement | MDEKNSVSVDLPGEMKVLVSKEKNKDGKYDLIATVDKLELKGTSDKNNGSGVLEGVKADKSKVKLTISDDLGQTTLEVFKEDGKTLVSKKVTSK | 35 |
| Serotype 3-Pyrococcus furiosus | DKSSTEEKFNEKGEVSEKIITRADGTRLEYTGIKSDGSGKAKEVLKGFALEGTLTDEKTTLVVKEGTVTLSKNISKSGEVSVELNDTDSSAATK | |
| ferritin | KTAAWNSGTSTLTITVNSKKTKDLVFTKENTITVQQYDSNGTKLEGSAVEITKLDEIKNALKGSMLSERMLKALNDQLNRELYSAYLYFAMAAY | |
| | FEDLGLEGFANWMKAQAEEEIGHALRFYNYIYDRNGRVELDEIPKPPKEWESPLKAFEAAYEHEKFISKSIYELAALAEEEKDYSTRAFLEWFI | |
| | NEQVEEEASVKKILDKLKFAKDSPQILFMLDKELSARAPKLPGLLMQGGE | |
| OspA Serotype 2-Pyrococcus furiosus | MDEKNSASVDLPGEMKVLVSKEKDKDGKYSLKATVDKIELKGTSDKDNGSGVLEGTKDDKSKAKLTIADDLSKTTFELFKEDGKTLVSRKVSSK | 36 |
| ferritin | DKTSTDEMFNEKGELSAKTMTRENGTKLEYTEMKSDGTGKAKEVLKNFTLEGKVANDKVTLEVKEGTVTLSKEIAKSGEVTVALNDTNTTQATK | |
| | KTGAWDSKTSTLTISVNSKKTTQLVFTKQDTITVQKYDSAGTNLEGTAVEIKTLDELKNALKGSMLSERMLKALNDQLNRELYSAYLYFAMAAY | |
| | FEDLGLEGFANWMKAQAEEEIGHALRFYNYIYDRNGRVELDEIPKPPKEWESPLKAFEAAYEHEKFISKSIYELAALAEEEKDYSTRAFLEWFI | |
| | NEQVEEEASVKKILDKLKFAKDSPQILFMLDKELSARAPKLPGLLMQGGE | |
| OspA Serotype 3-Pyrococcus furiosus | MDEKNSVSVDLPGGMKVLVSKEKDKDGKYSLMATVEKLELKGTSDKSNGSGVLEGEKADKSKAKLTISQDLNQTTFEIFKEDGKTLVSRKVNSK | 37 |
| ferritin | DKSSTEEKFNDKGKLSEKVVTRANGTRLEYTEIKNDGSGKAKEVLKGFALEGTLTDGGETKLTVTEGTVTLSKNISKSGEITVALNDTETTPAD | |
| | KKTGEWKSDTSTLTISKNSQKPKQLVFTKENTITVQNYNRAGNALEGSPAEIKDLAELKAALKGSMLSERMLKALNDQLNRELYSAYLYFAMAA | |
| | YFEDLGLEGFANWMKAQAEEEIGHALRFYNYIYDRNGRVELDEIPKPPKEWESPLKAFEAAYEHEKFISKSIYELAALAEEEKDYSTRAFLEWF | |
| | INEQVEEEASVKKILDKLKFAKDSPQILFMLDKELSARAPKLPGLLMQGGE | |
| OspA Serotype 4-Pyrococcus furiosus | MDEKNSVSVDLPGEMKVLVSKEKDKDGKYSLMATVDKLELKGTSDKSNGSGTLEGEKSDKSKAKLTISEDLSKTTFEIFKEDGKTLVSKKVNSK | 38 |
| ferritin | DKSSIEEKFNAKGELSEKTILRANGTRLEYTEIKSDGTGKAKEVLKDFALEGTLAADKTTLKVTEGTVVLSKHIPNSGEITVELNDSNSTQATK | |
| | KTGKWDSNTSTLTISVNSKKTKNIVFTKEDTITVQKYDSAGTNLEGNAVEIKTLDELKNALKGSMLSERMLKALNDQLNRELYSAYLYFAMAAY | |
| | FEDLGLEGFANWMKAQAEEEIGHALRFYNYIYDRNGRVELDEIPKPPKEWESPLKAFEAAYEHEKFISKSIYELAALAEEEKDYSTRAFLEWFI | |
| | NEQVEEEASVKKILDKLKFAKDSPQILFMLDKELSARAPKLPGLLMQGGE | |
| OspA Serotype 5-Pyrococcus furiosus | MDEKNSVSVDLPGGMKVLVSKEKDKDGKYSLMATVEKLELKGTSDKNNGSGTLEGEKTDKSKVKLTIAEDLSKTTFEIFKEDGKTLVSKKVTLK | 39 |
| ferritin | DKSSTEEKFNEKGEISEKTIVRANGTRLEYTDIKSDGSGKAKEVLKDFTLEGTLAADGKTTLKVTEGTVVLSKNILKSGEITVALDDSDTTQAT | |
| | KKTGKWDSKTSTLTISVNSQKTKNLVFTKEDTITVQKYDSAGTNLEGKAVEITTLEKLKDALKGSMLSERMLKALNDQLNRELYSAYLYFAMAA | |
| | YFEDLGLEGFANWMKAQAEEEIGHALRFYNYIYDRNGRVELDEIPKPPKEWESPLKAFEAAYEHEKFISKSIYELAALAEEEKDYSTRAFLEWF | |
| | INEQVEEEASVKKILDKLKFAKDSPQILFMLDKELSARAPKLPGLLMQGGE | |
| OspA Serotype 6-Pyrococcus furiosus | MDEKNSVSVDLPGGMTVLVSKEKDKDGKYSLEATVDKLELKGTSDKNNGSGTLEGEKTDKSKVKSTIADDLSQTKFEIFKEDGKTLVSKKVTLK | 40 |
| ferritin | DKSSTEEKFNGKGETSEKTIVRANGTRLEYTDIKSDGSGKAKEVLKDFTLEGTLAADGKTTLKVTEGTVVLSKNILKSGEITAALDDSDTTRAT | |
| | KKTGKWDSKTSTLTISVNSQKTKNLVFTKEDTITVQRYDSAGTNLEGKAVEITTLKELKNALKGSMLSERMLKALNDQLNRELYSAYLYFAMAA | |
| | YFEDLGLEGFANWMKAQAEEEIGHALRFYNYIYDRNGRVELDEIPKPPKEWESPLKAFEAAYEHEKFISKSIYELAALAEEEKDYSTRAFLEWF | |
| | INEQVEEEASVKKILDKLKFAKDSPQILFMLDKELSARAPKLPGLLMQGGE | |
| OspA Serotype 7-Pyrococcus furiosus | MDEKNSVSVDLPGEMKVLVSKEKDKDGKYSLEATVDKLELKGTSDKNNGSGVLEGVKAAKSKAKLTIADDLSQTKFEIFKEDGKTLVSKKVTLK | 41 |
| ferritin | DKSSTEEKFNDKGKLSEKVVTRANGTRLEYTEIQNDGSGKAKEVLKSLTLEGTLTADGETKLTVEAGTVTLSKNISESGEITVELKDTETTPAD | |
| | KKSGTWDSKTSTLTISKNSQKTKQLVFTKENTITVQKYNTAGTKLEGSPAEIKDLEALKAALKGSMLSERMLKALNDQLNRELYSAYLYFAMAA | |
| | YFEDLGLEGFANWMKAQAEEEIGHALRFYNYIYDRNGRVELDEIPKPPKEWESPLKAFEAAYEHEKFISKSIYELAALAEEEKDYSTRAFLEWF | |
| | INEQVEEEASVKKILDKLKFAKDSPQILFMLDKELSARAPKLPGLLMQGGE | |
| OspA Serotype 1; LFA-1 replacement | MDEKNSVSVDLPGEMKVLVSKEKNKDGKYDLIATVDKLELKGTSDKNNGSGVLEGVKADKSKVKLTISDDLGQTTLEVFKEDGKTLVSKKVTSK | 42 |
| RD2-Trichoplusia Ni ferritin | DKSSTEEKFNEKGEVSEKIITRADGTRLEYTGIKSDGSGKAKEVLKGYTLEGQLSDEKTTLVVKEGTVTLSKNISKSGEVSVELNDTDSSAATK | |
| | KTAAWNSGTSTLTITVNSKKTKDLVFTKENTITVQQYDSNGTKLEGSAVEITKLDEIKNALKGSTQCNVNPVQIPKDWITMHRSCRNSMRQQIQ | |
| | MEVGASLQYLAMGAHFSKDVVNRPGFAQLFFDAASEEREHAMKLIEYLLMRGELTNDVSSLLQVRPPTRSSWKGGVEALEHALSMESDVTKSIR | |
| | NVIKACEDDSEFNDYHLVDYLTGDFLEEQYKGQRDLAGKASTLKKLMDRHEALGEFIFDKKLLGIDV | |
| OspA Serotype 1; LFA-1 replacement | MDEKNSVSVDLPGEMKVLVSKEKNKDGKYDLIATVDKLELKGTSDKNNGSGVLEGVKADKSKVKLTISDDLGQTTLEVFKEDGKTLVSKKVTSK | 43 |
| RD1-Trichoplusia Ni ferritin | DKSSTEEKFNEKGEVSEKIITRADGTRLEYTGIKSDGSGKAKEVLKGYDLKGELSSEKTTLVVKEGTVTLSKNISKSGEVSVELNDTDSSAATK | |
| | KTAAWNSGTSTLTITVNSKKTKDLVFTKENTITVQQYDSNGTKLEGSAVEITKLDEIKNALKGSTQCNVNPVQIPKDWITMHRSCRNSMRQQIQ | |
| | MEVGASLQYLAMGAHFSKDVVNRPGFAQLFFDAASEEREHAMKLIEYLLMRGELTNDVSSLLQVRPPTRSSWKGGVEALEHALSMESDVTKSIR | |
| | NVIKACEDDSEFNDYHLVDYLTGDFLEEQYKGQRDLAGKASTLKKLMDRHEALGEFIFDKKLLGIDV | |
| OspA Serotype 1; LFA-1 replacement | MDEKNSVSVDLPGEMKVLVSKEKNKDGKYDLIATVDKLELKGTSDKNNGSGVLEGVKADKSKVKLTISDDLGQTTLEVFKEDGKTLVSKKVTSK | 44 |
| Serotype 2-Trichoplusia Ni ferritin | DKSSTEEKFNEKGEVSEKIITRADGTRLEYTGIKSDGSGKAKEVLKGFTLEGKVANEKTTLVVKEGTVTLSKNISKSGEVSVELNDTDSSAATK | |
| | KTAAWNSGTSTLTITVNSKKTKDLVFTKENTITVQQYDSNGTKLEGSAVEITKLDEIKNALKGSTQCNVNPVQIPKDWITMHRSCRNSMRQQIQ | |
| | MEVGASLQYLAMGAHFSKDVVNRPGFAQLFFDAASEEREHAMKLIEYLLMRGELTNDVSSLLQVRPPTRSSWKGGVEALEHALSMESDVTKSIR | |
| | NVIKACEDDSEFNDYHLVDYLTGDFLEEQYKGQRDLAGKASTLKKLMDRHEALGEFIFDKKLLGIDV | |
| OspA Serotype1; LFA-1 replacement | MDEKNSVSVDLPGEMKVLVSKEKNKDGKYDLIATVDKLELKGTSDKNNGSGVLEGVKADKSKVKLTISDDLGQTTLEVFKEDGKTLVSKKVTSK | 45 |
| Serotype 3-Trichoplusia Ni ferritin | DKSSTEEKFNEKGEVSEKIITRADGTRLEYTGIKSDGSGKAKEVLKGFALEGTLTDEKTTLVVKEGTVTLSKNISKSGEVSVELNDTDSSAATK | |
| | KTAAWNSGTSTLTITVNSKKTKDLVFTKENTITVQQYDSNGTKLEGSAVEITKLDEIKNALKGSTQCNVNPVQIPKDWITMHRSCRNSMRQQIQ | |
| | MEVGASLQYLAMGAHFSKDVVNRPGFAQLFFDAASEEREHAMKLIEYLLMRGELTNDVSSLLQVRPPTRSSWKGGVEALEHALSMESDVTKSIR | |
| | NVIKACEDDSEFNDYHLVDYLTGDFLEEQYKGQRDLAGKASTLKKLMDRHEALGEFIFDKKLLGIDV | |
| OspA Serotype 2-Trichoplusia Ni | MDEKNSASVDLPGEMKVLVSKEKDKDGKYSLKATVDKIELKGTSDKDNGSGVLEGTKDDKSKAKLTIADDLSKTTFELFKEDGKTLVSRKVSSK | 46 |
| ferritin | DKTSTDEMFNEKGELSAKTMTRENGTKLEYTEMKSDGTGKAKEVLKNFTLEGKVANDKVTLEVKEGTVTLSKEIAKSGEVTVALNDTNTTQATK | |
| | KTGAWDSKTSTLTISVNSKKTTQLVFTKQDTITVQKYDSAGTNLEGTAVEIKTLDELKNALKGSTQCNVNPVQIPKDWITMHRSCRNSMRQQIQ | |
| | MEVGASLQYLAMGAHFSKDVVNRPGFAQLFFDAASEEREHAMKLIEYLLMRGELTNDVSSLLQVRPPTRSSWKGGVEALEHALSMESDVTKSIR | |
| | NVIKACEDDSEFNDYHLVDYLTGDFLEEQYKGQRDLAGKASTLKKLMDRHEALGEFIFDKKLLGIDV | |
| OspA Serotype 3 Trichoplusia Ni | MDEKNSVSVDLPGGMKVLVSKEKDKDGKYSLMATVEKLELKGTSDKSNGSGVLEGEKADKSKAKLTISQDLNQTTFEIFKEDGKTLVSRKVNSK | 47 |
| ferritin | DKSSTEEKFNDKGKLSEKVVTRANGTRLEYTEIKNDGSGKAKEVLKGFALEGTLTDGGETKLTVTEGTVTLSKNISKSGEITVALNDTETTPAD | |
| | KKTGEWKSDTSTLTISKNSQKPKQLVFTKENTITVQNYNRAGNALEGSPAEIKDLAELKAALKGSTQCNVNPVQIPKDWITMHRSCRNSMRQQI | |
| | QMEVGASLQYLAMGAHFSKDVVNRPGFAQLFFDAASEEREHAMKLIEYLLMRGELTNDVSSLLQVRPPTRSSWKGGVEALEHALSMESDVTKSI | |
| | RNVIKACEDDSEFNDYHLVDYLTGDFLEEQYKGQRDLAGKASTLKKLMDRHEALGEFIFDKKLLGIDV | |
| OspA Serotype 4-Trichoplusia Ni | MDEKNSVSVDLPGEMKVLVSKEKDKDGKYSLMATVDKLELKGTSDKSNGSGTLEGEKSDKSKAKLTISEDLSKTTFEIFKEDGKTLVSKKVNSK | 48 |
| ferritin | DKSSIEEKFNAKGELSEKTILRANGTRLEYTEIKSDGTGKAKEVLKDFALEGTLAADKTTLKVTEGTVVLSKHIPNSGEITVELNDSNSTQATK | |
| | KTGKWDSNTSTLTISVNSKKTKNIVFTKEDTITVQKYDSAGTNLEGNAVEIKTLDELKNALKGSTQCNVNPVQIPKDWITMHRSCRNSMRQQIQ | |
| | MEVGASLQYLAMGAHFSKDVVNRPGFAQLFFDAASEEREHAMKLIEYLLMRGELTNDVSSLLQVRPPTRSSWKGGVEALEHALSMESDVTKSIR | |
| | NVIKACEDDSEFNDYHLVDYLTGDFLEEQYKGQRDLAGKASTLKKLMDRHEALGEFIFDKKLLGIDV | |
| OspA Serotype 5-Trichoplusia Ni | MDEKNSVSVDLPGGMKVLVSKEKDKDGKYSLMATVEKLELKGTSDKNNGSGTLEGEKTDKSKVKLTIAEDLSKTTFEIFKEDGKTLVSKKVTLK | 49 |
| ferritin | DKSSTEEKFNEKGEISEKTIVRANGTRLEYTDIKSDGSGKAKEVLKDFTLEGTLAADGKTTLKVTEGTVVLSKNILKSGEITVALDDSDTTQAT | |
| | KKTGKWDSKTSTLTISVNSQKTKNLVFTKEDTITVQKYDSAGTNLEGKAVEITTLEKLKDALKGSTQCNVNPVQIPKDWITMHRSCRNSMRQQI | |
| | QMEVGASLQYLAMGAHFSKDVVNRPGFAQLFFDAASEEREHAMKLIEYLLMRGELTNDVSSLLQVRPPTRSSWKGGVEALEHALSMESDVTKSI | |
| | RNVIKACEDDSEFNDYHLVDYLTGDFLEEQYKGQRDLAGKASTLKKLMDRHEALGEFIFDKKLLGIDV | |
| OspA Serotype 6-Trichoplusia Ni | MDEKNSVSVDLPGGMTVLVSKEKDKDGKYSLEATVDKLELKGTSDKNNGSGTLEGEKTDKSKVKSTIADDLSQTKFEIFKEDGKTLVSKKVTLK | 50 |
| ferritin | DKSSTEEKFNGKGETSEKTIVRANGTRLEYTDIKSDGSGKAKEVLKDFTLEGTLAADGKTTLKVTEGTVVLSKNILKSGEITAALDDSDTTRAT | |
| | KKTGKWDSKTSTLTISVNSQKTKNLVFTKEDTITVQRYDSAGTNLEGKAVEITTLKELKNALKGSTQCNVNPVQIPKDWITMHRSCRNSMRQQI | |
| | QMEVGASLQYLAMGAHFSKDVVNRPGFAQLFFDAASEEREHAMKLIEYLLMRGELTNDVSSLLQVRPPTRSSWKGGVEALEHALSMESDVTKSI | |
| | RNVIKACEDDSEFNDYHLVDYLTGDFLEEQYKGQRDLAGKASTLKKLMDRHEALGEFIFDKKLLGIDV | |
| OspA Serotype 7-Trichoplusia Ni | MDEKNSVSVDLPGEMKVLVSKEKDKDGKYSLEATVDKLELKGTSDKNNGSGVLEGVKAAKSKAKLTIADDLSQTKFEIFKEDGKTLVSKKVTLK | 51 |
| ferritin | DKSSTEEKFNDKGKLSEKVVTRANGTRLEYTEIQNDGSGKAKEVLKSLTLEGTLTADGETKLTVEAGTVTLSKNISESGEITVELKDTETTPAD | |
| | KKSGTWDSKTSTLTISKNSQKTKQLVFTKENTITVQKYNTAGTKLEGSPAEIKDLEALKAALKGSTQCNVNPVQIPKDWITMHRSCRNSMRQQI | |
| | QMEVGASLQYLAMGAHFSKDVVNRPGFAQLFFDAASEEREHAMKLIEYLLMRGELTNDVSSLLQVRPPTRSSWKGGVEALEHALSMESDVTKSI | |
| | RNVIKACEDDSEFNDYHLVDYLTGDFLEEQYKGQRDLAGKASTLKKLMDRHEALGEFIFDKKLLGIDV | |
| Serotype 1 LFA-1 replacement RD2 | MDSKGSSQKGSRLLLLLVVSNLLLPQGVLAMDEKNSVSVDLPGEMKVLVSKEKNKDGKYDLIATVDKLELKGTSDKNNGSGVLEGVKADKSKVK | 52 |
| OspA-ferritin | LTISDDLGQTTLEVFKEDGKTLVSKKVTSKDKSSTEEKFNEKGEVSEKIITRADGTRLEYTGIKSDGSGKAKEVLKGYTLEGQLSDEKTTLVVK | |
| | EGTVTLSKNISKSGEVSVELNDTDSSAATKKTAAWNSGTSTLTITVNSKKTKDLVFTKENTITVQQYDSNGTKLEGSAVEITKLDEIKNALKGS | |
| | 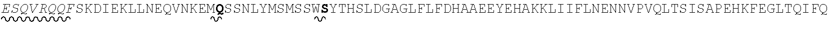 | |
| | 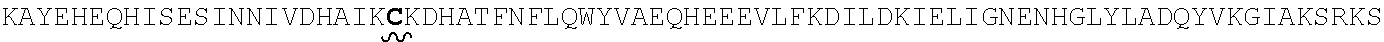 | |
| Serotype 1 Non-glycosylated LFA-1 | MDSKGSSQKGSRLLLLLVVSNLLLPQGVLAMDEKNSVSVDLPGEMKVLVSKEKNKDGKYDLIATVDKLELKGTSDKNNGSGVLEGVKADKSKVK | 53 |
| replacement RD2 OspA-ferritin | LTISDDLGQTTLEVFKEDGKTLVSKKVTSKDKSSTEEKFNEKGEVSEKIITRADGTRLEYTGIKSDGSGKAKEVLKGYTLEGQLSDEKTTLVVK | |
| | EGTVTLSKNISKSGEVSVELNDTDSSAATKKTAAWNSGTSTLTITVNSKKTKDLVFTKENTITVQQYDSNGTKLEGSAVEITKLDEIKNALKGS | |
| | 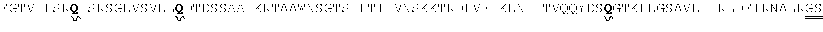 | |
| | KAYEHEQHISESINNIVDHAIKCKDHATFNFLQWVAEQHEEEVLFKDILDKIELIGNENHGLYLADQYVKGIAKSRKS | |
| OspA-ferritin Serotype 1 with LFA-1 | MDSKGSSQKGSRLLLLLVVSNLLLPQGVLAMDEKNSVSVDLPGEMKVLVSKEKNKDGKYDLIATVDKLELKGTSDKNQGSGVLEGVKADKSKVK | 54 |
| site replaced with Serotype 2 | LTISDDLGQTTLEVFKEDGKTLVSKKVTSKDKSSTEEKFNEKGEVSEKIITRADGTRLEYTGIKSDGSGKAKEVLKGFTLEGKVANEKTTLVVK | |
| | EGTVTLSKNISKSGEVSVELNDTDSSAATKKTAAWNSGTSTLTITVNSKKTKDLVFTKENTITVQQYDSNGTKLEGSAVEITKLDEIKNALKGS | |
| | 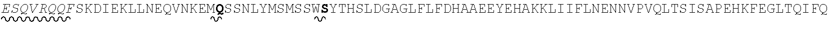 | |
| | 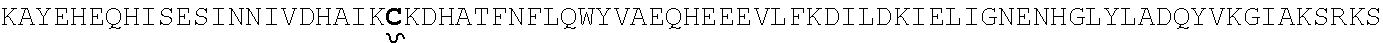 | |
| Non-glycosylated OspA-ferritin Serotype 1 with LFA-1 site | 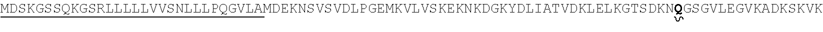 | 55 |
| replaced with Serotype 2 | LTISDDLGQTTLEVFKEDGKTLVSKKVTSKDKSSTEEKFNEKGEVSEKIITRADGTRLEYTGIKSDGSGKAKEVLKGFTLEGKVANEKTTLVVK | |
| | EGTVTLSQISKSGEVSVELQDTDSSAATKKTAAWNSGTSTLTITVNSKKTKDLVFTKENTITVQQYDSQGTKLEGSAVEITKLDEIKNALKGS | |
| | 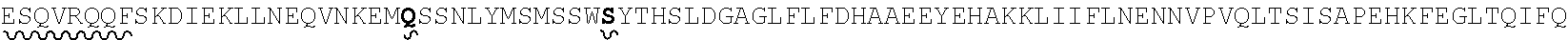 | |
| | 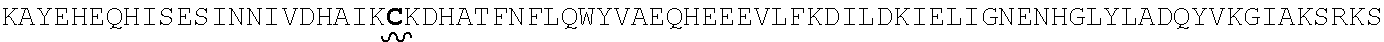 | |
| OspA-ferritin Serotype 1 with | MDSKGSSQKGSRLLLLLVVSNLLLPQGVLAMDEKNSVSVDLPGEMKVLVSKEKNKDGKYDLIATVDKLELKGTSDKNNGSGVLEGVKADKSKVK | 56 |
| LFA-1 site replaced with Serotype 3 | LTISDDLGQTTLEVFKEDGKTLVSKKVTSKDKSSTEEKFNEKGEVSEKIITRADGTRLEYTGIKSDGSGKAKEVLKGFALEGTLTDEKTTLVVK | |
| | EGTVTLSKNISKSGEVSVELNDTDSSAATKKTAAWNSGTSTLTITVNSKKTKDLVFTKENTITVQQYDSNGTKLEGSAVEITKLDEIKNALKGS | |
| | 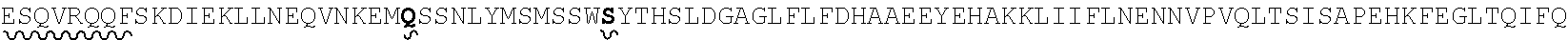 | |
| | 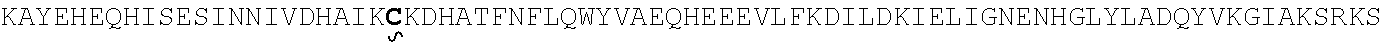 | |
| Non-glycosylated OspA-ferritin Serotype 1 with LFA-1 site | 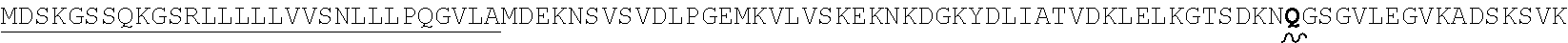 | 57 |
| replaced with Serotype 3 | LTISDDLGQTTLEVFKEDGKTLVSKKVTSKDKSSTEEKFNEKGEVSEKIITRADGTRLEYTGIKSDGSGKAKEVLKGFALEGTLTDEKTTLVVK | |
| | 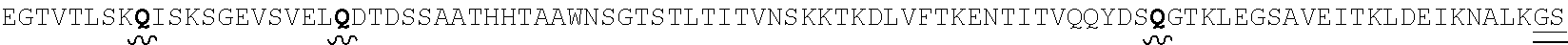 | |
| | 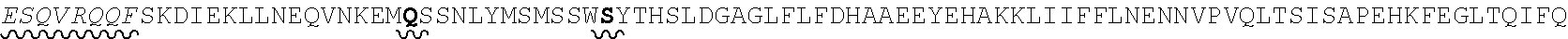 | |
| | 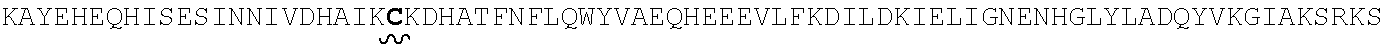 | |
| OspA-ferritin Serotype 1 with LFA-1 | MDSKGSSQKGSRLLLLLVVSNLLLPQGVLAMDEKNSVSVDLPGEMKVLVSKEKNKDGKYDLIATVDKLELKGTSDKNNGSGVLEGVKADKSKVK | 58 |
| site replaced with RD1 | LTISDDLGQTTLEVFKEDGKTLVSKKVTSKDKSSTEEKFNEKGEVSEKIITRADGTRLEYTGIKSDGSGKAKEVLKGYDLKGELSSEKTTLVVK | |
| | EGTVTLSKNISKSGEVSVELNDTDSSAATKKTAAWNSGTSTLTITVNSKKTKDLVFTKENTITVQQYDSNGTKLEGSAVEITKLDEIKNALKGS | |
| | 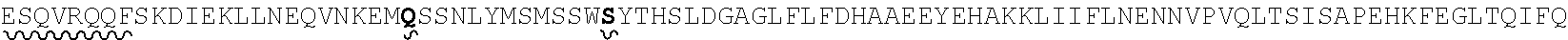 | |
| | 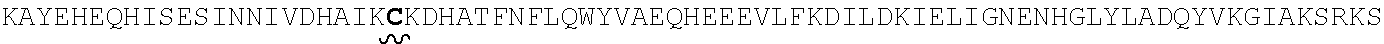 | |
| Non-glycosylated OspA-ferritin Serotype 1 with LFA-1 site replaced | 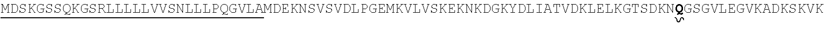 | 59 |
| with RD1 | LTISDDLGQTTLEVFKEDGKTLVSKKVTSKDKSSTEEKFNEKGEVSEKIITRADGTRLEYTGIKSDGSGKAKEVLKGYDLKGELSSEKTTLVVK | |
| | 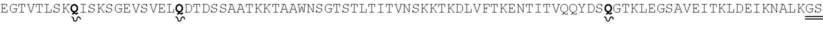 | |
| | 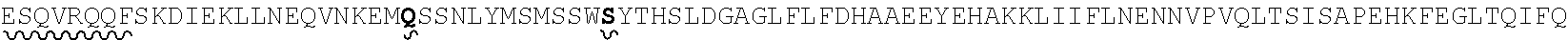 | |
| | 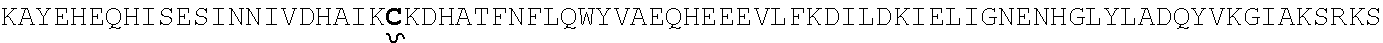 | |
| Non-glycosylated, OspA-ferritin Serotype 1, LFA-1 replacement | 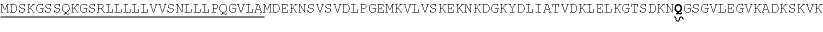 | 60 |
| RD2, lx GGGS linker (SEQ ID NO: 226) | LTISDDLGQTTLEVFKEDGKTLVSKKVTSKDKSSTEEKFNEKGEVSEKIITRADGTRLEYTGIKSDGSGKAKEVLKGYTLEGQLSDEKTTLVVK | |
| | 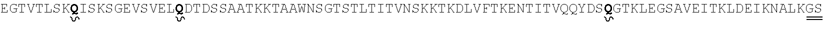 | |
| | 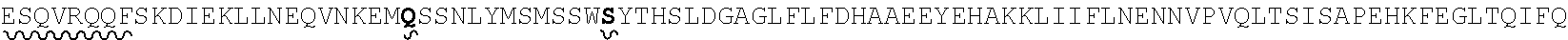 | |
| | 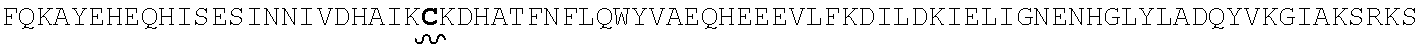 | |
| Non-glycosylated,OspA-ferritin Serotype 1, LFA-1 replacement | 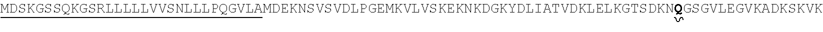 | 61 |
| RD2, 2x GGGS linker (SEQ ID NO: 91) | LTISDDLGQTTLEVFKEDGKTLVSKKVTSKDKSSTEEKFNEKGEVSEKIITRADGTRLEYTGIKSDGSGKAKEVLKGYTLEGQLSDEKTTLVVK | |
| | 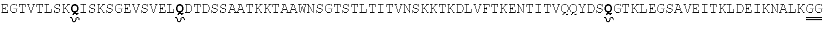 | |
| | 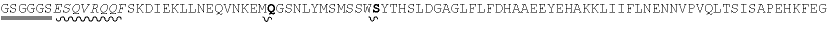 | |
| | 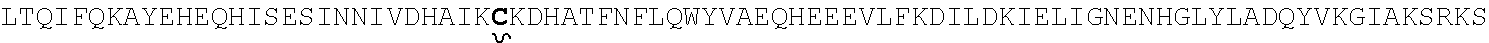 | |
| Non-glycosylated, OspA-ferritin Serotype 1, LFA-1 replacement | 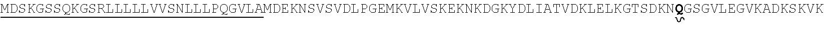 | 62 |
| RD2, 5 x GGGS linker (SEQ ID NO: 92) | LTISDDLGQTTLEVFKEDGKTLVSKKVTSKDKSSTEEKFNEKGEVSEKIITRADGTRLEYTGIKSDGSGKAKEVLKGYTLEGQLSDEKTTLVVK | |
| | 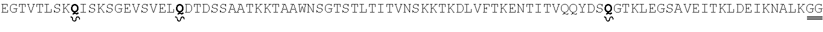 | |
| | 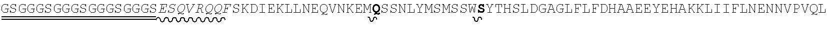 | |
| | 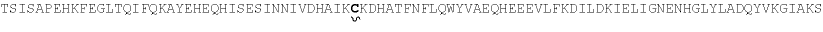 | |
| | RKS | |
| Non-glycosylated (Alanine) OspA- ferritin, LFA1 replacement RD2, | 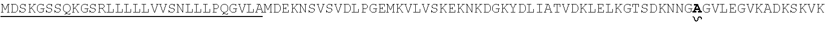 | 63 |
| Serotype 1 | LTISDDLGQTTLEVFKEDGKTLVSKKVTSKDKSSTEEKFNEKGEVSEKIITRADGTRLEYTGIKSDGSGKAKEVLKGYTLEGQLSDEKTTLVVK | |
| | 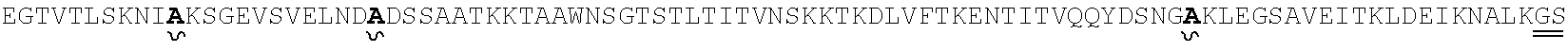 | |
| | 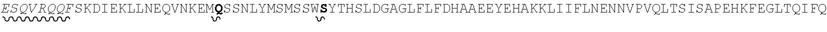 | |
| | 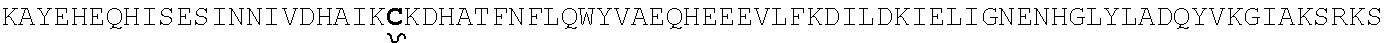 | |
| OspA-ferritin Serotype 1 | MDSKGSSQKGSRLLLLLVVSNLLLPQGVLAMDEKNSVSVDLPGEMKVLVSKEKNKDGKYDLIATVDKLELKGTSDKNNGSGVLEGVKADKSKVK | 64 |
| | LTISDDLGQTTLEVFKEDGKTLVSKKVTSKDKSSTEEKFNEKGEVSEKIITRADGTRLEYTGIKSDGSGKAKEVLKGYVLEGTLTAEKTTLVVK | |
| | EGTVTLSKNISKSGEVSVELNDTDSSAATKKTAAWNSGTSTLTITVNSKKTKDLVFTKENTITVQQYDSNGTKLEGSAVEITKLDEIKNALKGS | |
| | 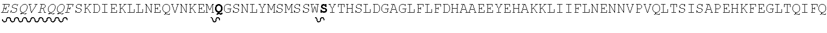 | |
| | 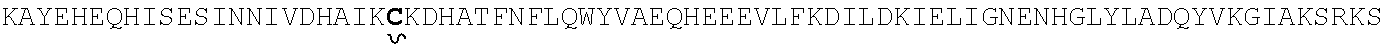 | |
| Serotype 2 OspA-ferritin | MDSKGSSQKGSRLLLLLVVSNLLLPQGVLAMDEKNSASVDLPGEMKVLVSKEKDKDGKYSLKATVDKIELKGTSDKDNGSGVLEGTKDDKSKAK | 65 |
| | LTIADDLSKTTFELFKEDGKTLVSRKVSSKDKTSTDEMFNEKGELSAKTMTRENGTKLEYTEMKSDGTGKAKEVLKNFTLEGKVANDKVTLEVK | |
| | EGTVTLSKEIAKSGEVTVALNDTNTTQATKKTGAWDSKTSTLTISVNSKKTTQLVFTKQDTITVQKYDSAGTNLEGTAVEIKTLDELKNALKGS | |
| | 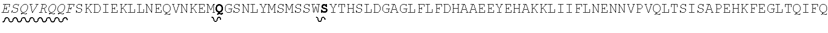 | |
| | 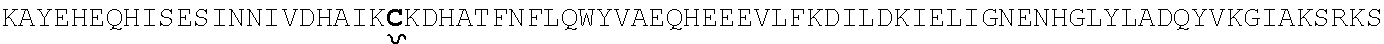 | |
| Non-glycosylated OspA-ferritin Serotype 2 | 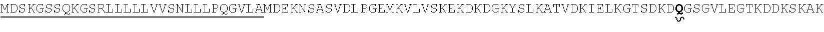 | 66 |
| | 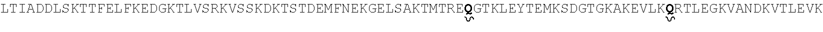 | |
| | 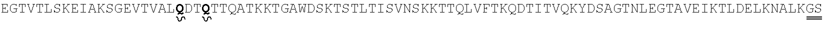 | |
| | 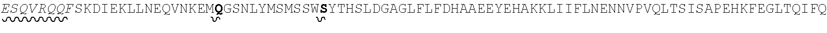 | |
| | 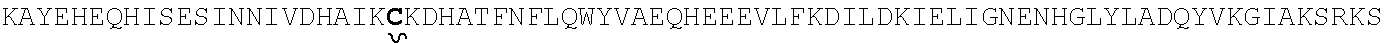 | |
| Serotype 3 OspA-ferritin | MDSKGSSQKGSRLLLLLVVSNLLLPQGVLAMDEKNSVSVDLPGGMKVLVSKEKDKDGKYSLMATVEKLELKGTSDKSNGSGVLEGEKADKSKAK | 67 |
| | LTISQDLNQTTFEIFKEDGKTLVSRKVNSKDKSSTEEKFNDKGKLSEKVVTRANGTRLEYTEIKNDGSGKAKEVLKGFALEGTLTDGGETKLTV | |
| | TEGTVTLSKNISKSGEITVALNDTETTPADKKTGEWKSDTSTLTISKNSQKPKQLVFTKENTITVQNYNRAGNALEGSPAEIKDLAELKAALKG | |
| | 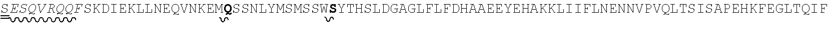 | |
| | 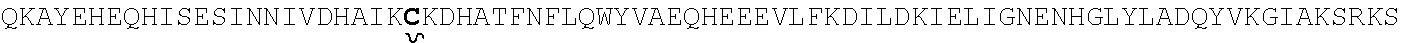 | |
| Non-glycosylated OspA-ferritin Serotype 3 | 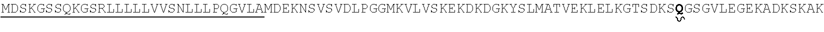 | 68 |
| | 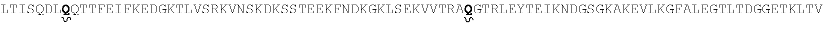 | |
| | 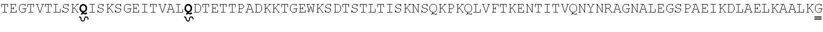 | |
| | 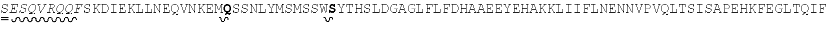 | |
| | 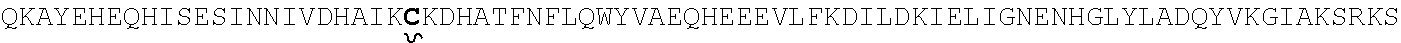 | |
| Serotype 4 OspA-ferritin | MDSKGSSQKGSRLLLLLVVSNLLLPQGVLAMDEKNSVSVDLPGEMKVLVSKEKDKDGKYSLMATVDKLELKGTSDKSNGSGTLEGEKSDKSKAK | 69 |
| | LTISEDLSKTTFEIFKEDGKTLVSKKVNSKDKSSIEEKFNAKGELSEKTILRANGTRLEYTEIKSDGTGKAKEVLKDFALEGTLAADKTTLKVT | |
| | EGTVVLSKHIPNSGEITVELNDSNSTQATKKTGKWDSNTSTLTISVNSKKTKNIVFTKEDTITVQKYDSAGTNLEGNAVEIKTLDELKNALKGS | |
| | 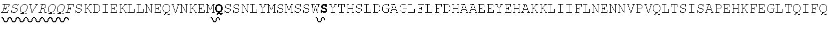 | |
| | 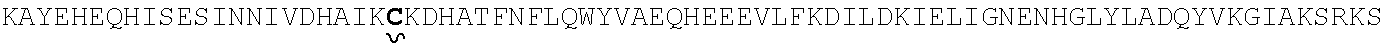 | |
| Non-glycosylated OspA-ferritin Serotype 4 | 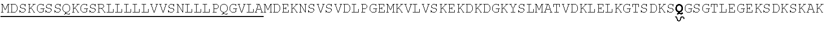 | 70 |
| | 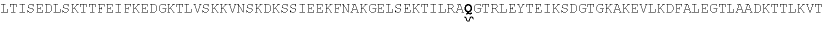 | |
| | 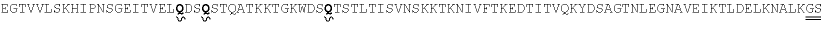 | |
| | 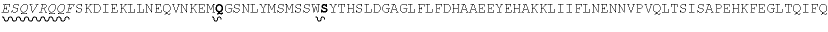 | |
| | 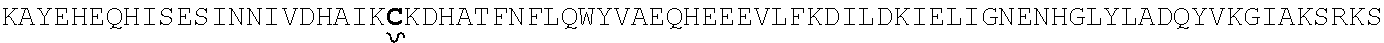 | |
| OspA-ferritin Serotype 5 | MDSKGSSQKGSRLLLLLVVSNLLLPQGVLAMDEKNSVSVDLPGGMKVLVSKEKDKDGKYSLMATVEKLELKGTSDKNNGSGTLEGEKTDKSKVK | 71 |
| | LTIAEDLSKTTFEIFKEDGKTLVSKKVTLKDKSSTEEKFNEKGEISEKTIVRANGTRLEYTDIKSDGSGKAKEVLKDFTLEGTLAADGKTTLKV | |
| | TEGTVVLSKNILKSGEITVALDDSDTTQATKKTGKWDSKTSTLTISVNSQKTKNLVFTKEDTITVQKYDSAGTNLEGKAVEITTLEKLKDALKG | |
| | 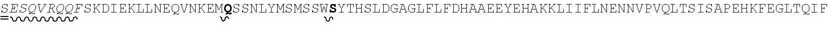 | |
| | 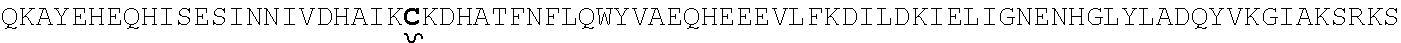 | |
| Serotype 5 Non-glycosylated OspA- ferritin | 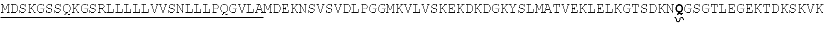 | 72 |
| | 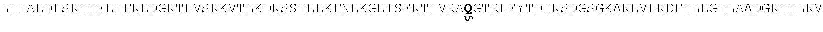 | |
| | TEGTVVLSKNILKSGEITVALDDSDTTQATKKTGKWDSKTSTLTISVNSQKTKNLVFTKEDTITVQKYDSAGTNLEGKAVEITTLEKLKDALKG | |
| | 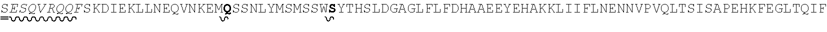 | |
| | 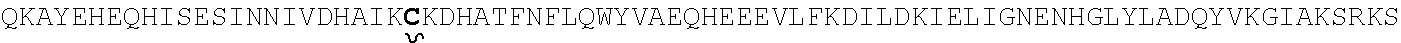 | |
| Serotype 6 Non-glycosylated OspA- ferritin | 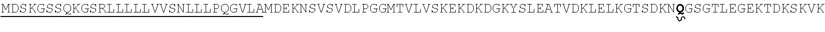 | 73 |
| | 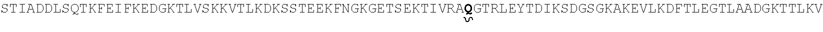 | |
| | TEGTVVLSKNILKSGEITAALDDSDTTRATKKTGKWDSKTSTLTISVNSQKTKNLVFTKEDTITVQRYDSAGTNLEGKAVEITTLKELKNALKG | |
| | 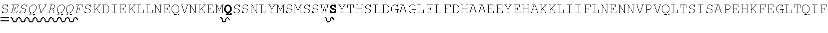 | |
| | 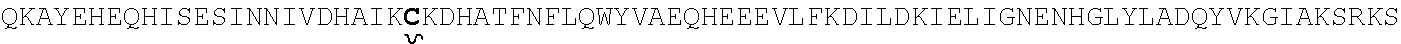 | |
| Serotype 6 | MDSKGSSQKGSRLLLLLVVSNLLLPQGVLAMDEKNSVSVDLPGGMTVLVSKEKDKDGKYSLEATVDKLELKGTSDKNNGSGTLEGEKTDKSKVK | 74 |
| OspA- | STIADDLSQTKFEIFKEDGKTLVSKKVTLKDKSSTEEKFNGKGETSEKTIVRANGTRLEYTDIKSDGSGKAKEVLKDFTLEGTLAADGKTTLKV | |
| ferritin | TEGTVVLSKNILKSGEITAALDDSDTTRATKKTGKWDSKTSTLTISVNSQKTKNLVFTKEDTITVQRYDSAGTNLEGKAVEITTLKELKNALKG | |
| | 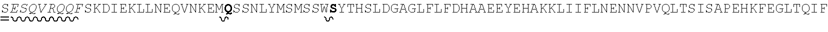 | |
| | 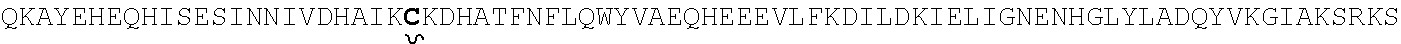 | |
| OspA-ferritin Serotype 7 | MDSKGSSQKGSRLLLLLVVSNLLLPQGVLAMDEKNSVSVDLPGEMKVLVSKEKDKDGKYSLEATVDKLELKGTSDKNNGSGVLEGVKAAKSKAK | 75 |
| | LTIADDLSQTKFEIFKEDGKTLVSKKVTLKDKSSTEEKFNDKGKLSEKVVTRANGTRLEYTEIQNDGSGKAKEVLKSLTLEGTLTADGETKLTV | |
| | EAGTVTLSKNISESGEITVELKDTETTPADKKSGTWDSKTSTLTISKNSQKTKQLVFTKENTITVQKYNTAGTKLEGSPAEIKDLEALKAALKG | |
| | 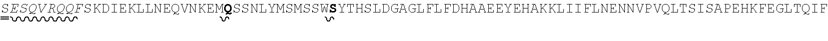 | |
| | 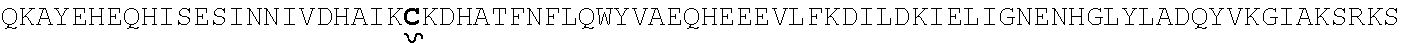 | |
| Serotype 7 Non-glycosylated OspA- ferritin | 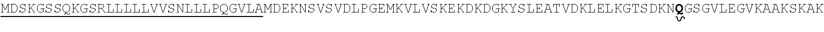 | 76 |
| | 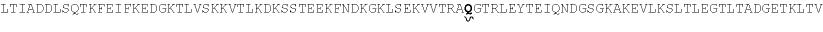 | |
| | 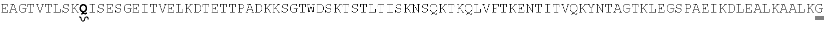 | |
| | 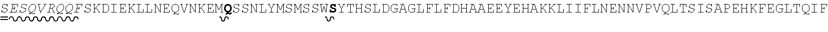 | |
| | 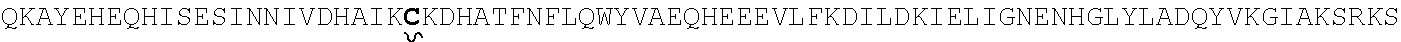 | |
| Amino acids 165-173 of OspA | YVLEGTLTA | 77 |
| serotype 1 | | |
| Sequence of hLFA-1 having homology | YVIEGTSKQ | 78 |
| to amino acids 165-173 of OspA | | |
| serotype 1 | | |
| S2 sequence from OspA serotype 2 | FTLEGKVAN | 79 |
| S3 sequence from OspA serotype 3 | FALEGTLTD | 80 |
| RD2 | YTLEGQLSD | 81 |
| RD1 | YDLKGELSS | 82 |
| Exemplary OspA serotype 1 (strain WP_010890378.1) | 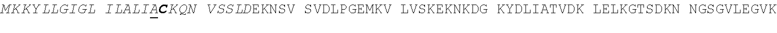 | 83 |
| | ADKSKVKLTI SDDLGQTTLE VFKEDGKTLV SKKVTSKDKS STEEKFNEKG EVSEKIITRA DGTRLEYTGI KSDGSGKAKE | |
| | VLKGYVLEGT LTAEKTTLVV KEGTVTLSKN ISKSGEVSVE LNDTDSSAAT KKTAAWNSGT STLTITVNSK KTKDLVFTKE | |
| | NTITVQQYDS NGTKLEGSAV EITKLDEIKN ALK | |
| Exemplary OspA serotype 2 (strain WP_011703777.1) | 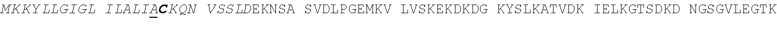 | 84 |
| | DDKSKAKLTI ADDLSKTTFE LFKEDGKTLV SRKVSSKDKT STDEMFNEKG ELSAKTMTRE NGTKLEYTEM KSDGTGKAKE | |
| | VLKNFTLEGK VANDKVTLEV KEGTVTLSKE IAKSGEVTVA LNDTNTTQAT KKTGAWDSKT STLTISVNSK KTTQLVFTKQ | |
| | DTITVQKYDS AGTNLEGTAV EIKTLDELKN ALK | |
| Exemplary OspA serotype 3 (strain CAA56549.1) | 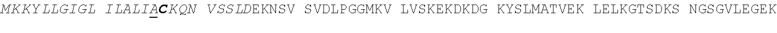 | 85 |
| | ADKSKAKLTI SQDLNQTTFE IFKEDGKTLV SRKVNSKDKS STEEKFNDKG KLSEKVVTRA NGTRLEYTEI KNDGSGKAKE | |
| | VLKGFALEGT LTDGGETKLT VTEGTVTLSK NISKSGEITV ALNDTETTPA DKKTGEWKSD TSTLTISKNS QKPKQLVFTK | |
| | ENTITVQNYN RAGNALEGSP AEIKDLAELK AALK | |
| Exemplary OspA serotype 4 (strain WP_011187157.1) | 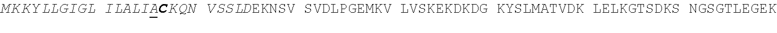 | 86 |
| | SDKSKAKLTI SEDLSKTTFE IFKEDGKTLV SKKVNSKDKS SIEEKFNAKG ELSEKTILRA NGTRLEYTEI KSDGTGKAKE | |
| | VLKDFALEGT LAADKTTLKV TEGTVVLSKH IPNSGEITVE LNDSNSTQAT KKTGKWDSNT STLTISVNSK KTKNIVFTKE | |
| | DTITVQKYDS AGTNLEGNAV EIKTLDELKN ALK | |
| Exemplary OspA serotype 5 (strain CAA59727.1) | 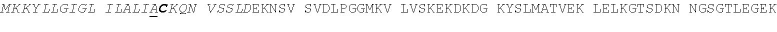 | 87 |
| | TDKSKVKLTI AEDLSKTTFE IFKEDGKTLV SKKVTLKDKS STEEKFNEKG EISEKTIVRA NGTRLEYTDI KSDGSGKAKE | |
| | VLKDFTLEGT LAADGKTTLK VTEGTVVLSK NILKSGEITV ALDDSDTTQA TKKTGKWDSK TSTLTISVNS QKTKNLVFTK | |
| | EDTITVQKYD SAGTNLEGKA VEITTLEKLK DALK | |
| Exemplary OspA serotype 6 (Strain CAA45010.1) | 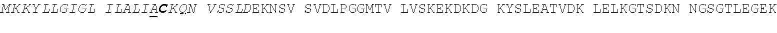 | 88 |
| | TDKSKVKSTI ADDLSQTKFE IFKEDGKTLV SKKVTLKDKS STEEKFNGKG ETSEKTIVRA NGTRLEYTDI KSDGSGKAKE | |
| | VLKDFTLEGT LAADGKTTLK VTEGTVVLSK NILKSGEITA ALDDSDTTRA TKKTGKWDSK TSTLTISVNS QKTKNLVFTK | |
| | EDTITVQRYD SAGTNLEGKA VEITTLKELK NALK | |
| Exemplary OspA serotype 7 (strain CAA56547.1) | 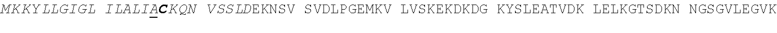 | 89 |
| | AAKSKAKLTI ADDLSQTKFE IFKEDGKTLV SKKVTLKDKS STEEKFNDKG KLSEKVVTRA NGTRLEYTEI QNDGSGKAKE | |
| | VLKSLTLEGT LTADGETKLT ENTITVQKYN TAGTKLEGSP AEIKDLEALK AALK | |
| H. pylori ferritin with 8 amino acid bull frog sequence at N-terminus | 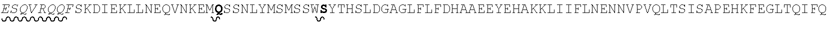 | 90 |
| | 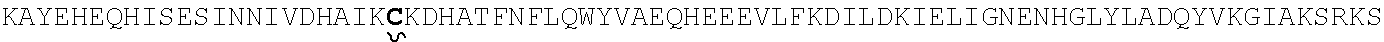 | |
| G52 linker | GGGSGGGS | 91 |
| G55 linker | GGGSGGGSGGGSGGGSGGGS | 92 |
| Exemplary lumazine synthase | MQIYEGKLTAEGLRFGIVASRFNHALVDRLVEGAIDCIVRHGGREEDITLVRVPGSWEIPVAAGELARKEDIDAVIAIGVLIRGATPHFDYIAS | 93 |
| | EVSKGLANLSLELRKPITFGVITADTLEQAIERAGTKHGNKGWEAALSAIEMANLFKSLR | |
| Serotype 1 RD2 OspA | MDEKNSVSVDLPGEMKVLVSKEKNKDGKYDLIATVDKLELKGTSDKNNGSGVLEGVKADKSKVKLTISDDLGQTTLEVFKEDGKTLVSKKVTSK | 94 |
| | DKSSTEEKFNEKGEVSEKIITRADGTRLEYTGIKSDGSGKAKEVLKGYTLEGQLSDEKTTLVVKEGTVTLSKNISKSGEVSVELNDTDSSAATK | |
| | KTAAWNSGTSTLTITVNSKKTKDLVFTKENTITVQQYDSNGTKLEGSAVEITKLDEIKNALK | |
| Serotype 1 RD1 OspA | MDEKNSVSVDLPGEMKVLVSKEKNKDGKYDLIATVDKLELKGTSDKNNGSGVLEGVKADKSKVKLTISDDLGQTTLEVFKEDGKTLVSKKVTSK | 95 |
| | DKSSTEEKFNEKGEVSEKIITRADGTRLEYTGIKSDGSGKAKEVLKGYDLKGELSSEKTTLVVKEGTVTLSKNISKSGEVSVELNDTDSSAATK | |
| | KTAAWNSGTSTLTITVNSKKTKDLVFTKENTITVQQYDSNGTKLEGSAVEITKLDEIKNALK | |
| Serotype 1 OspA; LFA-1 replacement | MDEKNSVSVDLPGEMKVLVSKEKNKDGKYDLIATVDKLELKGTSDKNNGSGVLEGVKADKSKVKLTISDDLGQTTLEVFKEDGKTLVSKKVTSK | 96 |
| Serotype 2 | DKSSTEEKFNEKGEVSEKIITRADGTRLEYTGIKSDGSGKAKEVLKGFTLEGKVANEKTTLVVKEGTVTLSKNISKSGEVSVELNDTDSSAATK | |
| | KTAAWNSGTSTLTITVNSKKTKDLVFTKENTITVQQYDSNGTKLEGSAVEITKLDEIKNALK | |
| Serotype 1 OspA; LFA-1 replacement | MDEKNSVSVDLPGEMKVLVSKEKNKDGKYDLIATVDKLELKGTSDKNNGSGVLEGVKADKSKVKLTISDDLGQTTLEVFKEDGKTLVSKKVTSK | 97 |
| Serotype 3 | DKSSTEEKFNEKGEVSEKIITRADGTRLEYTGIKSDGSGKAKEVLKGFALEGTLTDEKTTLVVKEGTVTLSKNISKSGEVSVELNDTDSSAATK | |
| | KTAAWNSGTSTLTITVNSKKTKDLVFTKENTITVQQYDSNGTKLEGSAVEITKLDEIKNALK | |
| Serotype 1 Non-glycosylated LFA-1 replacement RD2 OspA | 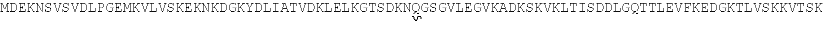 | 98 |
| | 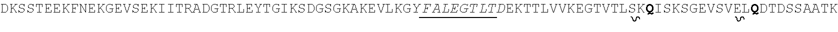 | |
| | 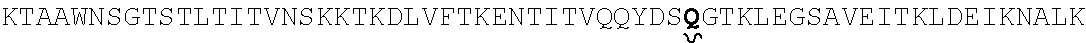 | |
| Non-glycosylated OspA Serotype 1 with LFA-1 site replaced with | 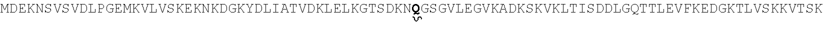 | 99 |
| Serotype 2 | 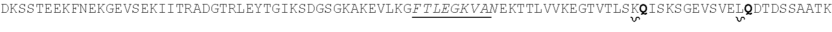 | |
| | 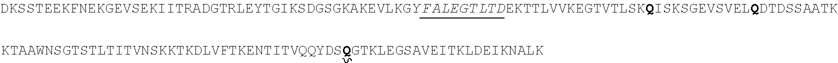 | |
| Non-glycosylated OspA Serotype 1 with LFA-1 site replaced with | 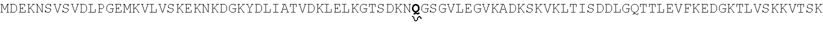 | 100 |
| Serotype 3 | | |
| | 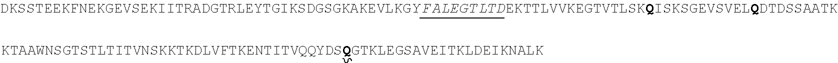 | |
| Non-glycosylated OspA Serotype 1 with LFA-1 site replaced with RD1 | 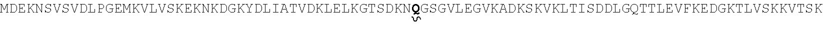 | 101 |
| | 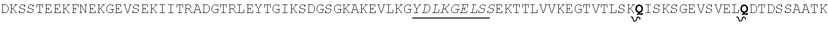 | |
| | 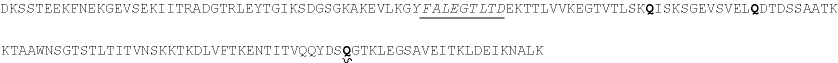 | |
| Modified Serotype 1 OspA (X = | MDEKNSVSVDLPGEMKVLVSKEKNKDGKYDLIATVDKLELKGTSDKNZGSGVLEGVKADKSKVKLTISDDLGQTTLEVFKEDGKTLVSKKVTSK | 102 |
| any amino acid; XXXXXXXXX is | DKSSTEEKFNEKGEVSEKIITRADGTRLEYTGIKSDGSGKAKEVLKGXXXXXXXXXEKTTLVVKEGTVTLSKZISKSGEVSVELZDTDSSAATK | |
| not SEQ ID NO: 77; Z = N or Q) | KTAAWNSGTSTLTITVNSKKTKDLVFTKENTITVQQYDSZGTKLEGSAVEITKLDEIKNALK | |
| | Not Used | 103- |
| | | 200 |
| bfpFerritin-N19Q/C31S/S26C | ESQVRQQFSKDIEKLLNEQVNKEMQSSNLYMCMSSWSYTHSLDGAGLFLFDHAAEEYEHAKKLIIFLNENNVPVQLTSISAPEHKFEGLTQIFQ | 201 |
| | KAYEHEQHISESINNIVDHAIKSKDHATFNFLQWYVAEQHEEEVLFKDILDKIELIGNENHGLYLADQYVKGIAKSRKS | |
| bfpFerritin-N19Q/C31S/S72C | ESQVRQQFSKDIEKLLNEQVNKEMQSSNLYMSMSSWSYTHSLDGAGLFLFDHAAEEYEHAKKLIIFLNENNVPVQLTCISAPEHKFEGLTQIFQ | 202 |
| | KAYEHEQHISESINNIVDHAIKSKDHATFNFLQWYVAEQHEEEVLFKDILDKIELIGNENHGLYLADQYVKGIAKSRKS | |
| bfpFerritin-N19Q/C31S/A75C | ESQVRQQFSKDIEKLLNEQVNKEMQSSNLYMSMSSWSYTHSLDGAGLFLFDHAAEEYEHAKKLIIFLNENNVPVQLTSISCPEHKFEGLTQIFQ | 203 |
| | KAYEHEQHISESINNIVDHAIKSKDHATFNFLQWYVAEQHEEEVLFKDILDKIELIGNENHGLYLADQYVKGIAKSRKS | |
| bfpFerritin-N19Q/C31S/K79C | ESQVRQQFSKDIEKLLNEQVNKEMQSSNLYMSMSSWSYTHSLDGAGLFLFDHAAEEYEHAKKLIIFLNENNVPVQLTSISAPEHCFEGLTQIFQ | 204 |
| | KAYEHEQHISESINNIVDHAIKSKDHATFNFLQWYVAEQHEEEVLFKDILDKIELIGNENHGLYLADQYVKGIAKSRKS | |
| bfpFerritin-N19Q/C31S/S100C | ESQVRQQFSKDIEKLLNEQVNKEMQSSNLYMSMSSWSYTHSLDGAGLFLFDHAAEEYEHAKKLIIFLNENNVPVQLTSISAPEHKFEGLTQIFQ | 205 |
| | KAYEHEQHISECINNIVDHAIKSKDHATFNFLQWYVAEQHEEEVLFKDILDKIELIGNENHGLYLADQYVKGIAKSRKS | |
| bfpFerritin-N19Q/C31S/S111C | ESQVRQQFSKDIEKLLNEQVNKEMQSSNLYMSMSSWSYTHSLDGAGLFLFDHAAEEYEHAKKLIIFLNENNVPVQLTSISAPEHKFEGLTQIFQ | 206 |
| | KAYEHEQHISESINNIVDHAIKCKDHATFNFLQWYVAEQHEEEVLFKDILDKIELIGNENHGLYLADQYVKGIAKSRKS | |
| bfpFerritin-N19Q/C31S/E12C | ESQVRQQFSKDIEKLLNCQVNKEMQSSNLYMSMSSWSYTHSLDGAGLFLFDHAAEEYEHAKKLIIFLNENNVPVQLTSISAPEHKFEGLTQIFQ | 207 |
| | KAYEHEQHISESINNIVDHAIKSKDHATFNFLQWYVAEQHEEEVLFKDILDKIELIGNENHGLYLADQYVKGIAKSRKS | |
| Exemplary H. pylori Ferritin | ESQVRQQFSKDIEKLLNEQVNKEMNSSNLYMSMSSWCYTHSLDGAGLFLFDHAAEEYEHAKKLIIFLNENNVPVQLTSISAPEHKFEGLTQIFQ | 208 |
| with bullfrog linker | KAYEHEQHISESINNIVDHAIKSKDHATFNFLQWYVAEQHEEEVLFKDILDKIELIGNENHGLYLADQYVKGIAKSRKS | |
| Exemplary wild-type H. pylori | LSKDIIKLLNEQVNKEMNSSNLYMSMSSWCYTHSLDGAGLFLFDHAAEEYEHAKKLIIFLNENNVPVQLTSISAPEHKFEGLTQIFQKAYEHEQ | 209 |
| ferritin (GenBank Accession | HISESINNIVDHAIKSKDHATFNFLQWYVAEQHEEEVLFKDILDKIELIGNENHGLYLADQYVKGIAKSRKS | |
| AAD06160.1) (without bullfrog | | |
| linker or N-terminal Met) | | |
| CpG (ISS-1018) | TGACTGTGAACGTTCGAGATGA | 210 |
| Trichoplusia ni heavy chain ferritin | TQCNVNPVQIPKDWITMHRSCRNSMRQQIQMEVGASLQYLAMGAHFSKDVVNRPGFAQLFFDAASEEREHAMKLIEYLLMRGELTNDVSSLLQV | 211 |
| | RPPTRSSWKGGVEALEHALSMESDVTKSIRNVIKACEDDSEFNDYHLVDYLTGDFLEEQYKGQRDLAGKASTLKKLMDRHEALGEF | |
| | IFDKKLLGIDV | |
| Trichoplusia ni light chain ferritin | ADTCYNDVALDCGITSNSLALPRCNAVYGEYGSHGNVATELQAYAKLHLERSYDYLLSAAYFNNYQTNRAGFSKLFKKLSDEAWSKTIDIIKHV | 212 |
| | TKRGDKMNFDQHSTMKTERKNYTAENHELEALAKALDTQKELAERAFYIHREATRNSQHLHDPEIAQYLEEEFIEDHAEKIRTLAGHTSDLKKF | |
| | ITANNGHDLSLALYVFDEYLQKTV | |
| Pyrococcus furiosus ferritin | MLSERMLKALNDQLNRELYSAYLYFAMAAYFEDLGLEGFANWMKAQAEEEIGHALRFYNY | 213 |
| | IYDRNGRVELDEIPKPPKEWESPLKAFEAAYEHEKFISKSIYELAALAEEEKDYSTRAFL | |
| | EWFINEQVEEEASVKKILDKLKFAKDSPQILFMLDKELSARAPKLPGLLMQGGE | |
| human heavy chain ferritin | MTTASTSQVRQNYHQDSEAAINRQINLELYASYVYLSMSYYFDRDDVALKNFAKYFLHQSHEEREHAEKLMKLQNQRGGRIFLQDIKKPDCDDW | 214 |
| | ESGLNAMECALHLEKNVQQSLLELHKLATDKNDPHLCDFIETHYLNEQVKAIKELGDHVTNLRKMGAPESGLAEYLFDKHTLGDSDQES | |
| human light chain ferritin (signal | MDSKGSSQKGSRLLLLLVVSNLLLPQGVLASSQIRQNYSTDVEAAVNSLVNLYLQASYTYLSLGFYFDRDDVALEGVSHFFRELAEEKREGYER | 215 |
| peptide is underlined) | LLKMQNQRGGRALFQDIKKPAEDEWGKTPDAMKAAMALEKKLNQALLDLHALGSARTDPHLCDFLETHFLDEEVKLIKKMGDHLTNLHRLGGPE | |
| | AGLGEYLFERLTLKHD | |
| lumazine synthase from Aquifex | MQIYEGKLTAEGLRFGIVASRFNHALVDRLVEGAIDCIVRHGGREEDITLVRVPGSWEIPVAAGELARKEDIDAVIAIGVLIRGATPHFDYIAS | 216 |
| aeolicus | EVSKGLANLSLELRKPITFGVITADTLEQAIERAGTKHGNKGWEAALSAIEMANLFKSLR | |
| bullfrog linker | 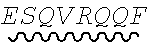 | 217 |
| Cysteine-Thrombin-His Linker | CLVPRGSLEHHHHHH | 218 |
| E. coli 6,7-dimethyl-8-ribityl- | MNIIEANVATPDARVAITIARFNNFINDSLLEGAIDALKRIGQVKDENITVVWVPGAYELPLAAGALAKTGKYDAVIALGTVIRGGTAHFEYVA | 219 |
| lumazine synthase | GGASNGLAHVAQDSEIPVAFGVLTTESIEQAIERAGTKAGNKGAEAALTALEMINVLKAIKA | |
| 16 amino acid linker | GGGGSGGGGSGGGGSG | 220 |
| 28 amino acid linker | GGSGSGSNSSASSGASSGGASGGSGGSG | 221 |
| 46 amino acid linker | GGSGSASSGASASGSSNGSGSGSGSNSSASSGASSGGASGGSGGSG | 222 |
| FR1 | GGSGSASAEAAAKEAAAKAGGSGGSG | 223 |
| FR2 | GGSGSASAEAAAKEAAAKEAAAKASGGSGGSG | 224 |
| 47 amino acid linker comprising a | SGGGSGSASSGASASGSSCSGSGSGSSSASSGASSGGASGGGSGGSG | 225 |
| C for conjugation | | |

### EXAMPLES

The following examples are provided to illustrate certain disclosed embodiments and are not to be construed as limiting the scope of this disclosure in any way.

### 1. Preparation of OspA-Ferritin Antigenic Polypeptides

Antigenic polypeptides comprising OspA and ferritin were generated.

OspA was synthesized by Genescript from the following sequences: Borrelia burgdorferi strain B31 (Serotype 1) NBCI sequence ID WP_010890378.1, Borrelia afzelii strain PKO (Serotype 2) NCBI sequence: WP_011703777.1, Borrelia garinii strain PBr (Serotype 3) GenBank: CAA56549.1, Borrelia bavariensis (Serotype 4) NCBI sequence WP_011187157.1, Borrelia garinii (Serotype 5) GenBank CAA59727.1, Borrelia garinii (serotype 6) GenBank: CAA45010.1, and Borrelia garinii (Serotype 7) GenBank CAA56547.1. The H. pylori ferritin with an inserted N-terminal bull frog ferritin sequence was synthesized by Genescript, in which the bull frog ferritin sequence is similar to that of a previous study (see Kanekiyo, M., et al., Cell 162(5):1090-100 (2015)). The pet21a vector was used to express both His-tagged OspA and OspA-ferritin nanoparticles in E. coli. A mammalian expression vector similar to that used previously was used for expression in Expi293 cells (see Xu, L., et al., Science 358(6359):85-90 (2017)).

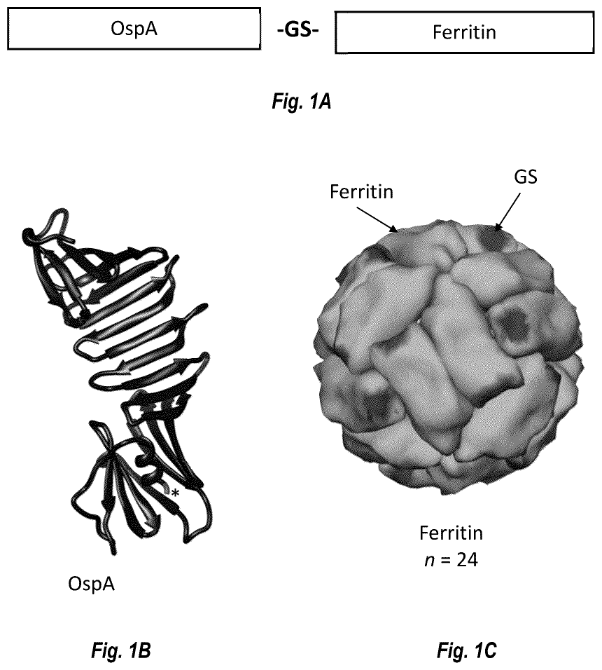

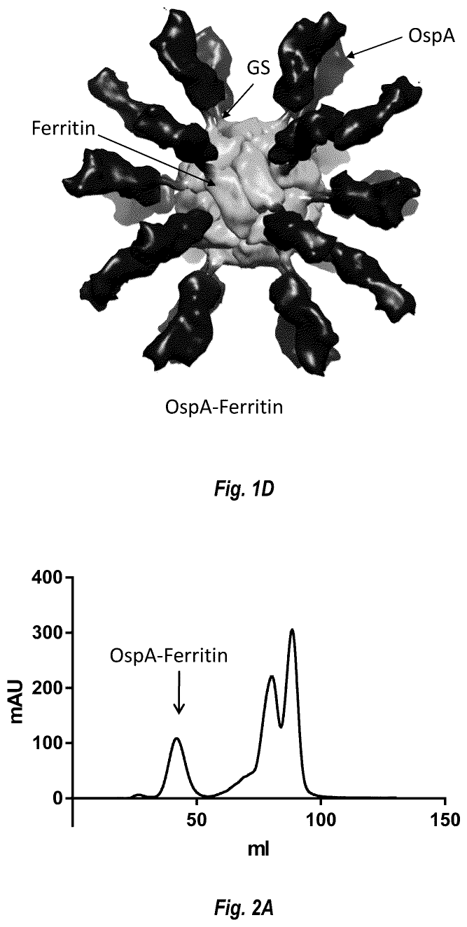

OspA-ferritin nanoparticles were created by genetically fusing the ectodomain of OspA to the amino-terminus of ferritin to generate an antigenic polypeptide (FIG. 1A). OspA is a 31-kDa lipoprotein with an extended β-sheet structure made up of 21 consecutive antiparallel β-strands with only one carboxy-terminal α-helix (FIG. 1B) (see Kitahara, R., et al., Biophys J 102(4):916-26 (2012)). The carboxy-terminus of OspA also has an unusually large cavity (˜200 A) that represented a site compatible for linkage to ferritin using a glycine-serine sequence (FIG. 1B). The 24 subunits of ferritin assemble spontaneously into a hollow spherical nanoparticle (FIG. 1C). The ferritin used in this study contains the amino-terminal sequence of bullfrog ferritin fused to Helicobacter pylori ferritin to create a chimera minimally related to human ferritin (see Kanekiyo 2015). The amino-terminal bullfrog ferritin sequence projects radially from the nanoparticle core (see Trikha, J., et al. J Mol Biol 248(5):949-67 (1995)), facilitating the presentation of OspA evenly on the nanoparticle surface.

Three additional changes were made to the ferritin structure to improve its functionality: N19Q, C31S and S111C. The N19Q substitution removed a potential amino-terminal glycosylation site. The S111C substitution introduces a surface-exposed cysteine on the ferritin that can be used to conjugate adjuvants with, for example, click chemistry. Finally, cysteine 31 was modified to serine so that only one cysteine would be modified by conjugation. Display of OspA on the nanoparticle surface provides a 24-mer antigenic nanoparticle (FIG. 1D).

For purification from E. coli, we used BL21 Star (DE3) (Invitrogen Cat #C601003). We induced the protein with 100 μM IPTG overnight at 16° C. The cell pellet was lysed using sonication in Tris buffer pH 8, 50 mM NaCl. The filter sterilized supernatant was purified on an anion exchange column (HiTrap Q HP, GE), by collecting OspA-ferritin from the flow-through. Endotoxin was then removed by a 1% Triton X114 extraction that was repeated 6 times. The aqueous phase was then concentrated using an Amicon 100 MW cutoff filter (Millipore Cat #UFC910096) and nanoparticles were then further purified on a 120 ml Superose 6 preparatory SEC column at 4° C. For purification from mammalian cell culture, Expi293 cells were transfected with plasmid DNA using FectoPRO transfection Reagent (Polyplus, Cat #116-100) per manufacturer's instructions. Transfected cells were cultured on day 5 and the supernatant was collected and filtered. Endotoxin-free protocols were followed using endotoxin-free reagents and glassware. Q sepharose Fast flow beads (GE, Cat #17-0510-01) were prepared with 50 mM Tris pH7, 50 mM NaCl and applied to the filter-sterilized supernatant by gravity flow. The flow-through was collected and concentrated to 4ml using Amicon 100 MW cutoff filter. Nanoparticles were then further purified on a 120 ml Superose 6 preparatory SEC column at room temperature.

For purification of His6-tagged (SEQ ID NO: 227) OspA, serotype 1, 4, 5, and 7 OspA were purified from E. coli BL21 (DE3) (Invitrogen Cat #C600003), and serotype 2 and 3 were purified from Expi293 cells. These constructs lacked the transmembrane domain, comprised a C-terminal His₆-tag (SEQ ID NO: 227), and were otherwise wild-type. For E. coli purification, protein was induced at 500 μM IPTG for 5 hours and cells were pelleted and frozen at −20° C. Pellet was resuspended in 1% Triton in TBS buffer with Complete Protease Inhibitor (Sigma-Aldrich, Cat #11697498001) and sonicated to lyse cells. The supernatant was filter sterilized. For mammalian cell culture, the supernatant was collected at day 5 after transfection and filter sterilized. The supernatant was run on a GE HiTrap HP 5 ml column (Cat #17-5248-02) attached to an AKTA Pure FPLC. The column was washed and loaded and washed again with 20 mM imidizole in TBS. Final protein was eluted with 250 mM imidizole in TBS.

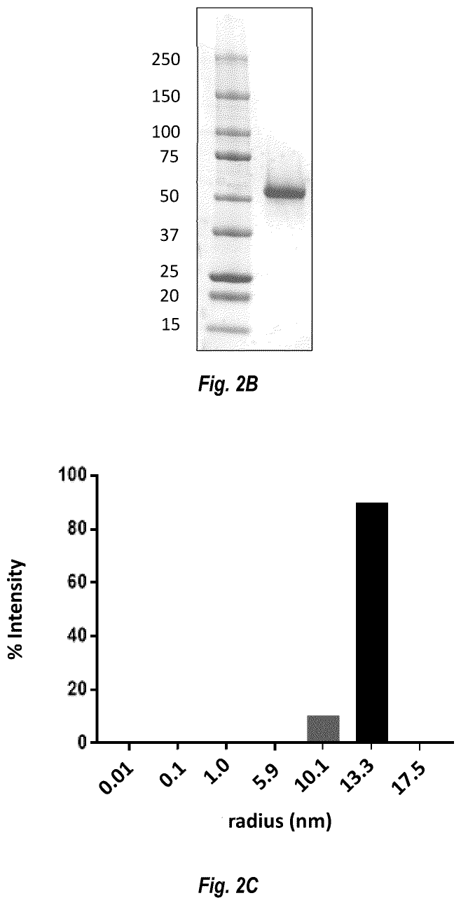

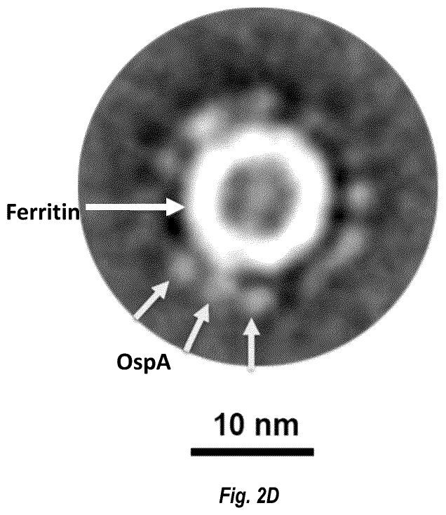

OspA serotype 1 was expressed from B. burgdorferi strain B31 fused to ferritin in a transformed human renal epithelial cell line, Expi293 (FIGS. 2A-2D; SEQ ID NO: 52). The formation of the nanoparticles and the purity of the protein were confirmed by size exclusion column chromatography (SEC) and SDS-PAGE (FIGS. 2A and 2B, respectively). SEC analysis revealed a single symmetrical peak of the expected retention time (FIG. 2A).

Dynamic light scattering (DLS) analysis was also performed. Purified nanoparticles were loaded into a black 384 well plate with a clear bottom (Corning, Cat #3540) at a concentration ˜0.4 μg/ml. Samples were read with a DynaPro plater Reader II (Wyatt) at a control temperature of 25° C. DLS documented a particle size of 13 nm with low polydispersity (7.4%) that is pure and without aggregates (FIG. 2C). Elimination of the transmembrane domain of OspA (aa 1-25) that contains the lipidation site improved the ease of purification. The OspA sequence contains four potential amino-linked glycosylation sites, and OspA-ferritin purified from mammalian cells migrated at a higher molecular weight consistent with the addition of glycans (FIG. 2B).

Transmission electron microscopy negative stain imaging and 2D class averaging analysis was performed on the OspA-ferritin nanoparticles (FIG. 2D). A sample of OspA-ferritin nanoparticles was diluted 300-fold in 1×TBS and imaged over a layer of continuous carbon supported by nitro-cellulose on a 400-mesh copper grid. The grids were prepared by applying 3 μl of sample suspension to a cleaned grid, blotting away with filter paper, and immediately stained with uranyl formate. Electron microscopy was performed using an FEI Tecnai T12 electron microscope equipped with an FEI Eagle 4k×4K CCD camera. High magnification images were acquired at magnification of 67,000 (0.16 nm/pixel). The images were acquired at a nominal underfocus of −1.9 μm to −0.8 μm and electron doses of ˜30 e⁻/Å2. Individual particles in the 67,000× high magnification images were selected using automated picking protocols (see Lander, G. C., et al., J Struct Biol, 166(1):95-102 (2009)). A reference-free alignment strategy was used based on the XMIPP processing package (see Sorzano, C. O., et al., J Struct Biol 148(2): p. 194-204 (2004)). Algorithms in this package align the selected particles and sort them into self-similar groups or classes.

The ferritin nanoparticle appeared as a strong circular density with a hollow center in the middle (FIG. 2D). Each nanoparticle was surrounded by numerous, short, uniform spikes of OspA that appear slightly oblong in shape. The particles have an overall diameter ranging from ˜194-220 Å, with the ferritin core of 125 Å diameter. The spikes extended uniformly in size, shape and orientation from the particle surface up to 45 Å in length. The OspA spikes were ˜30Å in width and tapered to minimal density at the glycine-serine linker of ferritin.

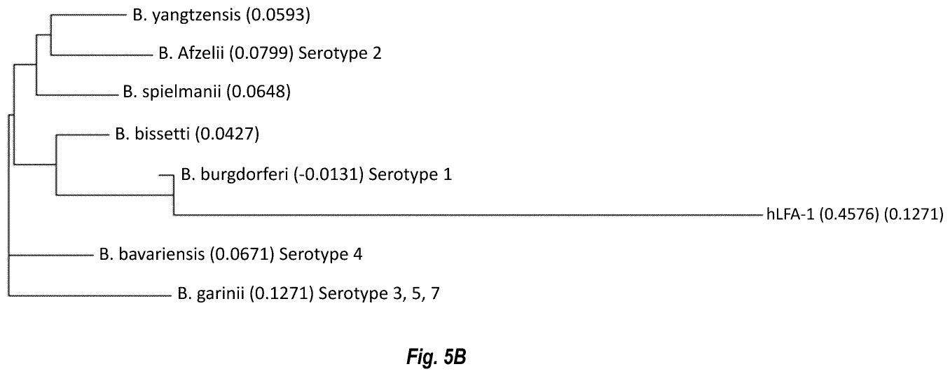

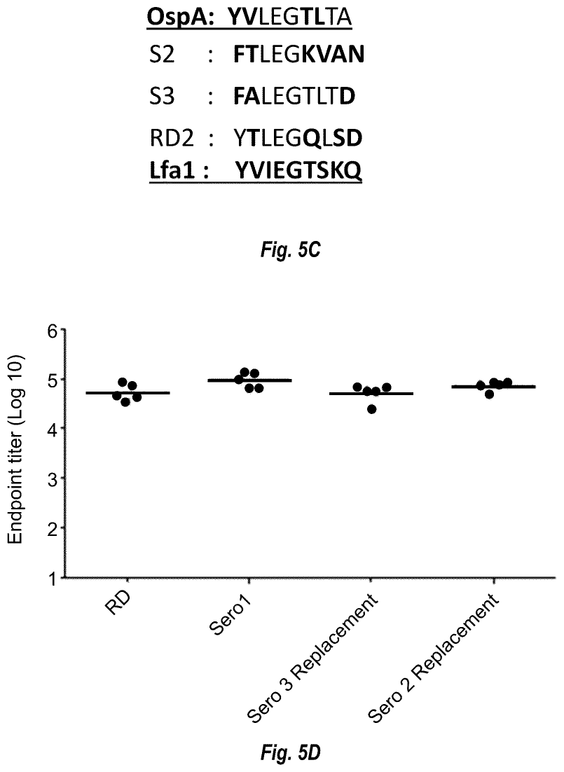

When the LYMErix™ vaccine was discontinued in 2002, the concern was raised that the vaccine contained an epitope (amino acids 165-173 of SEQ ID NO: 83) with homology to a nonapeptide segment (SEQ ID NO: 78) from the human leukocyte function-associated antigen-1 (hLFA-1, see Gross, D. M., et al., Science 281(5377): p. 703-6 (1998)) (FIG. 5A). Amino acids 165-173 of SEQ ID NO: 83 are referred to as the hLFA-1homology site. OspA serotype 1 is the only serotype that contains this sequence homology (FIG. 5B). To avoid any potential concerns related to this sequence, the hLFA-1 homology site was replaced with either the corresponding OspA serotype 2 (SEQ ID NO: 79) or serotype 3 (SEQ ID NO: 80) nonapeptide sequences, or point substitutions were introduced that reduced similarity to hLFA-1 and were intended to prevent the generation of antibodies that bind to hLFA-1 (RD2, SEQ ID NO: 81) (FIG. 5C). For the point substitutions, surface-exposed amino acids were substituted to reduce similarity to hLFA-1 while avoiding or minimizing destabilization of the β-sheet structure.

The immunogenicity of the hLFA-1 nanoparticles with a modified hLFA-1 homology site was tested in mice to compare the immune response relative to an OspA-ferritin nanoparticle without such modification (FIG. 5D). The antibody titers elicited by the nanoparticles with a modified hLFA-1 homology site were robust and not significantly different from the nanoparticles with an unmodified hLFA-1 homology site.

### 2. Characterization of Immunogenicity of OspA-Ferritin Nanoparticles

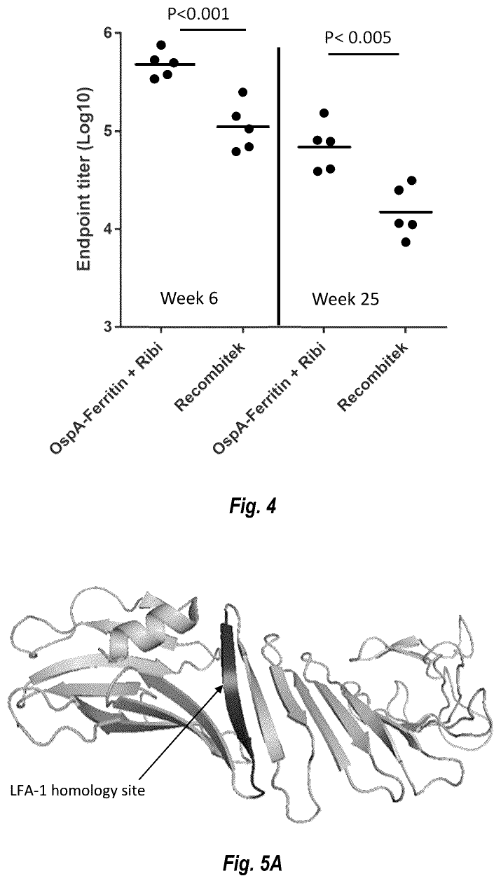

To assess the immunogenicity of OspA-ferritin nanoparticles, C3H mice were immunized twice with serotype 1 OspA-ferritin nanoparticles in the presence of Ribi adjuvant or RECOMBITEK® Lyme (liquid suspension of purified Outer surface protein A (OspA) of Borrelia burgdorferi), a canine vaccine in which the OspA is full-length, lipidated, recombinant, and of serotype 1 (FIG. 4). C3H/HeN mice were vaccinated intramuscularly at week zero and week 4. ELISAs were run on serum from 2 weeks post 2^{nd} dose. Ribi (Sigma adjuvant system Cat #S6322-1vl) was resuspended in 1 ml of PBS and vortexed for 1 minute and then added in equal volume to antigen prior to immunization.

The antibody response was determined using an enzyme-linked immunosorbent assay (ELISA) to recombinant OspA. Briefly, 96-well plates were coated with 1 μg/ml of OspA-His diluted in PBS and incubated overnight at 4° C. The OspA-His was removed and the plates were blocked with 5% skim milk dissolved in PBST. After removing the blocking reagent, the primary serum samples were added after being serially diluted in PBST. The primary samples were added in equal volume to blocking solution for a final 50% blocking solution concentration. After a 1-hour incubation, the plates were washed with PBST and incubated with Goat anti-mouse IgG, HRP-linked secondary antibody (1:5,000 dilution in blocking solution) for 1 hour at room temperature. The secondary antibody was aspirated and washed and the plates were incubated with Sure Blue TMB peroxidase substrate (KPL, Gaithersburg, MD) followed by equal volume of stop solution (0.5 N sulfuric acid). Absorbance was measured at 450 nm.

Immunization with OspA-ferritin induced endpoint titers 4.4-fold higher than RECOMBITEK® Lyme at week six (p<0.001). The antibody titer at week 25 is also significantly higher than RECOMBITEK® Lyme (p<0.005) (FIG. 4).

### 3. Glycosylation Mutants; Evaluation of Efficacy

To evaluate the protective efficacy of this OspA-ferritin, a challenge model was used in which immunized or control mice were infected by ticks carrying B. burgdorferi (see Rosa, P. A., et al. Nat Rev Microbiol 3(2): p. 129-43 (2005)).

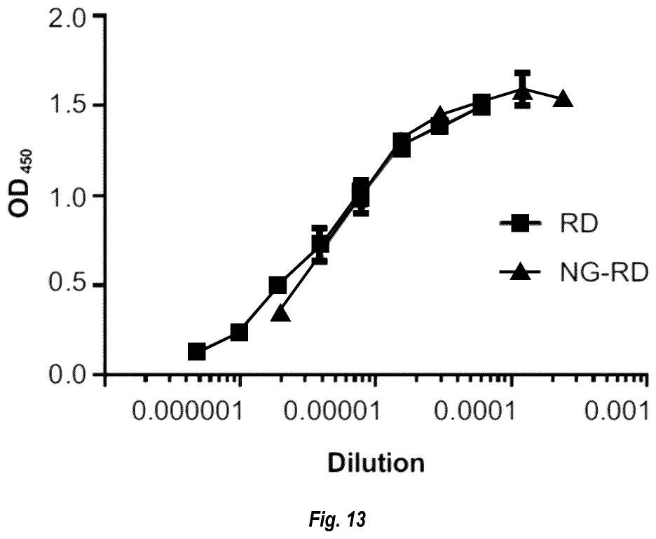

C3H/HeN mice were vaccinated intramuscularly with 1 μg of either OspA-ferritin nanoparticle mixed with Adda Vax™ 1:1 or 1 μg of ferritin nanoparticle. Mice were vaccinated at week zero and week 4. A serotype 1 OspA-ferritin nanoparticle with rationally designed modifications to the hLFA-1 homology site and modifications to remove all potential N-glycosylation sites (SEQ ID NO: 53) was used to immunize mice since natural bacterially expressed OspA is not glycosylated at these positions. The sequence contained the following N>Q substitutions to prevent glycosylation: N71Q, N190Q, N202Q, and N251Q. Its immunogenicity was similar to the glycosylated nanoparticle (FIG. 13).

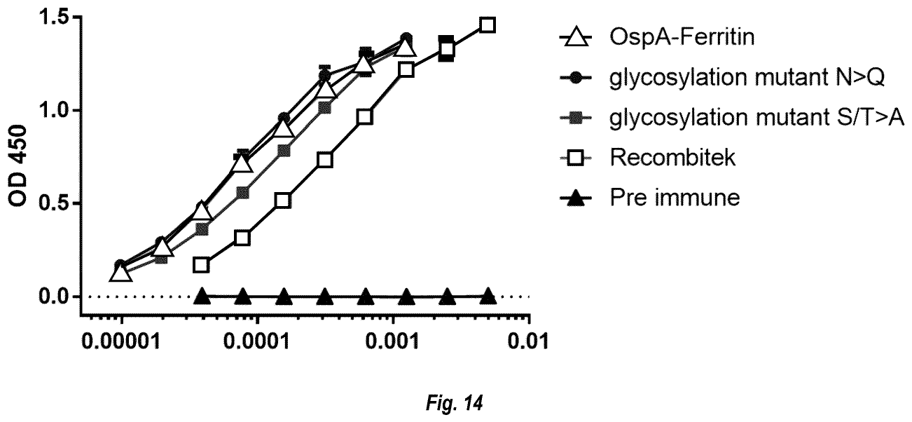

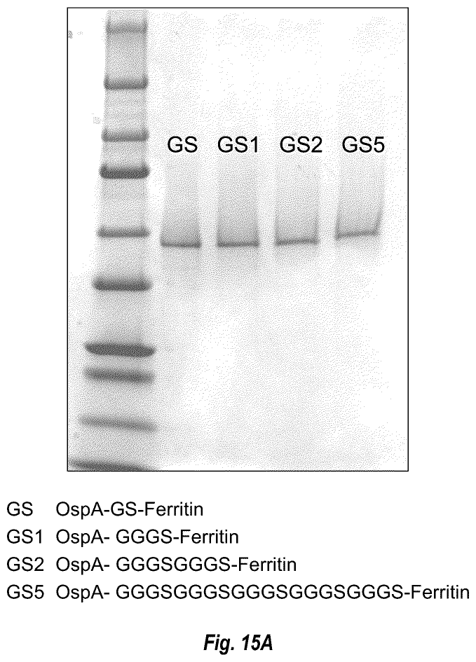

Glycosylation mutants of OspA-ferritin were also tested when the glycosylation site serine/threonines were mutated to an alanine (SEQ ID NO: 63). Both this construct and the N>Q construct discussed above gave a strong immune response as compared to the OspA-ferritin with wild-type glycosylation sites (SEQ ID NO: 52) and were superior to the RECOMBITEK® Lyme control (FIG. 14).

In a further experiment to evaluate the protective efficacy of OspA-ferritin nanoparticles in a tick challenge model, Ixodes scapularis tick larvae were obtained from National Tick Research and Education Center, Oklahoma State University (Stillwater, OK). B. burgdorferi-infected nymphs were generated by allowing uninfected larvae to feed to repletion on B. burgdorferi strain N40-infected SCID mice. The engorged larvae were collected and allowed to molt into nymphs in 4-6 weeks at room temperature and high relative humidity. Prevalence of B. burgdorferi infection in fed larvae was determined by culture of a portion of the recovered ticks from each batch.

Mice were immunized twice with 1 μg doses of OspA-ferritin (SEQ ID NO: 53) with Adda Vax™ adjuvant or control ferritin at week 0 and week 4, and a comparison group was immunized in parallel with RECOMBITEK® Lyme. Mice were challenged at week 6 (i.e., 2 weeks after the second vaccination dose), by allowing 5 to 6 B. burgdorferi infected nymphal ticks to feed to repletion. The fed nymphs were collected and assayed for B. burgdorferi infection by culture in BSK media. Two weeks after challenge, the mice were sacrificed and assayed for B. burgdorferi infection by culture of the ear, ankle and heart culture. Presence of B. burgdorferi was determined by observing the cultures by dark field microscopy. A mouse was defined as infected with B. burgdorferi if one or more organ cultures were found positive by darkfield microscopy. Negative cultures were also tested by PCR specific to B. burgdorferi.

Mice were sacrificed two weeks later. Tissue samples from the heart, ankle and ear were cultured in BSK media with antibiotics for B. burgdorferi for 6 wks. Negative samples were tested by PCR for the presence of B. burgdorferi. All negative cultures were also PCR negative. Protection was calculated as a percentage of uninfected mice.

The composition comprising OspA-ferritin and Adda Vax™ adjuvant showed no infection (0/4) in contrast to negative control ferritin, where 4 of 5 animals were infected (Table 2; p<0.01).

TABLE 2
Protective efficacy of OspA-ferritin nanoparticles

| Antigen | Mice/group | # mice infected | % Infected |
|---|---|---|---|
| Control particle | 5 | 4 | 80 |
| OspA-ferritin + | 4 | 0 | 0 |
| AddaVax ™ | | | |
| RECOMBITEK ® | 4 | 0 | 0 |
| Lyme | | | |

### 4. Evaluation of Efficacy of OspA-Ferritin Conjugated to Immune-Stimulatory Moieties

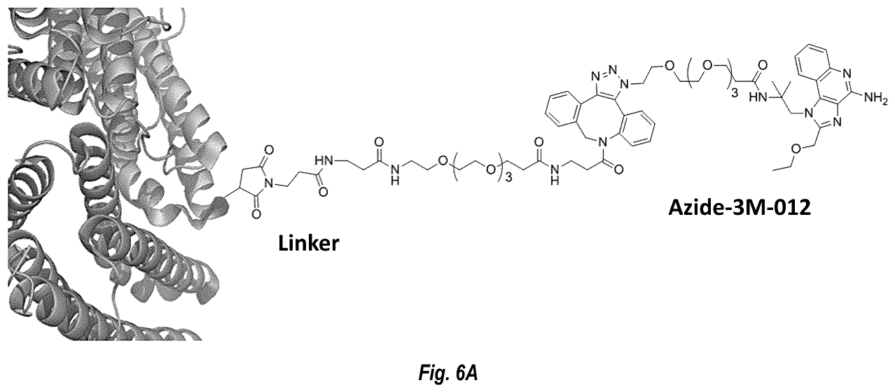

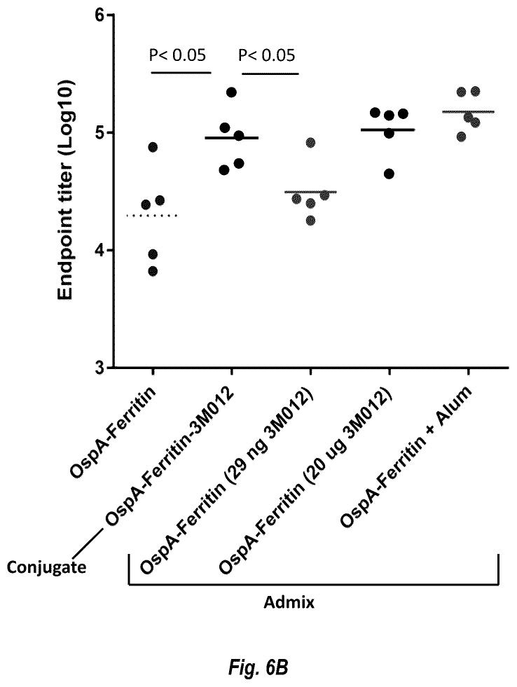

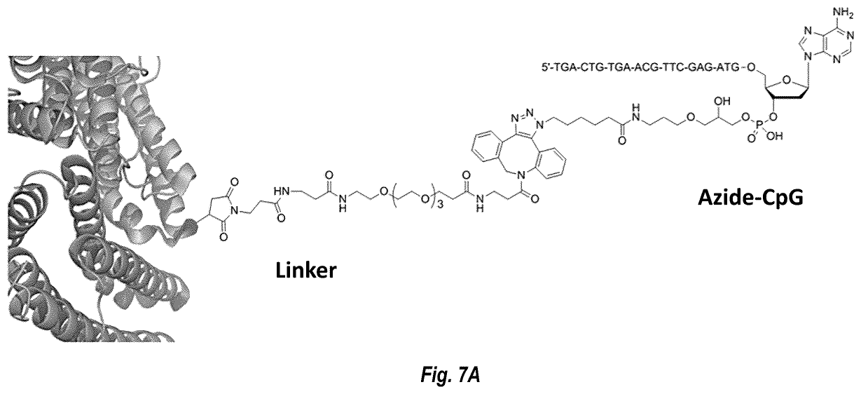

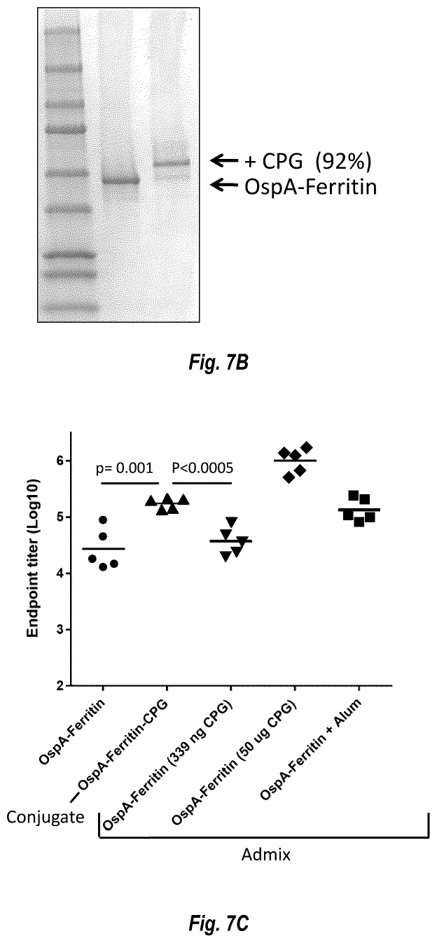

A self-adjuvanting construct was generated by engineering a cysteine (S111C) on the surface of the ferritin nanoparticle that allows direct conjugation of immune-stimulatory moieties such as TLR agonists (FIG. 6A) or CpG (SEQ ID NO: 210; ISS-1018, FIG. 7A) through click chemistry. The procedure for direct conjugation was as follows: Mammalian produced material was reduced to remove cysteinylation with 10 mM TCEP (Amresco K831-10G) in 50 mM Tris pH 8.5 for 1 hr. The protein was then dialyzed into 100 mM Tris pH 8, 50 mM NaCl to remove the TCEP. The E. coli produced material does not need to be reduced. A DBCO-PEG4-Malemide linker (Sigma-Aldrich cat #760676-5 mg) was resuspended at 5 mg/ml in DMSO. 2.5 mg of linker was added to 3 mg of protein in 10 ml volume (final DMSO concentration was 5%). Linker was incubated with the reduced protein for 30 minutes at room temperature. An Ambicon 100 MW cutoff filter concentrator was used to remove excess linker by buffer exchange (Millipore Cat #UFC910096). Azide-PEG4-3M-012 (synthesized in house) and Azide-CPG (ISS-1018 custom synthesized by IDT) were used for the final click chemistry step. 0.5 mg of adjuvant was added to 0.5 mg of protein for final conjugation step and incubated at 37° C. for 6 hours then 4° C. overnight. Excess adjuvant was removed by buffer exchange using an Ambicon 100 MW cutoff filter concentrator. Conjugation efficiency was confirmed by mass spectrometry for 3M-012 and SDS-PAGE analysis for CPG.

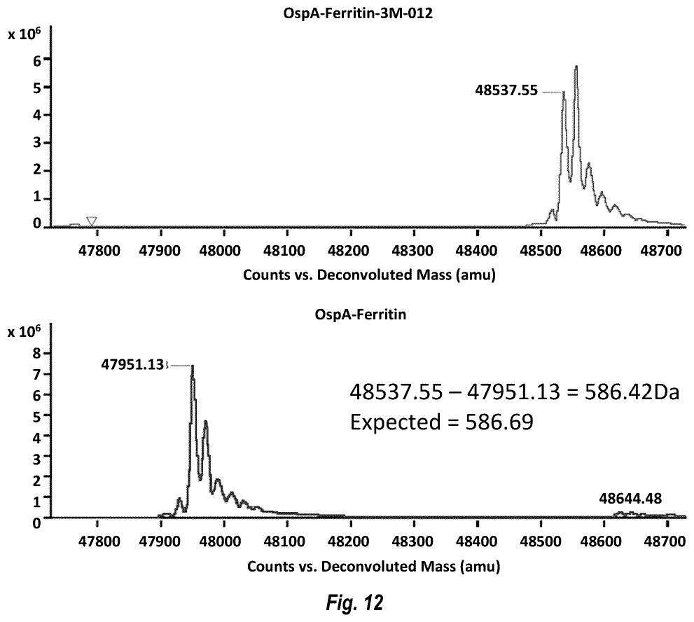

The TLR 7/8 agonist 3M-012, which has previously been shown to increase antibody responses when directly conjugated to the HIV Gag protein (see Wille-Reece, U., et al., Proc Natl Acad Sci USA 102(42): p. 15190-4 (2005)), was used. A two-step, click chemistry approach was used to attach 3M-012 to the nanoparticle of SEQ ID NO: 53. First, the DBCO-PEG4-maleimide linker was connected to the cysteine and then a modified 3M-012 with a PEG4-Azide linker was then added through copper-free azide-alkyne cycloadditions (FIG. 6A). >99% conjugation efficiency was confirmed by mass spectrometry with a mass shift of 587 Daltons (FIG. 12). In addition to azide-3M-012,azide-CPG was also successfully added (FIG. 7A), for which conjugation could be confirmed by gel shift (FIG. 7B).

Nearly complete conjugation of ferritin was observed, suggesting that most nanoparticles carried 24 molecules of agonists. The immunogenicity of the conjugated OspA ferritin nanoparticles was then assessed in mice. C3H/HeN mice were vaccinated intramuscularly at week zero and week 4. ELISAs were run on serum from 2 weeks post 2nd dose. Alum (Alyhydrogel '85 2%; Brenntag—Cat #21645-51-2) was added in equal volume to antigen prior to immunization. Ribi (Sigma adjuvant system Cat #S6322-1vl) was resuspended in 1 ml of PBS and vortexed for 1 minute and then added in equal volume to antigen prior to immunization.

Mice immunized with 3M-012 conjugated particles produced 4.5-fold higher OspA antibody responses than the unconjugated material (FIG. 6B, p<0.05). The antibody responses elicited by 3M-012 conjugated particles were higher than the particles mixed with molar equivalent amount of 3M-012 (29 ng) and comparable to particles mixed with 1,000-fold higher dose (20 μg) of 3M-012 or a standard Alum (Alyhydrogel '85 2%; Brenntag—Cat #21645-51-2) adjuvant.

A similar enhancement of antibody production was observed with CPG-conjugated OspA-ferritin nanoparticle (SEQ ID NO: 53) with a 6.3-fold increase in the immune response compared to unconjugated particles, and a 4.7-fold increase relative to an equivalent amount of unconjugated CPG mixed with nanoparticle (FIG. 7C).

Thus, targeted delivery of adjuvant conjugated to a OspA-ferritin nanoparticle allows substantial reduction in the amount of adjuvant while stimulating an effective and specific antibody response.

### 5. Evaluation of Immunogenicity of OspA-Ferritin Nanoparticles Comprising Different Serotypes

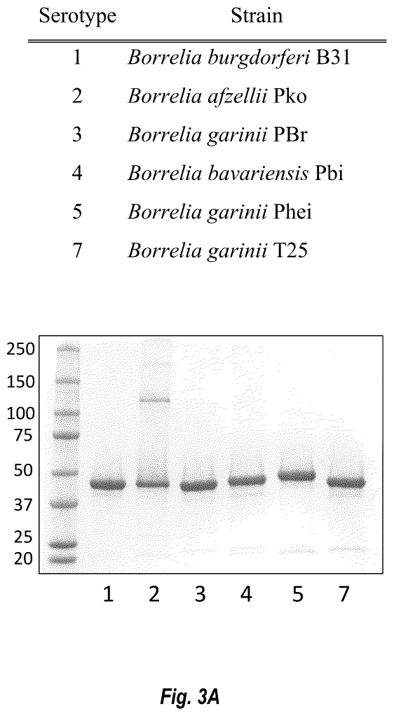

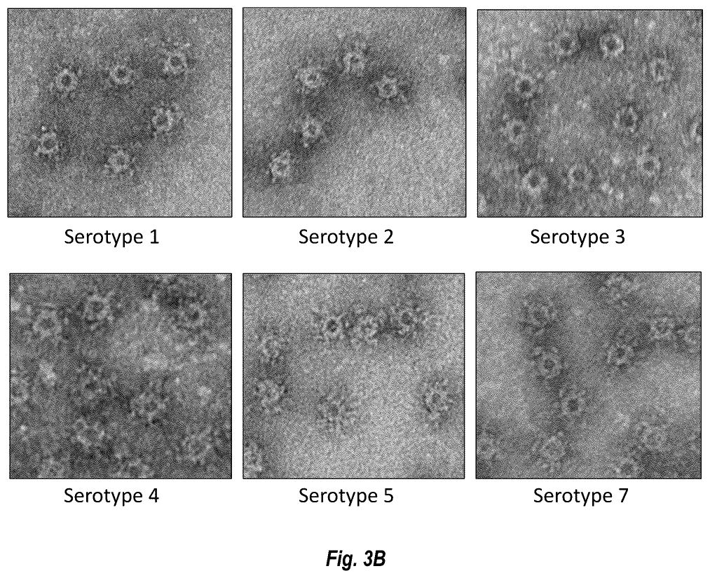

While the serotype 1 OspA strain B. burgdorferi causes disease in the United States, B. afzelii (serotype 2), B. garinii (serotype 3, 5, 6, 7), and B. bavariensis (serotype 4) cause disease in Europe, Asia, and elsewhere. To generate a broadly cross-protective composition, OspA-ferritin nanoparticles were designed for serotypes 1, 2, 3, 4, 5, and 7. (SEQ ID NOS: 1, 5, 6, 7, 8, and 10). These particles were expressed and purified from E. coli using anion exchange and size exclusion chromatography (FIG. 3A). All OspA-ferritin antigenic polypeptides have the expected molecular weight of 47 kDa and DLS analysis and transmission electron microscopy also confirmed the formation of all six OspA nanoparticles (FIG. 3B).

A six-component composition was generated by combining each of serotypes 1-5 and 7 of OspA-ferritin in equimolar proportions.

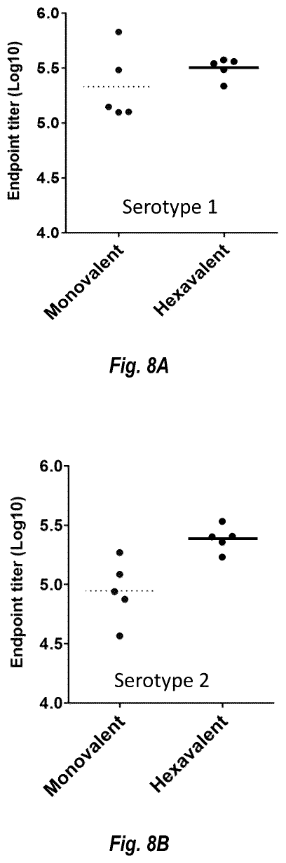

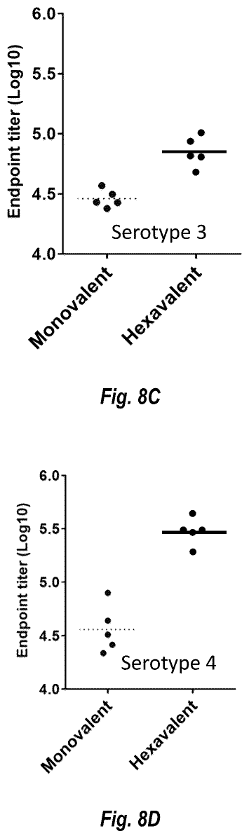

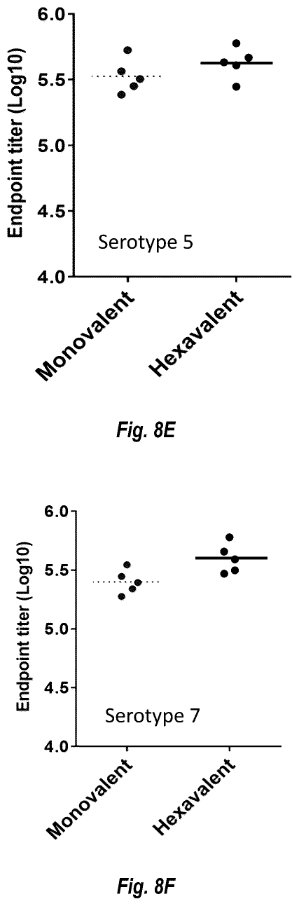

The immunogenicity of this six-component composition (i.e., hexavalent) with Alum was compared in mice to the single-serotype particles (i.e., monovalent) with the same adjuvant (FIGS. 8A-8F). The monovalent composition was given at 1 μg dose, and the hexavalent composition was given at 1 μg for each serotype (total of 6 μg dose). Alum (Alyhydrogel '85 2%; Brenntag—Cat #21645-51-2) was added in equal volume to antigen prior to immunization. The six-component composition induced a robust antibody response against all six of OspA serotypes 1-5 and 7. Moreover, the response to a single serotype control was similar to the mixture, indicating a lack of interference among the six-serotype combination. An improved immune response was seen against serotype 4 with the hexavalent composition relative to the single component composition (see FIG. 8D, serotype 4).

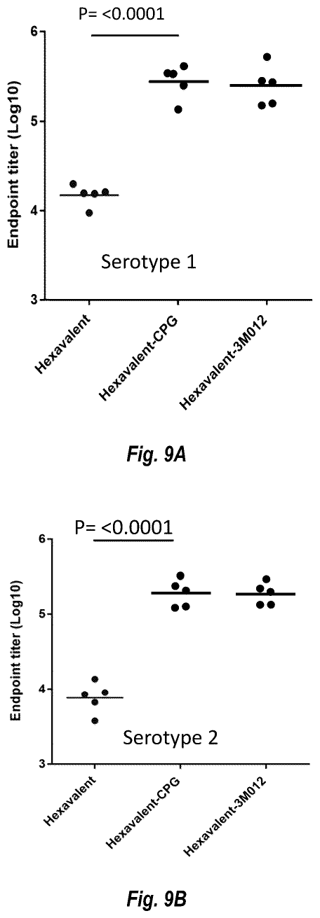

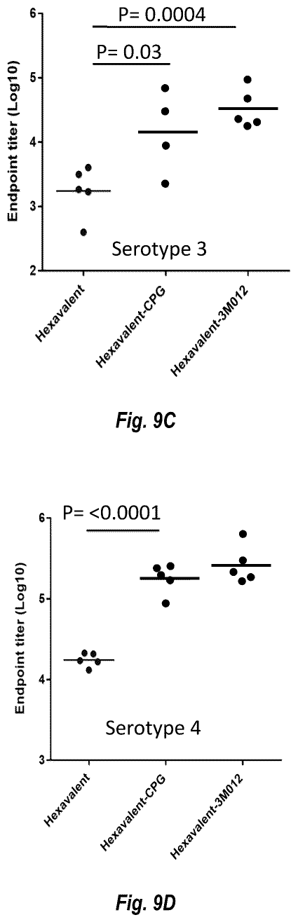

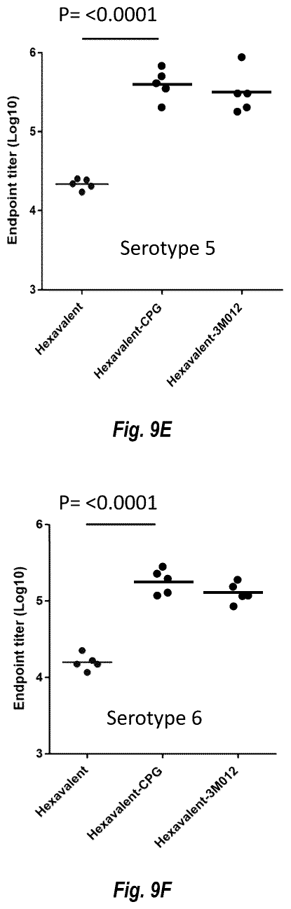

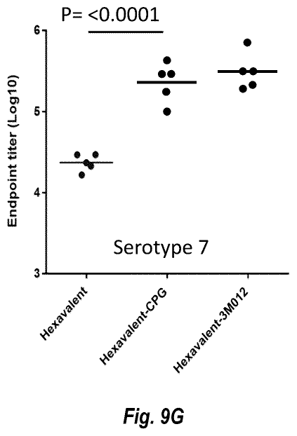

Having established that the hexavalent composition was immunogenic, and in some cases superior to the monovalent composition, 3M-012 and CpG conjugates of each of the six OspA-ferritin nanoparticles were prepared. Two six-component conjugated compositions were created by combining the six OspA-ferritin nanoparticles conjugated to 3M-012 and, separately, by combining the six OspA-ferritin nanoparticles conjugated to CpG. The CpG-conjugated and 3M-012-conjugated hexavalent compositions showed a significant increase in antibody response in mice over the unconjugated hexavalent composition for all seven serotypes of OspA found world-wide (FIG. 9A-9G), indicating that the hexavalent formulation also conferred protection against serotype 6 even though no OspA serotype 6 polypeptide was in the composition.

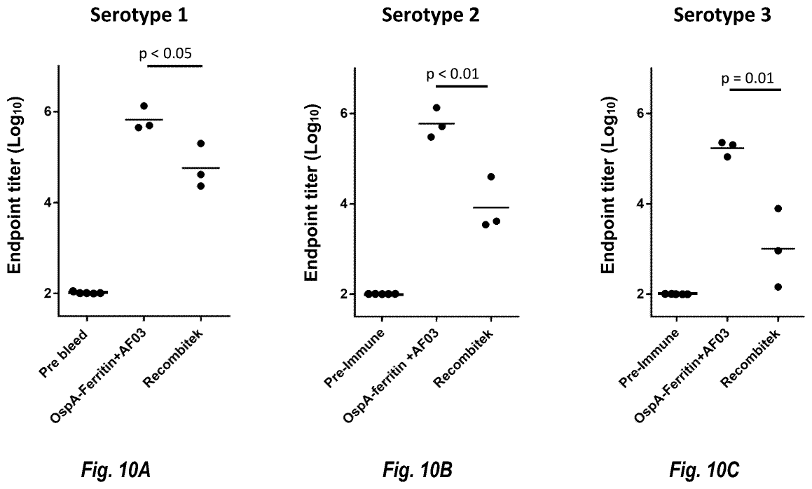

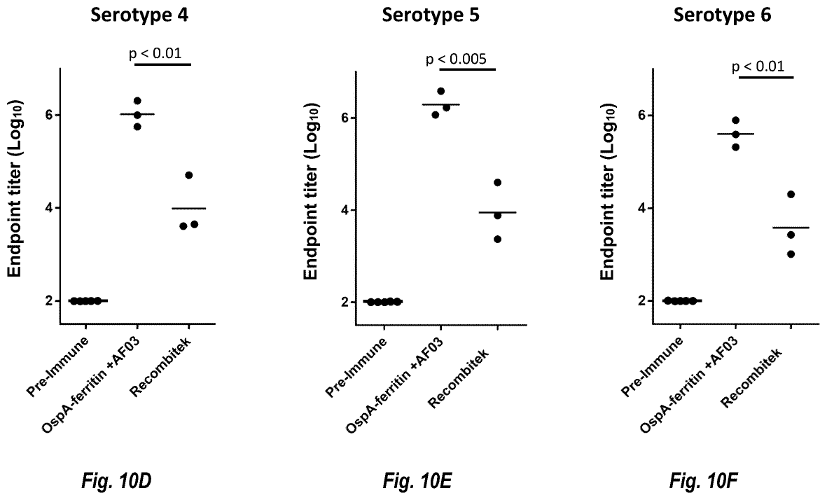

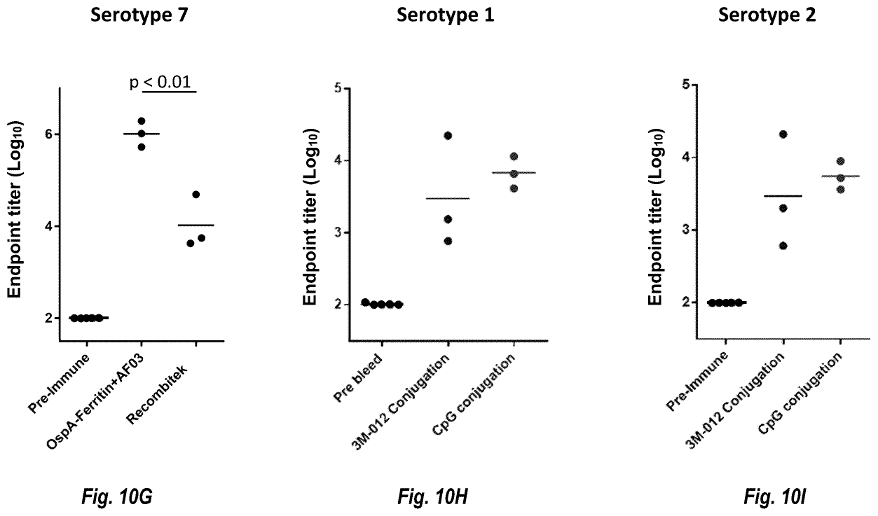

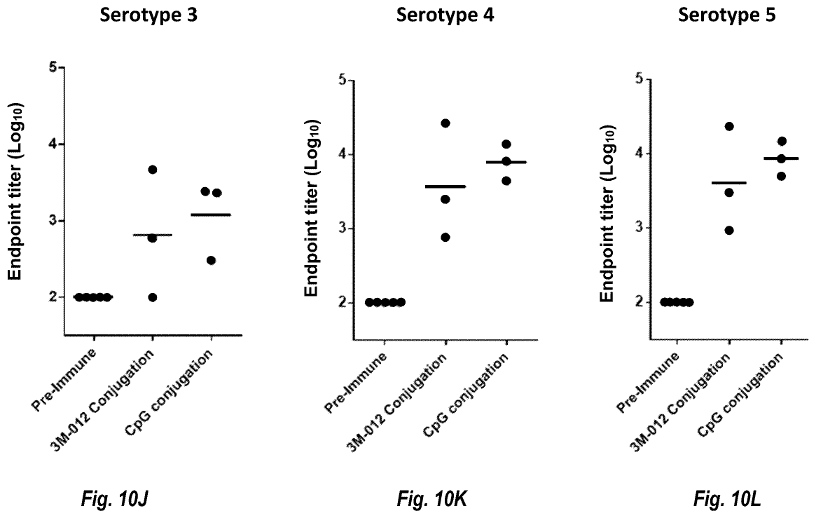

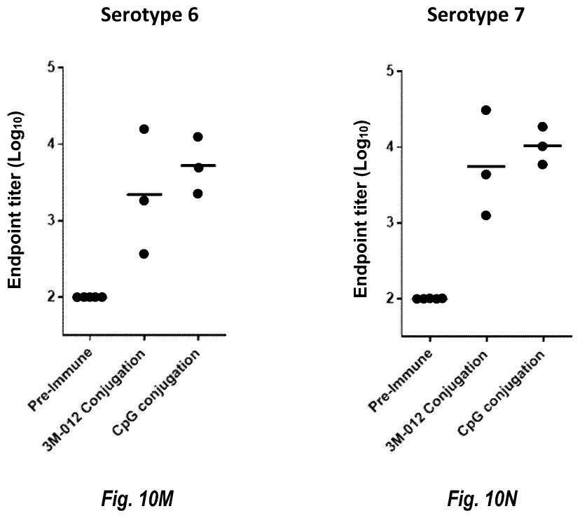

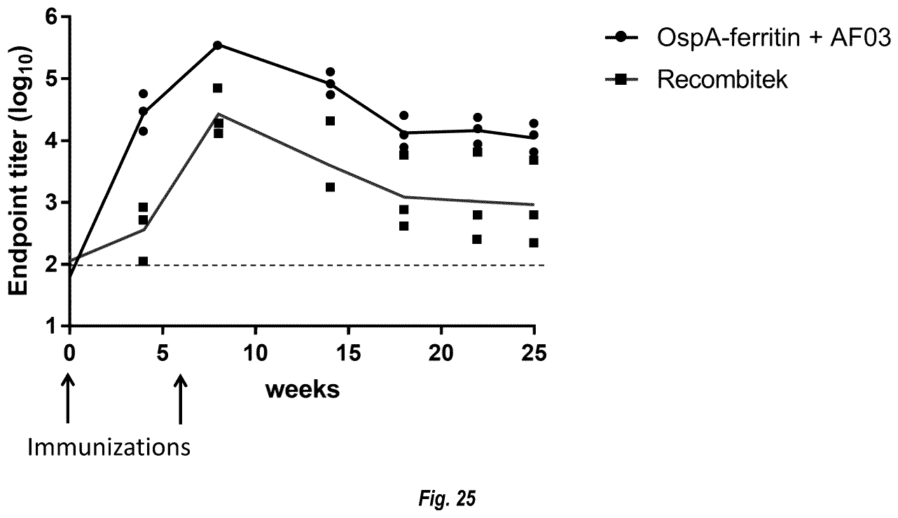

When tested in non-human primates (NHP [Rhesus monkeys]), the hexavalent nanoparticle composition (unconjugated) with AF03 adjuvant outperformed the RECOMBITEK® Lyme control 11 to 200-fold higher Ab titer against all seven circulating Borrelia serotypes (FIGS. 10A-10G). Similar to mice, hexavalent composition elicited high titer Ab response in the presence of adjuvant. The 3M-012 and CpG-conjugated compositions induced a similar response as RECOMBITEK® Lyme control in NHP (compare FIGS. 10A-G with 10H-N, respectively). Antibody titers for the hexavalent vaccine in NHP were robust out to 19 weeks after the boost dose and retained an advantage over the RECOMBITEK® Lyme control (FIG. 25).

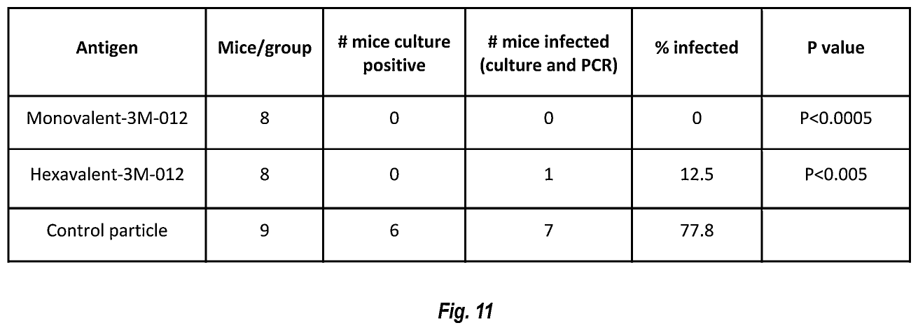

Conjugated compositions were also tested in a tick challenge model. Mice were vaccinated with 1 μg dose of antigens at week 0 and week 4. The monovalent composition contained 1 μg of OspA-ferritin serotype 1 conjugated to 3M-012. The hexavalent composition included OspA from serotypes 1, 2, 3, 4, 5, and 7 at 1 μg each conjugated to 3M-012. Mice were challenged with 5-6 ticks infected with Borrelia burgdorferi N40 strain (serotype 1) for 5 days two weeks after the second immunization and sacrificed two weeks later. Tissue samples from the heart, ankle and ear were cultured in BSK media with antibiotics for B. burgdorferi for 6 weeks. Negative samples were tested by PCR for the presence of B. burgdorferi. Positive samples were positive for either culture or PCR (FIG. 11).

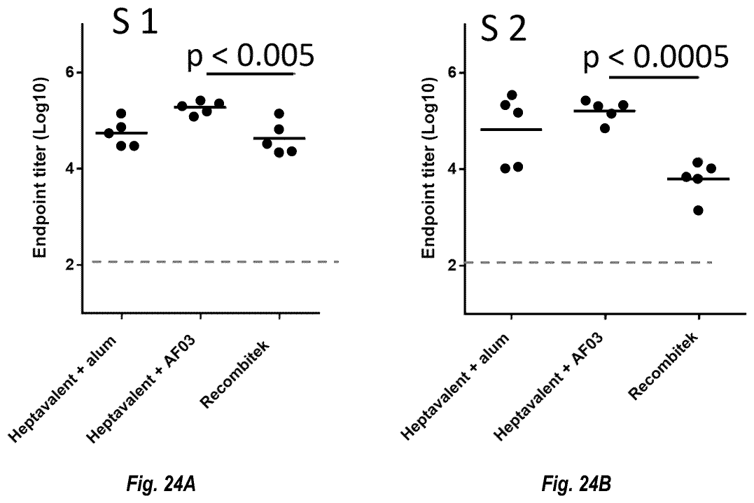

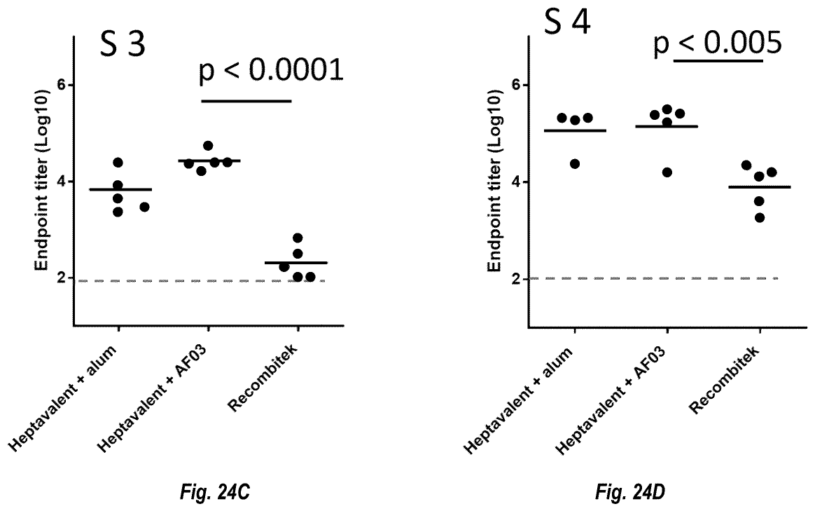

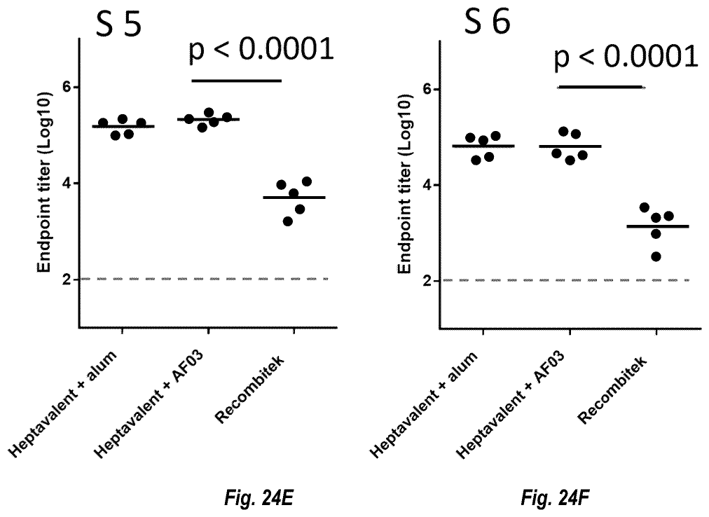

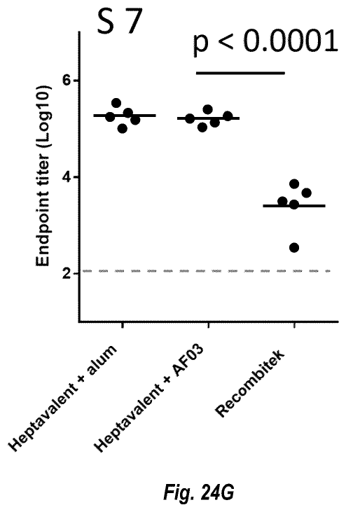

We additionally tested a heptavalent vaccine containing all seven serotypes in mice. Mice were immunized intramuscularly at week 0 and week 4 with heptavalent OspA-ferritin nanoparticle compositions of 1 μg each of OspA-ferritin nanoparticles corresponding to OspA serotypes 1-7 (total 7 μg) adjuvanted with either alum or AF03, or with RECOMBITEK® Lyme (1 μg dose). Antibody response was analyzed 2 weeks after immunization via endpoint titer measured by ELISA. A robust immune response was demonstrated as compared to RECOMBITEK® (FIG. 24A-G).

Thus, OspA-ferritin nanoparticles elicited high titer antibody responses to the seven major serotypes. Further, a seven-component Lyme vaccine candidate offers the potential to control the global spread of Lyme disease.

### 6. Characterization of OspA-Ferritin Constructs with Different Flexible Linkers

Several different linkers were tested to provide flexibility between OspA and ferritin. The constructs ranged from one to five—GGGS (SEQ ID NO: 226)—sequences. The various linker constructs were purified and formed nanoparticles of uniform size.

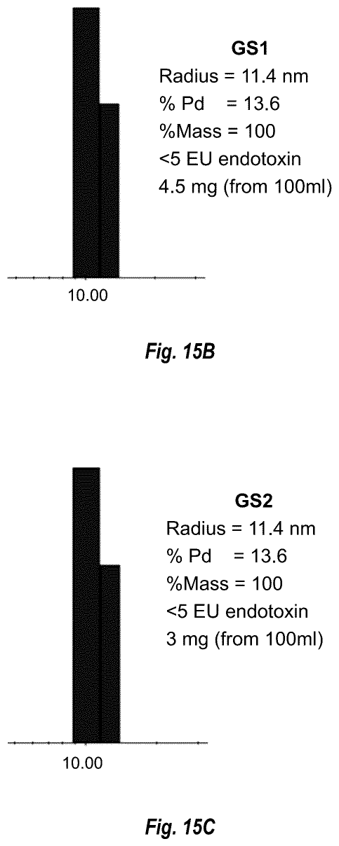

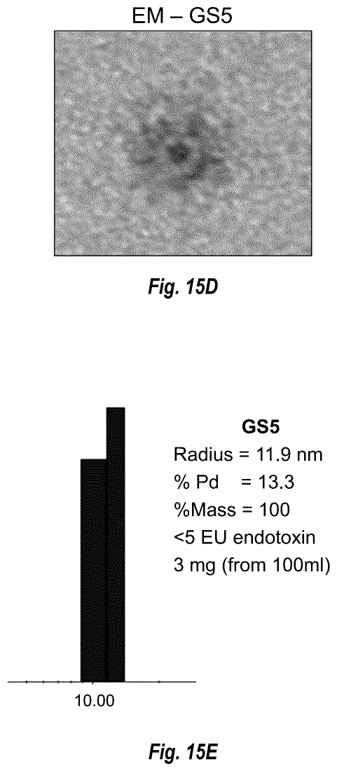

OspA-linker-ferritin constructs comprising GS1 (GGGS (SEQ ID NO: 226)), GS2 (SEQ ID NO: 91), or GS5 (SEQ ID NO: 92) linkers could all be expressed (FIG. 15A) and showed consistent DLS (FIGS. 15B, 15C, and 15E) and EM (FIG. 15D) profiles.

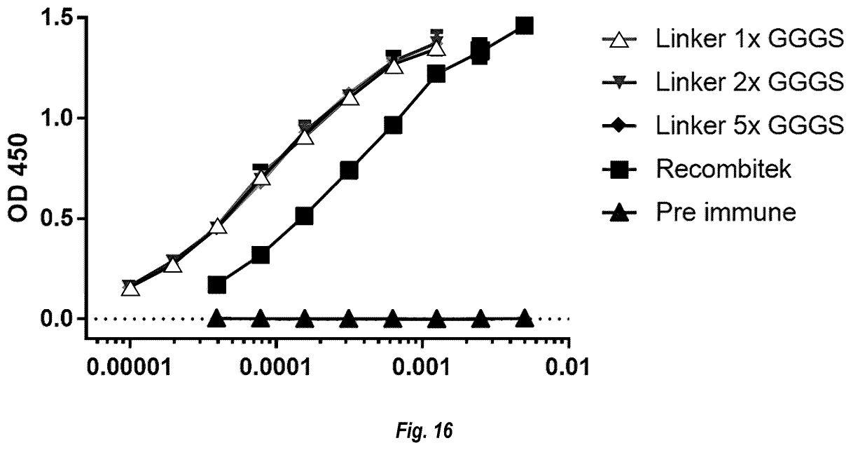

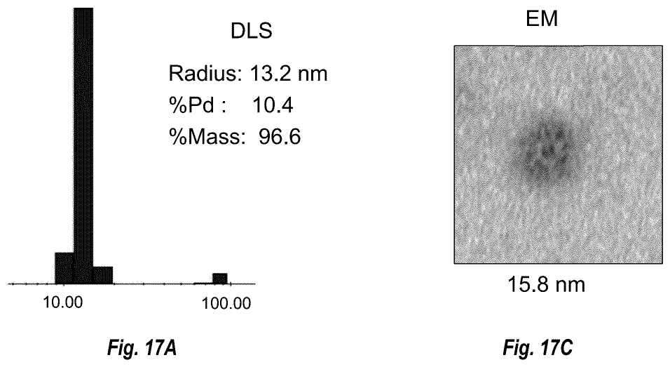

Further, the different—GGGS (SEQ ID NO: 226)—linker constructs (Linker 1x GGGS (SEQ ID NO: 226) [SEQ ID NO: 60], Linker 2x GGGS (SEQ ID NO: 91) [SEQ ID NO: 61], and Linker 5x GGGS (SEQ ID NO: 92) [SEQ ID NO: 62]) all showed strong immune responses in C3H mice (FIG. 16).

### 7. Characterization of Lumazine Synthase OspA Nanoparticles

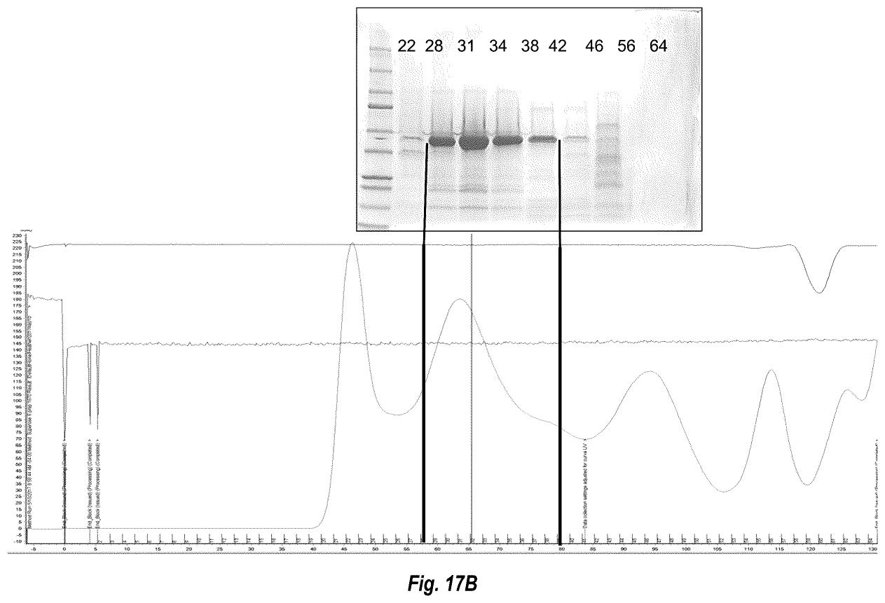

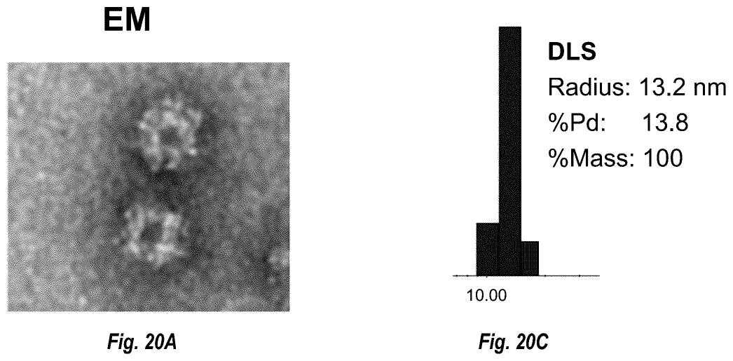

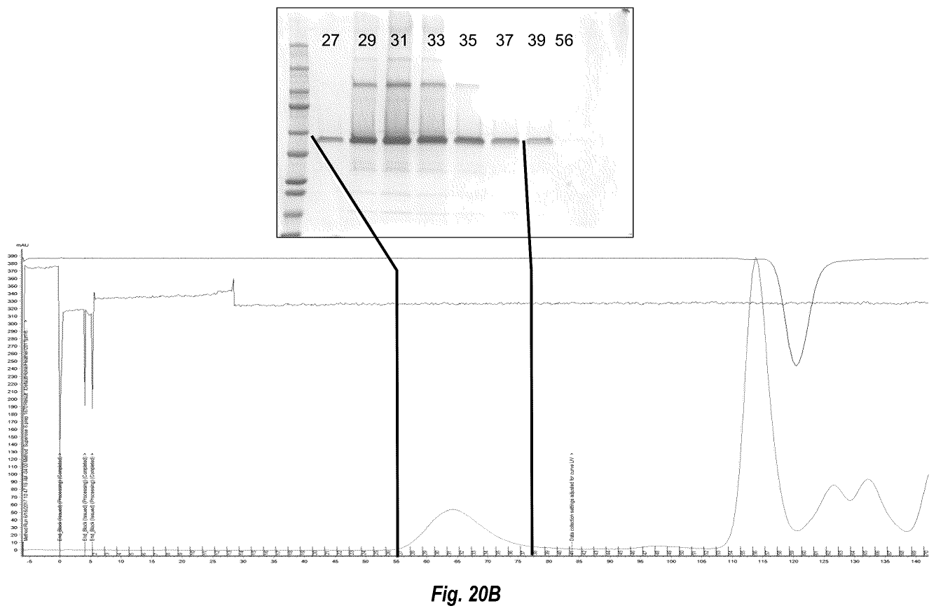

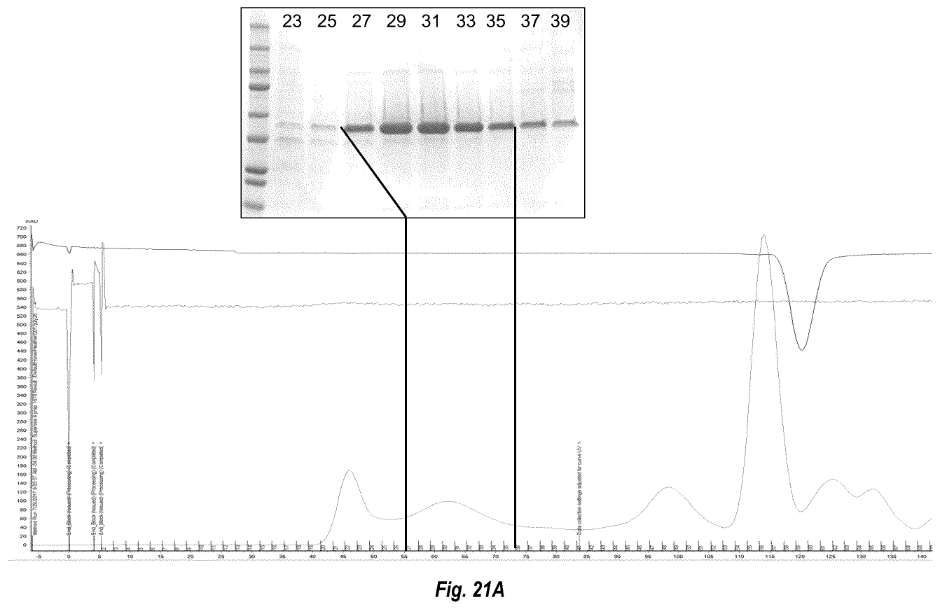

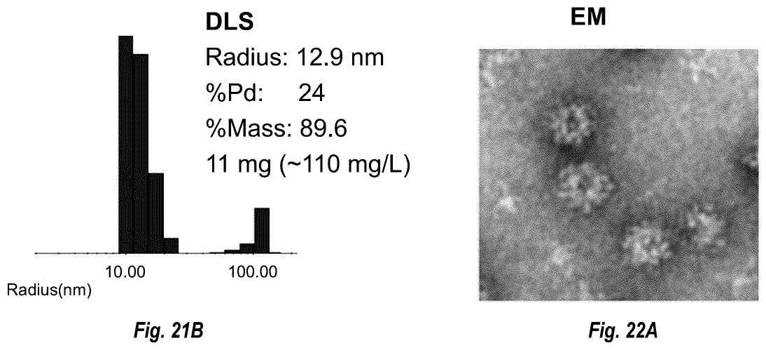

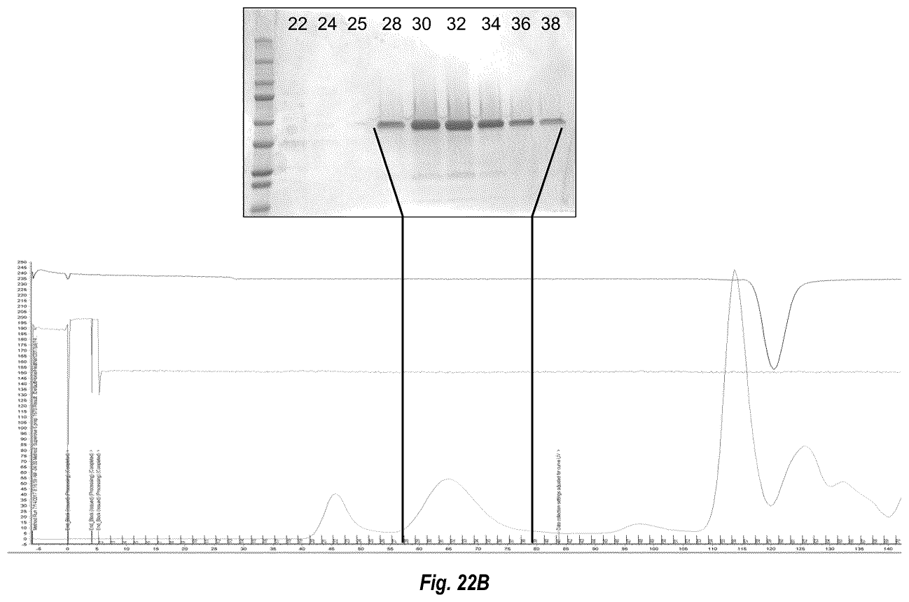

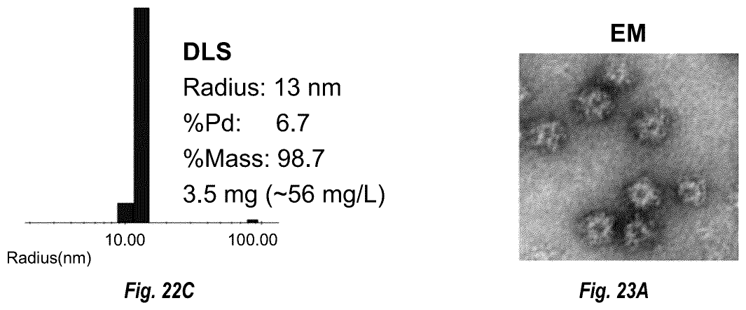

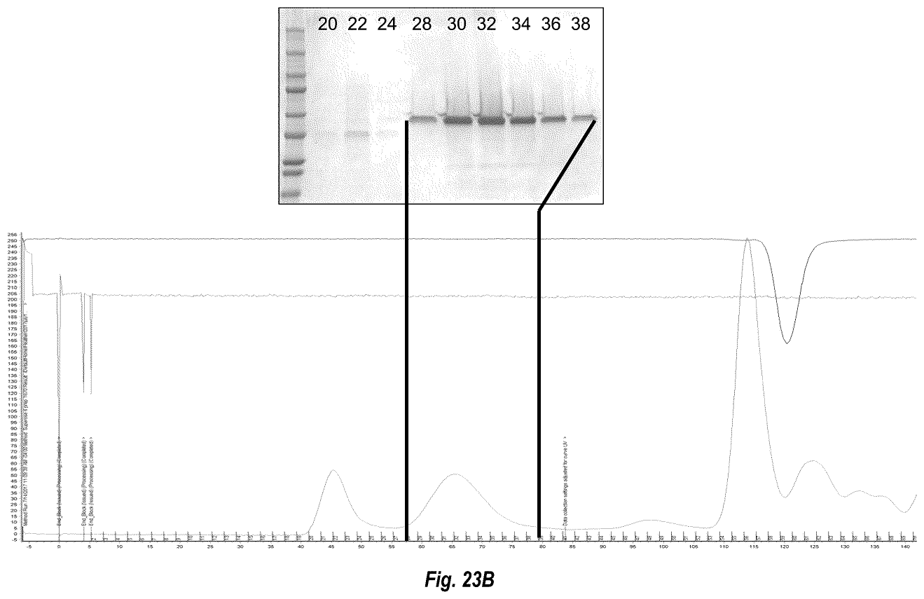

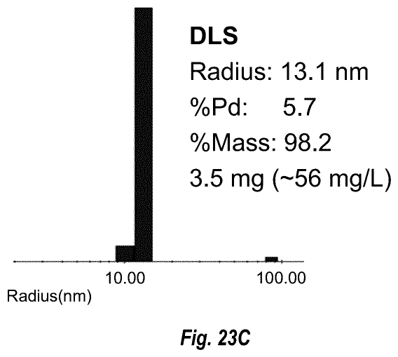

Another nanoparticle, lumazine synthase from Aquifex aeolicus, was investigated for antigenic display of OspA. OspA-lumazine synthase particles comprising different serotypes were purified easily from E. coli cells by anion exchange and size exclusion chromatography. Constructs were generated and characterized that comprised OspA serotype 1 (SEQ ID NO: 12, FIGS. 19A-19C); OspA serotype 2 (SEQ ID NO: 16 ,FIGS. 20A-20C); OspA serotype 3 (SEQ ID NO: 17, FIGS. 21A-21B); OspA serotype 4 (SEQ ID NO: 18, FIGS. 17A-17C); OspA serotype 5 (SEQ ID NO: 19, FIGS. 22A-22C); and OspA serotype 7 (SEQ ID NO: 21, FIGS. 23A-23C). The OspA-lumazine synthase particles formed a 15.8 nm particle by EM and were uniform in size by DLS.

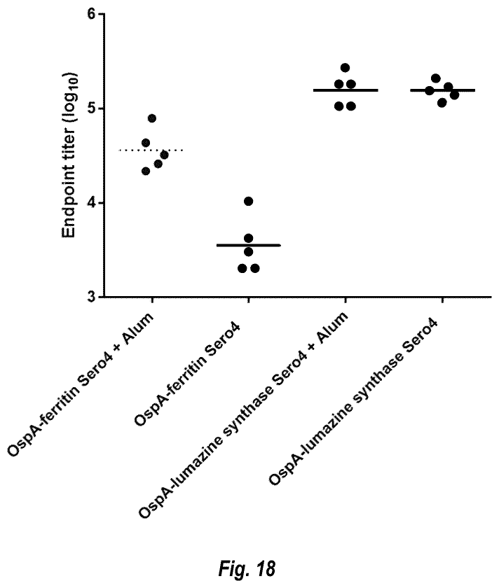

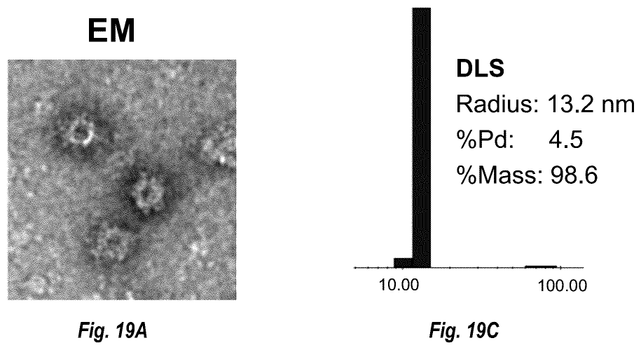

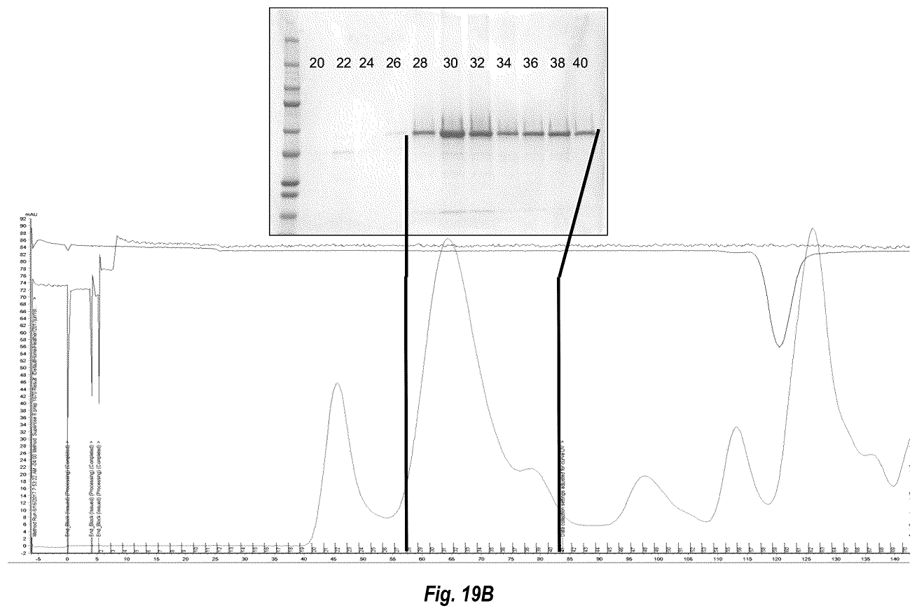

OspA serotype 4 lumazine synthase particles (SEQ ID NO: 18) were tested in mice for immunogenicity (FIG. 18). The OspA lumazine synthase particles with and without Alum gave a strong immune response that appeared at least as robust as that of a similar OspA serotype 4 ferritin nanoparticle (SEQ ID NO: 7).

Thus, antigenic polypeptides comprising lumazine synthase and an OspA polypeptide can also be used to elicit anti-OspA antibody responses.

1.-38. (canceled)

39. An antigenic OspA polypeptide comprising an outer surface protein A (OspA) serotype 1 polypeptide of Borrelia, wherein the OspA serotype 1 polypeptide of Borrelia does not comprise the sequence of SEQ ID NO: 77, and comprises a modification to reduce or eliminate glycosylation.

40. The antigenic OspA polypeptide of claim 39, wherein the OspA serotype 1 polypeptide of Borrelia (a) lacks a transmembrane domain or a portion of a transmembrane domain, and/or (b) is non-lipidated.

41. The antigenic OspA polypeptide of claim 39, wherein one or more of the amino acids of SEQ ID NO: 77 is replaced with the corresponding amino acid(s) of a non-serotype 1 OspA, optionally wherein the non-serotype 1 OspA is serotype 2, 3, 4, 5, 6, or 7 OspA.

42. The antigenic OspA polypeptide of claim 39, wherein the OspA serotype 1 polypeptide of Borrelia comprises a sequence with at least 85%, 90%, 95%, 97%, 98%, 99%, 99.5%, or 100% identity to the OspA serotype I polypeptide of Borrelia sequence of any one of SEQ ID NOS: 1-10 or 12-76.

43. The antigenic OspA polypeptide of claim 39, wherein the modification comprises a substitution of at least one asparagine.

44. The antigenic OspA polypeptide of claim 43, wherein the at least one asparagine comprises any one, two, three, or more of N71, N190, N202, and N251 corresponding to the amino acid sequence of SEQ ID NO: 83 as determined by pair-wise or structural alignment.

45. The antigenic OspA polypeptide of claim 43, wherein the one or more asparagines are substituted with glutamine.

46. The antigenic OspA polypeptide of claim 39, wherein the OspA serotype 1 polypeptide of Borrelia lacks an N-glycosylation site.

47. A nucleic acid encoding the antigenic OspA polypeptide of claim 39, optionally wherein the nucleic acid is an mRNA.

48. A method of eliciting an immune response to Borrelia or protecting a subject against Lyme Disease comprising administering the antigenic OspA polypeptide of claim 39 to a subject.

49. A nucleic acid encoding an antigenic OspA polypeptide, the antigenic OspA polypeptide comprising (a) an OspA serotype 1 polypeptide of Borrelia, and (b) a ferritin protein, wherein the OspA serotype 1 polypeptide of Borrelia does not comprise the sequence of SEQ ID NO: 77.

50. The nucleic acid of claim 49, wherein the ferritin protein comprises a mutation replacing a surface-exposed amino acid with a cysteine.

51. The nucleic acid of claim 49, wherein the ferritin protein comprises one or more of E12C, S26C, S72C, A75C, K79C, S100C, and S111C mutations of H. pylori ferritin or one or more corresponding mutations in a non-H. pylori ferritin as determined by pairwise or structural alignment.

52. The nucleic acid of claim 49, wherein the ferritin protein comprises a mutation replacing a surface-exposed asparagine with a non-asparagine amino acid, optionally wherein the asparagine is at position 19 of H. pylori ferritin, or an analogous position in a non-H. pylori ferritin as determined by pairwise or structural alignment.

53. The nucleic acid of claim 49, wherein the ferritin protein comprises a mutation replacing an internal cysteine with a non-cysteine amino acid, optionally wherein the internal cysteine is at position 31 of H. pylori ferritin, or a position that corresponds to position 31 of H. pylori ferritin as determined by pair-wise or structural alignment.

54. The nucleic acid of claim 49, wherein the OspA serotype 1 polypeptide of Borrelia comprises a sequence with at least 85%, 90%, 95%, 97%, 98%, 99%, 99.5%, or 100% identity to the OspA polypeptide of Borrelia sequence of any one of SEQ ID NOS: 1-76, or 94-102.

55. A method of eliciting an immune response to Borrelia or protecting a subject against Lyme Disease comprising administering the nucleic acid of claim 49 to a subject.

56. An antigenic OspA polypeptide comprising (a) an OspA serotype 1 polypeptide of Borrelia, and (b) a lumazine synthase protein, wherein the polypeptide does not comprise the sequence of SEQ ID NO: 77.

57. A nucleic acid encoding the antigenic OspA polypeptide of claim 56, optionally wherein the nucleic acid is an mRNA.

58. A method of eliciting an immune response to Borrelia or protecting a subject against Lyme Disease comprising administering the antigenic OspA polypeptide of claim 56 to a subject.


SEQUENCE LISTING (228 sequences)

SEQ ID NO: 1 - protein, 425 aa, synthetic construct
MDEKNSVSVDLPGEMKVLVSKEKNKDGKYDLIATVDKLELKGTSDKNNGSGVLEGVKADKSKVKLTISDDLGQTTLEVFKEDGKTLVSKKVTSKDKSSTEEKFNEKGEVSEKIITRADGTRLEYTGIKSDGSGKAKEVLKGYTLEGQLSDEKTTLVVKEGTVTLSKNISKSGEVSVELNDTDSSAATKKTAAWNSGTSTLTITVNSKKTKDLVFTKENTITVQQYDSNGTKLEGSAVEITKLDEIKNALKGSESQVRQQFSKDIEKLLNEQVNKEMQSSNLYMSMSSWSYTHSLDGAGLFLFDHAAEEYEHAKKLIIFLNENNVPVQLTSISAPEHKFEGLTQIFQKAYEHEQHISESINNIVDHAIKCKDHATFNFLQWYVAEQHEEEVLFKDILDKIELIGNENHGLYLADQYVKGIAKSRKS

SEQ ID NO: 2 - protein, 425 aa, synthetic construct
MDEKNSVSVDLPGEMKVLVSKEKNKDGKYDLIATVDKLELKGTSDKNNGSGVLEGVKADKSKVKLTISDDLGQTTLEVFKEDGKTLVSKKVTSKDKSSTEEKFNEKGEVSEKIITRADGTRLEYTGIKSDGSGKAKEVLKGYDLKGELSSEKTTLVVKEGTVTLSKNISKSGEVSVELNDTDSSAATKKTAAWNSGTSTLTITVNSKKTKDLVFTKENTITVQQYDSNGTKLEGSAVEITKLDEIKNALKGSESQVRQQFSKDIEKLLNEQVNKEMQSSNLYMSMSSWSYTHSLDGAGLFLFDHAAEEYEHAKKLIIFLNENNVPVQLTSISAPEHKFEGLTQIFQKAYEHEQHISESINNIVDHAIKCKDHATFNFLQWYVAEQHEEEVLFKDILDKIELIGNENHGLYLADQYVKGIAKSRKS

SEQ ID NO: 3 - protein, 425 aa, synthetic construct
MDEKNSVSVDLPGEMKVLVSKEKNKDGKYDLIATVDKLELKGTSDKNNGSGVLEGVKADKSKVKLTISDDLGQTTLEVFKEDGKTLVSKKVTSKDKSSTEEKFNEKGEVSEKIITRADGTRLEYTGIKSDGSGKAKEVLKGFTLEGKVANEKTTLVVKEGTVTLSKNISKSGEVSVELNDTDSSAATKKTAAWNSGTSTLTITVNSKKTKDLVFTKENTITVQQYDSNGTKLEGSAVEITKLDEIKNALKGSESQVRQQFSKDIEKLLNEQVNKEMQSSNLYMSMSSWSYTHSLDGAGLFLFDHAAEEYEHAKKLIIFLNENNVPVQLTSISAPEHKFEGLTQIFQKAYEHEQHISESINNIVDHAIKCKDHATFNFLQWYVAEQHEEEVLFKDILDKIELIGNENHGLYLADQYVKGIAKSRKS

SEQ ID NO: 4 - protein, 425 aa, synthetic construct
MDEKNSVSVDLPGEMKVLVSKEKNKDGKYDLIATVDKLELKGTSDKNNGSGVLEGVKADKSKVKLTISDDLGQTTLEVFKEDGKTLVSKKVTSKDKSSTEEKFNEKGEVSEKIITRADGTRLEYTGIKSDGSGKAKEVLKGFALEGTLTDEKTTLVVKEGTVTLSKNISKSGEVSVELNDTDSSAATKKTAAWNSGTSTLTITVNSKKTKDLVFTKENTITVQQYDSNGTKLEGSAVEITKLDEIKNALKGSESQVRQQFSKDIEKLLNEQVNKEMQSSNLYMSMSSWSYTHSLDGAGLFLFDHAAEEYEHAKKLIIFLNENNVPVQLTSISAPEHKFEGLTQIFQKAYEHEQHISESINNIVDHAIKCKDHATFNFLQWYVAEQHEEEVLFKDILDKIELIGNENHGLYLADQYVKGIAKSRKS

SEQ ID NO: 5 - protein, 425 aa, synthetic construct
MDEKNSASVDLPGEMKVLVSKEKDKDGKYSLKATVDKIELKGTSDKDNGSGVLEGTKDDKSKAKLTIADDLSKTTFELFKEDGKTLVSRKVSSKDKTSTDEMFNEKGELSAKTMTRENGTKLEYTEMKSDGTGKAKEVLKNFTLEGKVANDKVTLEVKEGTVTLSKEIAKSGEVTVALNDTNTTQATKKTGAWDSKTSTLTISVNSKKTTQLVFTKQDTITVQKYDSAGTNLEGTAVEIKTLDELKNALKGSESQVRQQFSKDIEKLLNEQVNKEMQSSNLYMSMSSWSYTHSLDGAGLFLFDHAAEEYEHAKKLIIFLNENNVPVQLTSISAPEHKFEGLTQIFQKAYEHEQHISESINNIVDHAIKCKDHATFNFLQWYVAEQHEEEVLFKDILDKIELIGNENHGLYLADQYVKGIAKSRKS

SEQ ID NO: 6 - protein, 426 aa, synthetic construct
MDEKNSVSVDLPGGMKVLVSKEKDKDGKYSLMATVEKLELKGTSDKSNGSGVLEGEKADKSKAKLTISQDLNQTTFEIFKEDGKTLVSRKVNSKDKSSTEEKFNDKGKLSEKVVTRANGTRLEYTEIKNDGSGKAKEVLKGFALEGTLTDGGETKLTVTEGTVTLSKNISKSGEITVALNDTETTPADKKTGEWKSDTSTLTISKNSQKPKQLVFTKENTITVQNYNRAGNALEGSPAEIKDLAELKAALKGSESQVRQQFSKDIEKLLNEQVNKEMQSSNLYMSMSSWSYTHSLDGAGLFLFDHAAEEYEHAKKLIIFLNENNVPVQLTSISAPEHKFEGLTQIFQKAYEHEQHISESINNIVDHAIKCKDHATFNFLQWYVAEQHEEEVLFKDILDKIELIGNENHGLYLADQYVKGIAKSRKS

SEQ ID NO: 7 - protein, 425 aa, synthetic construct
MDEKNSVSVDLPGEMKVLVSKEKDKDGKYSLMATVDKLELKGTSDKSNGSGTLEGEKSDKSKAKLTISEDLSKTTFEIFKEDGKTLVSKKVNSKDKSSIEEKFNAKGELSEKTILRANGTRLEYTEIKSDGTGKAKEVLKDFALEGTLAADKTTLKVTEGTVVLSKHIPNSGEITVELNDSNSTQATKKTGKWDSNTSTLTISVNSKKTKNIVFTKEDTITVQKYDSAGTNLEGNAVEIKTLDELKNALKGSESQVRQQFSKDIEKLLNEQVNKEMQSSNLYMSMSSWSYTHSLDGAGLFLFDHAAEEYEHAKKLIIFLNENNVPVQLTSISAPEHKFEGLTQIFQKAYEHEQHISESINNIVDHAIKCKDHATFNFLQWYVAEQHEEEVLFKDILDKIELIGNENHGLYLADQYVKGIAKSRKS

SEQ ID NO: 8 - protein, 426 aa, synthetic construct
MDEKNSVSVDLPGGMKVLVSKEKDKDGKYSLMATVEKLELKGTSDKNNGSGTLEGEKTDKSKVKLTIAEDLSKTTFEIFKEDGKTLVSKKVTLKDKSSTEEKFNEKGEISEKTIVRANGTRLEYTDIKSDGSGKAKEVLKDFTLEGTLAADGKTTLKVTEGTVVLSKNILKSGEITVALDDSDTTQATKKTGKWDSKTSTLTISVNSQKTKNLVFTKEDTITVQKYDSAGTNLEGKAVEITTLEKLKDALKGSESQVRQQFSKDIEKLLNEQVNKEMQSSNLYMSMSSWSYTHSLDGAGLFLFDHAAEEYEHAKKLIIFLNENNVPVQLTSISAPEHKFEGLTQIFQKAYEHEQHISESINNIVDHAIKCKDHATFNFLQWYVAEQHEEEVLFKDILDKIELIGNENHGLYLADQYVKGIAKSRKS

SEQ ID NO: 9 - protein, 426 aa, synthetic construct
MDEKNSVSVDLPGGMTVLVSKEKDKDGKYSLEATVDKLELKGTSDKNNGSGTLEGEKTDKSKVKSTIADDLSQTKFEIFKEDGKTLVSKKVTLKDKSSTEEKFNGKGETSEKTIVRANGTRLEYTDIKSDGSGKAKEVLKDFTLEGTLAADGKTTLKVTEGTVVLSKNILKSGEITAALDDSDTTRATKKTGKWDSKTSTLTISVNSQKTKNLVFTKEDTITVQRYDSAGTNLEGKAVEITTLKELKNALKGSESQVRQQFSKDIEKLLNEQVNKEMQSSNLYMSMSSWSYTHSLDGAGLFLFDHAAEEYEHAKKLIIFLNENNVPVQLTSISAPEHKFEGLTQIFQKAYEHEQHISESINNIVDHAIKCKDHATFNFLQWYVAEQHEEEVLFKDILDKIELIGNENHGLYLADQYVKGIAKSRKS

SEQ ID NO: 10 - protein, 426 aa, synthetic construct
MDEKNSVSVDLPGEMKVLVSKEKDKDGKYSLEATVDKLELKGTSDKNNGSGVLEGVKAAKSKAKLTIADDLSQTKFEIFKEDGKTLVSKKVTLKDKSSTEEKFNDKGKLSEKVVTRANGTRLEYTEIQNDGSGKAKEVLKSLTLEGTLTADGETKLTVEAGTVTLSKNISESGEITVELKDTETTPADKKSGTWDSKTSTLTISKNSQKTKQLVFTKENTITVQKYNTAGTKLEGSPAEIKDLEALKAALKGSESQVRQQFSKDIEKLLNEQVNKEMQSSNLYMSMSSWSYTHSLDGAGLFLFDHAAEEYEHAKKLIIFLNENNVPVQLTSISAPEHKFEGLTQIFQKAYEHEQHISESINNIVDHAIKCKDHATFNFLQWYVAEQHEEEVLFKDILDKIELIGNENHGLYLADQYVKGIAKSRKS

SEQ ID NO: 11 - protein, 265 aa, synthetic construct
MDEKNSVSVDLPGEMKVLVSKEKDKDGKYSLMATVDKLELKGTSDKSNGSGTLEGEKSDKSKAKLTISEDLSKTTFEIFKEDGKTLVSKKVNSKDKSSIEEKFNAKGELSEKTILRANGTRLEYTEIKSDGTGKAKEVLKDFALEGTLAADKTTLKVTEGTVVLSKHIPNSGEITVELNDSNSTQATKKTGKWDSNTSTLTISVNSKKTKNIVFTKEDTITVQKYDSAGTNLEGNAVEIKTLDELKNALKCLVPRGSLEHHHHHH

SEQ ID NO: 12 - protein, 408 aa, synthetic construct
MDEKNSVSVDLPGEMKVLVSKEKNKDGKYDLIATVDKLELKGTSDKNNGSGVLEGVKADKSKVKLTISDDLGQTTLEVFKEDGKTLVSKKVTSKDKSSTEEKFNEKGEVSEKIITRADGTRLEYTGIKSDGSGKAKEVLKGYTLEGQLSDEKTTLVVKEGTVTLSKNISKSGEVSVELNDTDSSAATKKTAAWNSGTSTLTITVNSKKTKDLVFTKENTITVQQYDSNGTKLEGSAVEITKLDEIKNALKGGGSMQIYEGKLTAEGLRFGIVASRFNHALVDRLVEGAIDCIVRHGGREEDITLVRVPGSWEIPVAAGELARKEDIDAVIAIGVLIRGATPHFDYIASEVSKGLANLSLELRKPITFGVITADTLEQAIERAGTKHGNKGWEAALSAIEMANLFKSLR

SEQ ID NO: 13 - protein, 408 aa, synthetic construct
MDEKNSVSVDLPGEMKVLVSKEKNKDGKYDLIATVDKLELKGTSDKNNGSGVLEGVKADKSKVKLTISDDLGQTTLEVFKEDGKTLVSKKVTSKDKSSTEEKFNEKGEVSEKIITRADGTRLEYTGIKSDGSGKAKEVLKGYDLKGELSSEKTTLVVKEGTVTLSKNISKSGEVSVELNDTDSSAATKKTAAWNSGTSTLTITVNSKKTKDLVFTKENTITVQQYDSNGTKLEGSAVEITKLDEIKNALKGGGSMQIYEGKLTAEGLRFGIVASRFNHALVDRLVEGAIDCIVRHGGREEDITLVRVPGSWEIPVAAGELARKEDIDAVIAIGVLIRGATPHFDYIASEVSKGLANLSLELRKPITFGVITADTLEQAIERAGTKHGNKGWEAALSAIEMANLFKSLR

SEQ ID NO: 14 - protein, 408 aa, synthetic construct
MDEKNSVSVDLPGEMKVLVSKEKNKDGKYDLIATVDKLELKGTSDKNNGSGVLEGVKADKSKVKLTISDDLGQTTLEVFKEDGKTLVSKKVTSKDKSSTEEKFNEKGEVSEKIITRADGTRLEYTGIKSDGSGKAKEVLKGFTLEGKVANEKTTLVVKEGTVTLSKNISKSGEVSVELNDTDSSAATKKTAAWNSGTSTLTITVNSKKTKDLVFTKENTITVQQYDSNGTKLEGSAVEITKLDEIKNALKGGGSMQIYEGKLTAEGLRFGIVASRFNHALVDRLVEGAIDCIVRHGGREEDITLVRVPGSWEIPVAAGELARKEDIDAVIAIGVLIRGATPHFDYIASEVSKGLANLSLELRKPITFGVITADTLEQAIERAGTKHGNKGWEAALSAIEMANLFKSLR

SEQ ID NO: 15 - protein, 408 aa, synthetic construct
MDEKNSVSVDLPGEMKVLVSKEKNKDGKYDLIATVDKLELKGTSDKNNGSGVLEGVKADKSKVKLTISDDLGQTTLEVFKEDGKTLVSKKVTSKDKSSTEEKFNEKGEVSEKIITRADGTRLEYTGIKSDGSGKAKEVLKGFALEGTLTDEKTTLVVKEGTVTLSKNISKSGEVSVELNDTDSSAATKKTAAWNSGTSTLTITVNSKKTKDLVFTKENTITVQQYDSNGTKLEGSAVEITKLDEIKNALKGGGSMQIYEGKLTAEGLRFGIVASRFNHALVDRLVEGAIDCIVRHGGREEDITLVRVPGSWEIPVAAGELARKEDIDAVIAIGVLIRGATPHFDYIASEVSKGLANLSLELRKPITFGVITADTLEQAIERAGTKHGNKGWEAALSAIEMANLFKSLR

SEQ ID NO: 16 - protein, 408 aa, synthetic construct
MDEKNSASVDLPGEMKVLVSKEKDKDGKYSLKATVDKIELKGTSDKDNGSGVLEGTKDDKSKAKLTIADDLSKTTFELFKEDGKTLVSRKVSSKDKTSTDEMFNEKGELSAKTMTRENGTKLEYTEMKSDGTGKAKEVLKNFTLEGKVANDKVTLEVKEGTVTLSKEIAKSGEVTVALNDTNTTQATKKTGAWDSKTSTLTISVNSKKTTQLVFTKQDTITVQKYDSAGTNLEGTAVEIKTLDELKNALKGGGSMQIYEGKLTAEGLRFGIVASRFNHALVDRLVEGAIDCIVRHGGREEDITLVRVPGSWEIPVAAGELARKEDIDAVIAIGVLIRGATPHFDYIASEVSKGLANLSLELRKPITFGVITADTLEQAIERAGTKHGNKGWEAALSAIEMANLFKSLR

SEQ ID NO: 17 - protein, 409 aa, synthetic construct
MDEKNSVSVDLPGGMKVLVSKEKDKDGKYSLMATVEKLELKGTSDKSNGSGVLEGEKADKSKAKLTISQDLNQTTFEIFKEDGKTLVSRKVNSKDKSSTEEKFNDKGKLSEKVVTRANGTRLEYTEIKNDGSGKAKEVLKGFALEGTLTDGGETKLTVTEGTVTLSKNISKSGEITVALNDTETTPADKKTGEWKSDTSTLTISKNSQKPKQLVFTKENTITVQNYNRAGNALEGSPAEIKDLAELKAALKGGGSMQIYEGKLTAEGLRFGIVASRFNHALVDRLVEGAIDCIVRHGGREEDITLVRVPGSWEIPVAAGELARKEDIDAVIAIGVLIRGATPHFDYIASEVSKGLANLSLELRKPITFGVITADTLEQAIERAGTKHGNKGWEAALSAIEMANLFKSLR

SEQ ID NO: 18 - protein, 408 aa, synthetic construct
MDEKNSVSVDLPGEMKVLVSKEKDKDGKYSLMATVDKLELKGTSDKSNGSGTLEGEKSDKSKAKLTISEDLSKTTFEIFKEDGKTLVSKKVNSKDKSSIEEKFNAKGELSEKTILRANGTRLEYTEIKSDGTGKAKEVLKDFALEGTLAADKTTLKVTEGTVVLSKHIPNSGEITVELNDSNSTQATKKTGKWDSNTSTLTISVNSKKTKNIVFTKEDTITVQKYDSAGTNLEGNAVEIKTLDELKNALKGGGSMQIYEGKLTAEGLRFGIVASRFNHALVDRLVEGAIDCIVRHGGREEDITLVRVPGSWEIPVAAGELARKEDIDAVIAIGVLIRGATPHFDYIASEVSKGLANLSLELRKPITFGVITADTLEQAIERAGTKHGNKGWEAALSAIEMANLFKSLR

SEQ ID NO: 19 - protein, 409 aa, synthetic construct
MDEKNSVSVDLPGGMKVLVSKEKDKDGKYSLMATVEKLELKGTSDKNNGSGTLEGEKTDKSKVKLTIAEDLSKTTFEIFKEDGKTLVSKKVTLKDKSSTEEKFNEKGEISEKTIVRANGTRLEYTDIKSDGSGKAKEVLKDFTLEGTLAADGKTTLKVTEGTVVLSKNILKSGEITVALDDSDTTQATKKTGKWDSKTSTLTISVNSQKTKNLVFTKEDTITVQKYDSAGTNLEGKAVEITTLEKLKDALKGGGSMQIYEGKLTAEGLRFGIVASRFNHALVDRLVEGAIDCIVRHGGREEDITLVRVPGSWEIPVAAGELARKEDIDAVIAIGVLIRGATPHFDYIASEVSKGLANLSLELRKPITFGVITADTLEQAIERAGTKHGNKGWEAALSAIEMANLFKSLR

SEQ ID NO: 20 - protein, 409 aa, synthetic construct
MDEKNSVSVDLPGGMTVLVSKEKDKDGKYSLEATVDKLELKGTSDKNNGSGTLEGEKTDKSKVKSTIADDLSQTKFEIFKEDGKTLVSKKVTLKDKSSTEEKFNGKGETSEKTIVRANGTRLEYTDIKSDGSGKAKEVLKDFTLEGTLAADGKTTLKVTEGTVVLSKNILKSGEITAALDDSDTTRATKKTGKWDSKTSTLTISVNSQKTKNLVFTKEDTITVQRYDSAGTNLEGKAVEITTLKELKNALKGGGSMQIYEGKLTAEGLRFGIVASRFNHALVDRLVEGAIDCIVRHGGREEDITLVRVPGSWEIPVAAGELARKEDIDAVIAIGVLIRGATPHFDYIASEVSKGLANLSLELRKPITFGVITADTLEQAIERAGTKHGNKGWEAALSAIEMANLFKSLR

SEQ ID NO: 21 - protein, 409 aa, synthetic construct
MDEKNSVSVDLPGEMKVLVSKEKDKDGKYSLEATVDKLELKGTSDKNNGSGVLEGVKAAKSKAKLTIADDLSQTKFEIFKEDGKTLVSKKVTLKDKSSTEEKFNDKGKLSEKVVTRANGTRLEYTEIQNDGSGKAKEVLKSLTLEGTLTADGETKLTVEAGTVTLSKNISESGEITVELKDTETTPADKKSGTWDSKTSTLTISKNSQKTKQLVFTKENTITVQKYNTAGTKLEGSPAEIKDLEALKAALKGGGSMQIYEGKLTAEGLRFGIVASRFNHALVDRLVEGAIDCIVRHGGREEDITLVRVPGSWEIPVAAGELARKEDIDAVIAIGVLIRGATPHFDYIASEVSKGLANLSLELRKPITFGVITADTLEQAIERAGTKHGNKGWEAALSAIEMANLFKSLR

SEQ ID NO: 22 - protein, 435 aa, synthetic construct
MDEKNSVSVDLPGEMKVLVSKEKNKDGKYDLIATVDKLELKGTSDKNNGSGVLEGVKADKSKVKLTISDDLGQTTLEVFKEDGKTLVSKKVTSKDKSSTEEKFNEKGEVSEKIITRADGTRLEYTGIKSDGSGKAKEVLKGYTLEGQLSDEKTTLVVKEGTVTLSKNISKSGEVSVELNDTDSSAATKKTAAWNSGTSTLTITVNSKKTKDLVFTKENTITVQQYDSNGTKLEGSAVEITKLDEIKNALKGSMTTASTSQVRQNYHQDSEAAINRQINLELYASYVYLSMSYYFDRDDVALKNFAKYFLHQSHEEREHAEKLMKLQNQRGGRIFLQDIKKPDCDDWESGLNAMECALHLEKNVNQSLLELHKLATDKNDPHLCDFIETHYLNEQVKAIKELGDHVTNLRKMGAPESGLAEYLFDKHTLGDSDNES

SEQ ID NO: 23 - protein, 435 aa, synthetic construct
MDEKNSVSVDLPGEMKVLVSKEKNKDGKYDLIATVDKLELKGTSDKNNGSGVLEGVKADKSKVKLTISDDLGQTTLEVFKEDGKTLVSKKVTSKDKSSTEEKFNEKGEVSEKIITRADGTRLEYTGIKSDGSGKAKEVLKGYDLKGELSSEKTTLVVKEGTVTLSKNISKSGEVSVELNDTDSSAATKKTAAWNSGTSTLTITVNSKKTKDLVFTKENTITVQQYDSNGTKLEGSAVEITKLDEIKNALKGSMTTASTSQVRQNYHQDSEAAINRQINLELYASYVYLSMSYYFDRDDVALKNFAKYFLHQSHEEREHAEKLMKLQNQRGGRIFLQDIKKPDCDDWESGLNAMECALHLEKNVNQSLLELHKLATDKNDPHLCDFIETHYLNEQVKAIKELGDHVTNLRKMGAPESGLAEYLFDKHTLGDSDNES

SEQ ID NO: 24 - protein, 435 aa, synthetic construct
MDEKNSVSVDLPGEMKVLVSKEKNKDGKYDLIATVDKLELKGTSDKNNGSGVLEGVKADKSKVKLTISDDLGQTTLEVFKEDGKTLVSKKVTSKDKSSTEEKFNEKGEVSEKIITRADGTRLEYTGIKSDGSGKAKEVLKGFTLEGKVANEKTTLVVKEGTVTLSKNISKSGEVSVELNDTDSSAATKKTAAWNSGTSTLTITVNSKKTKDLVFTKENTITVQQYDSNGTKLEGSAVEITKLDEIKNALKGSMTTASTSQVRQNYHQDSEAAINRQINLELYASYVYLSMSYYFDRDDVALKNFAKYFLHQSHEEREHAEKLMKLQNQRGGRIFLQDIKKPDCDDWESGLNAMECALHLEKNVNQSLLELHKLATDKNDPHLCDFIETHYLNEQVKAIKELGDHVTNLRKMGAPESGLAEYLFDKHTLGDSDNES

SEQ ID NO: 25 - protein, 435 aa, synthetic construct
MDEKNSVSVDLPGEMKVLVSKEKNKDGKYDLIATVDKLELKGTSDKNNGSGVLEGVKADKSKVKLTISDDLGQTTLEVFKEDGKTLVSKKVTSKDKSSTEEKFNEKGEVSEKIITRADGTRLEYTGIKSDGSGKAKEVLKGFALEGTLTDEKTTLVVKEGTVTLSKNISKSGEVSVELNDTDSSAATKKTAAWNSGTSTLTITVNSKKTKDLVFTKENTITVQQYDSNGTKLEGSAVEITKLDEIKNALKGSMTTASTSQVRQNYHQDSEAAINRQINLELYASYVYLSMSYYFDRDDVALKNFAKYFLHQSHEEREHAEKLMKLQNQRGGRIFLQDIKKPDCDDWESGLNAMECALHLEKNVNQSLLELHKLATDKNDPHLCDFIETHYLNEQVKAIKELGDHVTNLRKMGAPESGLAEYLFDKHTLGDSDNES

SEQ ID NO: 26 - protein, 435 aa, synthetic construct
MDEKNSASVDLPGEMKVLVSKEKDKDGKYSLKATVDKIELKGTSDKDNGSGVLEGTKDDKSKAKLTIADDLSKTTFELFKEDGKTLVSRKVSSKDKTSTDEMFNEKGELSAKTMTRENGTKLEYTEMKSDGTGKAKEVLKNFTLEGKVANDKVTLEVKEGTVTLSKEIAKSGEVTVALNDTNTTQATKKTGAWDSKTSTLTISVNSKKTTQLVFTKQDTITVQKYDSAGTNLEGTAVEIKTLDELKNALKGSMTTASTSQVRQNYHQDSEAAINRQINLELYASYVYLSMSYYFDRDDVALKNFAKYFLHQSHEEREHAEKLMKLQNQRGGRIFLQDIKKPDCDDWESGLNAMECALHLEKNVNQSLLELHKLATDKNDPHLCDFIETHYLNEQVKAIKELGDHVTNLRKMGAPESGLAEYLFDKHTLGDSDNES

SEQ ID NO: 27 - protein, 436 aa, synthetic construct
MDEKNSVSVDLPGGMKVLVSKEKDKDGKYSLMATVEKLELKGTSDKSNGSGVLEGEKADKSKAKLTISQDLNQTTFEIFKEDGKTLVSRKVNSKDKSSTEEKFNDKGKLSEKVVTRANGTRLEYTEIKNDGSGKAKEVLKGFALEGTLTDGGETKLTVTEGTVTLSKNISKSGEITVALNDTETTPADKKTGEWKSDTSTLTISKNSQKPKQLVFTKENTITVQNYNRAGNALEGSPAEIKDLAELKAALKGSMTTASTSQVRQNYHQDSEAAINRQINLELYASYVYLSMSYYFDRDDVALKNFAKYFLHQSHEEREHAEKLMKLQNQRGGRIFLQDIKKPDCDDWESGLNAMECALHLEKNVNQSLLELHKLATDKNDPHLCDFIETHYLNEQVKAIKELGDHVTNLRKMGAPESGLAEYLFDKHTLGDSDNES

SEQ ID NO: 28 - protein, 435 aa, synthetic construct
MDEKNSVSVDLPGEMKVLVSKEKDKDGKYSLMATVDKLELKGTSDKSNGSGTLEGEKSDKSKAKLTISEDLSKTTFEIFKEDGKTLVSKKVNSKDKSSIEEKFNAKGELSEKTILRANGTRLEYTEIKSDGTGKAKEVLKDFALEGTLAADKTTLKVTEGTVVLSKHIPNSGEITVELNDSNSTQATKKTGKWDSNTSTLTISVNSKKTKNIVFTKEDTITVQKYDSAGTNLEGNAVEIKTLDELKNALKGSMTTASTSQVRQNYHQDSEAAINRQINLELYASYVYLSMSYYFDRDDVALKNFAKYFLHQSHEEREHAEKLMKLQNQRGGRIFLQDIKKPDCDDWESGLNAMECALHLEKNVNQSLLELHKLATDKNDPHLCDFIETHYLNEQVKAIKELGDHVTNLRKMGAPESGLAEYLFDKHTLGDSDNES

SEQ ID NO: 29 - protein, 436 aa, synthetic construct
MDEKNSVSVDLPGGMKVLVSKEKDKDGKYSLMATVEKLELKGTSDKNNGSGTLEGEKTDKSKVKLTIAEDLSKTTFEIFKEDGKTLVSKKVTLKDKSSTEEKFNEKGEISEKTIVRANGTRLEYTDIKSDGSGKAKEVLKDFTLEGTLAADGKTTLKVTEGTVVLSKNILKSGEITVALDDSDTTQATKKTGKWDSKTSTLTISVNSQKTKNLVFTKEDTITVQKYDSAGTNLEGKAVEITTLEKLKDALKGSMTTASTSQVRQNYHQDSEAAINRQINLELYASYVYLSMSYYFDRDDVALKNFAKYFLHQSHEEREHAEKLMKLQNQRGGRIFLQDIKKPDCDDWESGLNAMECALHLEKNVNQSLLELHKLATDKNDPHLCDFIETHYLNEQVKAIKELGDHVTNLRKMGAPESGLAEYLFDKHTLGDSDNES

SEQ ID NO: 30 - protein, 436 aa, synthetic construct
MDEKNSVSVDLPGGMTVLVSKEKDKDGKYSLEATVDKLELKGTSDKNNGSGTLEGEKTDKSKVKSTIADDLSQTKFEIFKEDGKTLVSKKVTLKDKSSTEEKFNGKGETSEKTIVRANGTRLEYTDIKSDGSGKAKEVLKDFTLEGTLAADGKTTLKVTEGTVVLSKNILKSGEITAALDDSDTTRATKKTGKWDSKTSTLTISVNSQKTKNLVFTKEDTITVQRYDSAGTNLEGKAVEITTLKELKNALKGSMTTASTSQVRQNYHQDSEAAINRQINLELYASYVYLSMSYYFDRDDVALKNFAKYFLHQSHEEREHAEKLMKLQNQRGGRIFLQDIKKPDCDDWESGLNAMECALHLEKNVNQSLLELHKLATDKNDPHLCDFIETHYLNEQVKAIKELGDHVTNLRKMGAPESGLAEYLFDKHTLGDSDNES

SEQ ID NO: 31 - protein, 436 aa, synthetic construct
MDEKNSVSVDLPGEMKVLVSKEKDKDGKYSLEATVDKLELKGTSDKNNGSGVLEGVKAAKSKAKLTIADDLSQTKFEIFKEDGKTLVSKKVTLKDKSSTEEKFNDKGKLSEKVVTRANGTRLEYTEIQNDGSGKAKEVLKSLTLEGTLTADGETKLTVEAGTVTLSKNISESGEITVELKDTETTPADKKSGTWDSKTSTLTISKNSQKTKQLVFTKENTITVQKYNTAGTKLEGSPAEIKDLEALKAALKGSMTTASTSQVRQNYHQDSEAAINRQINLELYASYVYLSMSYYFDRDDVALKNFAKYFLHQSHEEREHAEKLMKLQNQRGGRIFLQDIKKPDCDDWESGLNAMECALHLEKNVNQSLLELHKLATDKNDPHLCDFIETHYLNEQVKAIKELGDHVTNLRKMGAPESGLAEYLFDKHTLGDSDNES

SEQ ID NO: 32 - protein, 426 aa, synthetic construct
MDEKNSVSVDLPGEMKVLVSKEKNKDGKYDLIATVDKLELKGTSDKNNGSGVLEGVKADKSKVKLTISDDLGQTTLEVFKEDGKTLVSKKVTSKDKSSTEEKFNEKGEVSEKIITRADGTRLEYTGIKSDGSGKAKEVLKGYTLEGQLSDEKTTLVVKEGTVTLSKNISKSGEVSVELNDTDSSAATKKTAAWNSGTSTLTITVNSKKTKDLVFTKENTITVQQYDSNGTKLEGSAVEITKLDEIKNALKGSMLSERMLKALNDQLNRELYSAYLYFAMAAYFEDLGLEGFANWMKAQAEEEIGHALRFYNYIYDRNGRVELDEIPKPPKEWESPLKAFEAAYEHEKFISKSIYELAALAEEEKDYSTRAFLEWFINEQVEEEASVKKILDKLKFAKDSPQILFMLDKELSARAPKLPGLLMQGGE

SEQ ID NO: 33 - protein, 426 aa, synthetic construct
MDEKNSVSVDLPGEMKVLVSKEKNKDGKYDLIATVDKLELKGTSDKNNGSGVLEGVKADKSKVKLTISDDLGQTTLEVFKEDGKTLVSKKVTSKDKSSTEEKFNEKGEVSEKIITRADGTRLEYTGIKSDGSGKAKEVLKGYDLKGELSSEKTTLVVKEGTVTLSKNISKSGEVSVELNDTDSSAATKKTAAWNSGTSTLTITVNSKKTKDLVFTKENTITVQQYDSNGTKLEGSAVEITKLDEIKNALKGSMLSERMLKALNDQLNRELYSAYLYFAMAAYFEDLGLEGFANWMKAQAEEEIGHALRFYNYIYDRNGRVELDEIPKPPKEWESPLKAFEAAYEHEKFISKSIYELAALAEEEKDYSTRAFLEWFINEQVEEEASVKKILDKLKFAKDSPQILFMLDKELSARAPKLPGLLMQGGE

SEQ ID NO: 34 - protein, 426 aa, synthetic construct
MDEKNSVSVDLPGEMKVLVSKEKNKDGKYDLIATVDKLELKGTSDKNNGSGVLEGVKADKSKVKLTISDDLGQTTLEVFKEDGKTLVSKKVTSKDKSSTEEKFNEKGEVSEKIITRADGTRLEYTGIKSDGSGKAKEVLKGFTLEGKVANEKTTLVVKEGTVTLSKNISKSGEVSVELNDTDSSAATKKTAAWNSGTSTLTITVNSKKTKDLVFTKENTITVQQYDSNGTKLEGSAVEITKLDEIKNALKGSMLSERMLKALNDQLNRELYSAYLYFAMAAYFEDLGLEGFANWMKAQAEEEIGHALRFYNYIYDRNGRVELDEIPKPPKEWESPLKAFEAAYEHEKFISKSIYELAALAEEEKDYSTRAFLEWFINEQVEEEASVKKILDKLKFAKDSPQILFMLDKELSARAPKLPGLLMQGGE

SEQ ID NO: 35 - protein, 426 aa, synthetic construct
MDEKNSVSVDLPGEMKVLVSKEKNKDGKYDLIATVDKLELKGTSDKNNGSGVLEGVKADKSKVKLTISDDLGQTTLEVFKEDGKTLVSKKVTSKDKSSTEEKFNEKGEVSEKIITRADGTRLEYTGIKSDGSGKAKEVLKGFALEGTLTDEKTTLVVKEGTVTLSKNISKSGEVSVELNDTDSSAATKKTAAWNSGTSTLTITVNSKKTKDLVFTKENTITVQQYDSNGTKLEGSAVEITKLDEIKNALKGSMLSERMLKALNDQLNRELYSAYLYFAMAAYFEDLGLEGFANWMKAQAEEEIGHALRFYNYIYDRNGRVELDEIPKPPKEWESPLKAFEAAYEHEKFISKSIYELAALAEEEKDYSTRAFLEWFINEQVEEEASVKKILDKLKFAKDSPQILFMLDKELSARAPKLPGLLMQGGE

SEQ ID NO: 36 - protein, 426 aa, synthetic construct
MDEKNSASVDLPGEMKVLVSKEKDKDGKYSLKATVDKIELKGTSDKDNGSGVLEGTKDDKSKAKLTIADDLSKTTFELFKEDGKTLVSRKVSSKDKTSTDEMFNEKGELSAKTMTRENGTKLEYTEMKSDGTGKAKEVLKNFTLEGKVANDKVTLEVKEGTVTLSKEIAKSGEVTVALNDTNTTQATKKTGAWDSKTSTLTISVNSKKTTQLVFTKQDTITVQKYDSAGTNLEGTAVEIKTLDELKNALKGSMLSERMLKALNDQLNRELYSAYLYFAMAAYFEDLGLEGFANWMKAQAEEEIGHALRFYNYIYDRNGRVELDEIPKPPKEWESPLKAFEAAYEHEKFISKSIYELAALAEEEKDYSTRAFLEWFINEQVEEEASVKKILDKLKFAKDSPQILFMLDKELSARAPKLPGLLMQGGE

SEQ ID NO: 37 - protein, 427 aa, synthetic construct
MDEKNSVSVDLPGGMKVLVSKEKDKDGKYSLMATVEKLELKGTSDKSNGSGVLEGEKADKSKAKLTISQDLNQTTFEIFKEDGKTLVSRKVNSKDKSSTEEKFNDKGKLSEKVVTRANGTRLEYTEIKNDGSGKAKEVLKGFALEGTLTDGGETKLTVTEGTVTLSKNISKSGEITVALNDTETTPADKKTGEWKSDTSTLTISKNSQKPKQLVFTKENTITVQNYNRAGNALEGSPAEIKDLAELKAALKGSMLSERMLKALNDQLNRELYSAYLYFAMAAYFEDLGLEGFANWMKAQAEEEIGHALRFYNYIYDRNGRVELDEIPKPPKEWESPLKAFEAAYEHEKFISKSIYELAALAEEEKDYSTRAFLEWFINEQVEEEASVKKILDKLKFAKDSPQILFMLDKELSARAPKLPGLLMQGGE

SEQ ID NO: 38 - protein, 426 aa, synthetic construct
MDEKNSVSVDLPGEMKVLVSKEKDKDGKYSLMATVDKLELKGTSDKSNGSGTLEGEKSDKSKAKLTISEDLSKTTFEIFKEDGKTLVSKKVNSKDKSSIEEKFNAKGELSEKTILRANGTRLEYTEIKSDGTGKAKEVLKDFALEGTLAADKTTLKVTEGTVVLSKHIPNSGEITVELNDSNSTQATKKTGKWDSNTSTLTISVNSKKTKNIVFTKEDTITVQKYDSAGTNLEGNAVEIKTLDELKNALKGSMLSERMLKALNDQLNRELYSAYLYFAMAAYFEDLGLEGFANWMKAQAEEEIGHALRFYNYIYDRNGRVELDEIPKPPKEWESPLKAFEAAYEHEKFISKSIYELAALAEEEKDYSTRAFLEWFINEQVEEEASVKKILDKLKFAKDSPQILFMLDKELSARAPKLPGLLMQGGE

SEQ ID NO: 39 - protein, 427 aa, synthetic construct
MDEKNSVSVDLPGGMKVLVSKEKDKDGKYSLMATVEKLELKGTSDKNNGSGTLEGEKTDKSKVKLTIAEDLSKTTFEIFKEDGKTLVSKKVTLKDKSSTEEKFNEKGEISEKTIVRANGTRLEYTDIKSDGSGKAKEVLKDFTLEGTLAADGKTTLKVTEGTVVLSKNILKSGEITVALDDSDTTQATKKTGKWDSKTSTLTISVNSQKTKNLVFTKEDTITVQKYDSAGTNLEGKAVEITTLEKLKDALKGSMLSERMLKALNDQLNRELYSAYLYFAMAAYFEDLGLEGFANWMKAQAEEEIGHALRFYNYIYDRNGRVELDEIPKPPKEWESPLKAFEAAYEHEKFISKSIYELAALAEEEKDYSTRAFLEWFINEQVEEEASVKKILDKLKFAKDSPQILFMLDKELSARAPKLPGLLMQGGE

SEQ ID NO: 40 - protein, 427 aa, synthetic construct
MDEKNSVSVDLPGGMTVLVSKEKDKDGKYSLEATVDKLELKGTSDKNNGSGTLEGEKTDKSKVKSTIADDLSQTKFEIFKEDGKTLVSKKVTLKDKSSTEEKFNGKGETSEKTIVRANGTRLEYTDIKSDGSGKAKEVLKDFTLEGTLAADGKTTLKVTEGTVVLSKNILKSGEITAALDDSDTTRATKKTGKWDSKTSTLTISVNSQKTKNLVFTKEDTITVQRYDSAGTNLEGKAVEITTLKELKNALKGSMLSERMLKALNDQLNRELYSAYLYFAMAAYFEDLGLEGFANWMKAQAEEEIGHALRFYNYIYDRNGRVELDEIPKPPKEWESPLKAFEAAYEHEKFISKSIYELAALAEEEKDYSTRAFLEWFINEQVEEEASVKKILDKLKFAKDSPQILFMLDKELSARAPKLPGLLMQGGE

SEQ ID NO: 41 - protein, 427 aa, synthetic construct
MDEKNSVSVDLPGEMKVLVSKEKDKDGKYSLEATVDKLELKGTSDKNNGSGVLEGVKAAKSKAKLTIADDLSQTKFEIFKEDGKTLVSKKVTLKDKSSTEEKFNDKGKLSEKVVTRANGTRLEYTEIQNDGSGKAKEVLKSLTLEGTLTADGETKLTVEAGTVTLSKNISESGEITVELKDTETTPADKKSGTWDSKTSTLTISKNSQKTKQLVFTKENTITVQKYNTAGTKLEGSPAEIKDLEALKAALKGSMLSERMLKALNDQLNRELYSAYLYFAMAAYFEDLGLEGFANWMKAQAEEEIGHALRFYNYIYDRNGRVELDEIPKPPKEWESPLKAFEAAYEHEKFISKSIYELAALAEEEKDYSTRAFLEWFINEQVEEEASVKKILDKLKFAKDSPQILFMLDKELSARAPKLPGLLMQGGE

SEQ ID NO: 42 - protein, 443 aa, synthetic construct
MDEKNSVSVDLPGEMKVLVSKEKNKDGKYDLIATVDKLELKGTSDKNNGSGVLEGVKADKSKVKLTISDDLGQTTLEVFKEDGKTLVSKKVTSKDKSSTEEKFNEKGEVSEKIITRADGTRLEYTGIKSDGSGKAKEVLKGYTLEGQLSDEKTTLVVKEGTVTLSKNISKSGEVSVELNDTDSSAATKKTAAWNSGTSTLTITVNSKKTKDLVFTKENTITVQQYDSNGTKLEGSAVEITKLDEIKNALKGSTQCNVNPVQIPKDWITMHRSCRNSMRQQIQMEVGASLQYLAMGAHFSKDVVNRPGFAQLFFDAASEEREHAMKLIEYLLMRGELTNDVSSLLQVRPPTRSSWKGGVEALEHALSMESDVTKSIRNVIKACEDDSEFNDYHLVDYLTGDFLEEQYKGQRDLAGKASTLKKLMDRHEALGEFIFDKKLLGIDV

SEQ ID NO: 43 - protein, 443 aa, synthetic construct
MDEKNSVSVDLPGEMKVLVSKEKNKDGKYDLIATVDKLELKGTSDKNNGSGVLEGVKADKSKVKLTISDDLGQTTLEVFKEDGKTLVSKKVTSKDKSSTEEKFNEKGEVSEKIITRADGTRLEYTGIKSDGSGKAKEVLKGYDLKGELSSEKTTLVVKEGTVTLSKNISKSGEVSVELNDTDSSAATKKTAAWNSGTSTLTITVNSKKTKDLVFTKENTITVQQYDSNGTKLEGSAVEITKLDEIKNALKGSTQCNVNPVQIPKDWITMHRSCRNSMRQQIQMEVGASLQYLAMGAHFSKDVVNRPGFAQLFFDAASEEREHAMKLIEYLLMRGELTNDVSSLLQVRPPTRSSWKGGVEALEHALSMESDVTKSIRNVIKACEDDSEFNDYHLVDYLTGDFLEEQYKGQRDLAGKASTLKKLMDRHEALGEFIFDKKLLGIDV

SEQ ID NO: 44 - protein, 443 aa, synthetic construct
MDEKNSVSVDLPGEMKVLVSKEKNKDGKYDLIATVDKLELKGTSDKNNGSGVLEGVKADKSKVKLTISDDLGQTTLEVFKEDGKTLVSKKVTSKDKSSTEEKFNEKGEVSEKIITRADGTRLEYTGIKSDGSGKAKEVLKGFTLEGKVANEKTTLVVKEGTVTLSKNISKSGEVSVELNDTDSSAATKKTAAWNSGTSTLTITVNSKKTKDLVFTKENTITVQQYDSNGTKLEGSAVEITKLDEIKNALKGSTQCNVNPVQIPKDWITMHRSCRNSMRQQIQMEVGASLQYLAMGAHFSKDVVNRPGFAQLFFDAASEEREHAMKLIEYLLMRGELTNDVSSLLQVRPPTRSSWKGGVEALEHALSMESDVTKSIRNVIKACEDDSEFNDYHLVDYLTGDFLEEQYKGQRDLAGKASTLKKLMDRHEALGEFIFDKKLLGIDV

SEQ ID NO: 45 - protein, 443 aa, synthetic construct
MDEKNSVSVDLPGEMKVLVSKEKNKDGKYDLIATVDKLELKGTSDKNNGSGVLEGVKADKSKVKLTISDDLGQTTLEVFKEDGKTLVSKKVTSKDKSSTEEKFNEKGEVSEKIITRADGTRLEYTGIKSDGSGKAKEVLKGFALEGTLTDEKTTLVVKEGTVTLSKNISKSGEVSVELNDTDSSAATKKTAAWNSGTSTLTITVNSKKTKDLVFTKENTITVQQYDSNGTKLEGSAVEITKLDEIKNALKGSTQCNVNPVQIPKDWITMHRSCRNSMRQQIQMEVGASLQYLAMGAHFSKDVVNRPGFAQLFFDAASEEREHAMKLIEYLLMRGELTNDVSSLLQVRPPTRSSWKGGVEALEHALSMESDVTKSIRNVIKACEDDSEFNDYHLVDYLTGDFLEEQYKGQRDLAGKASTLKKLMDRHEALGEFIFDKKLLGIDV

SEQ ID NO: 46 - protein, 443 aa, synthetic construct
MDEKNSASVDLPGEMKVLVSKEKDKDGKYSLKATVDKIELKGTSDKDNGSGVLEGTKDDKSKAKLTIADDLSKTTFELFKEDGKTLVSRKVSSKDKTSTDEMFNEKGELSAKTMTRENGTKLEYTEMKSDGTGKAKEVLKNFTLEGKVANDKVTLEVKEGTVTLSKEIAKSGEVTVALNDTNTTQATKKTGAWDSKTSTLTISVNSKKTTQLVFTKQDTITVQKYDSAGTNLEGTAVEIKTLDELKNALKGSTQCNVNPVQIPKDWITMHRSCRNSMRQQIQMEVGASLQYLAMGAHFSKDVVNRPGFAQLFFDAASEEREHAMKLIEYLLMRGELTNDVSSLLQVRPPTRSSWKGGVEALEHALSMESDVTKSIRNVIKACEDDSEFNDYHLVDYLTGDFLEEQYKGQRDLAGKASTLKKLMDRHEALGEFIFDKKLLGIDV

SEQ ID NO: 47 - protein, 444 aa, synthetic construct
MDEKNSVSVDLPGGMKVLVSKEKDKDGKYSLMATVEKLELKGTSDKSNGSGVLEGEKADKSKAKLTISQDLNQTTFEIFKEDGKTLVSRKVNSKDKSSTEEKFNDKGKLSEKVVTRANGTRLEYTEIKNDGSGKAKEVLKGFALEGTLTDGGETKLTVTEGTVTLSKNISKSGEITVALNDTETTPADKKTGEWKSDTSTLTISKNSQKPKQLVFTKENTITVQNYNRAGNALEGSPAEIKDLAELKAALKGSTQCNVNPVQIPKDWITMHRSCRNSMRQQIQMEVGASLQYLAMGAHFSKDVVNRPGFAQLFFDAASEEREHAMKLIEYLLMRGELTNDVSSLLQVRPPTRSSWKGGVEALEHALSMESDVTKSIRNVIKACEDDSEFNDYHLVDYLTGDFLEEQYKGQRDLAGKASTLKKLMDRHEALGEFIFDKKLLGIDV

SEQ ID NO: 48 - protein, 443 aa, synthetic construct
MDEKNSVSVDLPGEMKVLVSKEKDKDGKYSLMATVDKLELKGTSDKSNGSGTLEGEKSDKSKAKLTISEDLSKTTFEIFKEDGKTLVSKKVNSKDKSSIEEKFNAKGELSEKTILRANGTRLEYTEIKSDGTGKAKEVLKDFALEGTLAADKTTLKVTEGTVVLSKHIPNSGEITVELNDSNSTQATKKTGKWDSNTSTLTISVNSKKTKNIVFTKEDTITVQKYDSAGTNLEGNAVEIKTLDELKNALKGSTQCNVNPVQIPKDWITMHRSCRNSMRQQIQMEVGASLQYLAMGAHFSKDVVNRPGFAQLFFDAASEEREHAMKLIEYLLMRGELTNDVSSLLQVRPPTRSSWKGGVEALEHALSMESDVTKSIRNVIKACEDDSEFNDYHLVDYLTGDFLEEQYKGQRDLAGKASTLKKLMDRHEALGEFIFDKKLLGIDV

SEQ ID NO: 49 - protein, 444 aa, synthetic construct
MDEKNSVSVDLPGGMKVLVSKEKDKDGKYSLMATVEKLELKGTSDKNNGSGTLEGEKTDKSKVKLTIAEDLSKTTFEIFKEDGKTLVSKKVTLKDKSSTEEKFNEKGEISEKTIVRANGTRLEYTDIKSDGSGKAKEVLKDFTLEGTLAADGKTTLKVTEGTVVLSKNILKSGEITVALDDSDTTQATKKTGKWDSKTSTLTISVNSQKTKNLVFTKEDTITVQKYDSAGTNLEGKAVEITTLEKLKDALKGSTQCNVNPVQIPKDWITMHRSCRNSMRQQIQMEVGASLQYLAMGAHFSKDVVNRPGFAQLFFDAASEEREHAMKLIEYLLMRGELTNDVSSLLQVRPPTRSSWKGGVEALEHALSMESDVTKSIRNVIKACEDDSEFNDYHLVDYLTGDFLEEQYKGQRDLAGKASTLKKLMDRHEALGEFIFDKKLLGIDV

SEQ ID NO: 50 - protein, 444 aa, synthetic construct
MDEKNSVSVDLPGGMTVLVSKEKDKDGKYSLEATVDKLELKGTSDKNNGSGTLEGEKTDKSKVKSTIADDLSQTKFEIFKEDGKTLVSKKVTLKDKSSTEEKFNGKGETSEKTIVRANGTRLEYTDIKSDGSGKAKEVLKDFTLEGTLAADGKTTLKVTEGTVVLSKNILKSGEITAALDDSDTTRATKKTGKWDSKTSTLTISVNSQKTKNLVFTKEDTITVQRYDSAGTNLEGKAVEITTLKELKNALKGSTQCNVNPVQIPKDWITMHRSCRNSMRQQIQMEVGASLQYLAMGAHFSKDVVNRPGFAQLFFDAASEEREHAMKLIEYLLMRGELTNDVSSLLQVRPPTRSSWKGGVEALEHALSMESDVTKSIRNVIKACEDDSEFNDYHLVDYLTGDFLEEQYKGQRDLAGKASTLKKLMDRHEALGEFIFDKKLLGIDV

SEQ ID NO: 51 - protein, 444 aa, synthetic construct
MDEKNSVSVDLPGEMKVLVSKEKDKDGKYSLEATVDKLELKGTSDKNNGSGVLEGVKAAKSKAKLTIADDLSQTKFEIFKEDGKTLVSKKVTLKDKSSTEEKFNDKGKLSEKVVTRANGTRLEYTEIQNDGSGKAKEVLKSLTLEGTLTADGETKLTVEAGTVTLSKNISESGEITVELKDTETTPADKKSGTWDSKTSTLTISKNSQKTKQLVFTKENTITVQKYNTAGTKLEGSPAEIKDLEALKAALKGSTQCNVNPVQIPKDWITMHRSCRNSMRQQIQMEVGASLQYLAMGAHFSKDVVNRPGFAQLFFDAASEEREHAMKLIEYLLMRGELTNDVSSLLQVRPPTRSSWKGGVEALEHALSMESDVTKSIRNVIKACEDDSEFNDYHLVDYLTGDFLEEQYKGQRDLAGKASTLKKLMDRHEALGEFIFDKKLLGIDV

SEQ ID NO: 52 - protein, 455 aa, synthetic construct
MDSKGSSQKGSRLLLLLVVSNLLLPQGVLAMDEKNSVSVDLPGEMKVLVSKEKNKDGKYDLIATVDKLELKGTSDKNNGSGVLEGVKADKSKVKLTISDDLGQTTLEVFKEDGKTLVSKKVTSKDKSSTEEKFNEKGEVSEKIITRADGTRLEYTGIKSDGSGKAKEVLKGYTLEGQLSDEKTTLVVKEGTVTLSKNISKSGEVSVELNDTDSSAATKKTAAWNSGTSTLTITVNSKKTKDLVFTKENTITVQQYDSNGTKLEGSAVEITKLDEIKNALKGSESQVRQQFSKDIEKLLNEQVNKEMQSSNLYMSMSSWSYTHSLDGAGLFLFDHAAEEYEHAKKLIIFLNENNVPVQLTSISAPEHKFEGLTQIFQKAYEHEQHISESINNIVDHAIKCKDHATFNFLQWYVAEQHEEEVLFKDILDKIELIGNENHGLYLADQYVKGIAKSRKS

SEQ ID NO: 53 - protein, 455 aa, synthetic construct
MDSKGSSQKGSRLLLLLVVSNLLLPQGVLAMDEKNSVSVDLPGEMKVLVSKEKNKDGKYDLIATVDKLELKGTSDKNQGSGVLEGVKADKSKVKLTISDDLGQTTLEVFKEDGKTLVSKKVTSKDKSSTEEKFNEKGEVSEKIITRADGTRLEYTGIKSDGSGKAKEVLKGYTLEGQLSDEKTTLVVKEGTVTLSKQISKSGEVSVELQDTDSSAATKKTAAWNSGTSTLTITVNSKKTKDLVFTKENTITVQQYDSQGTKLEGSAVEITKLDEIKNALKGSESQVRQQFSKDIEKLLNEQVNKEMQSSNLYMSMSSWSYTHSLDGAGLFLFDHAAEEYEHAKKLIIFLNENNVPVQLTSISAPEHKFEGLTQIFQKAYEHEQHISESINNIVDHAIKCKDHATFNFLQWYVAEQHEEEVLFKDILDKIELIGNENHGLYLADQYVKGIAKSRKS

SEQ ID NO: 54 - protein, 455 aa, synthetic construct
MDSKGSSQKGSRLLLLLVVSNLLLPQGVLAMDEKNSVSVDLPGEMKVLVSKEKNKDGKYDLIATVDKLELKGTSDKNNGSGVLEGVKADKSKVKLTISDDLGQTTLEVFKEDGKTLVSKKVTSKDKSSTEEKFNEKGEVSEKIITRADGTRLEYTGIKSDGSGKAKEVLKGFTLEGKVANEKTTLVVKEGTVTLSKNISKSGEVSVELNDTDSSAATKKTAAWNSGTSTLTITVNSKKTKDLVFTKENTITVQQYDSNGTKLEGSAVEITKLDEIKNALKGSESQVRQQFSKDIEKLLNEQVNKEMQSSNLYMSMSSWSYTHSLDGAGLFLFDHAAEEYEHAKKLIIFLNENNVPVQLTSISAPEHKFEGLTQIFQKAYEHEQHISESINNIVDHAIKCKDHATFNFLQWYVAEQHEEEVLFKDILDKIELIGNENHGLYLADQYVKGIAKSRKS

SEQ ID NO: 55 - protein, 455 aa, synthetic construct
MDSKGSSQKGSRLLLLLVVSNLLLPQGVLAMDEKNSVSVDLPGEMKVLVSKEKNKDGKYDLIATVDKLELKGTSDKNQGSGVLEGVKADKSKVKLTISDDLGQTTLEVFKEDGKTLVSKKVTSKDKSSTEEKFNEKGEVSEKIITRADGTRLEYTGIKSDGSGKAKEVLKGFTLEGKVANEKTTLVVKEGTVTLSKQISKSGEVSVELQDTDSSAATKKTAAWNSGTSTLTITVNSKKTKDLVFTKENTITVQQYDSQGTKLEGSAVEITKLDEIKNALKGSESQVRQQFSKDIEKLLNEQVNKEMQSSNLYMSMSSWSYTHSLDGAGLFLFDHAAEEYEHAKKLIIFLNENNVPVQLTSISAPEHKFEGLTQIFQKAYEHEQHISESINNIVDHAIKCKDHATFNFLQWYVAEQHEEEVLFKDILDKIELIGNENHGLYLADQYVKGIAKSRKS

SEQ ID NO: 56 - protein, 455 aa, synthetic construct
MDSKGSSQKGSRLLLLLVVSNLLLPQGVLAMDEKNSVSVDLPGEMKVLVSKEKNKDGKYDLIATVDKLELKGTSDKNNGSGVLEGVKADKSKVKLTISDDLGQTTLEVFKEDGKTLVSKKVTSKDKSSTEEKFNEKGEVSEKIITRADGTRLEYTGIKSDGSGKAKEVLKGFALEGTLTDEKTTLVVKEGTVTLSKNISKSGEVSVELNDTDSSAATKKTAAWNSGTSTLTITVNSKKTKDLVFTKENTITVQQYDSNGTKLEGSAVEITKLDEIKNALKGSESQVRQQFSKDIEKLLNEQVNKEMQSSNLYMSMSSWSYTHSLDGAGLFLFDHAAEEYEHAKKLIIFLNENNVPVQLTSISAPEHKFEGLTQIFQKAYEHEQHISESINNIVDHAIKCKDHATFNFLQWYVAEQHEEEVLFKDILDKIELIGNENHGLYLADQYVKGIAKSRKS

SEQ ID NO: 57 - protein, 455 aa, synthetic construct
MDSKGSSQKGSRLLLLLVVSNLLLPQGVLAMDEKNSVSVDLPGEMKVLVSKEKNKDGKYDLIATVDKLELKGTSDKNQGSGVLEGVKADKSKVKLTISDDLGQTTLEVFKEDGKTLVSKKVTSKDKSSTEEKFNEKGEVSEKIITRADGTRLEYTGIKSDGSGKAKEVLKGFALEGTLTDEKTTLVVKEGTVTLSKQISKSGEVSVELQDTDSSAATKKTAAWNSGTSTLTITVNSKKTKDLVFTKENTITVQQYDSQGTKLEGSAVEITKLDEIKNALKGSESQVRQQFSKDIEKLLNEQVNKEMQSSNLYMSMSSWSYTHSLDGAGLFLFDHAAEEYEHAKKLIIFLNENNVPVQLTSISAPEHKFEGLTQIFQKAYEHEQHISESINNIVDHAIKCKDHATFNFLQWYVAEQHEEEVLFKDILDKIELIGNENHGLYLADQYVKGIAKSRKS

SEQ ID NO: 58 - protein, 455 aa, synthetic construct
MDSKGSSQKGSRLLLLLVVSNLLLPQGVLAMDEKNSVSVDLPGEMKVLVSKEKNKDGKYDLIATVDKLELKGTSDKNNGSGVLEGVKADKSKVKLTISDDLGQTTLEVFKEDGKTLVSKKVTSKDKSSTEEKFNEKGEVSEKIITRADGTRLEYTGIKSDGSGKAKEVLKGYDLKGELSSEKTTLVVKEGTVTLSKNISKSGEVSVELNDTDSSAATKKTAAWNSGTSTLTITVNSKKTKDLVFTKENTITVQQYDSNGTKLEGSAVEITKLDEIKNALKGSESQVRQQFSKDIEKLLNEQVNKEMQSSNLYMSMSSWSYTHSLDGAGLFLFDHAAEEYEHAKKLIIFLNENNVPVQLTSISAPEHKFEGLTQIFQKAYEHEQHISESINNIVDHAIKCKDHATFNFLQWYVAEQHEEEVLFKDILDKIELIGNENHGLYLADQYVKGIAKSRKS

SEQ ID NO: 59 - protein, 455 aa, synthetic construct
MDSKGSSQKGSRLLLLLVVSNLLLPQGVLAMDEKNSVSVDLPGEMKVLVSKEKNKDGKYDLIATVDKLELKGTSDKNQGSGVLEGVKADKSKVKLTISDDLGQTTLEVFKEDGKTLVSKKVTSKDKSSTEEKFNEKGEVSEKIITRADGTRLEYTGIKSDGSGKAKEVLKGYDLKGELSSEKTTLVVKEGTVTLSKQISKSGEVSVELQDTDSSAATKKTAAWNSGTSTLTITVNSKKTKDLVFTKENTITVQQYDSQGTKLEGSAVEITKLDEIKNALKGSESQVRQQFSKDIEKLLNEQVNKEMQSSNLYMSMSSWSYTHSLDGAGLFLFDHAAEEYEHAKKLIIFLNENNVPVQLTSISAPEHKFEGLTQIFQKAYEHEQHISESINNIVDHAIKCKDHATFNFLQWYVAEQHEEEVLFKDILDKIELIGNENHGLYLADQYVKGIAKSRKS

SEQ ID NO: 60 - protein, 457 aa, synthetic construct
MDSKGSSQKGSRLLLLLVVSNLLLPQGVLAMDEKNSVSVDLPGEMKVLVSKEKNKDGKYDLIATVDKLELKGTSDKNQGSGVLEGVKADKSKVKLTISDDLGQTTLEVFKEDGKTLVSKKVTSKDKSSTEEKFNEKGEVSEKIITRADGTRLEYTGIKSDGSGKAKEVLKGYTLEGQLSDEKTTLVVKEGTVTLSKQISKSGEVSVELQDTDSSAATKKTAAWNSGTSTLTITVNSKKTKDLVFTKENTITVQQYDSQGTKLEGSAVEITKLDEIKNALKGGGSESQVRQQFSKDIEKLLNEQVNKEMQSSNLYMSMSSWSYTHSLDGAGLFLFDHAAEEYEHAKKLIIFLNENNVPVQLTSISAPEHKFEGLTQIFQKAYEHEQHISESINNIVDHAIKCKDHATFNFLQWYVAEQHEEEVLFKDILDKIELIGNENHGLYLADQYVKGIAKSRKS

SEQ ID NO: 61 - protein, 461 aa, synthetic construct
MDSKGSSQKGSRLLLLLVVSNLLLPQGVLAMDEKNSVSVDLPGEMKVLVSKEKNKDGKYDLIATVDKLELKGTSDKNQGSGVLEGVKADKSKVKLTISDDLGQTTLEVFKEDGKTLVSKKVTSKDKSSTEEKFNEKGEVSEKIITRADGTRLEYTGIKSDGSGKAKEVLKGYTLEGQLSDEKTTLVVKEGTVTLSKQISKSGEVSVELQDTDSSAATKKTAAWNSGTSTLTITVNSKKTKDLVFTKENTITVQQYDSQGTKLEGSAVEITKLDEIKNALKGGGSGGGSESQVRQQFSKDIEKLLNEQVNKEMQSSNLYMSMSSWSYTHSLDGAGLFLFDHAAEEYEHAKKLIIFLNENNVPVQLTSISAPEHKFEGLTQIFQKAYEHEQHISESINNIVDHAIKCKDHATFNFLQWYVAEQHEEEVLFKDILDKIELIGNENHGLYLADQYVKGIAKSRKS

SEQ ID NO: 62 - protein, 473 aa, synthetic construct
MDSKGSSQKGSRLLLLLVVSNLLLPQGVLAMDEKNSVSVDLPGEMKVLVSKEKNKDGKYDLIATVDKLELKGTSDKNQGSGVLEGVKADKSKVKLTISDDLGQTTLEVFKEDGKTLVSKKVTSKDKSSTEEKFNEKGEVSEKIITRADGTRLEYTGIKSDGSGKAKEVLKGYTLEGQLSDEKTTLVVKEGTVTLSKQISKSGEVSVELQDTDSSAATKKTAAWNSGTSTLTITVNSKKTKDLVFTKENTITVQQYDSQGTKLEGSAVEITKLDEIKNALKGGGSGGGSGGGSGGGSGGGSESQVRQQFSKDIEKLLNEQVNKEMQSSNLYMSMSSWSYTHSLDGAGLFLFDHAAEEYEHAKKLIIFLNENNVPVQLTSISAPEHKFEGLTQIFQKAYEHEQHISESINNIVDHAIKCKDHATFNFLQWYVAEQHEEEVLFKDILDKIELIGNENHGLYLADQYVKGIAKSRKS

SEQ ID NO: 63 - protein, 455 aa, synthetic construct
MDSKGSSQKGSRLLLLLVVSNLLLPQGVLAMDEKNSVSVDLPGEMKVLVSKEKNKDGKYDLIATVDKLELKGTSDKNNGAGVLEGVKADKSKVKLTISDDLGQTTLEVFKEDGKTLVSKKVTSKDKSSTEEKFNEKGEVSEKIITRADGTRLEYTGIKSDGSGKAKEVLKGYTLEGQLSDEKTTLVVKEGTVTLSKNIAKSGEVSVELNDADSSAATKKTAAWNSGTSTLTITVNSKKTKDLVFTKENTITVQQYDSNGAKLEGSAVEITKLDEIKNALKGSESQVRQQFSKDIEKLLNEQVNKEMQSSNLYMSMSSWSYTHSLDGAGLFLFDHAAEEYEHAKKLIIFLNENNVPVQLTSISAPEHKFEGLTQIFQKAYEHEQHISESINNIVDHAIKCKDHATFNFLQWYVAEQHEEEVLFKDILDKIELIGNENHGLYLADQYVKGIAKSRKS

SEQ ID NO: 64 - protein, 455 aa, synthetic construct
MDSKGSSQKGSRLLLLLVVSNLLLPQGVLAMDEKNSVSVDLPGEMKVLVSKEKNKDGKYDLIATVDKLELKGTSDKNNGSGVLEGVKADKSKVKLTISDDLGQTTLEVFKEDGKTLVSKKVTSKDKSSTEEKFNEKGEVSEKIITRADGTRLEYTGIKSDGSGKAKEVLKGYVLEGTLTAEKTTLVVKEGTVTLSKNISKSGEVSVELNDTDSSAATKKTAAWNSGTSTLTITVNSKKTKDLVFTKENTITVQQYDSNGTKLEGSAVEITKLDEIKNALKGSESQVRQQFSKDIEKLLNEQVNKEMQSSNLYMSMSSWSYTHSLDGAGLFLFDHAAEEYEHAKKLIIFLNENNVPVQLTSISAPEHKFEGLTQIFQKAYEHEQHISESINNIVDHAIKCKDHATFNFLQWYVAEQHEEEVLFKDILDKIELIGNENHGLYLADQYVKGIAKSRKS

SEQ ID NO: 65 - protein, 455 aa, synthetic construct
MDSKGSSQKGSRLLLLLVVSNLLLPQGVLAMDEKNSASVDLPGEMKVLVSKEKDKDGKYSLKATVDKIELKGTSDKDNGSGVLEGTKDDKSKAKLTIADDLSKTTFELFKEDGKTLVSRKVSSKDKTSTDEMFNEKGELSAKTMTRENGTKLEYTEMKSDGTGKAKEVLKNFTLEGKVANDKVTLEVKEGTVTLSKEIAKSGEVTVALNDTNTTQATKKTGAWDSKTSTLTISVNSKKTTQLVFTKQDTITVQKYDSAGTNLEGTAVEIKTLDELKNALKGSESQVRQQFSKDIEKLLNEQVNKEMQSSNLYMSMSSWSYTHSLDGAGLFLFDHAAEEYEHAKKLIIFLNENNVPVQLTSISAPEHKFEGLTQIFQKAYEHEQHISESINNIVDHAIKCKDHATFNFLQWYVAEQHEEEVLFKDILDKIELIGNENHGLYLADQYVKGIAKSRKS

SEQ ID NO: 66 - protein, 455 aa, synthetic construct
MDSKGSSQKGSRLLLLLVVSNLLLPQGVLAMDEKNSASVDLPGEMKVLVSKEKDKDGKYSLKATVDKIELKGTSDKDQGSGVLEGTKDDKSKAKLTIADDLSKTTFELFKEDGKTLVSRKVSSKDKTSTDEMFNEKGELSAKTMTREQGTKLEYTEMKSDGTGKAKEVLKQFTLEGKVANDKVTLEVKEGTVTLSKEIAKSGEVTVALQDTQTTQATKKTGAWDSKTSTLTISVNSKKTTQLVFTKQDTITVQKYDSAGTNLEGTAVEIKTLDELKNALKGSESQVRQQFSKDIEKLLNEQVNKEMQSSNLYMSMSSWSYTHSLDGAGLFLFDHAAEEYEHAKKLIIFLNENNVPVQLTSISAPEHKFEGLTQIFQKAYEHEQHISESINNIVDHAIKCKDHATFNFLQWYVAEQHEEEVLFKDILDKIELIGNENHGLYLADQYVKGIAKSRKS

SEQ ID NO: 67 - protein, 456 aa, synthetic construct
MDSKGSSQKGSRLLLLLVVSNLLLPQGVLAMDEKNSVSVDLPGGMKVLVSKEKDKDGKYSLMATVEKLELKGTSDKSNGSGVLEGEKADKSKAKLTISQDLNQTTFEIFKEDGKTLVSRKVNSKDKSSTEEKFNDKGKLSEKVVTRANGTRLEYTEIKNDGSGKAKEVLKGFALEGTLTDGGETKLTVTEGTVTLSKNISKSGEITVALNDTETTPADKKTGEWKSDTSTLTISKNSQKPKQLVFTKENTITVQNYNRAGNALEGSPAEIKDLAELKAALKGSESQVRQQFSKDIEKLLNEQVNKEMQSSNLYMSMSSWSYTHSLDGAGLFLFDHAAEEYEHAKKLIIFLNENNVPVQLTSISAPEHKFEGLTQIFQKAYEHEQHISESINNIVDHAIKCKDHATFNFLQWYVAEQHEEEVLFKDILDKIELIGNENHGLYLADQYVKGIAKSRKS

SEQ ID NO: 68 - protein, 456 aa, synthetic construct
MDSKGSSQKGSRLLLLLVVSNLLLPQGVLAMDEKNSVSVDLPGGMKVLVSKEKDKDGKYSLMATVEKLELKGTSDKSQGSGVLEGEKADKSKAKLTISQDLQQTTFEIFKEDGKTLVSRKVNSKDKSSTEEKFNDKGKLSEKVVTRAQGTRLEYTEIKNDGSGKAKEVLKGFALEGTLTDGGETKLTVTEGTVTLSKQISKSGEITVALQDTETTPADKKTGEWKSDTSTLTISKNSQKPKQLVFTKENTITVQNYNRAGNALEGSPAEIKDLAELKAALKGSESQVRQQFSKDIEKLLNEQVNKEMQSSNLYMSMSSWSYTHSLDGAGLFLFDHAAEEYEHAKKLIIFLNENNVPVQLTSISAPEHKFEGLTQIFQKAYEHEQHISESINNIVDHAIKCKDHATFNFLQWYVAEQHEEEVLFKDILDKIELIGNENHGLYLADQYVKGIAKSRKS

SEQ ID NO: 69 - protein, 455 aa, synthetic construct
MDSKGSSQKGSRLLLLLVVSNLLLPQGVLAMDEKNSVSVDLPGEMKVLVSKEKDKDGKYSLMATVDKLELKGTSDKSNGSGTLEGEKSDKSKAKLTISEDLSKTTFEIFKEDGKTLVSKKVNSKDKSSIEEKFNAKGELSEKTILRANGTRLEYTEIKSDGTGKAKEVLKDFALEGTLAADKTTLKVTEGTVVLSKHIPNSGEITVELNDSNSTQATKKTGKWDSNTSTLTISVNSKKTKNIVFTKEDTITVQKYDSAGTNLEGNAVEIKTLDELKNALKGSESQVRQQFSKDIEKLLNEQVNKEMQSSNLYMSMSSWSYTHSLDGAGLFLFDHAAEEYEHAKKLIIFLNENNVPVQLTSISAPEHKFEGLTQIFQKAYEHEQHISESINNIVDHAIKCKDHATFNFLQWYVAEQHEEEVLFKDILDKIELIGNENHGLYLADQYVKGIAKSRKS

SEQ ID NO: 70 - protein, 455 aa, synthetic construct
MDSKGSSQKGSRLLLLLVVSNLLLPQGVLAMDEKNSVSVDLPGEMKVLVSKEKDKDGKYSLMATVDKLELKGTSDKSQGSGTLEGEKSDKSKAKLTISEDLSKTTFEIFKEDGKTLVSKKVNSKDKSSIEEKFNAKGELSEKTILRAQGTRLEYTEIKSDGTGKAKEVLKDFALEGTLAADKTTLKVTEGTVVLSKHIPNSGEITVELQDSQSTQATKKTGKWDSQTSTLTISVNSKKTKNIVFTKEDTITVQKYDSAGTNLEGNAVEIKTLDELKNALKGSESQVRQQFSKDIEKLLNEQVNKEMQSSNLYMSMSSWSYTHSLDGAGLFLFDHAAEEYEHAKKLIIFLNENNVPVQLTSISAPEHKFEGLTQIFQKAYEHEQHISESINNIVDHAIKCKDHATFNFLQWYVAEQHEEEVLFKDILDKIELIGNENHGLYLADQYVKGIAKSRKS

SEQ ID NO: 71 - protein, 456 aa, synthetic construct
MDSKGSSQKGSRLLLLLVVSNLLLPQGVLAMDEKNSVSVDLPGGMKVLVSKEKDKDGKYSLMATVEKLELKGTSDKNNGSGTLEGEKTDKSKVKLTIAEDLSKTTFEIFKEDGKTLVSKKVTLKDKSSTEEKFNEKGEISEKTIVRANGTRLEYTDIKSDGSGKAKEVLKDFTLEGTLAADGKTTLKVTEGTVVLSKNILKSGEITVALDDSDTTQATKKTGKWDSKTSTLTISVNSQKTKNLVFTKEDTITVQKYDSAGTNLEGKAVEITTLEKLKDALKGSESQVRQQFSKDIEKLLNEQVNKEMQSSNLYMSMSSWSYTHSLDGAGLFLFDHAAEEYEHAKKLIIFLNENNVPVQLTSISAPEHKFEGLTQIFQKAYEHEQHISESINNIVDHAIKCKDHATFNFLQWYVAEQHEEEVLFKDILDKIELIGNENHGLYLADQYVKGIAKSRKS

SEQ ID NO: 72 - protein, 456 aa, synthetic construct
MDSKGSSQKGSRLLLLLVVSNLLLPQGVLAMDEKNSVSVDLPGGMKVLVSKEKDKDGKYSLMATVEKLELKGTSDKNQGSGTLEGEKTDKSKVKLTIAEDLSKTTFEIFKEDGKTLVSKKVTLKDKSSTEEKFNEKGEISEKTIVRAQGTRLEYTDIKSDGSGKAKEVLKDFTLEGTLAADGKTTLKVTEGTVVLSKNILKSGEITVALDDSDTTQATKKTGKWDSKTSTLTISVNSQKTKNLVFTKEDTITVQKYDSAGTNLEGKAVEITTLEKLKDALKGSESQVRQQFSKDIEKLLNEQVNKEMQSSNLYMSMSSWSYTHSLDGAGLFLFDHAAEEYEHAKKLIIFLNENNVPVQLTSISAPEHKFEGLTQIFQKAYEHEQHISESINNIVDHAIKCKDHATFNFLQWYVAEQHEEEVLFKDILDKIELIGNENHGLYLADQYVKGIAKSRKS

SEQ ID NO: 73 - protein, 456 aa, synthetic construct
MDSKGSSQKGSRLLLLLVVSNLLLPQGVLAMDEKNSVSVDLPGGMTVLVSKEKDKDGKYSLEATVDKLELKGTSDKNQGSGTLEGEKTDKSKVKSTIADDLSQTKFEIFKEDGKTLVSKKVTLKDKSSTEEKFNGKGETSEKTIVRAQGTRLEYTDIKSDGSGKAKEVLKDFTLEGTLAADGKTTLKVTEGTVVLSKNILKSGEITAALDDSDTTRATKKTGKWDSKTSTLTISVNSQKTKNLVFTKEDTITVQRYDSAGTNLEGKAVEITTLKELKNALKGSESQVRQQFSKDIEKLLNEQVNKEMQSSNLYMSMSSWSYTHSLDGAGLFLFDHAAEEYEHAKKLIIFLNENNVPVQLTSISAPEHKFEGLTQIFQKAYEHEQHISESINNIVDHAIKCKDHATFNFLQWYVAEQHEEEVLFKDILDKIELIGNENHGLYLADQYVKGIAKSRKS

SEQ ID NO: 74 - protein, 456 aa, synthetic construct
MDSKGSSQKGSRLLLLLVVSNLLLPQGVLAMDEKNSVSVDLPGGMTVLVSKEKDKDGKYSLEATVDKLELKGTSDKNNGSGTLEGEKTDKSKVKSTIADDLSQTKFEIFKEDGKTLVSKKVTLKDKSSTEEKFNGKGETSEKTIVRANGTRLEYTDIKSDGSGKAKEVLKDFTLEGTLAADGKTTLKVTEGTVVLSKNILKSGEITAALDDSDTTRATKKTGKWDSKTSTLTISVNSQKTKNLVFTKEDTITVQRYDSAGTNLEGKAVEITTLKELKNALKGSESQVRQQFSKDIEKLLNEQVNKEMQSSNLYMSMSSWSYTHSLDGAGLFLFDHAAEEYEHAKKLIIFLNENNVPVQLTSISAPEHKFEGLTQIFQKAYEHEQHISESINNIVDHAIKCKDHATFNFLQWYVAEQHEEEVLFKDILDKIELIGNENHGLYLADQYVKGIAKSRKS

SEQ ID NO: 75 - protein, 456 aa, synthetic construct
MDSKGSSQKGSRLLLLLVVSNLLLPQGVLAMDEKNSVSVDLPGEMKVLVSKEKDKDGKYSLEATVDKLELKGTSDKNNGSGVLEGVKAAKSKAKLTIADDLSQTKFEIFKEDGKTLVSKKVTLKDKSSTEEKFNDKGKLSEKVVTRANGTRLEYTEIQNDGSGKAKEVLKSLTLEGTLTADGETKLTVEAGTVTLSKNISESGEITVELKDTETTPADKKSGTWDSKTSTLTISKNSQKTKQLVFTKENTITVQKYNTAGTKLEGSPAEIKDLEALKAALKGSESQVRQQFSKDIEKLLNEQVNKEMQSSNLYMSMSSWSYTHSLDGAGLFLFDHAAEEYEHAKKLIIFLNENNVPVQLTSISAPEHKFEGLTQIFQKAYEHEQHISESINNIVDHAIKCKDHATFNFLQWYVAEQHEEEVLFKDILDKIELIGNENHGLYLADQYVKGIAKSRKS

SEQ ID NO: 76 - protein, 456 aa, synthetic construct
MDSKGSSQKGSRLLLLLVVSNLLLPQGVLAMDEKNSVSVDLPGEMKVLVSKEKDKDGKYSLEATVDKLELKGTSDKNQGSGVLEGVKAAKSKAKLTIADDLSQTKFEIFKEDGKTLVSKKVTLKDKSSTEEKFNDKGKLSEKVVTRAQGTRLEYTEIQNDGSGKAKEVLKSLTLEGTLTADGETKLTVEAGTVTLSKQISESGEITVELKDTETTPADKKSGTWDSKTSTLTISKNSQKTKQLVFTKENTITVQKYNTAGTKLEGSPAEIKDLEALKAALKGSESQVRQQFSKDIEKLLNEQVNKEMQSSNLYMSMSSWSYTHSLDGAGLFLFDHAAEEYEHAKKLIIFLNENNVPVQLTSISAPEHKFEGLTQIFQKAYEHEQHISESINNIVDHAIKCKDHATFNFLQWYVAEQHEEEVLFKDILDKIELIGNENHGLYLADQYVKGIAKSRKS

SEQ ID NO: 77 - protein, 9 aa, synthetic construct
YVLEGTLTA

SEQ ID NO: 78 - protein, 9 aa, synthetic construct
YVIEGTSKQ

SEQ ID NO: 79 - protein, 9 aa, synthetic construct
FTLEGKVAN

SEQ ID NO: 80 - protein, 9 aa, synthetic construct
FALEGTLTD

SEQ ID NO: 81 - protein, 9 aa, synthetic construct
YTLEGQLSD

SEQ ID NO: 82 - protein, 9 aa, synthetic construct
YDLKGELSS

SEQ ID NO: 83 - protein, 273 aa, Borrelia burgdorferi
MKKYLLGIGLILALIACKQNVSSLDEKNSVSVDLPGEMKVLVSKEKNKDGKYDLIATVDKLELKGTSDKNNGSGVLEGVKADKSKVKLTISDDLGQTTLEVFKEDGKTLVSKKVTSKDKSSTEEKFNEKGEVSEKIITRADGTRLEYTGIKSDGSGKAKEVLKGYVLEGTLTAEKTTLVVKEGTVTLSKNISKSGEVSVELNDTDSSAATKKTAAWNSGTSTLTITVNSKKTKDLVFTKENTITVQQYDSNGTKLEGSAVEITKLDEIKNALK

SEQ ID NO: 84 - protein, 273 aa, Borrelia burgdorferi
MKKYLLGIGLILALIACKQNVSSLDEKNSASVDLPGEMKVLVSKEKDKDGKYSLKATVDKIELKGTSDKDNGSGVLEGTKDDKSKAKLTIADDLSKTTFELFKEDGKTLVSRKVSSKDKTSTDEMFNEKGELSAKTMTRENGTKLEYTEMKSDGTGKAKEVLKNFTLEGKVANDKVTLEVKEGTVTLSKEIAKSGEVTVALNDTNTTQATKKTGAWDSKTSTLTISVNSKKTTQLVFTKQDTITVQKYDSAGTNLEGTAVEIKTLDELKNALK

SEQ ID NO: 85 - protein, 274 aa, Borrelia burgdorferi
MKKYLLGIGLILALIACKQNVSSLDEKNSVSVDLPGGMKVLVSKEKDKDGKYSLMATVEKLELKGTSDKSNGSGVLEGEKADKSKAKLTISQDLNQTTFEIFKEDGKTLVSRKVNSKDKSSTEEKFNDKGKLSEKVVTRANGTRLEYTEIKNDGSGKAKEVLKGFALEGTLTDGGETKLTVTEGTVTLSKNISKSGEITVALNDTETTPADKKTGEWKSDTSTLTISKNSQKPKQLVFTKENTITVQNYNRAGNALEGSPAEIKDLAELKAALK

SEQ ID NO: 86 - protein, 273 aa, Borrelia burgdorferi
MKKYLLGIGLILALIACKQNVSSLDEKNSVSVDLPGEMKVLVSKEKDKDGKYSLMATVDKLELKGTSDKSNGSGTLEGEKSDKSKAKLTISEDLSKTTFEIFKEDGKTLVSKKVNSKDKSSIEEKFNAKGELSEKTILRANGTRLEYTEIKSDGTGKAKEVLKDFALEGTLAADKTTLKVTEGTVVLSKHIPNSGEITVELNDSNSTQATKKTGKWDSNTSTLTISVNSKKTKNIVFTKEDTITVQKYDSAGTNLEGNAVEIKTLDELKNALK

SEQ ID NO: 87 - protein, 274 aa, Borrelia burgdorferi
MKKYLLGIGLILALIACKQNVSSLDEKNSVSVDLPGGMKVLVSKEKDKDGKYSLMATVEKLELKGTSDKNNGSGTLEGEKTDKSKVKLTIAEDLSKTTFEIFKEDGKTLVSKKVTLKDKSSTEEKFNEKGEISEKTIVRANGTRLEYTDIKSDGSGKAKEVLKDFTLEGTLAADGKTTLKVTEGTVVLSKNILKSGEITVALDDSDTTQATKKTGKWDSKTSTLTISVNSQKTKNLVFTKEDTITVQKYDSAGTNLEGKAVEITTLEKLKDALK

SEQ ID NO: 88 - protein, 274 aa, Borrelia burgdorferi
MKKYLLGIGLILALIACKQNVSSLDEKNSVSVDLPGGMTVLVSKEKDKDGKYSLEATVDKLELKGTSDKNNGSGTLEGEKTDKSKVKSTIADDLSQTKFEIFKEDGKTLVSKKVTLKDKSSTEEKFNGKGETSEKTIVRANGTRLEYTDIKSDGSGKAKEVLKDFTLEGTLAADGKTTLKVTEGTVVLSKNILKSGEITAALDDSDTTRATKKTGKWDSKTSTLTISVNSQKTKNLVFTKEDTITVQRYDSAGTNLEGKAVEITTLKELKNALK

SEQ ID NO: 89 - protein, 214 aa, Borrelia burgdorferi
MKKYLLGIGLILALIACKQNVSSLDEKNSVSVDLPGEMKVLVSKEKDKDGKYSLEATVDKLELKGTSDKNNGSGVLEGVKAAKSKAKLTIADDLSQTKFEIFKEDGKTLVSKKVTLKDKSSTEEKFNDKGKLSEKVVTRANGTRLEYTEIQNDGSGKAKEVLKSLTLEGTLTADGETKLTENTITVQKYNTAGTKLEGSPAEIKDLEALKAALK

SEQ ID NO: 90 - protein, 173 aa, synthetic construct
ESQVRQQFSKDIEKLLNEQVNKEMQSSNLYMSMSSWSYTHSLDGAGLFLFDHAAEEYEHAKKLIIFLNENNVPVQLTSISAPEHKFEGLTQIFQKAYEHEQHISESINNIVDHAIKCKDHATFNFLQWYVAEQHEEEVLFKDILDKIELIGNENHGLYLADQYVKGIAKSRKS

SEQ ID NO: 91 - protein, 8 aa, synthetic construct
GGGSGGGS

SEQ ID NO: 92 - protein, 20 aa, synthetic construct
GGGSGGGSGGGSGGGSGGGS

SEQ ID NO: 93 - protein, 154 aa, Aquifex aeolicus
MQIYEGKLTAEGLRFGIVASRFNHALVDRLVEGAIDCIVRHGGREEDITLVRVPGSWEIPVAAGELARKEDIDAVIAIGVLIRGATPHFDYIASEVSKGLANLSLELRKPITFGVITADTLEQAIERAGTKHGNKGWEAALSAIEMANLFKSLR

SEQ ID NO: 94 - protein, 250 aa, synthetic construct
MDEKNSVSVDLPGEMKVLVSKEKNKDGKYDLIATVDKLELKGTSDKNNGSGVLEGVKADKSKVKLTISDDLGQTTLEVFKEDGKTLVSKKVTSKDKSSTEEKFNEKGEVSEKIITRADGTRLEYTGIKSDGSGKAKEVLKGYTLEGQLSDEKTTLVVKEGTVTLSKNISKSGEVSVELNDTDSSAATKKTAAWNSGTSTLTITVNSKKTKDLVFTKENTITVQQYDSNGTKLEGSAVEITKLDEIKNALK

SEQ ID NO: 95 - protein, 250 aa, synthetic construct
MDEKNSVSVDLPGEMKVLVSKEKNKDGKYDLIATVDKLELKGTSDKNNGSGVLEGVKADKSKVKLTISDDLGQTTLEVFKEDGKTLVSKKVTSKDKSSTEEKFNEKGEVSEKIITRADGTRLEYTGIKSDGSGKAKEVLKGYDLKGELSSEKTTLVVKEGTVTLSKNISKSGEVSVELNDTDSSAATKKTAAWNSGTSTLTITVNSKKTKDLVFTKENTITVQQYDSNGTKLEGSAVEITKLDEIKNALK

SEQ ID NO: 96 - protein, 250 aa, synthetic construct
MDEKNSVSVDLPGEMKVLVSKEKNKDGKYDLIATVDKLELKGTSDKNNGSGVLEGVKADKSKVKLTISDDLGQTTLEVFKEDGKTLVSKKVTSKDKSSTEEKFNEKGEVSEKIITRADGTRLEYTGIKSDGSGKAKEVLKGFTLEGKVANEKTTLVVKEGTVTLSKNISKSGEVSVELNDTDSSAATKKTAAWNSGTSTLTITVNSKKTKDLVFTKENTITVQQYDSNGTKLEGSAVEITKLDEIKNALK

SEQ ID NO: 97 - protein, 250 aa, synthetic construct
MDEKNSVSVDLPGEMKVLVSKEKNKDGKYDLIATVDKLELKGTSDKNNGSGVLEGVKADKSKVKLTISDDLGQTTLEVFKEDGKTLVSKKVTSKDKSSTEEKFNEKGEVSEKIITRADGTRLEYTGIKSDGSGKAKEVLKGFALEGTLTDEKTTLVVKEGTVTLSKNISKSGEVSVELNDTDSSAATKKTAAWNSGTSTLTITVNSKKTKDLVFTKENTITVQQYDSNGTKLEGSAVEITKLDEIKNALK

SEQ ID NO: 98 - protein, 250 aa, synthetic construct
MDEKNSVSVDLPGEMKVLVSKEKNKDGKYDLIATVDKLELKGTSDKNQGSGVLEGVKADKSKVKLTISDDLGQTTLEVFKEDGKTLVSKKVTSKDKSSTEEKFNEKGEVSEKIITRADGTRLEYTGIKSDGSGKAKEVLKGYTLEGQLSDEKTTLVVKEGTVTLSKQISKSGEVSVELQDTDSSAATKKTAAWNSGTSTLTITVNSKKTKDLVFTKENTITVQQYDSQGTKLEGSAVEITKLDEIKNALK

SEQ ID NO: 99 - protein, 250 aa, synthetic construct
MDEKNSVSVDLPGEMKVLVSKEKNKDGKYDLIATVDKLELKGTSDKNQGSGVLEGVKADKSKVKLTISDDLGQTTLEVFKEDGKTLVSKKVTSKDKSSTEEKFNEKGEVSEKIITRADGTRLEYTGIKSDGSGKAKEVLKGFTLEGKVANEKTTLVVKEGTVTLSKQISKSGEVSVELQDTDSSAATKKTAAWNSGTSTLTITVNSKKTKDLVFTKENTITVQQYDSQGTKLEGSAVEITKLDEIKNALK

SEQ ID NO: 100 - protein, 250 aa, synthetic construct
MDEKNSVSVDLPGEMKVLVSKEKNKDGKYDLIATVDKLELKGTSDKNQGSGVLEGVKADKSKVKLTISDDLGQTTLEVFKEDGKTLVSKKVTSKDKSSTEEKFNEKGEVSEKIITRADGTRLEYTGIKSDGSGKAKEVLKGFALEGTLTDEKTTLVVKEGTVTLSKQISKSGEVSVELQDTDSSAATKKTAAWNSGTSTLTITVNSKKTKDLVFTKENTITVQQYDSQGTKLEGSAVEITKLDEIKNALK

SEQ ID NO: 101 - protein, 250 aa, synthetic construct
MDEKNSVSVDLPGEMKVLVSKEKNKDGKYDLIATVDKLELKGTSDKNQGSGVLEGVKADKSKVKLTISDDLGQTTLEVFKEDGKTLVSKKVTSKDKSSTEEKFNEKGEVSEKIITRADGTRLEYTGIKSDGSGKAKEVLKGYDLKGELSSEKTTLVVKEGTVTLSKQISKSGEVSVELQDTDSSAATKKTAAWNSGTSTLTITVNSKKTKDLVFTKENTITVQQYDSQGTKLEGSAVEITKLDEIKNALK

SEQ ID NO: 102 - protein, 250 aa, synthetic construct
  VARIANT 48: note = X = N or Q
  VARIANT 142..150: note = X = Any amino acid
  VARIANT 167: note = X = N or Q
  VARIANT 179: note = X = N or Q
  VARIANT 228: note = X = N or Q
MDEKNSVSVDLPGEMKVLVSKEKNKDGKYDLIATVDKLELKGTSDKNNGSGVLEGVKADKSKVKLTISDDLGQTTLEVFKEDGKTLVSKKVTSKDKSSTEEKFNEKGEVSEKIITRADGTRLEYTGIKSDGSGKAKEVLKGXXXXXXXXXEKTTLVVKEGTVTLSKNISKSGEVSVELNDTDSSAATKKTAAWNSGTSTLTITVNSKKTKDLVFTKENTITVQQYDSNGTKLEGSAVEITKLDEIKNALK

SEQ ID NO: 103-200 - skipped (98 entries)

SEQ ID NO: 201 - protein, 173 aa, synthetic construct
ESQVRQQFSKDIEKLLNEQVNKEMQSSNLYMCMSSWSYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 202 - protein, 173 aa, synthetic construct
ESQVRQQFSKDIEKLLNEQVNKEMQSSNLYMSMSSWSYTHSLDGAGLFLFDHAAEEYEHAKKLIIFLNENNVPVQLTCISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 203 - protein, 173 aa, synthetic construct
ESQVRQQFSKDIEKLLNEQVNKEMQSSNLYMSMSSWSYTHSLDGAGLFLFDHAAEEYEHAKKLIIFLNENNVPVQLTSISCPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 204 - protein, 173 aa, synthetic construct
ESQVRQQFSKDIEKLLNEQVNKEMQSSNLYMSMSSWSYTHSLDGAGLFLFDHAAEEYEHAKKLIIFLNENNVPVQLTSISAPEHCFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 205 - protein, 173 aa, synthetic construct
ESQVRQQFSKDIEKLLNEQVNKEMQSSNLYMSMSSWSYTHSLDGAGLFLFDHAAEEYEHAKKLIIFLNENNVPVQLTSISAPEHKFEGLTQIFQKAYEHEQHISECINNIVDHAIKSKDHATFNFLQWYVAEQHEEEVLFKDILDKIELIGNENHGLYLADQYVKGIAKSRKS

SEQ ID NO: 206 - protein, 173 aa, synthetic construct; identical to SEQ ID NO: 90

SEQ ID NO: 207 - protein, 173 aa, synthetic construct
ESQVRQQFSKDIEKLLNCQVNKEMQSSNLYMSMSSWSYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 208 - protein, 173 aa, synthetic construct
ESQVRQQFSKDIEKLLNEQVNKEMNSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 209 - protein, 166 aa, Helicobacter pylori
LSKDIIKLLNEQVNKEMNSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 210 - DNA, 22 bp, synthetic construct
  source 1..22: mol_type = other DNA
tgactgtgaacgttcgagatga

SEQ ID NO: 211 - protein, 191 aa, Trichoplusia ni
TQCNVNPVQIPKDWITMHRSCRNSMRQQIQMEVGASLQYLAMGAHFSKDVVNRPGFAQLFFDAASEEREHAMKLIEYLLMRGELTNDVSSLLQVRPPTRSSWKGGVEALEHALSMESDVTKSIRNVIKACEDDSEFNDYHLVDYLTGDFLEEQYKGQRDLAGKASTLKKLMDRHEALGEFIFDKKLLGIDV

SEQ ID NO: 212 - protein, 212 aa, Trichoplusia ni
ADTCYNDVALDCGITSNSLALPRCNAVYGEYGSHGNVATELQAYAKLHLERSYDYLLSAAYFNNYQTNRAGFSKLFKKLSDEAWSKTIDIIKHVTKRGDKMNFDQHSTMKTERKNYTAENHELEALAKALDTQKELAERAFYIHREATRNSQHLHDPEIAQYLEEEFIEDHAEKIRTLAGHTSDLKKFITANNGHDLSLALYVFDEYLQKTV

SEQ ID NO: 213 - protein, 174 aa, Pyrococcus furiosus
MLSERMLKALNDQLNRELYSAYLYFAMAAYFEDLGLEGFANWMKAQAEEEIGHALRFYNYIYDRNGRVELDEIPKPPKEWESPLKAFEAAYEHEKFISKSIYELAALAEEEKDYSTRAFLEWFINEQVEEEASVKKILDKLKFAKDSPQILFMLDKELSARAPKLPGLLMQGGE

SEQ ID NO: 214 - protein, 183 aa, Homo sapiens
MTTASTSQVRQNYHQDSEAAINRQINLELYASYVYLSMSYYFDRDDVALKNFAKYFLHQSHEEREHAEKLMKLQNQRGGRIFLQDIKKPDCDDWESGLNAMECALHLEKNVQQSLLELHKLATDKNDPHLCDFIETHYLNEQVKAIKELGDHVTNLRKMGAPESGLAEYLFDKHTLGDSDQES

SEQ ID NO: 215 - protein, 204 aa, Homo sapiens
MDSKGSSQKGSRLLLLLVVSNLLLPQGVLASSQIRQNYSTDVEAAVNSLVNLYLQASYTYLSLGFYFDRDDVALEGVSHFFRELAEEKREGYERLLKMQNQRGGRALFQDIKKPAEDEWGKTPDAMKAAMALEKKLNQALLDLHALGSARTDPHLCDFLETHFLDEEVKLIKKMGDHLTNLHRLGGPEAGLGEYLFERLTLKHD

SEQ ID NO: 216 - protein, 154 aa, Aquifex aeolicus; identical to SEQ ID NO: 93

SEQ ID NO: 217 - protein, 8 aa, unidentified
  REGION 1..8: note = Bullfrog linker sequence
ESQVRQQF

SEQ ID NO: 218 - protein, 15 aa, synthetic construct
CLVPRGSLEHHHHHH

SEQ ID NO: 219 - protein, 156 aa, Escherichia coli
MNIIEANVATPDARVAITIARFNNFINDSLLEGAIDALKRIGQVKDENITVVWVPGAYELPLAAGALAKTGKYDAVIALGTVIRGGTAHFEYVAGGASNGLAHVAQDSEIPVAFGVLTTESIEQAIERAGTKAGNKGAEAALTALEMINVLKAIKA

SEQ ID NO: 220 - protein, 16 aa, synthetic construct
GGGGSGGGGSGGGGSG

SEQ ID NO: 221 - protein, 28 aa, synthetic construct
GGSGSGSNSSASSGASSGGASGGSGGSG

SEQ ID NO: 222 - protein, 46 aa, synthetic construct
GGSGSASSGASASGSSNGSGSGSGSNSSASSGASSGGASGGSGGSG

SEQ ID NO: 223 - protein, 26 aa, synthetic construct
GGSGSASAEAAAKEAAAKAGGSGGSG

SEQ ID NO: 224 - protein, 32 aa, synthetic construct
GGSGSASAEAAAKEAAAKEAAAKASGGSGGSG

SEQ ID NO: 225 - protein, 47 aa, synthetic construct
SGGGSGSASSGASASGSSCSGSGSGSSSASSGASSGGASGGGSGGSG

SEQ ID NO: 226 - protein, 4 aa, synthetic construct
GGGS

SEQ ID NO: 227 - protein, 6 aa, synthetic construct
HHHHHH

SEQ ID NO: 228 - DNA, 21 bp, synthetic construct
  source 1..21: mol_type = other DNA
tgactgtgaacgttcgagatg